SEQUENCE LISTING (28 sequences)

SEQ ID NO: 1 - RNA, 10 nt, Homo sapiens
uggguggggg

SEQ ID NO: 2 - RNA, 10 nt, Homo sapiens
ugggugggug

SEQ ID NO: 3 - RNA, 10 nt, Homo sapiens
aucgguaaaa

SEQ ID NO: 4 - RNA, 10 nt, Homo sapiens
cagguaaugu

SEQ ID NO: 5 - RNA, 10 nt, Homo sapiens
gagguaauac

SEQ ID NO: 6 - RNA, 10 nt, Homo sapiens
gagguaauau

SEQ ID NO: 7 - RNA, 10 nt, Homo sapiens
gagguaaugu

SEQ ID NO: 8 - RNA, 10 nt, Homo sapiens
aagguaauaa

SEQ ID NO: 9 - RNA, 10 nt, Homo sapiens
aagguaaugu

SEQ ID NO: 10 - RNA, 11 nt, Homo sapiens
aagguaaugua

SEQ ID NO: 11 - RNA, 12 nt, Homo sapiens
cgggucauaauc

SEQ ID NO: 12 - RNA, 10 nt, Homo sapiens
aaggucuggg

SEQ ID NO: 13 - RNA, 10 nt, Homo sapiens
caggucugga

SEQ ID NO: 14 - RNA, 10 nt, Homo sapiens
caggucuggu

SEQ ID NO: 15 - RNA, 10 nt, Homo sapiens
gaggucuggu

SEQ ID NO: 16 - RNA, 10 nt, Homo sapiens
aacaugagga

SEQ ID NO: 17 - RNA, 10 nt, Homo sapiens
caagguaccu

SEQ ID NO: 18 - RNA, 11 nt, Homo sapiens
  modified_base (5)..(6): Pseudouracil
auacuuaccug

SEQ ID NO: 19 - RNA, 11 nt, Artificial Sequence
  Description of Artificial Sequence Synthetic oligonucleotide
ggaguaagucu

SEQ ID NO: 20 - RNA, 12 nt, Artificial Sequence
  Description of Artificial Sequence Synthetic oligonucleotide
gauacuuaccug

SEQ ID NO: 21 - RNA, 23 nt, Artificial Sequence
  Description of Artificial Sequence Synthetic oligonucleotide
ggaguaagucugauacuuaccug

SEQ ID NO: 22 - RNA, 21 nt, Artificial Sequence
  Description of Artificial Sequence Synthetic oligonucleotide
ucuaaggcguaagucugccag

SEQ ID NO: 23 - RNA, 21 nt, Artificial Sequence
  Description of Artificial Sequence Synthetic oligonucleotide
ucuaagcaguaagucugccag

SEQ ID NO: 24 - RNA, 11 nt, Homo sapiens
auacuuaccug

SEQ ID NO: 25 - RNA, 10 nt, Homo sapiens
caaguaagug

SEQ ID NO: 26 - RNA, 10 nt, Homo sapiens
cagguaagua

SEQ ID NO: 27 - RNA, 10 nt, Homo sapiens
  modified_base (5)..(6): Pseudouracil
auacuuacug

SEQ ID NO: 28 - RNA, 12 nt, Homo sapiens
  modified_base (12)..(12): a, c, u, g, unknown or other
caguaagucugn

(19) United States
(12) Patent Application Publication
(10) Pub. No.: US 2023/0152257 A1
(43) Pub. Date: May 18, 2023

(54) METHODS AND COMPOSITIONS FOR SCREENING AND IDENTIFICATION OF SPLICING

(71) Applicant: SKYHAWK THERAPEUTICS, INC., Waltham, MA (US)

(72) Inventors: Kathleen MCCARTHY, Sommerville, MA (US); Michael LUZZIO, Noank, CT (US)

(21) Appl. No.: 16/649,697

(22) PCT Filed: Sep. 25, 2018
(86) PCT No.: PCT/US18/52743

§ 371 Date: Mar. 23, 2020

Related U.S. Application Data
(60) Provisional application No. 62/562,941, filed on Sep. 25, 2017.

(51) Int. Cl. G01N 24/08; C12N 15/11; C07H 21/04; G01N 33/53; C12Q 1/6816; C12P 19/34

(52) U.S. Cl. CPC G01N 24/088; C12N 15/11; C07H 21/04; G01N 33/5308; C12Q 1/6816; C12P 19/34

(57) ABSTRACT

Provided herein are structure-based screening platforms and methods to identify small molecules that can bind polynucleotides and/or complexes formed by polynucleotides and proteins. Structure-based screening platforms and methods to characterize interactions of small molecules with polynucleotides and/or with complexes formed by polynucleotides and proteins are also provided herein. Methods and compositions to identify small molecules that can bind polynucleotides and/or polynucleotide-protein complexes involved in RNA splicing are also provided herein.

### CROSS-REFERENCE

This application is a U.S. National Phase Application under 35 U.S.C. § 371 of International Application No. PCT/US2018/052743, filed Nov. 7, 2018, which claims priority to U.S. Provisional Patent Application No. 62/562,941, filed Sep. 25, 2017, which is incorporated herein by reference in its entirety.

### BACKGROUND OF THE INVENTION

Protein-nucleic acid interactions are involved in many cellular functions, including transcription, RNA splicing, mRNA decay, and mRNA translation. Readily accessible synthetic molecules that can bind with high affinity to specific sequences and structural components of single- or double-stranded nucleic acids have the potential to interfere with these interactions in a controllable way, making them attractive tools for molecular biology and medicine.

The human transcriptome is composed of a vast RNA population that undergoes further diversification by splicing. Genome-wide studies highlight that 90% of genes are alternatively spliced in humans, making splicing of the main drivers of proteomic diversity and, consequently, determinant of cellular function. Unsurprisingly, given its extent, numerous splice isoforms have been described to be associated with several diseases including cancer. Interestingly, many of these splice isoforms involved in cancers are derived from the same gene and have antagonistic functions, e.g., pro- and anti-angiogenic, or pro- and anti-apoptotic (in their translated protein form). Thus, splicing could drive key regulatory processes in switching a cell from non-cancerous to cancerous particularly.

In addition, mutations affecting mRNA expression have been shown to cause up to half of all disease-causing gene alterations. This potentially represents the most frequent cause of hereditary disease. Of these mutations, the most common consequence is exon skipping. Detecting specific splice sites in this large sequence pool is the responsibility of the major and minor spliceosomes in collaboration with hundreds of additional splicing factors. Outside of the core splice site motifs, the bulk of the information required for splicing is thought to be contained in exonic and intronic cis-regulatory elements that function by recruitment of sequence-specific RNA-binding protein factors that either activate or repress the use of adjacent splice sites. This complexity makes splicing susceptible to sequence polymorphisms and deleterious mutations. Beyond this, the complex and dynamic process of splicing may require several key interactions to take place at particular kinetic points in time during the splicing process. Indeed, RNA mis-splicing underlies a growing number of human diseases with substantial societal consequences.

However, targeting RNA splicing, more specifically targeting RNA targets, is intractable due to limited available data such as 2-dimensional, and 3-dimensional structures of RNA, chemotypes that engender RNA binding affinity or selectivity, chemotypes that engender RNA binding affinity and selectivity at particular mRNA splicing hot spots, and identification of RNA structural elements that form small molecule binding pockets. Screening of small molecule libraries for binding RNA targets could generate data about chemotypes that engender RNA binding. However, few small molecule-screening collections are enriched in RNA binders; in fact, most libraries are biased with compounds that bind to proteins. In addition, several of the available RNA binder libraries are non-specific or selective to particular RNAs. To address these needs and others, the present disclosure in various embodiments provides a structure-based screening platform that can be used to identify small molecules that bind to RNA and/or RNA protein complex, design novel molecules that can fit into particular RNA binding pockets, and improve specificity and selectivity of small molecules towards disease-associated pre-mRNA splicing defects.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### SUMMARY OF THE INVENTION

In some aspects, the present disclosure provides a method comprising: providing a polynucleotide sample comprising a target polynucleotide; contacting to the target polynucleotide a first binding agent, a second binding agent, or both; wherein the target polynucleotide and the first binding agent form a first complex, wherein the second binding agent and the first complex form a second complex; and obtaining a nuclear magnetic resonance (NMR) spectrum of the first complex, the second complex, or both using a NMR device. In some embodiments, the target polynucleotide is a target ribonucleic acid (RNA). In some embodiments, the target RNA is a precursor messenger RNA (pre-mRNA) or a portion thereof. In some embodiments, the target polynucleotide contains a splice site or a portion thereof. In some embodiments, the splice site is a 5′ splice site, a cryptic 5′ splice site, a 3′ splice site, or a cryptic 3′ spice site, or any combinations thereof. In some embodiments, the target polynucleotide contains a branch point (BP), an exonic splicing enhancer (ESE), an exonic splicing silencer (ESS), an intronic splicing enhancer (ISE), an intronic splicing silencer (ISS), or a polypyrimidine tract, or any combinations thereof. In some embodiments, the target polynucleotide contains at least one intron or a fragment thereof. In some embodiments, the target polynucleotide contains at least one exon or a fragment thereof. In some embodiments, the target polynucleotide contains at least one exon-intron boundary. In some embodiments, the target polynucleotide is at least 8 nucleotides in length. In some embodiments, the target polynucleotide is at least 25 nucleotides in length. In some embodiments, the target polynucleotide is at most 1000 nucleotides in length. In some embodiments, the target polynucleotide is from 100 to 200 nucleotides in length. In some embodiments, the target polynucleotide comprises none or at least one nucleotide isotopically labeled with one or more atomic labels comprising 2H, 13C, 15N, 19F and 31P. In some embodiments, the first binding agent comprises a first polynucleotide, a first polypeptide, or a combination thereof. In some embodiments, the first polynucleotide is a first RNA. In some embodiments, the first RNA is a small nuclear RNA (snRNA) or a portion thereof. In some embodiments, the snRNA is U1 snRNA, U2 snRNA, U4 snRNA, U5 snRNA, U6 snRNA, U11 snRNA, U12 snRNA, U4atac snRNA, U5 snRNA, U6atac snRNA; or a portion thereof. In some embodiments, the first polypeptide is a protein component of a ribonucleoprotein or a portion thereof. In some embodiments, the ribonucleoprotein is a small nuclear ribonucleoprotein (snRNP) or a portion thereof. In some embodiments, the snRNP is U1 snRNP, U2 snRNP, U4 snRNP, U5 snRNP, U6 snRNP, U11 snRNP, U12 snRNP, U4atac snRNP, U5 snRNP, U6atac snRNP; or a portion thereof. In some embodiments, the first polypeptide is a protein or a portion thereof selected from the group comprising 9G8, A1 hnRNP, A2 hnRNP, ASD-1, ASD-2b, ASF, B1 hnRNP, C1 hnRNP, C2 hnRNAP, CBP20, CBP80, CELF, F hnRNP, FBP11, Fox-1, Fox-2, G hnRNP, H hnRNP, hnRNP 1, hnRNP 3, hnRNP C, hnRNP G, hnRNP K, hnRNP M, hnRNP U, Hu, HUR, I hnRNP, K hnRNP, KH-type splicing regulatory protein (KSRP), L hnRNP, M hnRNP, mBBP, muscle-blind like (MBNL), NF45, NFAR, Nova-1, Nova-2, nPTB, P54/SFRS11, polypyrimidine tract binding protein (PTB), PRP19 complex proteins, R hnRNP, RNPC1, SAM68, SC35, SF, SF1/BBP, SF2, SF3A, SF3B, SFRS10, Sm proteins, SR proteins, SRm300, SRp20, SRp30c, SRP35C, SRP36, SRP38, SRp40, SRp55, SRp75, SRSF, STAR, GSG, SUP-12, TASR-1, TASR-2, TIA, TIAR, TRA2, TRA2a/b, U hnRNP, U1 snRNP, U11 snRNP, U12 snRNP, U1-C, U2 snRNP, U2AF1-RS2, U2AF35, U2AF65, U4 snRNP, U5 snRNP, U6 snRNP, Urp, YB1, or any combination thereof. In some embodiments, the second binding agent is a small molecule. In some embodiments, the first binding agent comprises a small molecule. In some embodiments, the second binding agent comprises a second polynucleotide, a second polypeptide, or a combination thereof. In some embodiments, the second polynucleotide is a second RNA. In some embodiments, the second RNA is a small nuclear RNA (snRNA) or a portion thereof. In some embodiments, the snRNA is U1 snRNA, U2 snRNA, U4 snRNA, U5 snRNA, U6 snRNA, U11 snRNA, U12 snRNA, U4atac snRNA, U5 snRNA, U6atac snRNA; or a portion thereof. In some embodiments, the second polypeptide is a protein component of a ribonucleoprotein or a portion thereof. In some embodiments, the ribonucleoprotein is a small nuclear ribonucleoprotein (snRNP) or a portion thereof. In some embodiments, the snRNP is U1 snRNP, U2 snRNP, U4 snRNP, U5 snRNP, U6 snRNP, U11 snRNP, U12 snRNP, U4atac snRNP, U5 snRNP, U6atac snRNP or a portion thereof. In some embodiments, the second polypeptide is a protein or a portion thereof selected from the group comprising 9G8, A1 hnRNP, A2 hnRNP, ASD-1, ASD-2b, ASF, B1 hnRNP, C1 hnRNP, C2 hnRNAP, CBP20, CBP80, CELF, F hnRNP, FBP11, Fox-1, Fox-2, G hnRNP, H hnRNP, hnRNP 1, hnRNP 3, hnRNP C, hnRNP G, hnRNP K, hnRNP M, hnRNP U, Hu, HUR, I hnRNP, K hnRNP, KH-type splicing regulatory protein (KSRP), L hnRNP, M hnRNP, mBBP, muscle-blind like (MBNL), NF45, NFAR, Nova-1, Nova-2, nPTB, P54/SFRS11, polypyrimidine tract binding protein (PTB), PRP19 complex proteins, R hnRNP, RNPC1, SAM68, SC35, SF, SF1/BBP, SF2, SF3A, SF3B, SFRS10, Sm proteins, SR proteins, SRm300, SRp20, SRp30c, SRP35C, SRP36, SRP38, SRp40, SRp55, SRp75, SRSF, STAR, GSG, SUP-12, TASR-1, TASR-2, TIA, TIAR, TRA2, TRA2a/b, U hnRNP, U1 snRNP, U11 snRNP, U12 snRNP, U1-C, U2 snRNP, U2AF1-RS2, U2AF35, U2AF65, U4 snRNP, U5 snRNP, U6 snRNP, Urp, YB1, or any combination thereof. In some embodiments, the first complex comprises a binding pocket. In some embodiments, the binding pocket comprises a bulge, or a mutation, or a stem-loop, or any combinations thereof. In some embodiments, the binding pocket does not comprise a bulge, a mutation, or a stem-loop. In some embodiments, the bulge or the mutation causes a 3-dimensional structural change in the first polynucleotide. In some embodiments, the second binding agent binds to the binding pocket. In some embodiments, the target polynucleotide comprises a sequence encoded by a gene or a gene variant thereof selected from the group consisting of ABCA4, ABCB4, ABCD1, ACADSB, ADA, ADAMTS13, AGL, ALB, ALDH3A2, ALG6, APC, APOB, AR, ATM, ATP7A, ATR, B2M, BMP2K, BRCA1, BRCA2, BTK, C3, CAT, CD46, CDH1, CDH23, CFTR, CHM, COL11A1, COL11A2, COL1A1, COL1A2, COL2A1, COL3A1, COL4A5, COL6A1, COL6A3, COL7A1, COL9A2, COLQ, CUL4B, CYBB, CYP17, CYP19, CYP27, CYP27A1, DES, DMD, DYSF, EGFR, EMD, ETV4, F13A1, F5, F7, F8, FAH, FANCA, FANCC, FANCG, FBN1, FECH, FGA, FGFR2, FGG, FIX, FLNA, FOXM1, FRAS1, GALC, GH1, GHV, HADHA, HBA2, HBB, HEXA, HEXB, HLCS, HMBS, HMGCL, HNF1A, HPRT1, HPRT2, HSF4, HSPG2, HTT, IDS, IKBKAP, INSR, ITGB2, ITGB3, JAG1, KRAS, KRT5, L1CAM, LAMA3, LDLR, LMNA, LPL, MADD, MAPT, MLH1, MSH2, MST1R, MTHFR, MUT, MVK, NF1, NF2, OAT, OPA1, OTC, PAH, PBGD, PCCA, PDH1, PGK1, PHEX, PKD2, PKLR, PLEKHM1, PLKR, POMT2, PRDM1, PRKAR1A, PROC, PSEN1, PTCH1, PTEN, PYGM, RP6KA3, RPGR, RSK2, SBCAD, SCN5A, SERPINA1, SLC12A3, SLC6A8, SMN2, SPINK5, SPTA1, TP53, TRAPPC2, TSC1, TSC2, TSHB, UGT1A1, and USH2A.

In some embodiments, a first NMR spectrum is obtained for the first complex, and a second NMR spectrum is obtained for the second complex. In some embodiments, the method further comprises comparing the first and the second NMR spectrum. In some embodiments, the method further comprises selecting a second binding agent based on a comparison of the first and the second NMR spectrum. In some embodiments, the method further comprises determining a chemical shift of the first and the second NMR spectrums.

In some aspects, the present disclosure provides a method comprising: providing a polynucleotide sample comprising a target polynucleotide, wherein the target polynucleotide comprises a splice site, a branch point (BP), an exonic splicing enhancer (ESE), an exonic splicing silencer (ESS), an intronic splicing enhancer (ISE), an intronic splicing silencer (ISS), or a polypyrimidine tract, or any combinations thereof; contacting with the target polynucleotide a first binding agent; and obtaining a first NMR spectrum of the polynucleotide sample using a NMR device. In some embodiments, the target polynucleotide is a target RNA. In some embodiments, the target polynucleotide is a pre-mRNA or a portion thereof. In some embodiments, the target polynucleotide contains at least one exon or a fragment thereof. In some embodiments, the target polynucleotide contains at least one intron or a fragment thereof. In some embodiments, the target polynucleotide contains an exon-intron boundary. In some embodiments, the target polynucleotide contains a splice site. In some embodiments, the splice site is a 5′ splice site, a cryptic 5′ splice site, 3′ splice site, or a cryptic 3′ splice site, or a portion thereof. In some embodiments, the target polynucleotide is at least 8 nucleotides in length. In some embodiments, the target polynucleotide is at least 25 nucleotides in length. In some embodiments, the target polynucleotide is at most 1000 nucleotides in length. In some embodiments, the target polynucleotide comprises none or at least one nucleotide isotopically labeled with one or more atomic labels comprising ²H, ¹³C, ¹⁵N, ¹⁹F and ³¹P. In some embodiments, the first binding agent comprises a first polynucleotide, a first polypeptide, or a combination thereof. In some embodiments, the first polynucleotide is a first RNA. In some embodiments, the first RNA is a small nuclear RNA (snRNA) or a portion thereof. In some embodiments, the snRNA is U1 snRNA, U2 snRNA, U4 snRNA, U5 snRNA, U6 snRNA, U11 snRNA, U12 snRNA, U4atac snRNA, U5 snRNA, U6atac snRNA; or a portion thereof. In some embodiments, the first polypeptide is a protein component of a ribonucleoprotein or a portion thereof. In some embodiments, the ribonucleoprotein is a small nuclear ribonucleoprotein (snRNP) or a portion thereof. In some embodiments, the snRNP is U1 snRNP, U2 snRNP, U4 snRNP, U5 snRNP, U6 snRNP, U11 snRNP, U12 snRNP, U4atac snRNP, U5 snRNP, U6atac snRNP or a portion thereof. In some embodiments, the first polypeptide is a protein or a portion thereof selected from the group comprising 9G8, A1 hnRNP, A2 hnRNP, ASD-1, ASD-2b, ASF, B1 hnRNP, C1 hnRNP, C2 hnRNAP, CBP20, CBP80, CELF, F hnRNP, FBP11, Fox-1, Fox-2, G hnRNP, H hnRNP, hnRNP 1, hnRNP 3, hnRNP C, hnRNP G, hnRNP K, hnRNP M, hnRNP U, Hu, HUR, I hnRNP, K hnRNP, KH-type splicing regulatory protein (KSRP), L hnRNP, M hnRNP, mBBP, muscle-blind like (MBNL), NF45, NFAR, Nova-1, Nova-2, nPTB, P54/SFRS11, polypyrimidine tract binding protein (PTB), PRP19 complex proteins, R hnRNP, RNPC1, SAM68, SC35, SF, SF1/BBP, SF2, SF3A, SF3B, SFRS10, Sm proteins, SR proteins, SRm300, SRp20, SRp30c, SRP35C, SRP36, SRP38, SRp40, SRp55, SRp75, SRSF, STAR, GSG, SUP-12, TASR-1, TASR-2, TIA, TIAR, TRA2, TRA2a/b, U hnRNP, U1 snRNP, U11 snRNP, U12 snRNP, U1-C, U2 snRNP, U2AF1-RS2, U2AF35, U2AF65, U4 snRNP, U5 snRNP, U6 snRNP, Urp, YB1, or any combination thereof. In some embodiments, the target polynucleotide and the first binding agent form a first complex. In some embodiments, the first complex comprises a binding pocket. In some embodiments, the binding pocket comprises a bulge, or a mutation, or a stem-loop, or any combinations thereof. In some embodiments, the binding pocket does not comprise a bulge, a mutation, or a stem-loop. In some embodiments, the bulge or the mutation causes a 3-dimensional structural change in the first polynucleotide. In some embodiments, the method further comprises contacting with the first complex a second binding agent. In some embodiments, the second binding agent comprises one or more molecules selected from a group comprising a polynucleotide, a polypeptide, a protein, a small molecule, an ion, a salt, and an atom. In some embodiments, the second binding agent is a small molecule. In some embodiments, the small molecule is a library of small molecules. In some embodiments, the method further comprises obtaining a second NMR spectrum after contacting with the first complex the second binding agent. In some embodiments, the method further comprises comparing the first and the second NMR spectrum. In some embodiments, the method further comprises determining a chemical shift of the one or more atoms from the first and the second NMR spectrums. In some embodiments, the target polynucleotide the target polynucleotide comprises a sequence encoded by a gene or a gene variant thereof selected from the group consisting of ABCA4, ABCB4, ABCD1, ACADSB, ADA, ADAMTS13, AGL, ALB, ALDH3A2, ALG6, APC, APOB, AR, ATM, ATP7A, ATR, B2M, BMP2K, BRCA1, BRCA2, BTK, C3, CAT, CD46, CDH1, CDH23, CFTR, CHM, COL11A1, COL11A2, COL1A1, COL1A2, COL2A1, COL3A1, COL4A5, COL6A1, COL6A3, COL7A1, COL9A2, COLQ, CUL4B, CYBB, CYP17, CYP19, CYP27, CYP27A1, DES, DMD, DYSF, EGFR, EMD, ETV4, F13A1, F5, F7, F8, FAH, FANCA, FANCC, FANCG, FBN1, FECH, FGA, FGFR2, FGG, FIX, FLNA, FOXM1, FRAS1, GALC, GH1, GHV, HADHA, HBA2, HBB, HEXA, HEXB, HLCS, HMBS, HMGCL, HNF1A, HPRT1, HPRT2, HSF4, HSPG2, HTT, IDS, IKBKAP, INSR, ITGB2, ITGB3, JAG1, KRAS, KRT5, L1CAM, LAMA3, LDLR, LMNA, LPL, MADD, MAPT, MLH1, MSH2, MST1R, MTHFR, MUT, MVK, NF1, NF2, OAT, OPA1, OTC, PAH, PBGD, PCCA, PDH1, PGK1, PHEX, PKD2, PKLR, PLEKHM1, PLKR, POMT2, PRDM1, PRKAR1A, PROC, PSEN1, PTCH1, PTEN, PYGM, RP6KA3, RPGR, RSK2, SBCAD, SCN5A, SERPINA1, SLC12A3, SLC6A8, SMN2, SPINK5, SPTA1, TP53, TRAPPC2, TSC1, TSC2, TSHB, UGT1A1, and USH2A.

In some aspects, the present disclosure provides a method for selecting a binding agent to a polynucleotide, the method comprising: (a) providing a polynucleotide sample comprising a target polynucleotide; (b) obtaining a first NMR spectrum of the polynucleotide sample using a NMR device; (c) contacting with the polynucleotide sample a binding agent; (d) obtaining a second NMR spectrum of the polynucleotide sample after contacting with the binding agent; and (e) comparing the first and the second NMR spectrum; and (f) selecting the binding agent based on the comparison. In some embodiments, the binding agent comprises a small molecule, a polynucleotide, or a polypeptide, or any combinations thereof. In some embodiments, the binding agent comprises a library of small molecules. In some embodiments, the polynucleotide sample further comprises a first polynucleotide. In some embodiments, the target polynucleotide and the first polynucleotide are added with about equimolar amounts. In some embodiments, the first polynucleotide is a first RNA. In some embodiments, the first RNA is a small nuclear RNA (snRNA) or a portion thereof. In some embodiments, the snRNA is U1, U2, U4, U5, U6, U11, U12, U4atac, U5, or U6atac snRNA; or a portion thereof. In some embodiments, the target and the first polynucleotide form a duplex. In some embodiments, the duplex contains a binding pocket. In some embodiments, the binding pocket comprises a bulge, or a mutation, or a stem-loop, or any combinations thereof. In some embodiments, the binding pocket does not comprise a bulge, a mutation, or a stem-loop. In some embodiments, the target polynucleotide comprises a splice site, a branch point (BP), an exonic splicing enhancer (ESE), an exonic splicing silencer (ESS), an intronic splicing enhancer (ISE), an intronic splicing silencer (ISS), or a polypyrimidine tract, or a portion thereof. In some embodiments, the target polynucleotide contains at least one exon or a fragment thereof. In some embodiments, the target polynucleotide contains at least one intron or a fragment thereof. In some embodiments, the target polynucleotide contains at least one exon-intron boundary. In some embodiments, the target polynucleotide is at least 8 nucleotides in length. In some embodiments, the target polynucleotide is at least 25 nucleotides in length. In some embodiments, the target polynucleotide is at most 1000 nucleotides in length. In some embodiments, the target polynucleotide is from 100 to 200 nucleotides in length. In some embodiments, the target polynucleotide comprises none or at least one nucleotide isotopically labeled with one or more atomic labels comprising ²H, ¹³C, ¹⁵N, ¹⁹F and ³¹P. some embodiments, the method further comprises determining a chemical shift of the first or the second NMR spectrum. In some embodiments, the method further comprises determining a 3-dimensional atomic resolution structure of the polynucleotide and the bound small molecule. In some embodiments, the 3-dimensional atomic resolution structure is determined by structure prediction software. In some embodiments, the structure prediction software is Amos/Candid-program suite. In some embodiments, the structure prediction software is MC-fold|MC-Sym pipeline. In some embodiments, determining the 3-dimensional atomic resolution structure comprises generating a plurality of theoretical structural polynucleotide 2-dimensional models using the nucleotide sequence and one or more 2-dimensional structure prediction algorithms. In some embodiments, the method further comprises generating a plurality of theoretical structural polynucleotide 3-dimensional models using a 3-dimensional structure predicting algorithm using the plurality of theoretical structural polynucleotide 2-dimensional models and optionally one or more known and/or assumed polynucleotide 2-dimensional models. In some embodiments, the method further comprises generating a predicted chemical shift set for each of the plurality of theoretical structural polynucleotide 3-dimensional models. In some embodiments, the method further comprises comparing the predicted chemical shift set to the chemical shift(s). In some embodiments, the method further comprises selecting one or more theoretical structural polynucleotide 3-dimensional models having an agreement between the respective predicted chemical shift set and the chemical shift(s) as the one or more 3-dimensional atomic resolution structures. In some embodiments, the 2-dimensional structure prediction algorithm is a nearest neighbor algorithm. In some embodiments, the method further comprises the step: generating one or more refined 3-dimensional atomic resolution structures by refining the selected one or more theoretical structural polynucleotide 3-dimensional model using a modeling software that performs one or more functions comprising energy minimization and/or a molecular dynamics simulation. In some embodiments, the predicted chemical shift set is generated by comparing each theoretical structural polynucleotide 3-dimensional model with a NMR data-structure database. In some embodiments, generating the predicted chemical shift set comprises calculating a polynucleotide structural metric comprising atomic coordinates, stacking interactions, magnetic susceptibility, electromagnetic fields, or dihedral angles from one or more experimentally determined polynucleotide 3-dimensional structures. In some embodiments, the method further comprises using a regression algorithm to generate a set of mathematical functions or objects that describe relationships between experimental chemical shifts and the polynucleotide structural metric of the experimentally determined 3-dimensional polynucleotide structures. In some embodiments, the method further comprises calculating a polynucleotide structural metric for each of the theoretical structural polynucleotide 3-dimensional models. In some embodiments, the method further comprises inputting the polynucleotide structural metric for each of the theoretical structural polynucleotide 3-dimensional models into the set of mathematical functions or objects to generate the predicted chemical shift set. In some embodiments, the regression algorithm is machine learning algorithm comprising a Random Forest algorithm. In some embodiments, the NMR spectrum is obtained with a NMR spectrometer frequency ranging from about 1 GHz MHz to about 20 MHz. In some embodiments, the NMR spectrum is obtained with a NMR spectrometer frequency ranging from 500 MHz to 900 MHz. In some embodiments, the NMR device is AVANCE III. In some embodiments, the method further comprises determining a binding kinetics of a snRNA binding to the target polynucleotide with or without the binding agent selected from the step (f). In some embodiments, the method further comprises determining a binding kinetics of a snRNP binding to the target polynucleotide with or without the binding agent selected from the step (f). In some embodiments, the method further comprises comparing the binding kinetics determined with and without the binding agent selected from step (f). In some embodiments, the method further comprises selecting a first small molecule and a second small molecule. In some embodiments, the method further comprises determining a first binding kinetics of a snRNA binding to the target polynucleotide with or without the first small molecule, and a second binding kinetics of the snRNA binding to the target polynucleotide with or without the second small molecule. In some embodiments, the method further comprises comparing the first binding kinetics and the second binding kinetics. In some embodiments, the binding kinetics is determined by surface plasmon resonance (SPR), Bio-Layer Interferometry (BLI) technology (Octet Systems), isothermal titration calorimetry (ITC), or fluorescence anisotropy. In some embodiments, the method comprises determining a 2-dimensional model or a 3-dimensional structure of the first small molecule and the second small molecule. In some embodiments, the method comprises comparing the 2-dimensional model or the 3-dimensional structure of the first and the second small molecule.

In some aspects, the present disclosure provides a method comprising: identifying one or more binding pockets formed by a target polynucleotide and a first polynucleotide, wherein the target polynucleotide contains a sequence of a splice site, a branch point (BP), an exonic splicing enhancer (ESE), an exonic splicing silencer (ESS), an intronic splicing enhancer (ISE), an intronic splicing silencer (ISS), or a polypyrimidine tract, or any combinations thereof; and virtually screening one or more small molecules or fragments thereof against the one or more binding pockets, wherein the virtual screening process identifies putative small molecule or fragment hits. In some embodiments, identifying one or more binding pockets comprises solving a 3-dimensional atomic resolution structure comprising the target polynucleotide and the first polynucleotide. In some embodiments, the 3-dimensional atomic resolution structure is determined by a NMR spectrum. In some embodiments, the method further comprises testing one or more small molecule or fragment hits from the virtual screen using an experimental assay. In some embodiments, the experimental assay is surface plasmon resonance (SPR), Bio-Layer Interferometry (BLI) technology (Octet Systems), isothermal titration calorimetry (ITC), or fluorescence anisotropy. In some embodiments, the target polynucleotide is a RNA. In some embodiments, the target polynucleotide is a pre-mRNA. In some embodiments, the splice site is a 5′ splice site, a cryptic 5′ splice site, a 3′ splice site, or a cryptic 3′ splice site. In some embodiments, the target polynucleotide contains at least one intron or a fragment thereof. In some embodiments, the target polynucleotide contains at least one exon or a fragment thereof. In some embodiments, the target polynucleotide contains at least one exon-intron boundary. In some embodiments, the target polynucleotide is at least 8 nucleotides in length. In some embodiments, the target polynucleotide is at least 25 nucleotides in length. In some embodiments, the target polynucleotide is at most 1000 nucleotides in length. In some embodiments, the target polynucleotide is from 100 to 200 nucleotides in length. In some embodiments, the target polynucleotide comprises a sequence encoded by a gene or a gene variant thereof selected from the group consisting of ABCA4, ABCB4, ABCD1, ACADSB, ADA, ADAMTS13, AGL, ALB, ALDH3A2, ALG6, APC, APOB, AR, ATM, ATP7A, ATR, B2M, BMP2K, BRCA1, BRCA2, BTK, C3, CAT, CD46, CDH1, CDH23, CFTR, CHM, COL11A1, COL11A2, COL1A1, COL1A2, COL2A1, COL3A1, COL4A5, COL6A1, COL6A3, COL7A1, COL9A2, COLQ, CUL4B, CYBB, CYP17, CYP19, CYP27, CYP27A1, DES, DMD, DYSF, EGFR, EMD, ETV4, F13A1, F5, F7, F8, FAH, FANCA, FANCC, FANCG, FBN1, FECH, FGA, FGFR2, FGG, FIX, FLNA, FOXM1, FRAS1, GALC, GH1, GHV, HADHA, HBA2, HBB, HEXA, HEXB, HLCS, HMBS, HMGCL, HNF1A, HPRT1, HPRT2, HSF4, HSPG2, HTT, IDS, IKBKAP, INSR, ITGB2, ITGB3, JAG1, KRAS, KRT5, L1CAM, LAMA3, LDLR, LMNA, LPL, MADD, MAPT, MLH1, MSH2, MST1R, MTHFR, MUT, MVK, NF1, NF2, OAT, OPA1, OTC, PAH, PBGD, PCCA, PDH1, PGK1, PHEX, PKD2, PKLR, PLEKHM1, PLKR, POMT2, PRDM1, PRKAR1A, PROC, PSEN1, PTCH1, PTEN, PYGM, RP6KA3, RPGR, RSK2, SBCAD, SCN5A, SERPINA1, SLC12A3, SLC6A8, SMN2, SPINK5, SPTA1, TP53, TRAPPC2, TSC1, TSC2, TSHB, UGT1A1, and USH2A. In some embodiments, the method further comprises identifying a first putative small molecule or and a second putative small molecule. In some embodiments, the method further comprises determining a first binding kinetics of the first putative small molecule or fragment hit binding to the target polynucleotide, and a second binding kinetics of the second putative small molecule or fragment hit binding to the target polynucleotide. In some embodiments, the method further comprises comparing the first binding kinetics and the second binding kinetics, thereby selecting a stronger small molecule or fragment hit. In some embodiments, the binding kinetics are determined using surface plasmon resonance (SPR), Bio-Layer Interferometry (BLI) technology (Octet Systems), isothermal titration calorimetry (ITC), or fluorescence anisotropy.

In some aspects, the present disclosure provides a method of selecting a binding agent to a target polynucleotide, comprising: contacting to a sample containing the target polynucleotide a binding agent, wherein the target polynucleotide contains a splice site, a branch point (BP), an exonic splicing enhancer (ESE), an exonic splicing silencer (ESS), an intronic splicing enhancer (ISE), an intronic splicing silencer (ISS), or a polypyrimidine tract, or any combinations thereof, obtaining a structure of the binding agent and the target polynucleotide in a first assay; obtaining a binding kinetics of the binding agent in a second assay; and selecting the binding agent based on the structure and the binding kinetics. In some embodiments, the first assay and the second assay are the same. In some embodiments, the first assay and the second assay are NMR. In some embodiments, the first assay is NMR, and the second assay is surface plasmon resonance (SPR), Bio-Layer Interferometry (BLI) technology (Octet Systems), isothermal titration calorimetry (ITC), or fluorescence anisotropy. In some embodiments, the binding agent is a small molecule. In some embodiments, the sample further comprises a first polynucleotide. In some embodiments, the first polynucleotide is a RNA.

In some embodiments, the RNA is a small nuclear RNA (snRNA) or a portion thereof. In some embodiments, the snRNA is U1, U2, U4, U5, U6, U11, U12, U4atac, U5, or U6atac snRNA; or a portion thereof. In some embodiments, the target and the first polynucleotide form a duplex. In some embodiments, the duplex contains a binding pocket. In some embodiments, the binding pocket comprises a bulge, or a mutation, or a stem-loop, or any combinations thereof. In some embodiments, the binding pocket does not comprise a bulge, a mutation, or a stem-loop. In some embodiments, the sample further comprises a protein or a portion thereof. In some embodiments, the protein is a ribonucleoprotein. In some embodiments, the ribonucleoprotein is a small nuclear ribonucleoprotein (snRNP) or a portion thereof. In some embodiments, the snRNP is U1 snRNP, U2 snRNP, U4 snRNP, U5 snRNP, U6 snRNP, U11 snRNP, U12 snRNP, U4atac snRNP, U5 snRNP, U6atac snRNP or a portion thereof. In some embodiments, the protein is selected from the group comprising 9G8, A1 hnRNP, A2 hnRNP, ASD-1, ASD-2b, ASF, B1 hnRNP, C1 hnRNP, C2 hnRNAP, CBP20, CBP80, CELF, F hnRNP, FBP11, Fox-1, Fox-2, G hnRNP, H hnRNP, hnRNP 1, hnRNP 3, hnRNP C, hnRNP G, hnRNP K, hnRNP M, hnRNP U, Hu, HUR, I hnRNP, K hnRNP, KH-type splicing regulatory protein (KSRP), L hnRNP, M hnRNP, mBBP, muscle-blind like (MBNL), NF45, NFAR, Nova-1, Nova-2, nPTB, P54/SFRS11, polypyrimidine tract binding protein (PTB), PRP19 complex proteins, R hnRNP, RNPC1, SAM68, SC35, SF, SF1/BBP, SF2, SF3A, SF3B, SFRS10, Sm proteins, SR proteins, SRm300, SRp20, SRp30c, SRP35C, SRP36, SRP38, SRp40, SRp55, SRp75, SRSF, STAR, GSG, SUP-12, TASR-1, TASR-2, TIA, TIAR, TRA2, TRA2a/b, U hnRNP, U1 snRNP, U11 snRNP, U12 snRNP, U1-C, U2 snRNP, U2AF1-RS2, U2AF35, U2AF65, U4 snRNP, U5 snRNP, U6 snRNP, Urp, YB1, or any combination thereof.

In some embodiments, the target polynucleotide comprises GGA/gtgagu, AGA/gugagu, AGA/gugagu, AGA/gugagu, AGA/gugagu, AGA/gugagu, AGA/gugagc, AGA/gugagu, AGA/gugagu, GGA/gugagu, CGA/guccgu, GGAguaagu, GGA/guaagu, AGA/guaagu, AGA/guaagu, AGA/guaagu, AGA/guaagu, AGA/guaagu, AGA/guaagu, AGA/guaaga, AGA/guaagu, AGA/guaagu, AGA/guaagu, GGA/guaagu, AGA/guaagg, AGA/guaagu, AGA/guaagu, AGA/guaagu, GGA/guaagu, AGA/guaaga, AGA/guaagu, AGA/guaagu, AGA/guaagu, GGA/guaagg, AGA/guaagu, AGA/guaagu, GGA/guaagu, AGA/guaagu, AGA/guaaga, AGA/guaagu, AGA/guagau, UGA/gugaau, GGA/guuagu, AGA/guaggu, AGA/guaggu, GGA/guaggu, or AGA/gugcgu.

In some embodiments, the target polynucleotide comprises ACA/gugagg, AAA/auaagu, GAA/ggaagu, GAA/guaaau, GCA/guagga, CAA/gugagu, GUA/gugagu, GAA/guggg, CCA/guaaac, UUA/guaaau, CAA/guaaac, ACA/guaaau, GAA/guaaac, UCA/guaaac, UCA/guaaau, GCA/guaaau, ACA/guaaau, CAA/gcaag, CAA/guaagg, UCA/guaagu, AUA/gugaau, CAA/gugaaa, CCA/gugaga, UCA/gugauu, GAA/gugugu, GAA/uaaguu, CAA/guaugu, AAA/guaugu, CAA/guauuu, ACA/guuagu, GCA/guuagu, or ACA/guuuga.

In some embodiments, the target polynucleotide comprises CAA/guaacu, AUA/gucagu, GAA/gucugg, or AAA/guacau.

In some embodiments, the target polynucleotide comprises NNBgunnnn, NNBhunnnn, or NNBgvnnnn, wherein N/n is A, U, G or C; B is C, G, or U; h is a, c, or u; v is a, c or g.

In some embodiments, the target polynucleotide comprises NNBgurrrn, NNBguwwdn, NNBguvmvn, NNBguvbbn, NNBgukddn, NNBgubnbd, NNBhunngn, NNBhurmhd, or NNBgvdnvn, wherein N/n is A, U, G or C; B is C, G, or U; h is a, c, or u; v is a, c or g; r is a or g; m is a or c; d is a, g or u; k is g or u; w is a or u.

In some embodiments, the target polynucleotide comprises CAC/gugagc, UCC/gugagc, AGC/gugagu, AGC/gugagu, AGG/gugagg, GUG/gugagc, GAG/gugagg, CCG/gugagg, UUG/gugagc, GUG/gugagu, UUU/gugagc, UUU/gugagc, GAU/gugagg, AGU/gugagu, AGU/gugagu, AGU/gugagu, AGU/gugagu, AGC/guaagu, GGC/guaagu, AAC/guaagu, GGC/guaagu, AGC/guaagg, GGC/guaagu, AGC/guaagu, GGC/guaagu, GGC/guaagu, AGC/guaagu, GAG/guaaga, CAG/guaagu, AGU/guaagc, AAU/guaagc, AAU/guaagg, CCU/guaagc, AGU/guaagu, GGU/guaagu, AGU/guaagu, AGU/guaagu, AGU/guaagu, GAU/guaagu, UCC/gugaau, CCG/gugaau, ACG/gugaac, CUG/gugaau, AGG/gugaau, UUG/gugaau, CCG/gugaau, GAG/gugaag, CCU/gugaau, CGU/gugaau, CCU/gugaau, GAG/guagga, CAU/guaggg, UGG/guggau, CAG/guggau, UGG/guggau, CGG/gugggu, GCG/guggga, UGG/guggggg, UGG/gugggug, CGU/gugggu, AUC/gguaaaa, GGG/guaaau, GCG/guaaaa, CAG/guaaag, UGG/guaaag, AAG/guaaag, AAG/guaaau, CAG/guaaag, UAG/guaaag, UUG/guaaag, GAG/guaaag, CAG/guaaag, AUG/guaaaa, AAG/guaaag, CAG/guaaag, CAG/guaaaa, GAG/guaaag, AAG/guaaag, UGU/guaaau, GUU/guaaau, GUU/guaaau, UCU/guaaau, GCU/guaaau, GAU/guaaau, GCU/guaaau, UCU/guaaau, ACU/guaaau, CCU/guaaau, CCU/guaaau, ACU/guaaau, AAU/guaaau, AGG/guagac, UUG/guagau, CAG/guagag, AAG/guagag, AAU/gugagu, CAG/gugagc, AAG/gugggu, AAG/guaggg, CAG/guaggc, or AGC/guaggu.

In some embodiments, the target polynucleotide comprises CAG/guaau, CAG/guaaugu, CAG/guaaugu, CAG/guaaugu, CAG/guaaugu, GAG/guaauac, GAG/guaauau, GAG/guaaugu, AAG/guaauaa, AAG/guaaugu, AAG/guaaugu, AAG/guaaugua, AAG/guaaugu, AAG/guaaugu, GCU/guaauu, CCU/guaauu, GAU/guaauu, CAU/guaauu, AAU/guaauu, AGG/guauau, CAG/guauau, UAG/guauau, CAG/guauau, CGG/guauau, GAG/guauau, CGG/guauau, CAG/guauag, AAG/guauau, CAG/guauag, AAG/guauac, UAG/guauau, CAG/guauag, CAG/guauau, AAG/guuaag, AUC/guuaga, GCG/guuagu, AAG/guuagc, UGG/guuagu, GCG/guuagu, CUG/guuugu, CUG/guauga, CAG/guauga, UAG/guauga, AAG/guaugg, AAG/guauga, GAG/guaugg, CAG/guauga, CAG/guaugg, AAG/guaugg, UGG/guaugc, CAG/guaugu, AUG/guaugu, AAG/guaugu, AAG/guaugg, CAG/guaugg, GAG/guauga, CGG/guaugg, AAU/guaugu, AAG/guauuu, AUG/guauuu, UAG/guauug, AAG/guauuu, CAG/guauug, CAG/guauug, CAU/guauuu, ACU/guauu, AAG/guuuau, AAG/guuuaa, CAG/guuugg, CAG/guuugg, CAG/guuugc, AAG/guuugg, AAG/guuugg, or UGG/guaugc.

In some embodiments, the target polynucleotide comprises CCG/guaacu, UUG/guaaca, AUG/guaacc, GGG/guaacu, AAG/guaaca, AAG/guaacu, UUG/guaaca, GCU/guaacu, ACU/guaacu, GCU/guaacu, UAG/guaccc, AAG/guaccu, CAG/guaccg, UGG/guacca, CAG/gucaau, AAG/gucaau, AAG/gucaag, AUG/guacau, GGG/guacau, UUG/guacau, CAG/guacag, CAG/guacag, CAG/guacag, CAG/guacag, AAG/guacag, CAG/guacag, GAG/guacaa, AAG/guacag, CAG/guacaa, UGU/guacau, CAG/gugcac, GGG/gugcau, CUG/gugcau, UAG/gugcau, CAG/gugcag, CAG/gugcag, AGG/gugcaa, AAC/gugacu, UCC/gugacu, CCG/gugacu, GCG/gugacu, GGG/gugacg, GGG/gugacg, GCG/gugacu, AUG/gugacc, GAU/gugacu, GGC/gucagu, or UAG/gucaga.

In some embodiments, the target polynucleotide comprises AAG/guacgg, AAG/guacgg, AAG/guacug, AAG/guagcg, AAG/guagua, AAG/guagua, AAG/guagua, AAG/guagug, AAG/guauca, AAG/guaucg, AAG/guaucu, AAG/gucucu, AAG/gugccu, AAG/guggua, AAG/guguua, ACG/guagcu, AGC/guacgu, CAG/guacug, CAG/guagua, CAG/guagug, CAG/guagug, CAG/guaucc, CAG/gugcgc, or GAG/gugccu.

In some embodiments, the target polynucleotide comprises CGG/guguau, AAG/guguau, GAG/guguac, CAG/guguau, UAG/guguau, CAG/guguag, GAG/guguau, AAG/gugugc, CAG/guguga, AAG/gugugu, CAG/guguga, CAG/gugugu, UGG/gugugg, CUG/guguga, CGG/gugugu, GAG/gugugc, CAG/guguga, AAU/gugugu, CAG/gugugu, CAG/gugugu, GAG/gugugu, CAG/guuguu, CAG/guuguc, GUG/guugua, CAG/guuguu, AAC/gugauu, CAG/gugaua, AGG/gugauc, GUG/gugauc, CCU/gugauu, GAU/gugauu, CAC/guuggu, CAG/guuggc, AAG/guuagc, or CAG/guugau.

In some embodiments, the target polynucleotide comprises AUG/gucauu, CGG/gucauaauc, AAG/gucugu, AAG/gucuggg, CAG/gucugga, CAG/gucuggu, CAG/gucuga, GAG/gucuggu, AAG/gugucu, AAG/gugucu, AGG/gugucu, CUG/gugcuu, CAG/gucuuu, CAG/guugcu, GAG/gugcug, or CAG/gugcug.

In some embodiments, the target polynucleotide comprises CGC/auaagu, UUC/auaagu, UGG/auaagg, ACG/auaagg, GUU/auaagu, CCU/auaagu, UUU/auaagc, GAG/aucugg, AAC/augagga, GAC/augagg, ACC/augagu, GGG/augagu, AAG/augagc, CAG/augagg, GAG/augagg, GCG/augagu, AAG/gaugag, CCU/augagu, GAU/augagu, GAU/augagu, UAG/augcgu, CAG/auuggu, AAG/auuugu, ACG/cuaagc, CAG/cugugu, CUG/uuaag, GAG/uuaagu, AAG/uuaagg, AUU/uuaagc, CUG/uugaga, CAG/uuuggu, or GGG/auaagu.

In some embodiments, target polynucleotide comprises CAG/auaacu, GAG/cugcag, or AAG/uuaaua.

In some embodiments, the target polynucleotide comprises GCG/gagagu, AAG/ggaaaa, AUC/gguaaaa, AAG/gcaaaa, UGU/gcaagu, GAG/gcaggu, GAG/gcgugg, GAG/gcuccc, CAG/gcuggu, or AAG/gaugag.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 depicts an exemplary binding kinetics assay by BLI.

FIG. 2 depicts exemplary target RNA-RNA duplexes that can be used in various embodiments of the present disclosure.

FIG. 3 depicts exemplary results of cell-based assays testing the effect of selected small molecule binding agents described in the present disclosure.

FIGS. 4A-F depict exemplary binding events of a target polynucleotide binding to one or more binding agents for NMR or kinetics studies. Both first binding agent and second binding agent can comprise one or more molecules. In the case of more than one molecules are comprised in the binding agent, these molecules can be added simultaneously or sequentially.

FIG. 5A depicts a schematic of an SMN2 RNA duplex. The upper strand corresponds to U1 snRNA 5′-end. The strand at the bottom corresponds to the 5′-splice site of SMN2 exon7.

FIG. 5B depicts the structure of an example compound (Compound-A).

FIG. 5C depicts experimental NMR data showing an overlay of the ¹D ¹I-1 spectra of the RNA duplex (imino region) as a function of Compound A concentration (left) and an overlay of the 2D ¹H—¹H TOCSY spectra of the RNA (pyrimidine region) as a function of Compound A concentration (right). The ratio RNA duplex: Compound A are shown.

FIG. 6A depicts the planar structure of Compound A on which the name of the protons (or pseudoatoms) together with the observed chemical shifts are illustrated.

FIG. 6B depicts the planar structure of Compound A on which the intermolecular (nuclear Overhauser effects (NOEs) identified are illustrated.

FIG. 6C depicts experimental NMR data showing portions of the 2D ¹H—¹H NOESY on which intermolecular NOEs are annotated.

### DETAILED DESCRIPTION OF THE INVENTION

The terminology used herein is for the purpose of describing particular example embodiments only and is not intended to be limiting. As used herein, the singular forms “a”, “an” and “the” may be intended to include the plural forms as well, unless the context clearly indicates otherwise. Thus, for example, reference to “a binding agent” includes mixtures of binding agents; reference to “an NMR resonance” includes more than one resonance, and the like. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

In one aspect, provided herein is a method comprising: providing a polynucleotide sample comprising a target polynucleotide; contacting to the target polynucleotide a first binding agent, a second binding agent, or both; wherein the target polynucleotide and the first binding agent form a first complex, wherein the second binding agent and the first complex form a second complex; and obtaining a nuclear magnetic resonance (NMR) spectrum of the first complex, the second complex, or both using a NMR device. In some embodiments, the target polynucleotide is a target ribonucleic acid (RNA). In some embodiments, the target RNA is a precursor messenger RNA (pre-mRNA) or a portion thereof. In some embodiments, the target polynucleotide contains a splice site or a portion thereof. In some embodiments, the splice site is a 5′ splice site, a cryptic 5′ splice site, a 3′ splice site, or a cryptic 3′ spice site, or a portion thereof. In some embodiments, the target polynucleotide contains a branch point (BP), an exonic splicing enhancer (ESE), an exonic splicing silencer (ESS), an intronic splicing enhancer (ISE), an intronic splicing silencer (ISS), or a polypyrimidine tract, or any combinations thereof. In some embodiments, the target polynucleotide contains at least one intron or a fragment thereof. In some embodiments, the target polynucleotide contains at least one exon or a fragment thereof. In some embodiments, the target polynucleotide contains at least one exon-intron boundary. In some embodiments, the target polynucleotide is at least 8 nucleotides in length. In some embodiments, the target polynucleotide is at least 25 nucleotides in length. In some embodiments, the target polynucleotide is at most 1000 nucleotides in length. In some embodiments, the target polynucleotide is from 100 to 200 nucleotides in length. In some embodiments, the target polynucleotide comprises none or at least one nucleotide isotopically labeled with one or more atomic labels comprising ²H, ¹³C, ¹⁵N, ¹⁹F and ³¹P. In some embodiments, the first binding agent comprises a first polynucleotide, a first polypeptide, or a combination thereof. In some embodiments, the first polynucleotide is a first RNA. In some embodiments, the first RNA is a small nuclear RNA (snRNA) or a portion thereof. In some embodiments, the first polypeptide is a protein or a protein component of a protein-RNA complex. In some embodiments, the polypeptide is a protein or protein component of a trans-acting factor. In some embodiments, the polypeptide is a portion, e.g. a domain or subdomain, of a protein associated with RNA splicing. In some embodiments, the polypeptide is a protein component or a portion thereof of one of proteins selected from a group comprising SR, TRA2, SF, SRSF, U1 snRNP, U2 snRNP, U4 snRNP, U5 snRNP, U6 snRNP, U11 snRNP, U12 snRNP, U1-C, Sm proteins, FBP11, SF3A, SF3B, U2AF65, U2AF35, PRP19 complex proteins, hnRNP 1, hnRNP 3, hnRNP C, hnRNP G, hnRNP K, hnRNP M, hnRNP U, ASF, SF2, 9G8, SRP20, TRA2a/b, SRP36, SRP35C, SRP30C, SRP38, SRP40, SRP55, SRP75, HUR, NFAR, NF45, YB1, and junction complex proteins. Other exemplary proteins that are associated with RNA splicing include mBBP, polypyrimidine tract binding protein (PTB), nPTB, KH-type splicing regulatory protein (KSRP), SAM68, STAR/GSG, ASD-2b, ASD-1, SUP-12, RNPC1, ASF, snRNP auxiliary factor-35 (U2AF35), ASF/SF2, Nova-1/2, Fox-1/2, Muscle-blind like (MBNL), CELF, Hu, TIA, TIAR, and their aliases. In some embodiments, the first polypeptide is a protein component of a ribonucleoprotein or a portion thereof. In some embodiments, the ribonucleoprotein is a small nuclear ribonucleoprotein (snRNP) or a portion thereof. In some embodiments, the snRNP is U1 snRNP, U2 snRNP, U4 snRNP, U5 snRNP, U6 snRNP, U11 snRNP, U12 snRNP, U4atac snRNP, U5 snRNP, U6atac snRNP or a portion thereof. In some embodiments, the second binding agent is a small molecule. In some embodiments, the first binding agent comprises a small molecule. In some embodiments, the second binding agent comprises a second polynucleotide, a second polypeptide, or a combination thereof. In some embodiments, the second polynucleotide is a second RNA. In some embodiments, the second RNA is a small nuclear RNA (snRNA) or a portion thereof. In some embodiments, the second polypeptide is a protein component of a ribonucleoprotein or a portion thereof. In some embodiments, the ribonucleoprotein is a small nuclear ribonucleoprotein (snRNP) or a portion thereof. In some embodiments, the snRNP is U1 snRNP, U2 snRNP, U4 snRNP, U5 snRNP, U6 snRNP, U11 snRNP, U12 snRNP, U4atac snRNP, U5 snRNP, U6atac snRNP or a portion thereof. In some embodiments, the first complex comprises a binding pocket. In some embodiments, the binding pocket comprises a bulge, or a mutation, or a stem-loop, or any combinations thereof. In some embodiments, the binding pocket comprises a region or sequence adjacent to a stem-loop structure. In some embodiments, the binding pocket does not comprise a bulge, a mutation, or a stem-loop. In some embodiments, the bulge or the mutation causes a 3-dimensional structural change in the first polynucleotide. In some embodiments, a binding agent targeting the binding pocket can induce a 3-dimensional structural change upon binding to the binding pocket. In some embodiments, the second binding agent binds to the binding pocket. In some embodiments, the pre-mRNA comprises a sequence encoded by a gene or a gene variant thereof selected from the group consisting of ABCA4, ABCB4, ABCD1, ACADSB, ADA, ADAMTS13, AGL, ALB, ALDH3A2, ALG6, APC, APOB, AR, ATM, ATP7A, ATR, B2M, BMP2K, BRCA1, BRCA2, BTK, C3, CAT, CDH1, CDH23, CFTR, CHM, COL11A1, COL11A2, COL1A1, COL1A2, COL2A1, COL3A1, COL4A5, COL6A1, COL6A3, COL7A1, COL9A2, COLQ, CUL4B, CYBB, CYP17, CYP19, CYP27, CYP27A1, DES, DMD, DYSF, EGFR, EMD, ETV4, F13A1, F5, F7, F8, FAH, FANCA, FANCC, FANCG, FBN1, FECH, FGA, FGFR2, FGG, FIX, FLNA, FOXM1, FRAS1, GALC, GH1, GHV, HADHA, HBA2, HBB, HEXA, HEXB, HLCS, HMBS, HMGCL, HNF1A, HPRT1, HPRT2, HSF4, HSPG2, HTT, IDS, IKBKAP, INSR, ITGB2, ITGB3, JAG1, KRAS, KRT5, L1CAM, LAMA3, LDLR, LMNA, LPL, MADD, MAPT, MLH1, MSH2, MST1R, MTHFR, MUT, MVK, NF1, NF2, OAT, OPA1, OTC, PAH, PBGD, PCCA, PDH1, PGK1, PHEX, PKD2, PKLR, PLEKHM1, PLKR, POMT2, PRDM1, PRKAR1A, PROC, PSEN1, PTCH1, PTEN, PYGM, RP6KA3, RPGR, RSK2, SBCAD, SCN5A, SERPINA1, SLC12A3, SLC6A8, SMN2, SPINK5, SPTA1, TP53, TRAPPC2, TSC1, TSC2, TSHB, UGT1A1, CD46, and USH2A. In some embodiments, a first NMR spectrum is obtained for the first complex, and a second NMR spectrum is obtained for the second complex. In some embodiments, the method further comprises comparing the first and the second NMR spectrum. In some embodiments, the method further comprises selecting a second binding agent based on a comparison of the first and the second NMR spectrum. In some embodiments, the method further comprises determining a chemical shift of the first and the second NMR spectrums.

In one aspect, provided herein is a method comprising: providing a polynucleotide sample comprising a target polynucleotide, wherein the target polynucleotide comprises a splice site, a branch point (BP), an exonic splicing enhancer (ESE), an exonic splicing silencer (ESS), an intronic splicing enhancer (ISE), an intronic splicing silencer (ISS), or a polypyrimidine tract, or any combinations thereof contacting with the target polynucleotide a first binding agent; and obtaining a first NMR spectrum of the polynucleotide sample using a NMR device. In some embodiments, the target polynucleotide is a target RNA. In some embodiments, the target polynucleotide is a pre-mRNA or a portion thereof. In some embodiments, the target polynucleotide contains at least one exon or a fragment thereof. In some embodiments, the target polynucleotide contains at least one intron or a fragment thereof. In some embodiments, the target polynucleotide contains an exon-intron boundary. In some embodiments, the target polynucleotide contains a splice site or a portion thereof. In some embodiments, the splice site is a 5′ splice site, a cryptic 5′ splice site, 3′ splice site, or a cryptic 3′ splice site, or any combinations thereof. In some embodiments, the target polynucleotide is at least 8 nucleotides in length. In some embodiments, the target polynucleotide is at least 25 nucleotides in length. In some embodiments, the target polynucleotide is at most 1000 nucleotides in length. In some embodiments, the target polynucleotide comprises none or at least one nucleotide isotopically labeled with one or more atomic labels comprising ²H, ¹³C, ¹⁵N, ¹⁹F and ³¹P. In some embodiments, the first binding agent comprises a first polynucleotide, a first polypeptide, or a combination thereof. In some embodiments, the first polynucleotide is a first RNA. In some embodiments, the first RNA is a small nuclear RNA (snRNA) or a portion thereof. In some embodiments, the first polypeptide is a protein component of a ribonucleoprotein or a portion thereof. In some embodiments, the ribonucleoprotein is a small nuclear ribonucleoprotein (snRNP) or a portion thereof. In some embodiments, the snRNP is U1 snRNP, U2 snRNP, U4 snRNP, U5 snRNP, U6 snRNP, U11 snRNP, U12 snRNP, U4atac snRNP, U5 snRNP, U6atac snRNP or a portion thereof. In some embodiments, the polypeptide is a protein or protein component of a trans-acting factor. In some embodiments, the polypeptide is a portion, e.g. a domain or subdomain, of a protein associated with RNA splicing. In some embodiments, the polypeptide is a protein component or a portion thereof of one of proteins selected from a group comprising SR, TRA2, SF, SRSF, U1 snRNP, U2 snRNP, U4 snRNP, U5 snRNP, U6 snRNP, U11 snRNP, U12 snRNP, U1-C, Sm proteins, FBP11, SF3A, SF3B, U2AF65, U2AF35, PRP19 complex proteins, hnRNP 1, hnRNP 3, hnRNP C, hnRNP G, hnRNP K, hnRNP M, hnRNP U, ASF, SF2, 9G8, SRP20, TRA2a/b, SRP36, SRP35C, SRP30C, SRP38, SRP40, SRP55, SRP75, HUR, NFAR, NF45, YB1, and junction complex proteins. Other exemplary proteins that are associated with RNA splicing include mBBP, polypyrimidine tract binding protein (PTB), nPTB, KH-type splicing regulatory protein (KSRP), SAM68, STAR/GSG, ASD-2b, ASD-1, SUP-12, RNPC1, ASF, snRNP auxiliary factor-35 (U2AF35), ASF/SF2, Nova-1/2, Fox-1/2, Muscle-blind like (MBNL), CELF, Hu, TIA, TIAR, and their aliases. In some embodiments, the target polynucleotide and the first binding agent form a first complex. In some embodiments, the first complex comprises a binding pocket. In some embodiments, the binding pocket comprises a bulge, a mutation, or a stem-loop, or any combinations thereof. In some embodiments, the bulge or the mutation causes a 3-dimensional structural change in the first polynucleotide. In some embodiments, the method further comprises contacting with the first complex a second binding agent. In some embodiments, the second binding agent comprises one or more molecules selected from a group comprising a polynucleotide, a polypeptide, a protein, a small molecule, an ion, a salt, and an atom. In some embodiments, the second binding agent is a small molecule. In some embodiments, the small molecule is a library of small molecules. In some embodiments, the second binding agent further causes a detectable structural change in the first complex. In some embodiments, the method further comprises obtaining a second NMR spectrum after contacting with the first complex the second binding agent. In some embodiments, the method further comprises comparing the first and the second NMR spectrum. In some embodiments, the method further comprises determining a chemical shift of the one or more atoms from the first and the second NMR spectrums. In some embodiments, the target polynucleotide comprises a sequence encoded by a gene or a gene variant thereof selected from the group consisting of ABCA4, ABCB4, ABCD1, ACADSB, ADA, ADAMTS13, AGL, ALB, ALDH3A2, ALG6, APC, APOB, AR, ATM, ATP7A, ATR, B2M, BMP2K, BRCA1, BRCA2, BTK, C3, CAT, CDH1, CDH23, CFTR, CHM, COL11A1, COL11A2, COL1A1, COL1A2, COL2A1, COL3A1, COL4A5, COL6A1, COL6A3, COL7A1, COL9A2, COLQ, CUL4B, CYBB, CYP17, CYP19, CYP27, CYP27A1, DES, DMD, DYSF, EGFR, EMD, ETV4, F13A1, F5, F7, F8, FAH, FANCA, FANCC, FANCG, FBN1, FECH, FGA, FGFR2, FGG, FIX, FLNA, FOXM1, FRAS1, GALC, GH1, GHV, HADHA, HBA2, HBB, HEXA, HEXB, HLCS, HMBS, HMGCL, HNF1A, HPRT1, HPRT2, HSF4, HSPG2, HTT, IDS, IKBKAP, INSR, ITGB2, ITGB3, JAG1, KRAS, KRT5, L1CAM, LAMA3, LDLR, LMNA, LPL, MADD, MAPT, MLH1, MSH2, MST1R, MTHFR, MUT, MVK, NF1, NF2, OAT, OPA1, OTC, PAH, PBGD, PCCA, PDH1, PGK1, PHEX, PKD2, PKLR, PLEKHM1, PLKR, POMT2, PRDM1, PRKAR1A, PROC, PSEN1, PTCH1, PTEN, PYGM, RP6KA3, RPGR, RSK2, SBCAD, SCN5A, SERPINA1, SLC12A3, SLC6A8, SMN2, SPINK5, SPTA1, TP53, TRAPPC2, TSC1, TSC2, TSHB, UGT1A1, CD46, and USH2A.

In one aspect, provided herein is a method for selecting a binding agent to a polynucleotide, the method comprising: providing a polynucleotide sample comprising a target polynucleotide; obtaining a first NMR spectrum of the polynucleotide sample using a NMR device; contacting with the polynucleotide sample a binding agent; obtaining a second NMR spectrum of the polynucleotide sample after contacting with the binding agent; comparing the first and the second NMR spectrum; and selecting the binding agent based on the comparison. In some embodiments, the binding agent comprises a small molecule, a polynucleotide, or a protein, or any combinations thereof. In some embodiments, the polynucleotide sample further comprises a first polynucleotide. In some embodiments, the target polynucleotide and the first polynucleotide are added with about equimolar amounts. In some embodiments, the first polynucleotide is a first RNA. In some embodiments, the first RNA is a small nuclear RNA (snRNA) or a portion thereof. In some embodiments, the snRNA is U1-U12 snRNA or a portion thereof. In some embodiments, the target and the first polynucleotide form a duplex. In some embodiments, the duplex contains a binding pocket. In some embodiments, the binding pocket comprises a bulge, or a mutation, or a stem-loop, or any combinations thereof. In some embodiments, the binding pocket does not comprise a mutation, a bulge, or a stem-loop. In some embodiments, the target polynucleotide comprises a splice site, a branch point (BP), an exonic splicing enhancer (ESE), an exonic splicing silencer (ESS), an intronic splicing enhancer (ISE), an intronic splicing silencer (ISS), or a polypyrimidine tract, or any combinations thereof. In some embodiments, the target polynucleotide contains at least one exon or a fragment thereof. In some embodiments, the target polynucleotide contains at least one intron or a fragment thereof. In some embodiments, the target polynucleotide contains at least one exon-intron boundary. In some embodiments, the target polynucleotide is at least 8 nucleotides in length. In some embodiments, the target polynucleotide is at least 25 nucleotides in length. In some embodiments, the target polynucleotide is at most 1000 nucleotides in length. In some embodiments, the target polynucleotide is from 100 to 200 nucleotides in length. In some embodiments, the target polynucleotide comprises none or at least one nucleotide isotopically labeled with one or more atomic labels comprising ²H, ¹³C, ¹⁵N, ¹⁹F and ³¹P. In some embodiments, the method further comprises determining a chemical shift of the first or the second NMR spectrum. In some embodiments, the method further comprises determining a 3-dimensional atomic resolution structure of the polynucleotide and the bound or molecularly interacting small molecule. In some embodiments, the 3-dimensional atomic resolution structure is determined by structure prediction software. In some embodiments, the structure prediction software is Atnos/Candid-program suite. In some embodiments, the structure prediction software is MC-fold|MC-Sym pipeline. In some embodiments, determining the 3-dimensional atomic resolution structure comprises generating a plurality of theoretical structural polynucleotide 2-dimensional models using the nucleotide sequence and one or more 2-dimensional structure prediction algorithms. In some embodiments, the method further comprises generating a plurality of theoretical structural polynucleotide 3-dimensional models using a 3-dimensional structure predicting algorithm using the plurality of theoretical structural polynucleotide 2-dimensional models and optionally one or more known and/or assumed polynucleotide 2-dimensional models. In some embodiments, the method further comprises generating a predicted chemical shift set for each of the plurality of theoretical structural polynucleotide 3-dimensional models. In some embodiments, the method further comprises comparing the predicted chemical shift set to the chemical shift(s) of the one or more atoms. In some embodiments, the NMR device is used to perform resonance assignments and identify NOE-derived distances to drive structure calculations. In some embodiments, the method further comprises selecting one or more theoretical structural polynucleotide 3-dimensional model having an agreement between the respective predicted chemical shift set and the chemical shift(s) of the one or more atoms as the one or more 3-dimensional atomic resolution structures. In some embodiments, the 2-dimensional structure prediction algorithm is nearest neighbor algorithm. In some embodiments, the method further comprises the step: generating one or more refined 3-dimensional atomic resolution structures by refining the selected one or more theoretical structural polynucleotide 3-dimensional model using a modeling software that performs one or more functions comprising energy minimization and/or a molecular dynamics simulation. In some embodiments, the predicted chemical shift set is generated by comparing each theoretical structural polynucleotide 3-dimensional model with a NMR data-structure database. In some embodiments, generating the predicted chemical shift set comprises calculating a polynucleotide structural metric comprising atomic coordinates, stacking interactions, magnetic susceptibility, electromagnetic fields, or dihedral angles from one or more experimentally determined polynucleotide 3-dimensional structures. In some embodiments, the method further comprises using a regression algorithm to generate a set of mathematical functions or objects that describe relationships between experimental chemical shifts and the polynucleotide structural metric of the experimentally determined 3-dimensional polynucleotide structures. In some embodiments, the method further comprises calculating a polynucleotide structural metric for each of the theoretical structural polynucleotide 3-dimensional models. In some embodiments, the method further comprises inputting the polynucleotide structural metric for each of the theoretical structural polynucleotide 3-dimensional models into the set of mathematical functions or objects to generate the predicted chemical shift set. In some embodiments, the regression algorithm is machine learning algorithm comprising a Random Forest algorithm. In some embodiments, the NMR spectrum is obtained with a NMR spectrometer frequency ranging from about 1 GHz MHz to about 20 MHz. In some embodiments, the method further comprises the NMR spectrum is obtained with a NMR spectrometer frequency ranging from 500 MHz to 900 MHz. In some embodiments, the NMR device is AVANCE III. In some embodiments, the method further comprises determining the binding kinetics of the binding agent to the duplex. In some embodiments, the binding kinetics is determined by surface plasmon resonance (SPR), Bio-Layer Interferometry (BLI) technology (Octet Systems), isothermal titration calorimetry (ITC), or fluorescence anisotropy. In one aspect, provided herein is a method comprising: identifying one or more binding pockets formed by a first polynucleotide and a second polynucleotide, wherein the first polynucleotide contains a splice site, a branch point (BP), an exonic splicing enhancer (ESE), an exonic splicing silencer (ESS), an intronic splicing enhancer (ISE), an intronic splicing silencer (ISS), or a polypyrimidine tract, or any combinations thereof; and virtually screening one or more small molecules against the one or more binding pockets, wherein the virtual screening process identifies putative small molecule hits. In some embodiments, identifying one or more binding pockets comprises solving a 3-dimensional atomic resolution structure comprising the first polynucleotide and the second polynucleotide. In some embodiments the 3-dimensional atomic resolution structure is determined by a NMR spectrum. In some embodiments, the method further comprises testing one or more small molecule hits from the virtual screen using an experimental assay. In some embodiments, the experimental assay is surface plasmon resonance (SPR), Bio-Layer Interferometry (BLI) technology (Octet Systems), isothermal titration calorimetry (ITC), or fluorescence anisotropy. In some embodiments, the first polynucleotide is a RNA. In some embodiments, the first polynucleotide is a pre-mRNA. In some embodiments, the splice site is a 5′ splice site, a cryptic 5′ splice site, a 3′ splice site, or a cryptic 3′ splice site. In some embodiments, the first polynucleotide contains at least one intron or a fragment thereof. In some embodiments, the first polynucleotide contains at least one exon or a fragment thereof. In some embodiments, the first polynucleotide contains at least one exon-intron boundary. In some embodiments, the first polynucleotide is at least 8 nucleotides in length. In some embodiments, the first polynucleotide is at least 25 nucleotides in length. In some embodiments, the first polynucleotide is at most 1000 nucleotides in length. In some embodiments, the first polynucleotide is from 100 to 200 nucleotides in length. In some embodiments, the first polynucleotide comprises a sequence encoded by a gene or a gene variant thereof selected from the group consisting of ABCA4, ABCB4, ABCD1, ACADSB, ADA, ADAMTS13, AGL, ALB, ALDH3A2, ALG6, APC, APOB, AR, ATM, ATP7A, ATR, B2M, BMP2K, BRCA1, BRCA2, BTK, C3, CAT, CDH1, CDH23, CFTR, CHM, COL11A1, COL11A2, COL1A1, COL1A2, COL2A1, COL3A1, COL4A5, COL6A1, COL6A3, COL7A1, COL9A2, COLQ, CUL4B, CYBB, CYP17, CYP19, CYP27, CYP27A1, DES, DMD, DYSF, EGFR, EMD, ETV4, F13A1, F5, F7, F8, FAH, FANCA, FANCC, FANCG, FBN1, FECH, FGA, FGFR2, FGG, FIX, FLNA, FOXM1, FRAS1, GALC, GH1, GHV, HADHA, HBA2, HBB, HEXA, HEXB, HLCS, HMBS, HMGCL, HNF1A, HPRT1, HPRT2, HSF4, HSPG2, HTT, IDS, IKBKAP, INSR, ITGB2, ITGB3, JAG1, KRAS, KRT5, L1CAM, LAMA3, LDLR, LMNA, LPL, MADD, MAPT, MLH1, MSH2, MST1R, MTHFR, MUT, MVK, NF1, NF2, OAT, OPA1, OTC, PAH, PBGD, PCCA, PDH1, PGK1, PHEX, PKD2, PKLR, PLEKHM1, PLKR, POMT2, PRDM1, PRKAR1A, PROC, PSEN1, PTCH1, PTEN, PYGM, RP6KA3, RPGR, RSK2, SBCAD, SCN5A, SERPINA1, SLC12A3, SLC6A8, SMN2, SPINK5, SPTA1, TP53, TRAPPC2, TSC1, TSC2, TSHB, UGT1A1, CD46, and USH2A.

### Definitions

The term “polynucleotide” as used herein generally refers to a molecule comprising one or more nucleic acid subunits, or nucleotides, and can be used interchangeably with “nucleic acid” or “oligonucleotide”. A polynucleotide may include one or more nucleotides selected from adenosine (A), cytosine (C), guanine (G), thymine (T) and uracil (U), or variants thereof. A nucleotide generally includes a nucleoside and at least 1, 2, 3, 4, 5, 6, 7, 8, 9, 10, or more phosphate (PO₃) groups. A nucleotide can include a nucleobase, a five-carbon sugar (either ribose or deoxyribose), and one or more phosphate groups. Ribonucleotides are nucleotides in which the sugar is ribose. Deoxyribonucleotides are nucleotides in which the sugar is deoxyribose. A nucleotide can be a nucleoside monophosphate or a nucleoside polyphosphate. A nucleotide can be a deoxyribonucleoside polyphosphate, such as, e.g., a deoxyribonucleoside triphosphate (dNTP), which can be selected from deoxyadenosine triphosphate (dATP), deoxycytidine triphosphate (dCTP), deoxyguanosine triphosphate (dGTP), uridine triphosphate (dUTP) and deoxythymidine triphosphate (dTTP) dNTPs, that include detectable tags, such as luminescent tags or markers (e.g., fluorophores). A nucleotide can be isotopically labeled with, for example, ²H, ¹³C, ¹⁵N, ¹⁹F, and ³¹P. A nucleotide can include any subunit that can be incorporated into a growing nucleic acid strand. Such subunit can be an A, C, G, T, or U, or any other subunit that is specific to one or more complementary A, C, G, T or U, or complementary to a purine (i.e., A or G, or variant thereof) or a pyrimidine (i.e., C, T or U, or variant thereof). In some examples, a polynucleotide is deoxyribonucleic acid (DNA), ribonucleic acid (RNA), or derivatives or variants thereof. In some embodiments, a polynucleotide is a short interfering RNA (siRNA), a microRNA (miRNA), a plasmid DNA (pDNA), a short hairpin RNA (shRNA), small nuclear RNA (snRNA), messenger RNA (mRNA), precursor mRNA (pre-mRNA), antisense RNA (asRNA), to name a few, and encompasses both the nucleotide sequence and any structural embodiments thereof, such as single-stranded, double-stranded, triple-stranded, helical, hairpin, etc. In some cases, a polynucleotide molecule is circular. A polynucleotide can have various lengths. A nucleic acid molecule can have a length of at least about 10 bases, 20 bases, 30 bases, 40 bases, 50 bases, 100 bases, 200 bases, 300 bases, 400 bases, 500 bases, 1 kilobase (kb), 2 kb, 3, kb, 4 kb, 5 kb, 10 kb, 50 kb, or more. A polynucleotide can be isolated from a cell or a tissue. As embodied herein, the polynucleotide sequences may comprise isolated and purified DNA/RNA molecules, synthetic DNA/RNA molecules, synthetic DNA/RNA analogs.

Polynucleotides may include one or more nucleotide variants, including nonstandard nucleotide(s), non-natural nucleotide(s), nucleotide analog(s) and/or modified nucleotides. Examples of modified nucleotides include, but are not limited to diaminopurine, 5-fluorouracil, 5-bromouracil, 5-chlorouracil, 5-iodouracil, hypoxanthine, xantine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-D46-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid(v), 5-methyl-2-thiouracil, 3-(3-amino-3-N-2-carboxypropyl) uracil, (acp3)w, 2,6-diaminopurine and the like. In some cases, nucleotides may include modifications in their phosphate moieties, including modifications to a triphosphate moiety. Non-limiting examples of such modifications include phosphate chains of greater length (e.g., a phosphate chain having, 4, 5, 6, 7, 8, 9, 10 or more phosphate moieties) and modifications with thiol moieties (e.g., alpha-thiotriphosphate and beta-thiotriphosphates). Nucleic acid molecules may also be modified at the base moiety (e.g., at one or more atoms that typically are available to form a hydrogen bond with a complementary nucleotide and/or at one or more atoms that are not typically capable of forming a hydrogen bond with a complementary nucleotide), sugar moiety or phosphate backbone. Nucleic acid molecules may also contain amine-modified groups, such as amino ally 1-dUTP (aa-dUTP) and aminohexhylacrylamide-dCTP (aha-dCTP) to allow covalent attachment of amine reactive moieties, such as N-hydroxysuccinimide esters (NHS). Alternatives to standard DNA base pairs or RNA base pairs in the oligonucleotides of the present disclosure can provide higher density in bits per cubic mm, higher safety (resistant to accidental or purposeful synthesis of natural toxins), easier discrimination in photo-programmed polymerases, or lower secondary structure. Such alternative base pairs compatible with natural and mutant polymerases for de novo and/or amplification synthesis are described in Betz K, Malyshev D A, Lavergne T, Welte W, Diederichs K, Dwyer T J, Ordoukhanian P, Romesberg F E, Marx A. Nat. Chem. Biol. 2012 Jul; 8(7):612-4, which is herein incorporated by reference for all purposes.

The term “polynucleotide sample” includes a polynucleotide or a certain quantity (e.g., a number of moles or a concentration of polynucleotide) of the polynucleotide, optionally dissolved in a solvent, wherein the polynucleotides in the polynucleotide sample has one singular nucleotide sequence. In some examples, the polynucleotides in the polynucleotide sample may only have the same nucleotide, or the polynucleotide sample can contain polynucleotides synthesized with different nucleotides. In some examples, the polynucleotides are free of any labels. In some other examples, the polynucleotides are labeled with one or more atomic labels.

As used herein, the term “protein” refers to a long polymer of amino acid residues linked via peptide bonds and which may be composed of one or more polypeptide chains. More specifically, the term “protein” refers to a molecule composed of one or more chains of amino acids in a specific order; for example, the order as determined by the base sequence of nucleotides in the gene coding for the protein. Proteins are essential for the structure, function, and regulation of the body's cells, tissues, and organs, and each protein has unique functions. Examples are hormones, enzymes, antibodies, and any fragments thereof. In some cases, a protein can be a portion of the protein, for example, a domain, a subdomain, or a motif of the protein. In some cases, a protein can be a variant (or mutation) of the protein, wherein one or more amino acid residues are inserted into, deleted from, and/or substituted into the naturally occurring (or at least a known) amino acid sequence of the protein. A protein or a variant thereof can be naturally occurring or recombinant.

As used herein, the term “peptide” is a polymer in which the monomers are amino acids and which are joined together through amide bonds and alternatively referred to as a polypeptide. In the context of this specification it should be appreciated that the amino acids may be the L-optical isomer or the D-optical isomer. Peptides are two or more amino acid monomers long, and often can be more than 20 amino acid monomers long.

A binding pocket can refer to any location on a polynucleotide (e.g. RNA) with sufficient structural complexity (e.g. secondary or tertiary structure) that enables specific interactions of a binding agent on that location to influence the confirmation and structure of the RNA, such that it essential inhibits or activates a splicing process. A binding pocket can contain a bulge, a non-mutation single and duplex RNA, a stem-loop, or sequences adjacent to a stem-loop, mutation-containing single and duplex RNA. A binding pocket may or may not comprise a mutation. In some cases, a binding pocket comprises a sequence portion with a mutation upstream/downstream of the binding pocket, wherein such mutation impacts the structure of RNA at the binding pocket.

A “binding agent” as used herein refers to a molecule that can specifically bind to a nucleic acid molecule, a complex formed by two or more nucleic acid molecules, or a complex formed by a nucleic acid and protein. A binding agent may be a protein, peptide, nucleic acid, carbohydrate, lipid, or small molecular weight compound. A binding agent disclosed herein can modulate or correct RNA mis-splicing.

As used here, a “small molecular weight compound” can be used interchangeably with “small molecule” or “small organic molecule”. Small molecules refer to compounds other than peptides, oligonucleotides, or analogs thereof and typically have molecular weights of less than about 2,000 Daltons.

A ribonucleoprotein (RNP) refers to a nucleoprotein that contains RNA. It is an association that combines a ribonucleic acid and an RNA-binding protein together. Such a combination can also be referred to as a protein-RNA complex. These complexes can function in a number of biological functions that include DNA replication, regulating gene expression and regulating the metabolism of RNA. A few examples of RNPs include the ribosome, the enzyme telomerase, vault ribonucleoproteins, RNase P, heterogeneous nuclear RNPs (hnRNPs) and small nuclear RNPs (snRNPs).

Nascent RNA transcripts from protein-coding genes and mRNA processing intermediates, collectively referred to as pre-mRNA, are generally bound by proteins in the nuclei of eukaryotic cells. From the time nascent transcripts first emerge from RNA polymerase II until mature mRNAs are transported into the cytoplasm, the RNA molecules are associated with an abundant set of nuclear proteins. These proteins are the major protein components of hnRNPs, which contain heterogeneous nuclear RNA (hnRNA), a collective term referring to pre-mRNA and other nuclear RNAs of various sizes.

Splicing factors are proteins or protein complexes that function in splicing or splicing regulation. Splicing factors include those that may be required for constitutive splicing, regulated splicing and splicing of specific messages or groups of messages. A group of related proteins, the SR proteins, can function in constitutive pre-mRNA splicing and may also regulate alternative splice-site selection in a concentration-dependent manner. SR proteins have a modular structure that consists of one or two RNA-recognition motifs (RRMs) and a C-terminal rich in arginine and serine residues (RS domain). Their activity in alternative splicing may be antagonized by members of the hnRNP A/B family of proteins. Splicing factors can also include proteins that are associated with one or more snRNAs. SR proteins in human include SC35, SRp55, SRp40, SRm300, SFRS10, TASR-1, TASR-2, SF2/ASF, 9G8, SRp75, SRp30c, SRp20 and P54/SFRS11. Other splicing factors in human that can be involved in splice site selection include, but are not limited to, U2 snRNA auxiliary factors (e.g. U2AF65, U2AF35), Urp/U2AF1-RS2, SF1/BBP, CBP80, CBP 20, SF1 and PTB/hnRNP1. The hnRNP proteins in humans include, but are not limited to, A1, A2/B1, L, M, K, U, F, H, G, R, I and C1/C2. Splicing factors may be stably or transiently associated with a snRNP or with a transcript.

The term “intron” refers to both the DNA sequence within a gene and the corresponding sequence in the unprocessed RNA transcript. As part of the RNA processing pathway, introns are removed by RNA splicing either shortly after or concurrent with transcription. Introns are found in the genes of most organisms and many viruses. They can be located in a wide range of genes, including those that generate proteins, ribosomal RNA (rRNA), and transfer RNA (tRNA). An “exon” can be any part of a gene that encodes a part of the final mature RNA produced by that gene after introns have been removed by RNA splicing. The term “exon” refers to both the DNA sequence within a gene and to the corresponding sequence in RNA transcripts. A “spliceosome” is assembled from snRNAs and protein complexes. The spliceosome removes introns from a transcribed pre-mRNA.

As used herein, the term “target” or “target molecule” describes a molecule that can be selected from any biological molecule which is modulated by a binding agent bound to a recognition portion on the molecule. The modulation can be activation, inhibition, or any structural change. For example, in some embodiments of the present disclosure, a binding agent can bind to a target molecule (e.g. mRNA) and modulate RNA splicing to correct some defects in splicing. Target molecules encompassed by the present technology can include a diverse array of compounds including polynucleotides, proteins, polypeptides, oligopeptides, ribonucleoproteins, and nucleic acids, including RNA and DNA. In some cases, the target molecule can be target polynucleotide, target RNA, or target DNA. The recognition portion on a molecule refers to a structural portion that interacts with the binding agent. The recognition portion can be a binding pocket, (e.g. a binding pocket on the mRNA), formed by one or more molecules (e.g. RNA and RNA duplexes). In various embodiments provided herein, the binding pocket formed by a target polynucleotide comprises a bulge, or a mutation, or a stem-loop, or any combinations thereof, and can accommodate binding agents such as small molecules. In some embodiments, the binding pocket may not comprise a bulge, a mutation, or a stem-loop.

### Splicing

Splicing or RNA splicing typically refers to the editing of the nascent precursor messenger RNA (pre-mRNA) transcript into a mature messenger RNA (mRNA). Splicing is a biochemical process which includes the removal of introns followed by exon ligation. Sequential transesterification reactions are initiated by a nucleophilic attack of the 5′ splice site (5′ss) by the branch adenosine (branch point; BP) in the downstream intron resulting in the formation of an intron lariat intermediate with a 2′,5′-phosphodiester linkage. This is followed by a 5′ss-mediated attack on the 3′ splice site (3′ss), leading to the removal of the intron lariat and the formation of the spliced RNA product.

Splicing can be regulated by various cis-acting elements and trans-acting factors. Cis-acting elements are sequences of the mRNA and can include core consensus sequences and other regulatory elements. Core consensus sequences typically can refer to conserved RNA sequence motifs, including the 5′ss, 3′ss, polypyrimidine tract and BP region, which can function for spliceosome recruitment. Core consensus sequences can be referred to as construct scaffolds when used in vitro for experimentation. BP refers to a partially conserved sequence of pre-mRNA, generally less than 50 nucleotides upstream of the 3′ss. BP reacts with the 5′ss during the first step of the splicing reaction. Other regulatory cis-acting elements can include exonic splicing enhancer (ESE), exonic splicing silencer (ESS), intronic splicing enhancer (ISE), and intronic splicing silencer (ISS). Trans-acting factors can be proteins or ribonucleoproteins which bind to cis-acting elements.

Splice site identification and regulated splicing can be accomplished principally by two dynamic macromolecular machines, the major (U2-dependent) and minor (U12-dependent) spliceosomes. Each spliceosome contains five snRNPs: U1, U2, U4, U5 and U6 snRNPs for the major spliceosome (which processes ˜95.5% of all introns); and U11, U12, U4atac, U5 and U6atac snRNPs for the minor spliceosome. Spliceosome recognition of consensus sequence elements along with particular structural RNA features. Usually, the U1 snRNP binds to the GU sequence at the 5′ss of an intron. In addition, a number of proteins including U2 small nuclear RNA auxiliary factor 1 (U2AF35) and USAF2 (U2AF65) and splicing factor 1 (SF1, also known as branch point binding protein) may sometimes be required for major spliceosome assembly. U2AF1 can bind at the 3′ss of the intron, and U2AF2 can bind to the polypyrimidine tract. SF1 can bind to the intron BP sequence. The U2 snRNP displaces SF1 and binds to the branch point sequence and ATP is hydrolyzed. The U5/U4/U6 snRNP trimer binds, and the U5 snRNP binds exons at the 5′site, with U6 binding to U2. The U1 snRNP is then released, U5 shifts from exon to intron, and the U6 binds at the 5′ss. U4 then is released, and U6/U2 catalyzes transesterification reaction, making the 5′-end of the intron ligate to the “A” on intron and form a lariat. U5 binds exon at 3′ss, and the 5′site is cleaved, resulting in the formation of the lariat. The U2/U5/U6 remain bound to the lariat, and the 3′ site is cleaved and exons are ligated using ATP hydrolysis. The spliced RNA is released, the lariat is released and degraded, and the snRNPs are recycled. Spliceosome recognition of consensus sequence elements at the 5′ss, 3′ss and BP sites is one of the steps in the splicing pathway, and can be modulated by ESEs, ISEs, ESSs, and ISSs, which can be recognized by auxiliary splicing factors, including SR proteins and hnRNPs. Polypyrimidine tract-binding protein (PTBP, or also known as PTB or hnRNP1) can bind to the polypyrimidine tract of introns and may promote RNA looping.

Alternative splicing is a mechanism by which a single gene may eventually give rise to several different proteins. Alternative splicing can be accomplished by the concerted action of a variety of different proteins, termed “alternative splicing regulatory proteins,” that associate with the pre-mRNA, and cause distinct alternative exons to be included in the mature mRNA. These alternative forms of the gene's transcript can give rise to distinct isoforms of the specified protein. Sequences in pre-mRNA molecules that can bind to alternative splicing regulatory proteins can be found in introns or exons, including, but not limited to, ISS, ISE, ESS, ESE, and polypyrimidine tract. Many mutations or upstream signaling pathways can alter splicing patterns. For example, mutations can be cis-acting elements, and can be located in core consensus sequences (e.g. 5′ss, 3′ss and BP) or the regulatory elements that modulate spliceosome recruitment, including ESE, ESS, ISE, and ISS, or regions that modulate the RNA structure, such as in stem loops. Mutations can also reside in a sequence considered an alternative 5′ss that is activated and recognized by the splicing machinery as a result of a mutation, or a mutation within a 5′ss can cause the use of an alternative 5′ss. For example, mis-signaling can induce more or less of a trans-acting splicing factor to bind to pre-mRNAs and modulate their production of a particular mRNA isoform.

Cryptic splice site, for example, cryptic 5′ss and cryptic 3′ss, can refer to a splice site that is not normally recognized by the spliceosome and therefore are usually in the dormant state. Cryptic splice site can be recognized or activated either by mutations in cis-acting elements or trans-acting factors.

Splicing factors can be de-regulated in cancer, and in some cases, are themselves oncogenes or pseudo-oncogenes and can contribute to positive feedback loops driving cancer progression. For example, CD44 splice isoform switching in human and mouse epithelium is essential for epithelial-mesenchymal transition and breast cancer progression. FOXM1 is expressed in three distinct splice variants, which arise from the same gene through differential splicing of the two facultative exons. FoxM1B and FoxM1C are both transcriptionally active and proteins from these transcripts drive cancer cell cycle progression; whereas FoxM1A is transcriptionally inactive because the addition of an exon abolishes any transcriptional activity of FOXM1, acting as a dominant negative form when expressed; and can stop cancer cell cycle progression. Another example is IG20/MADD, which are two splice isoforms having apposing effects in cancer cells and mice, differing by a single exon. IG20 is an anti-apoptotic form that prevents TRAIL induced apoptosis whereas MADD is a pro-apoptotic form that induced TRAIL induced apoptosis. Indeed, RNA mis-splicing underlies a growing number of human diseases with substantial societal consequences.

However, targeting RNA splicing, more specifically targeting RNA targets, is intractable due to limited available data such as 2-dimensional, and 3-dimensional structures of RNA, chemotypes that engender RNA binding affinity or selectivity, chemotypes that engender RNA binding affinity and selectivity at particular mRNA splicing hot spots, and identification of RNA structural elements that form small molecule binding pockets. In addition, RNA splicing of the pre-mRNA, is heavily influenced by a kinetic component, such that, particular 3-dimensional structures are form by the RNA and/or RNA-protein complexes in particular moments in time. RNA splicing is a dynamic process, involving several trans acting protein factors that bind to the RNA and influence RNA secondary and tertiary structure. Thus, screening for specific and selective small molecular binding agents to correct RNA splicing, may sometimes require the use of tools that can accurately assess binding of multiple agents onto RNA, measure/confirm structural changes as a result of the binding agents, and, as a result, determine changes in molecular associations and sometimes kinetic affinities (dissociation constants) of particular key proteins onto particular key binding regions, or mRNA hot spots, that influence the direction of RNA splicing to include/exclude key regions of the RNA that drive isoform RNA expression. Thus, small molecule interactions with these 3-D binding pockets can influence and correct for RNA mis-expression in disease. Screening of small molecule libraries for binding RNA targets could generate data about chemotypes that engender RNA binding. However, few small molecule-screening collections are enriched in RNA binders; in fact, most libraries are biased with compounds that bind to proteins. In addition, several of the available RNA binder libraries are non-specific or selective to particular RNAs. To address these needs and others, the present disclosure in various embodiments provides a structure-based screening platform that can be used to identify small molecules that bind to RNA and/or RNA protein complex, design novel molecules that can fit into particular RNA binding pockets, and improve specificity and selectivity of small molecules towards disease-associated pre-mRNA splicing defects.

### Target Polynucleotide

The present disclosure in various embodiments provides a structure-based screening platform or method to identify small molecules that can bind polynucleotides and/or complexes formed by polynucleotides and proteins (i.e. polynucleotide-protein complexes) and influence the conformation of the RNA such that it influences the RNA expression. The present disclosure also provides methods to identify small molecules that can bind polynucleotides and/or polynucleotide-protein complexes involved in RNA splicing. The present disclosure also provides methods to identify small molecules that can influence the structure of the RNA and the binding affinity of the trans-acting proteins. In some embodiments, the target polynucleotide is RNA. In some embodiments, the target polynucleotide is mRNA. In some embodiments, the target polynucleotide is a pre-mRNA or a portion of the pre-mRNA. In some embodiments, the target polynucleotide contains a splice site or a portion thereof which includes a 5′ss, a cryptic 5′ss, a 3′ss, or a cryptic 3′ss. In some embodiments, the target polynucleotide comprises one or more other cis-acting elements or a portion thereof, including BP, ESE, ESS, ISE, ISS, and polypyrimidine tract. In some embodiments, the target polynucleotide comprises at least one intron or a fragment thereof. In some embodiments, the target polynucleotide comprises two, three, four, five, six, or more introns or fragments thereof. In some embodiments, the target polynucleotide comprises at least one exon or a fragment thereof. In some embodiments, the target polynucleotide comprises two, three, four, five, six, or more exons or fragments thereof. In some embodiments, the target polynucleotide comprises at least one exon-intron boundary. As used herein, the exon-intron boundary can refer to any polynucleotide that contains intron and exon sequences located at the boundary between an intron and an exon. In some embodiments, the exon-intron boundary may contain a complete sequence of an exon and a fragment sequence of an intron. In some other embodiments, the exon-intron boundary may contain a complete sequence of an intron and a fragment sequence of an exon. In some cases, the target polynucleotide contains both exon and intron sequences, and it is to be understood that the order of exon and intron can vary. For example, the exon can be on the 5′ end of the intron, or the exon can be on the 3′ end of the intron. In some embodiments, the exon-intron boundary comprises 5′ss. In some embodiments, the exon-intron boundary comprises 3′ss. The target polynucleotide can be in various lengths. For example, in some embodiments, the target polynucleotide is at least 5 nucleotides, at least 8 nucleotides, at least 10 nucleotides, at least 15 nucleotides, at least 20 nucleotides, at least 25 nucleotides, at least 30 nucleotides, at least 35 nucleotides, at least 40 nucleotides, at least 45 nucleotides, at least 50 nucleotides, at least 55 nucleotides, at least 60 nucleotides, at least 70 nucleotides, at least 75 nucleotides, at least 80 nucleotides, at least 85 nucleotides, at least 90 nucleotides, at least 95 nucleotides, at least 100 nucleotides, at least 200 nucleotides, at least 300 nucleotides, at least 400 nucleotides, or at least 500 nucleotides in length. In some embodiments, the target polynucleotide is at most 20 nucleotides, at most 50 nucleotides, at most 100 nucleotides, at most 150 nucleotides, at most 200 nucleotides, at most 300 nucleotides, at most 400 nucleotides, at most 500 nucleotides, at most 600 nucleotides, at most 700 nucleotides, at most 800 nucleotides, at most 900 nucleotides, or at most 1000 nucleotides in length. In some embodiments, the target polynucleotide is from 3 to 5 nucleotides, from 5 to 10 nucleotides, from 10-20 nucleotides, from 20 to 40 nucleotides, from 40 to 50 nucleotides, from 50 to 100 nucleotides, from 100 to 150 nucleotides, from 150 to 200 nucleotides, from 200 to 250 nucleotides, from 250 to 300 nucleotides, from 300 to 350 nucleotides, from 350 to 400 nucleotides, from 400 to 450 nucleotides, or from 450 to 500 nucleotides in length.

In some embodiments, the polynucleotide comprises a sequence encoded by a gene selected from the group consisting of ABCA4, ABCB4, ABCD1, ACADSB, ADA, ADAMTS13, AGL, ALB, ALDH3A2, ALG6, APC, APOB, AR, ATM, ATP7A, ATR, B2M, BMP2K, BRCA1, BRCA2, BTK, C3, CAT, CDH1, CDH23, CFTR, CHM, COL11A1, COL11A2, COL1A1, COL1A2, COL2A1, COL3A1, COL4A5, COL6A1, COL6A3, COL7A1, COL9A2, COLQ, CUL4B, CYBB, CYP17, CYP19, CYP27, CYP27A1, DES, DMD, DYSF, EGFR, EMD, ETV4, F13A1, F5, F7, F8, FAH, FANCA, FANCC, FANCG, FBN1, FECH, FGA, FGFR2, FGG, FIX, FLNA, FOXM1, FRAS1, GALC, GH1, GHV, HADHA, HBA2, HBB, HEXA, HEXB, HLCS, HMBS, HMGCL, HNF1A, HPRT1, HPRT2, HSF4, HSPG2, HTT, IDS, IKBKAP, INSR, ITGB2, ITGB3, JAG1, KRAS, KRT5, L1CAM, LAMA3, LDLR, LMNA, LPL, MADD, MAPT, MLH1, MSH2, MST1R, MTHFR, MUT, MVK, NF1, NF2, OAT, OPA1, OTC, PAH, PBGD, PCCA, PDH1, PGK1, PHEX, PKD2, PKLR, PLEKHM1, PLKR, POMT2, PRDM1, PRKAR1A, PROC, PSEN1, PTCH1, PTEN, PYGM, RP6KA3, RPGR, RSK2, SBCAD, SCN5A, SERPINA1, SLC12A3, SLC6A8, SMN2, SPINK5, SPTA1, TP53, TRAPPC2, TSC1, TSC2, TSHB, UGT1A1, CD46, and USH2A. In some embodiments, the polynucleotide is a pre-mRNA encoded by a genetic sequence with at least about 80%, 85%, 90%, 95%, 96%, 97%, 98%, 99% or 100% sequence identity to the above mentioned gene.

In some embodiments, the target polynucleotide may be labeled or modified on one or more nucleotides.

The present disclosure provides a platform screening method to identify small molecule binding agents to bind to polynucleotides and/or polynucleotide-protein complexes by nuclear magnetic resonance (NMR) spectroscopy. In some embodiments, the target polynucleotide is free of any label. In some embodiments, the target polynucleotides comprise no nucleotide that is isotopically labeled. In some other embodiments, the target polynucleotides comprise at least one nucleotide isotopically labeled with one or more atomic labels. In some embodiments, the target polynucleotides comprise two or more nucleotides that are isotopically labeled. Typically, the atomic labels used in NMR spectroscopy can include ²H, ¹³C, ¹⁵N, ¹⁹F, and ³¹F.

### Binding Agent

In various embodiments of the present disclosure, at least one binding agent is introduced in a sample containing a target polynucleotide. In some embodiments, the target polynucleotide itself may form a recognition portion or a binding pocket to accommodate a binding agent such as a small molecule. In some embodiments, the target polynucleotide forms a complex with the at least one binding agent to form a recognition portion or a binding pocket to accommodate additional binding agent(s). The binding agent disclosed herein can be a polynucleotide, a polypeptide, a ribonucleoprotein, a small molecule, or any combinations thereof. In some embodiments, the binding agent can be a mixture of binding agents. In some embodiments, two or more binding agents are introduced to the target polynucleotide. In some embodiments, two or more binding agents are introduced together with the target polynucleotide. In some embodiments, two or more binding agents can be introduced in sequential order to the target polynucleotide.

In some embodiments, the binding agent is a polynucleotide. In a preferred embodiment, the binding agent is a snRNA or a portion thereof. In some embodiments, the binding agent is U1 snRNA or a portion thereof. In some embodiments, the binding agent is U2 snRNA or a portion thereof. In some other embodiments, the binding agent is U1 snRNA, U2 snRNA, U4 snRNA, U5 snRNA, U6 snRNA, U11 snRNA, U12 snRNA, U4atac snRNA, U5 snRNA, U6atac snRNA, or any portions thereof. In some embodiments, the binding agent is a polypeptide. In some embodiments, the binding agent is a protein component of a ribonucleoprotein. In some embodiments, the binding agent is a domain, a motif, or any portion of a protein. In some embodiments, the binding agent can be a protein or a portion thereof selected from the group comprising U1 snRNP, U2 snRNP, U4 snRNP, U5 snRNP, U6 snRNP, U11 snRNP, U12 snRNP, U4atac snRNP, U5 snRNP, U6atac snRNP, or any combinations thereof. In some embodiments, the binding agent can be an auxiliary splicing factor or a portion thereof. Exemplary auxiliary splicing factors include, but are not limited to, SR proteins and hnRNPs. In some embodiments, the binding agent can be a protein or a portion thereof selected from the group comprising SC35, SRp55, SRp40, SRm300, SFRS10, TASR-1, TASR-2, SF2/ASF, 9G8, SRp75, SRp30c, SRp20, P54/SFRS11, U2AF65, U2AF35, Urp/U2AF1-RS2, SF1/BBP, CBP80, CBP 20, PTB/hnRNP I, A1 hnRNP, A2/B1 hnRNP, L hnRNP, M hnRNP, K hnRNP, U hnRNP, F hnRNP, H hnRNP, G hnRNP, R hnRNP, I hnRNP, C1/C2 hnRNP, or any combinations thereof. In some embodiments, the polypeptide is a protein or protein component of a trans-acting factor. In some embodiments, the polypeptide is a portion, e.g. a domain or subdomain, of a protein associated with RNA splicing. In some embodiments, the polypeptide is a protein component or a portion thereof of one of proteins selected from a group comprising SR, TRA2, SF, SRSF, U1 snRNP, U2 snRNP, U4 snRNP, U5 snRNP, U6 snRNP, U11 snRNP, U12 snRNP, U1-C, Sm proteins, FBP11, SF3A, SF3B, U2AF65, U2AF35, PRP19 complex proteins, hnRNP 1, hnRNP 3, hnRNP C, hnRNP G, hnRNP K, hnRNP M, hnRNP U, ASF, SF2, 9G8, SRP20, TRA2a/b, SRP36, SRP35C, SRP30C, SRP38, SRP40, SRP55, SRP75, HUR, NFAR, NF45, YB1, and junction complex proteins. Other exemplary proteins that are associated with RNA splicing include mBBP, polypyrimidine tract binding protein (PTB), nPTB, KH-type splicing regulatory protein (KSRP), SAM68, STAR/GSG, ASD-2b, ASD-1, SUP-12, RNPC1, ASF, snRNP auxiliary factor-35 (U2AF35), ASF/SF2, Nova-1/2, Fox-1/2, Muscle-blind like (MBNL), CELF, Hu, TIA, TIAR, and their aliases. In some embodiments, the protein is a protein variant, a mutant, or a portion of the protein. In some embodiments, the binding agent is a small molecule. In some embodiments, the binding agent is a library of small molecules. Various small molecule libraries can be used with the methods disclosed herein.

In some embodiments, a first binding agent is introduced to the target polynucleotide, thereby allowing the first binding agent and the target polynucleotide to form a first complex. In some embodiments, a second binding agent is introduced to the target polynucleotides, thereby contacting the first complex. In some embodiments, the second binding agent forms a second complex with the first complex. The complex can be a nucleic acid duplex, or a polynucleotide-protein complex, or a polynucleotide-small molecule complex. For example, a first binding agent comprising a polynucleotide can be introduced to a target polynucleotide to form a duplex, and a second binding agent comprising a polypeptide and a small molecule can then be introduced. For another example, a first binding agent comprising a polynucleotide can be introduced to a target polynucleotide to form a duplex, and a second binding agent comprising a small molecule can then be introduced. For yet another example, a first binding agent comprising a polypeptide can be introduced to a target polynucleotide, and a second binding agent comprising a small molecule can then be introduced. It is to be understood that there is no required order for introducing the binding agent to a target polynucleotide. In some embodiments, a binding agent can comprise more than one molecule, and those molecules can be introduced simultaneously or sequentially.

A binding pocket formed by a polynucleotide, or polynucleotide-polynucleotide complex, or polynucleotide-protein complex can be used to accommodate a binding agent such as a small molecule. In various embodiments, a target polynucleotide forms a binding pocket. In some embodiments, a target polynucleotide binds to additional polynucleotide to form a complex which comprises a binding pocket. In some embodiments, a target polynucleotide binds to a protein-RNA complex to form a binding pocket. In some embodiments, a binding pocket comprises a bulge, or a mutation, or a stem-loop, or any combinations thereof. In some embodiments, a binding pocket may not comprise a bulge, a mutation, or a stem-loop.

### Pre-mRNA Mutations and Mis-Splicing

Mutations in cis-acting elements of splicing can alter splicing patterns. Common mutations can be found in the core consensus sequences, including 5′ss, 3′ss, and BP regions, or other regulatory elements, including ESE, ESS, ISE, and ISS. Mutations in these cis-acting elements can result in multiple diseases. Exemplary diseases are included in Tables 1-3. The present disclosure provides methods to screen small molecule binding agents that can target pre-mRNA containing one or more mutations in the cis-acting elements. In some embodiments, the present disclosure provides methods to screen small molecule binding agents that can target pre-mRNA containing one or more mutations in the splice sites or BP regions. In some embodiments, the present disclosure provides methods to screen small molecule binding agents that can target pre-mRNA containing one or more mutations in other regulatory elements, for example, ESE, ESS, ISE, and ISS.

Mutations in cis-acting elements, and upstream mis-signaling, can induce 3-dimensional structural change in pre-mRNA. Mutations in cis-acting elements and upstream mis-signaling can induce 3-dimensional structural change in pre-mRNA when the pre-mRNA is bound to at least one snRNA, or at least one snRNP, or at least one other auxiliary splicing factor. In some embodiments, a binding pocket can be formed when the 5′ss is bound to U1 snRNA or a portion thereof. A binding pocket can contain a bulge, a non-mutation single-stranded or duplex RNA, a stem-loop, or sequences adjacent to a stem-loop, mutation-containing single and duplex RNA. A binding pocket may or may not comprise a mutation. In some cases, a binding pocket comprises a sequence portion with a mutation upstream/downstream of the binding pocket, wherein such mutation impacts the structure of RNA at the binding pocket. In some embodiments, a bulge can be formed when the 5′ss is bound to U1 snRNA or a portion thereof with or without other protein binding partners associated with splicing. In some embodiments, a bulge can be induced to form when 5′ss containing at least one mutation is bound to U1 snRNA or a portion thereof. In some embodiments, a mutation can induce the use of a cryptic 5′ss and create a bulge when it is bound to the U1 snRNA or a portion thereof. In some embodiments, a binding pocket can be formed when the 3′ss is bound to U2AF or a portion thereof. In some embodiments, a mutation can induce the use of a cryptic 3′ss and create a binding pocket when it is bound to the U2AF or a portion thereof. In some embodiments, a binding pocket can be formed when BP region is bound to U2 snRNA. The protein components of snRNP may or may not present to form such a binding pocket. Exemplary 5′ss sequences are summarized in Table 1. A polynucleotide in the methods disclosed herein can contain any one of the 5′ss sequences summarized in Table 1. In some embodiments, a small molecule can bind to the bulge.

In one aspect of the present disclosure, the binding pocket formed on the target polynucleotide comprises a bulge. In some embodiments, a bulge is naturally occurring. In some embodiments, a bulge is formed by non-canonical base-pairing between the splice site and the small nuclear RNA. For example, a bulge can be formed by non-canonical base-pairing between the 5′ss and any one of the U1-U12 snRNAs. The bulge can comprise 1 nucleotide, 2 nucleotide, 3 nucleotide, 4 nucleotide, 5 nucleotide, 6 nucleotide, 7 nucleotide, 8 nucleotide, 9 nucleotide, 10 nucleotide, 11 nucleotide, 12 nucleotide, 13 nucleotide, 14 nucleotide, or 15 nucleotide.

In some embodiments, 3-dimensional structural changes can be induced by a mutation or a mis-signaling upstream without bulge formation. In some embodiment, a bulge may be formed without any mutation in a splice site. More exemplary 5′ss mutations with or without bulge formation are summarized in Table 1. A polynucleotide in the methods disclosed herein can contain any one of the 5′ss sequences summarized in Table 1. In some embodiments, a recognition portion can be formed by a mutation in any of the cis-acting elements. In some embodiments, a small molecule can bind to a binding pocket that is induced by a mutation.

In some embodiments, a mutation in authentic 5′ss can activate usage of cryptic 5′ss during splicing. Exemplary mutated authentic 5′ss targets and corresponding activated cryptic splice site targets are summarized in Table 2.

In some embodiments, a mutation can be in one of the regulatory elements including ESE, ESS, ISE, and ISS.

In some embodiments, a target polynucleotide comprises a splice site, wherein the splice site comprises a sequence selected from the group consisting of NGAgunvrn, NHAdddddn, NNBnnnnnn, and NHAddmhvk; wherein N (or n) is A, U, G or C; B is C, G, or U; H is A, C, or U; d is a, g, or u; m is a or c; r is a or g; v is a, c or g; k is g or t.

In some embodiments, the target polynucleotide comprises a splice site, wherein the splice site comprises a sequence selected from the group consisting of NNBgunnnn, NNBhunrmn, or NNBgvnrmn, wherein N/n is A, U, G or C; B is C, G, or U; h is a, c, or t; v is a, c or g.

In some embodiments, the target polynucleotide comprises a splice site, wherein the splice site comprises a sequence selected from the group consisting of NNBgtrrm, NNBgtwwdn, NNBgtvmvn, NNBgtvbbn, NNBgtkddn, NNBgtbnbd, NNBhtnngn, NNBhtrmhd, or NNBgvdnvn, wherein N/n is A, U, G or C; B is C, G, or U; h is a, c, or u; v is a, c or g; r is a or g; m is a or c; d is a, g or u; k is g or u; w is a or u.

TABLE 1
Exemplary 5′ ss sequences and mutations
Splice Site Targets

| | | | | | | ΔG^{WT-} |
|---|---|---|---|---|---|---|
| | | | | | | ^{MUT}_{U1-bind} |
| | | | | | | (G^{WT}U1- |
| | | Splice Site | | | Mutation | bind- |
| Gene | Disease | Sequence | Description | Exon | Location | G^{MUT}_{U1-bind}) |
| ABCA4 | | GAGguaaag | Non-mutated 5′ bulge | 3 | | |
| ABCA4 | | CGGguaugg | Non-mutated 5′ bulge | 4 | | |
| ABCA4 | | AGUguaagc | Non-mutated 5′ bulge | 13 | | |
| ABCA4 | | CCAguaaac | IVS20 + 5G > A | 20 | +5 | |
| ABCA4 | | CAGgugcac | IVS28 + 5G > A | 28 | +5 | |
| ABCA4 | | AUGguacau | IVS40 + 5G > A | 40 | +5 | |
| ABCB4 | | AGAguaggu | Non-mutated 5′ bulge | 6 | | |
| ABCB4 | | AAGguacug | Non-mutated 5′ bulge | 11 | | |
| ABCB4 | | GGAguaggu | Non-mutated 5′ bulge | 20 | | |
| ABCD1 | X-linked | GAAguggg | IVS1 − 1G > A | 1 | −1 | |
| | adrenoleukodystrophy | | | | | |
| | (X-ALD) | | | | | |
| ACADM | Medium-chain | AAGguaaau | IVS7 + 6G > U | | | −1.1 |
| | acyl-coA DH | | Mutated 5′ bulge | | | |
| | deficiency | | | | | |
| ACADSB | | GGGgugcau | IVS3 + 3A > G | 3 | +3 | |
| ADA | | CCAgugaga | IVS5 + 6U > A | 5 | +6 | |
| ADAMTS | Thrombotic | AGGguagac | IVS13 + 5G > A | 13 | +5 | |
| 13 | thrombocytopenic | | | | | |
| | purpura | | | | | |
| AGL | | GGCguaagu | Non-mutated 5′ bulge | 1 | | |
| AGL | Glycogen Storage | CUGguauga | IVS6 + 3A > G | 6 | +3 | |
| | Disease Type III | | | | | |
| AGL | | AAGguagug | Non-mutated 5′ bulge | 28 | | |
| AGL | | AGAguaagu | Non-mutated 5′ bulge | 31 | | |
| ALB | Analbuminemia | AACaugagga | c.1652 + 1 G > A | 12 | +1 | |
| ALDH3A2 | | CAGgucuggu | Non-mutated 5′ bulge | 2 | | |
| ALDH3A2 | | AAGguuuau | IVS5 + 5G > A | 5 | +5 | |
| ALG6 | | UGUguaaau | IVS3 + 5G > A | 3 | +5 | |
| APC | | CAAguaugu | IVS9 + 3A > G | 9 | +3 | |
| APC | | CAAguauuu | IVS9 + 5G > U | 9 | +5 | |
| APC | | CAGguauau | IVS14 + 3A > G | 14 | +3 | |
| APOB | | AGAguaagu | Non-mutated 5′ bulge | 13 | | |
| APOB | Homozygous | AAGgcaaaa | IVS24 + 2 U > C | 24 | +2 | |
| | hypobetalipopro- | | | | | |
| | teinemia | | | | | |
| AR | Androgen | CUGuuaag | IVS4 + 1G > U | 4 | +1 | |
| | Sensitivity | | | | | |
| AR | | UUAguaaau | IVS6 + 5G > A | 6 | +5 | |
| ATM | | AAGguagua | Non-mutated 5′ bulge | 2 | | |
| ATM | | UAGguauau | IVS7 + 5\hat{ }dG > A | 7 | +5\hat{ }d | |
| ATM | | CAGguacag | Non-mutated 5′ bulge | 8 | | |
| ATM | | UUGguaaag | Non-mutated 5′ bulge | 9 | | |
| ATM | | AAGguuuaa | IVS9 + 3A > U | 9 | +3 | |
| ATM | | AUCguuaga | IVS21 + 3A > U | 21 | +3 | |
| ATM | | AUCgguaaaa | IVS21 + 5\hat{ }dG > A | 21 | +5d | |
| ATM | | AAGgucucu | Non-mutated 5′ bulge | 35 | | |
| ATM | | GAGguaaugu | Non-mutated 5′ bulge | 38 | | |
| ATM | Ataxia- | CAGauaacu | IVS45 + 1G > A | 45 | +1 | |
| | telangiectasia | | | | | |
| ATM | | GAGguaaag | Non-mutated 5′ bulge | 61 | | |
| ATP7A | | AAGguaaugu | Non-mutated 5′ bulge | 3 | | |
| ATP7A | Occipital Horn | GUUguaaau | IVS6 + 5G > A | 6 | +5 | |
| | Syndrome | | | | | |
| ATP7A | Menkes Disease | GUUauaagu | IVS6 + 1G > A | 6 | +1 | |
| ATP7A | | AAGguaaag | Non-mutated 5′ bulge | 10 | | |
| ATP7A | Occipital horn | AAGguuaag | IVS10 + 3A > U | 10 | +3 | 0 |
| | syndrome | | Mutated 5′ bulge | | | |
| ATP7A | Menkes Disease | CAGgucuuu | IVS11 + 3A > C (mouse | 11 | +3 | |
| | | | model), consistent with | | | |
| | | | patient | | | |
| ATP7A | | CAAguaaac | IVS17 + 5G > A | 17 | +5 | |
| ATP7A | | CUGguuugu | IVS21 + 3A > U | 21 | +3 | |
| ATR | | CAGguaung | Non-mutated 5′ bulge | 19 | | |
| ATR | | CAGgucuga | Non-mutated 5′ bulge | 28 | | |
| B2M | | AGCgugagu | Non-mutated 5′ bulge | 1 | | |
| BMP2K | Cancer target | CAAguaagg | Mutation inducing loss | 14 | | |
| | | | of U1snRNA affinity | | | |
| BRCA1 | Breast Cancer | UGGguaaag | Non-mutated 5′ bulge | 1 | | |
| BRCA1 | Breast Cancer | AAGguguau | IVS5 + 3A > G | 5 | +3 | |
| BRCA1 | Breast Cancer | AGGguauau | IVS5 - 2A > G | 5 | −2 | |
| BRCA1 | Breast Cancer | AAGgugugc | IVS13 + 6U > C | 13 | +6 | |
| BRCA1 | Breast Cancer | UUUgugagc | IVS16 + 6U > C | 16 | +6 | |
| BRCA1 | Breast Cancer | UCUguaaau | IVS18 + 5G > A | 18 | +5 | |
| BRCA1 | | ACAguaaau | IVS22 + 5G > A | 22 | +5 | |
| BRCA2 | Breast Cancer | CAGguguga | IVS5 + 3A > G | 5 | +3 | |
| BRCA2 | | UAGguauug | Non-mutated 5′ bulge | 14 | | |
| BRCA2 | | CAGguauga | Non-mutated 5′ bulge | 19 | | |
| BTK | | AAGguggua | Non-mutated 5′ bulge | 2 | | |
| BTK | | GAAguaaac | IVS6 + 5G > A | 6 | +5 | |
| BTK | | GAUgugagg | IVS14 + 6U > G | 14 | +6 | |
| C3 | Hereditary C3 | UGGauaagg | IVS18 + 1G > A | 18 | +1 | |
| | deficiency | | | | | |
| CAT | | UUGguagau | IVS4 + 5G > A | 4 | +5 | |
| CD46 | atypical hemolytic | AAGguaucu | Non-mutated | 13 | | |
| | uremic syndrome | | | | | |
| | (aHUS) | | | | | |
| CDH1 | | CAGguggau | IVS14 + 5G > A | 14 | +5 | |
| CDH23 | | ACGgugaac | IVS51 + 5G > A | 51 | +5 | |
| CDH23 | | AGCguaagg | Non-mutated 5′ bulge | 54 | | |
| CFTR | Cystic Fibrosis | CAUguaau | −1G > U | | | −5.4 |
| | | | Mutated 5′ bulge | | | |
| CFTR | Cystic Fibrosis | AAAguaug | −1G > A | | | −4.6 |
| | | | Mutated 5′ bulge | | | |
| CFTR | Cystic Fibrosis | AAGuuaaua | IVS4 + 1G > U | 4 | +1 | |
| CFTR | Cystic Fibrosis | ACAguuagu | IVS6b + 3\hat{ }d | 6b | +3\hat{ }d | |
| CFTR | | CAGguaaugu | Non-mutated 5′ bulge | 8 | | |
| CFTR | Cystic Fibrosis | AAAguaugu | c.1766 − 1G > A | 12 | −1 | |
| CFTR | Cystic Fibrosis | AAUguaugu | c.1766 − 1G > U | 12 | −1 | |
| CFTR | | AAGguauuu | IVS12 + 5G > U | 12 | +5 | |
| CFTR | Cystic Fibrosis | AAGgugugu | c.1766 + 3A > G | 12 | +3 | |
| CFTR | Cystic Fibrosis | AAGgucugu | c.1766 + 3A > C | 12 | +3 | |
| CFTR | Cystic Fibrosis | AAGguauga | Non-mutated 5′ bulge | 19 | | |
| CFTR | Cystic Fibrosis | CACgugagc | IVS21 − 1G > C | 20 | −1 | |
| CHM | | UAGgucaga | IVS13 + 3A > C | 13 | +3 | |
| CLCN1 | Myotonia | CAGguuaag | IVS1 + 3A > U | | | 0 |
| | congenita | | Mutated 5′ bulge | | | |
| COL11A1 | | GAGguaauac | Non-mutated 5′ bulge | 7 | | |
| COL11A1 | | AGCguaagu | Non-mutated 5′ bulge | 8 | | |
| COL11A1 | | AGAguaagu | Non-mutated 5′ bulge | 29 | | |
| COL11A1 | | AAGguauca | Non-mutated 5′ bulge | 34 | | |
| COL11A1 | | GGCguaagu | Non-mutated 5′ bulge | 50 | | |
| COL11A1 | | GGCgucagu | IVS50 + 3A > C | 50 | +3 | |
| COL11A1 | | GGAguaagu | Non-mutated 5′ bulge | 64 | | |
| COL11A2 | | CCUgugaau | IVS53 + 5G > A | 53 | +5 | |
| COL1A1 | | GGAguaagu | Non-mutated 5′ bulge | 5 | | |
| COL1A1 | Severe type III | UCAguaaac | IVS8 + 5G > A | 8 | +5 | |
| | osteogenesis | | | | | |
| | imperfecta | | | | | |
| COL1A1 | Severe type III | CCUaugagu | IVS8 + 1G > A | 8 | +1 | |
| | osteogenesis | | | | | |
| | imperfecta | | | | | |
| COL1A1 | | AGAgugagu | Non-mutated 5′ bulge | 11 | | |
| COL1A1 | | GCUguaaau | IVS14 + 5G > A | 14 | +5 | |
| COL1A1 | | AGCgugagu | Non-mutated 5′ bulge | 19 | | |
| COL1A1 | | AGAguaagu | Non-mutated 5′ bulge | 30 | | |
| COL1A2 | Osteogenesis | AGAguagau | IVS21 + 5G > A | 21 | +5 | −3.3 |
| | imperfecta | | Mutated 5′ bulge | | | |
| COL1A2 | | GAUguaaau | IVS9 + 5G > A | 9 | +5 | |
| COL1A2 | | AGAguaggu | Non-mutated 5′ bulge | 21 | | |
| COL1A2 | | AGAguaagu | Non-mutated 5′ bulge | 23 | | |
| COL1A2 | | CGGgugggu | IVS26 + 3A > G | 26 | +3 | |
| COL1A2 | | AGAguaagu | Non-mutated 5′ bulge | 30 | | |
| COL1A2 | | CGUgugaau | IVS33 + 5G > A | 33 | +5 | |
| COL1A2 | | CGUgugggu | IVS33 + 4A > G | 33 | +4 | |
| COL1A2 | | GCUguaaau | IVS40 + 5G > A | 40 | +5 | |
| COL2A1 | | GUGguugua | Non-mutated 5′ bulge | 2 | | |
| COL2A1 | | GGAguaagu | Non-mutated 5′ bulge | 7 | | |
| COL2A1 | | AGAguaagu | Non-mutated 5′ bulge | 13 | | |
| COL2A1 | | CCUgugauu | IVS20 + 5G > U | 20 | +5 | |
| COL2A1 | | UCUguaaau | IVS24 + 5G > A | 24 | +5 | |
| COL2A1 | | AGAguaagu | Non-mutated 5′ bulge | 49 | | |
| COL3A1 | Ehlers-Danlos | CCUguaagc | IVS7 + 6U > C | 7 | +6 | |
| | syndrome | | | | | |
| COL3A1 | | UCAguaaau | IVS8 + 5G > A | 8 | +5 | |
| COL3A1 | | AGAguaagu | Non-mutated 5′ bulge | 10 | | |
| COL3A1 | | GCAguuagu | IVS14 + 3G > U | 14 | +3 | |
| COL3A1 | Ehlers-Danlos | CCUauaagu | IVS16 + 1G > A | 16 | +1 | |
| | syndrome IV | | | | | |
| COL3A1 | Ehlers-Danlos | CGCauaagu | IVS20 + 1G > A | 20 | +1 | |
| | syndrome IV | | | | | |
| COL3A1 | | GAUgugauu | IVS25 + 5G > U | 25 | +5 | |
| COL3A1 | | ACUguaaau | IVS27 + 5G > A | 27 | +5 | |
| COL3A1 | | ACUguauu | IVS27 + 5G > U | 27 | +5 | |
| COL3A1 | | AAGguagua | Non-mutated 5′ bulge | 29 | | |
| COL3A1 | | GCUguaauu | IVS37 + 5G > U | 37 | +5 | |
| COL3A1 | | CCUguaaau | IVS38 + 5G > A | 38 | +5 | |
| COL3A1 | | CCUguaauu | IVS38 + 5G > U | 38 | +5 | |
| COL3A1 | | GAUgugacu | IVS42 + 5G > C | 42 | +5 | |
| COL3A1 | Ehlers-Danlos | GAUaugagu | IVS42 + 1G > A | 42 | +1 | |
| | syndrome IV | | | | | |
| COL3A1 | | CCUguaaau | IVS45 + 5G > A | 45 | +5 | |
| COL3A1 | | AGAguaagu | Non-mutated 5′ bulge | 46 | | |
| COL4A5 | | AGAguaagu | Non-mutated 5′ bulge | 4 | | |
| COL4A5 | | AGAguaagu | Non-mutated 5′ bulge | 15 | | |
| COL4A5 | | AAGgucuggg | Non-mutated 5′ bulge | 28 | | |
| COL4A5 | | CAGgugcug | Non-mutated 5′ bulge | 39 | | |
| COL4A5 | | CAGguaaag | Non-mutated 5′ bulge | 52 | | |
| COL6A1 | Mild Bethlem | GGGaugagu | IVS3 + 1G > A | 3 | +1 | |
| | myopathy | | | | | |
| COL6A3 | | AAGguaugg | Non-mutated 5′ bulge | 4 | | |
| COL6A3 | | CAGguaugg | Non-mutated 5′ bulge | 6 | | |
| COL6A3 | | AAGguaegg | Non-mutated 5′ bulge | 14 | | |
| COL6A3 | | AAAguacau | IVS29 + 5G > A | 29 | +5 | |
| COL6A3 | | AGUguaagu | Non-mutated 5′ bulge | 38 | | |
| COL7A1 | Recessive | AGGgugauc | IVS3 − 2A > G | 3 | −2 | |
| | dystrophic | | | | | |
| | epidermolysis | | | | | |
| | bullosa | | | | | |
| COL7A1 | | CAGguauag | Non-mutated 5′ bulge | 23 | | |
| COL7A1 | | CAGguuugg | Non-mutated 5′ bulge | 24 | | |
| COL7A1 | | CAGguuugg | Non-mutated 5′ bulge | 27 | | |
| COL7A1 | Dominant | AGGgugagg | Exon73 del[−98: −71] | 73 | del[−98: −71] | |
| | dystrophic | | | | | |
| | epidermolysis | | | | | |
| | bullosa | | | | | |
| COL7A1 | Recessive | GUAgugagu | IVS95 − 1G > A | 95 | −1 | |
| | dystrophic | | | | | |
| | epidermolysis | | | | | |
| | bullosa | | | | | |
| COL9A2 | | CCGgugagg | IVS3 + 6U > G | 3 | +6 | |
| COL9A2 | | CCGgugacu | IVS3 + 5G > C | 3 | +5 | |
| COLQ | Congenital | UGGguggggg | IVS16 + 3A > G | 16 | +3 | |
| | acetylcholinesterase | | | | | |
| | deficiency | | | | | |
| CREBBP | Rubinstein-Taybi | AAGguuca | +3A > U | | +3 | −0.5 |
| | syndrome | | Mutated 5′ bulge | | | |
| CSTB | Epilepsy: | AAAguaga | −1G > A | | −1 | −4.6 |
| | progressive myoclonus | | Mutated 5′ bulge | | | |
| CUL4B | | CAGguaaaa | Non-mutated 5′ bulge | 14 | | |
| CYBB | | GGGguaaau | IVS2 + 5G > A | 2 | +5 | |
| CYBB | | GCGguaaaa | IVS3 + 5G > A | 3 | +5 | |
| CYBB | | AAGguuagc | IVS5 + 3A > U | 5 | +3 | |
| CYBB | | UGAgugaau | IVS6 + 5G > A | 6 | +5 | |
| CYP17 | | UCAgugauu | IVS2 + 5G > U | 2 | +5 | |
| CYP17 | | CUGgugaau | IVS7 + 5G > A | 7 | +5 | |
| CYP19 | Placental | UGUgcaagu | IVS6 + 2U > C | 6 | +2 | |
| | aromatase | | | | | |
| | deficiency | | | | | |
| CYP27 | | AACgugauu | IVS7 + 5G > U | 7 | +5 | |
| CYP27A1 | Cerebrotendinous | GAGguagga | IVS6 - 2C > A | 6 | -2 | |
| | xanthomatosis | | | | | |
| CYP27A1 | Cerebrotendinous | GCAguagga | IVS6 − 1G > A | 6 | −1 | |
| | xanthomatosis | | | | | |
| DES | | GAGguguac | IVS3 + 3A > G | 3 | +3 | |
| DMD | | GAUguaagu | Non-mutated 5′ bulge | 5 | | |
| DMD | | CAGguaaag | Non-mutated 5′ bulge | 8 | | |
| DMD | | CAGgugugu | Non-mutated 5′ bulge | 14 | | |
| DMD | | AUGgucauu | IVS19 + 3A > C | 19 | +3 | |
| DMD | | AGAguaaga | Non-mutated 5′ bulge | 24 | | |
| DMD | Duchenne and | AAGggaaaa | IVS26 + 2U > G | 26 | +2 | |
| | Becker muscular | | | | | |
| | dystrophy | | | | | |
| DMD | | CAGguauau | c.4250U > A | 31 | | |
| DMD | | CAGguauau | Non-mutated 5′ bulge | 31 | | |
| DMD | | CAAguaacu | IVS62 + 5G > C | 62 | +5 | |
| DMD | | GCUguaacu | IVS64 + 5G > C | 64 | +5 | |
| DMD | Duchenne and | GCUguaacu | IVS64 + 5G > C | 64 | +5 | |
| | Becker muscular | | | | | |
| | dystrophy | | | | | |
| DMD | | GAUguaauu | IVS66 + 5G > U | 66 | +5 | |
| DMD | | CCGguaacu | IVS69 + 5G > C | 69 | +5 | |
| DMD | | AACgugacu | IVS70 + 5G > C | 70 | +5 | |
| DYSF | | AGAgugcgu | Non-mutated 5′ bulge | 13 | | |
| DYSF | | UGUguacau | IVS45 + 5G > A | 45 | +5 | |
| EGFR | Cancer target | AACguaagu | | 4 | | |
| EGFR | | ACAguuuga | Non-mutated 5′ bulge | 9 | | |
| EGFR | | GUGgugagu | Non-mutated 5′ bulge | 22 | | |
| EMD | | UAGguaccc | IVS1 + 5G > C | 1 | +5 | |
| ETV4 | Ovarian Cancer | GAGcugcag | Non-mutated 5′ bulge | 5 | | |
| F13A1 | | UUGgugagc | IVS3 + 6G > U | 3 | +6 | |
| F13A1 | | UUGgugaau | IVS3 + 5G > A | 3 | +5 | |
| F5 | | AAGguaacu | Non-mutated 5′ bulge | 1 | | |
| F5 | Severe factor V | CAUguauuu | IVS10 − 1G > U | 10 | −1 | |
| | deficiency | | | | | |
| F5 | | AAGguuugg | Non-mutated 5′ bulge | 13 | | |
| F5 | | UGGguuagu | IVS19 + 3A > U | 19 | +3 | |
| F5 | | AAGgucaag | Non-mutated 5′ bulge | 23 | | |
| F5 | | AAGguagag | Non-mutated 5′ bulge | 24 | | |
| F7 | FVII deficiency | UGGguggau | IVS7 + 5G > A | 7 | +5 | |
| F7 | FVII deficiency | UGGgugggug | IVS7 + 7A > G | 7 | +7 | |
| F7 | FVII deficiency | UGGguacca | IVS7del[+3: +6] | 7 | del[+3: +6] | |
| F8 | | AGGgugaau | IVS3 + 5G > A | 3 | +5 | |
| F8 | | CAGgugugu | IVS6 + 3A > G | 6 | +3 | |
| F8 | | CAGguguga | IVS14 + 3A > G | 14 | +3 | |
| F8 | | AUAgugaau | IVS19 + 5G > A | 19 | +5 | |
| F8 | | AUGguauuu | IVS22 + 5G > U | 22 | +5 | |
| F8 | | AUAgucagu | IVS23 + 3A > C | 23 | +3 | |
| FAH | | AAGguaugu | Non-mutated 5′ bulge | 11 | | |
| FAH | Tyrosinemia type | CCGgugaau | IVS12 + 5G > A | 12 | +5 | |
| | I, Chronic | | | | | |
| | Tyrosinemia Type | | | | | |
| | I | | | | | |
| FANCA | | AGAguaaga | Non-mutated 5′ bulge | 4 | | |
| FANCA | | AAGguagcg | Non-mutated 5′ bulge | 6 | | |
| FANCA | Fanconi Anemia | CUGgugcau | IVS7 + 5G > A | 7 | +5 | |
| FANCA | | CUGgugcuu | IVS7 + 5G > U | 7 | +5 | |
| FANCA | | GAGgugcug | Non-mutated 5′ bulge | 10 | | |
| FANCA | | CGAguccgu | IVS16 + 3A > C | 16 | +3 | |
| FANCC | | AAUgugugu | IVS4 + 4A > U | 4 | +4 | |
| FANCG | | CAGgugaua | IVS4 + 3A > G | 4 | +3 | |
| FBN1 | Marfan Syndrome | UUGguacau | IVS11 + 5G > A | 11 | +5 | |
| FBN1 | | GAGguaugg | Non-mutated 5′ bulge | 13 | | |
| FBN1 | | AAGguaauaa | Non-mutated 5′ bulge | 14 | | |
| FBN1 | | CAGgucaau | IVS25 + 5G > A | 25 | +5 | |
| FBN1 | Marfan Syndrome | CAUguaanu | IVS37 + 5G > U | 37 | +5 | |
| FBN1 | Marfan Syndrome | UAGgugcau | IVS46 + 5G > A | 46 | +5 | |
| FBN1 | Marfan syndrome | UAGaugcgu | IVS46 + 1G > A | 46 | +1 | |
| FBN1 | | AAGguaaag | Non-mutated 5′ bulge | 60 | | |
| FECH | Protoporphyria: | UAGguauc | −3A > U | | | 0 |
| | erythropoietic | | Mutated 5′ bulge | | | |
| FECH | | GAGguanga | Non-mutated 5′ bulge | 2 | | |
| FECH | | CAGguaugg | Non-mutated 5′ bulge | 4 | | |
| FECH | | AAGgugucu | IVS10 + 3A > G | 10 | +3 | |
| FECH | | AAGguaucu | Non-mutated 5′ bulge | 10 | | |
| FGA | | UGGgugugg | IVS1 + 3A > G | 1 | +3 | |
| FGA | Common | GAGuuaagu | IVS4 + 1G > U | 4 | +1 | |
| | congenital | | | | | |
| | afibrinogenemia | | | | | |
| FGFR2 | | AGAguaagu | Non-mutated 5′ bulge | 3 | | |
| FGFR2 | | CAGguguau | IVS3c + 3A > G | 3c | +3 | |
| FGG | | GCAguaaau | IVS1 + 5G > A | 1 | +5 | |
| FGG | | CAAgugaaa | IVS3 + 5G > A | 3 | +5 | |
| FIX | Haemophilia B | CGGgucauaauc | c.519A > G | 5 | -2 | |
| | deficiency | | | | | |
| | (coagulation factor | | | | | |
| | IX deficiency) | | | | | |
| FLNA | | AGAguaagu | Non-mutated 5′ bulge | 19 | | |
| FOXM1 | | AAGguaaugu | Non-mutated 5′ bulge | 4 | | |
| FOXM1 | Cancer target | UCAguaagu | | 9 | | |
| FRAS1 | | AAGguacgg | Non-mutated 5′ bulge | 3 | | |
| FRAS1 | | GGAgugagu | Non-mutated 5′ bulge | 5 | | |
| FRAS1 | | AAGguauuu | Non-mutated 5′ bulge | 8 | | |
| FRAS1 | | AAGguaucg | Non-mutated 5′ bulge | 17 | | |
| FRAS1 | | AGCguaggu | Non-mutated 5′ bulge | 22 | | |
| FRAS1 | | AGAguaagu | Non-mutated 5′ bulge | 24 | | |
| FRAS1 | | CAGguacaa | Non-mutated 5′ bulge | 53 | | |
| GALC | | GGAguuagu | Non-mutated 5′ bulge | 5 | | |
| GH1 | | UCCgugagc | IVS3 + 6U > C | 3 | +6 | |
| GH1 | | UCCgugaau | IVS3 + 5G > A | 3 | +5 | |
| GH1 | | UCCgugacu | IVS3 + 5G > C | 3 | +5 | |
| GH1 | | GGGgugacg | IVS4 + 5G > C | 4 | +5 | |
| GH1 | | GGGgugacg | IVS4 + 5G > A | 4 | +5 | |
| GHV | Mutation in | UUUauaagc | IVS2 + 1G > A | 2 | +1 | |
| | placenta | | | | | |
| HADHA | | AAGgugucu | IVS3 + 3A > G | 3 | +3 | |
| HADHA | | AGUguaagu | Non-mutated 5′ bulge | 18 | | |
| HBA2 | Alpha-thalassemia | GAGgcuccc | IVS1 del[+2: +6] | 1 | del[+2: +6] | |
| HBB | Beta-thalassemia | CAGguuguu | IVS1 + 5G > U | 1 | +5 | |
| HBB | Beta-thalassemia | CACguuggu | IVS1−1G > C | 1 | −1 | |
| HBB | Beta-thalassemia | CAGguuggc | IVS1 + 6U > C | 1 | +6 | |
| HBB | Beta-thalassemia | CAGauuggu | IVS1 + 1G > A | 1 | +1 | |
| HBB | Beta-thalassemia | CAGuuuggu | IVS1 + 1G > U | 1 | +1 | |
| HBB | Beta-thalassemia | CAGgcuggu | IVS1 + 2U > C | 1 | +2 | |
| HBB | Beta-thalassemia | CAGguugau | IVS1 + 5G > A | 1 | +5 | |
| HBB | Beta-thalassemia | CAGguugcu | IVS1 + 5G > C | 1 | +5 | |
| HBB | Beta-thalassemia | AGGgugucu | IVS2 del[+4: +5] | 2 | del[+4: +5] | |
| HEXA | | ACAguaaau | IVS4 + 5G > A | 4 | +5 | |
| HEXA | | CUGguguga | IVS8 + 3A > G | 8 | +3 | |
| HEXA | Tay-Sachs | GACaugagg | IVS9 + 1G > A | 9 | +1 | |
| | Syndrome | | | | | |
| HEXB | Sandhoff disease | UUGguaaca | IVS8 + 5G > C | 8 | +5 | |
| HLCS | | AAGgucaau | IVS10 + 5G > A | 10 | +5 | |
| HMBS | | GCGguuagu | IVS1 + 3G > U | 1 | +3 | |
| HMBS | | GCGgugacu | IVS1 + 5G > C | 1 | +5 | |
| HMGCL | Hereditary HL | ACGcuaagc | IVS7 + 1G > C | 7 | +1 | |
| | deficiency | | | | | |
| HNF1A | | AGCguaagu | Non-mutated 5′ bulge | 2 | | |
| HPRT1 | Somatic mutations | GUGgugagc | IVS1del[-2: +34] | 1 | del[−2: +34] | |
| | in kidney tubular | | | | | |
| | epithelial cells | | | | | |
| HPRT1 | Somatic mutations | GUGgugauc | IVS1 + 5G > U | 1 | +5 | |
| | in kidney tubular | | | | | |
| | epithelial cells | | | | | |
| HPRT1 | Lesch-Nyhan | GAAggaagu | IVS5 + 2U > G | 5 | +2 | |
| | syndrome | | | | | |
| HPRT1 | Lesch-Nyhan | GAAgugugu | IVS5 + 3: 4AA > GU | 5 | +3 | |
| | syndrome | | | | | |
| HPRT1 | Lesch-Nyhan | GAAguaaau | IVS5 + 5G > A | 5 | +5 | |
| | syndrome | | | | | |
| HPRT1 | Lesch-Nyhan | GAAuaaguu | IVS5del[G1] | 5 | del[1] | |
| | syndrome | | | | | |
| HPRT1 | | ACUguaaau | IVS7 + 5G > A | 7 | +5 | |
| HPRT1 | | ACUguaacu | IVS7 + 5G > C | 7 | +5 | |
| HPRT1 | Hypoxanthine | AAUguaagc | IVS8 + 6U > C | 8 | +6 | |
| | phosphoribosyltran | | Mutation inducing loss | | | |
| | sferase deficiency | | of U1snRNA affinity | | | |
| HPRT1 | Hypoxanthine | AAUguaagg | IVS8 + 6U > G | 8 | +6 | |
| | phosphoribosyltran | | | | | |
| | sferase deficiency | | | | | |
| HPRT1 | | AAUguaaau | IVS8 + 5G > A | 8 | +5 | |
| HPRT1 | | AAUguaauu | IVS8 + 5G > U | 8 | +5 | |
| HPRT2 | Primary | GGGauaagu | IVS1 + 1G > A | 1 | +1 | |
| | Hyperthyroidism | | | | | |
| HSF4 | | CAGguagug | IVS12 + 4A > G | 12 | +4 | |
| HSPG2 | | AGAgugagu | Non-mutated 5′ bulge | 30 | | |
| HSPG2 | | AGAguaagu | Non-mutated 5′ bulge | 40 | | |
| HSPG2 | | CAGguacag | Non-mutated 5′ bulge | 61 | | |
| HTT | | CAGguacug | Non-mutated 5′ bulge | 25 | | |
| HTT | | AAGguaaau | Non-mutated 5′ bulge | 32 | | |
| HTT | | AGAguaagu | Non-mutated 5′ bulge | 51 | | |
| IDS | | AUGguaacc | IVS7 + 5G > C | 7 | +5 | |
| IDS | Mucopolysaccharidosis | AUUuuaagc | IVS7−1: +1GG > UU | 7 | −1 | |
| | type II | | | | | |
| | (Hunter syndrome) | | | | | |
| IKBKAP | Familial | CAAguaagc | IVS20 + 6U > C | 20 | +6 | |
| | Dysautonomia | | Mutation inducing loss | | | |
| | | | of U1snRNA affinity | | | |
| IKBKAP | | CAGguaugu | Non-mutated 5′ bulge | 27 | | |
| IKBKAP | | AGCguacgu | Non-mutated 5′ bulge | 33 | | |
| INSR | Breast Cancer | GGCguaagu | Non-mutated 5′ bulge | 7 | | |
| INSR | | AGUguaagu | Non-mutated 5′ bulge | 20 | | |
| ITGB2 | Leukocyte | UUCauaagu | IVS7 + 1G > A | 7 | +1 | |
| | adhesion | | | | | |
| | deficiency | | | | | |
| ITGB3 | Glanzmann | GAUaugagu | IVS4 + 1G > A | 4 | +1 | |
| | thrombasthenia | | | | | |
| ITGB4 | | GAGgugccu | Non-mutated 5′ bulge | 4 | | |
| ITGB4 | | CAGguagua | Non-mutated 5′ bulge | 33 | | |
| JAG1 | | CGGgugugu | IVS11 + 3A > G | 11 | +3 | |
| JAG1 | | AGAgugagu | Non-mutated 5′ bulge | 18 | | |
| KRAS | Cancer target | CAGguaagu | Splice switching on | 4a | | |
| | | | isoforms | | | |
| KRT5 | Dowling-Meara | AAGaugagc | IVS1 + 1G > A | 1 | +1 | |
| | epidermolysis | | | | | |
| | bullosa simplex | | | | | |
| L1CAM | | AAUgugagu | Non-mutated 5′ bulge | 2 | | |
| L1CAM | | AGAguaaga | Non-mutated 5′ bulge | 14 | | |
| L1CAM | | CAGgugagc | Non-mutated 5′ bulge | 27 | | |
| LAMA2 | Muscular | GAGgugca | +3A > G | | | −0.1 |
| | dystrophy: | | Mutated 5′ bulge | | | |
| | merosin deficient | | | | | |
| LAMA3 | | CAGguaaag | Non-mutated 5′ bulge | 16 | | |
| LAMA3 | | AAGguaaugu | Non-mutated 5′ bulge | 26 | | |
| LAMA3 | | CAGguagug | Non-mutated 5′ bulge | 27 | | |
| LAMA3 | | AGCguaagu | Non-mutated 5′ bulge | 31 | | |
| LAMA3 | | CAGguaccg | Non-mutated 5′ bulge | 40 | | |
| LAMA3 | | AAGguaaugu | Non-mutated 5′ bulge | 45 | | |
| LAMA3 | | AGAgugagu | Non-mutated 5′ bulge | 50 | | |
| LAMA3 | | GAGguacaa | Non-mutated 5′ bulge | 57 | | |
| LAMA3 | | UGGguaugc | Non-mutated 5′ bulge | 64 | | |
| LDLR | Familial | GAGgcgugg | IVS12 + 2U > C | 12 | +2 | |
| | hypercholesterolemia | | | | | |
| LMNA | Hutchinson- | CAGgugggu | 1824C > U | | | |
| | Gilford progeria | (crypuic) | Cryptic splice site | | | |
| | syndrome (HGPS) | | activated by mutation | | | |
| | | | not in authentic ss | | | |
| LMNA | Hutchinson- | CAGgugagc | 1822G > A | | | |
| | Gilford progeria | (crypuic) | Cryptic splice site | | | |
| | syndrome (HGPS) | | activated by mutation | | | |
| | | | not in authentic ss | | | |
| LMNA | Hutchinson- | CAGguggac | 1823G > A | | | |
| | Gilford progeria | (crypuic) | Cryptic splice site | | | |
| | syndrome (HGPS) | | activated by mutation | | | |
| | | | not in authentic ss | | | |
| LMNA | Hutchinson- | CAGguaggc | 1821G > A | | | |
| | Gilford progeria | (crypuic) | Cryptic splice site | | | |
| | syndrome (HGPS) | | activated by mutation | | | |
| | | | not in authentic ss | | | |
| LMNA | Hutchinson- | ACGgucagu | 1868C > G | | | |
| | Gilford progeria | (crypuic) | Cryptic splice site | | | |
| | syndrome (HGPS) | | activated by mutation | | | |
| | | | not in authentic ss | | | |
| LMNA | Hutchinson- | CAAgugagu | c.1968−1G > A | 10 | +1 | |
| | Gilford progeria | | Mutation in 5′ss site | | | |
| | syndrome (HGPS) | | weakens site, causes | | | |
| | | | usage of cryptic splice | | | |
| | | | site | | | |
| LPL | Familial | ACGauaagg | IVS2 + 1G > A | 2 | +1 | |
| | hypercholesterolemia | | | | | |
| MADD | | AAGguacag | Non-mutated 5′ bulge | 3 | | |
| MADD | Cancer, MADD, | AAGgugggu | Non-mutated 5′ bulge | 16 | | |
| | Glioblastoma | | | | | |
| MADD | | AGAguaagg | Non-mutated 5′ bulge | 21 | | |
| MAPT | Frontotemporal | AGUguaagu | IVS10 + 3G > A | 10 | +3 | 0.1 |
| | dementia with | | Mutated 5′ bulge | | | |
| | Parkinsonism | | | | | |
| MAPT | | AGUgugagu | Non-mutated 5′ bulge | 11 | | |
| MLH1 | Colorectal cancer: | CGGguaau | −2A > G | | | −0.3 |
| | non-polyposis | | Mutated 5′ bulge | | | |
| MLH1 | Colorectal cancer: | CAAguaau | −1G > A | | | −5.4 |
| | non-polyposis | | Mutated 5′ bulge | | | |
| MLH1 | Hereditary | CAGgugcag | IVS6 + 3A > G | 6 | +3 | −0.1 |
| | nonpolyposis | | Mutated 5′ bulge | | | |
| | colorectal cancer; | | | | | |
| | Colorectal cancer: | | | | | |
| | non-polyposis | | | | | |
| MLH1 | Hereditary | CAGgugcag | IVS18 + 3A > G | 18 | +3 | |
| | nonpolyposis | | | | | |
| | colorectal cancer | | | | | |
| MLH1 | | CAGguauag | Non-mutated 5′ bulge | 4 | | |
| MLH1 | | CAGguacag | Non-mutated 5′ bulge | 6 | | |
| MLH1 | | CAGguaaugu | Non-mutated 5′ bulge | 10 | | |
| MLH1 | | CAGguacag | Non-mutated 5′ bulge | 18 | | |
| MSH2 | | AAGguaaca | Non-mutated 5′ bulge | 7 | | |
| MSH2 | | CAGguuugc | Non-mutated 5′ bulge | 10 | | |
| MST1R | Cancer, RON | CAGguaggc | Non-mutated | 11 | | |
| | tyrosine kinase, | | | | | |
| | breast and colon | | | | | |
| | tumors | | | | | |
| MTHFR | Severe deficiency | CAGaugagg | IVS4 + 1G > A | 4 | +1 | |
| | of MTHFR | | | | | |
| MUT | | AAGguauac | Non-mutated 5′ bulge | 3 | | |
| MUT | | AAGguguua | ISV8 + 3A > G | 8 | +3 | |
| MUT | | GAGguaauau | Non-mutated 5′ bulge | 10 | | |
| MVK | | CAGguaucc | Non-mutated 5′ bulge | 4 | | |
| NF1 | Neurofibromatosis, | UAGguguau | IVS11 + 3A > G | 11 | +3 | 0.2 |
| | Neurofibromatosis | | Mutated 5′ bulge | | | |
| | type I | | | | | |
| NF1 | | GGGguaacu | IVS3 + 5G > C | 3 | +5 | |
| NF1 | Neurofibromatosis | CGGguguau | IVS7 + 5G > A | 7 | +5 | |
| | type I, | | | | | |
| | Neurofibromatosis | | | | | |
| | type II | | | | | |
| NF1 | | UAGguauau | Non-mutated 5′ bulge | 15 | | |
| NF1 | | CAGguaaag | Non-mutated 5′ bulge | 21 | | |
| NF1 | Neurofibromatosis | GAGguaaga | IVS27bdel[+1: +10] | 27b | del[+1: +10] | |
| | type I | | | | | |
| NF1 | Neurofibromatosis | AAAauaagu | IVS28 + 1G > A | 28 | +1 | |
| | type I | | | | | |
| NF1 | | UAGguaaag | Non-mutated 5′ bulge | 34 | | |
| NF1 | Neurofibromatosis | CAAGguaccu | c.6724 − 4C > U | 36 | −4 | |
| NF1 | Neurofibromatosis | AAGgugccu | IVS36 + 3A > G | 36 | +3 | |
| NF2 | Neurofibromatosis | GAGgugagg | IVS12 del[−14: +2] | 12 | del[−14: +2] | |
| | type II | | | | | |
| NF2 | Neurofibromatosis | GAGaugagg | IVS12 + 1G > A | 12 | +1 | |
| | type II | | | | | |
| OAT | | CAGguuguc | Non-mutated 5′ bulge | 5 | | |
| OPA1 | | CGGguauau | IVS8 + 5G > A | 8 | +5 | |
| OTC | | GAGgugugc | IVS7 + 3A > G | 7 | +3 | |
| PAH | | CAGguguga | IVS5 + 3A > G | 5 | +3 | |
| PAH | | AGAguaagu | Non-mutated 5′ bulge | 6 | | |
| PAH | | CAGguguga | IVS10 + 3A > G | 10 | +3 | |
| PBGD | Acute intermittent | GCGaugagu | IVS1 + 1G > A | 1 | +1 | |
| | porphyria | | | | | |
| PBGD | Acute intermittent | GCGgagagu | IVS1 + 2U > A | 1 | +2 | |
| | porphyria | | | | | |
| PBGD | Acute intermittent | GCGgugacu | IVS1 + 5G > C | 1 | +5 | |
| | porphyria | | | | | |
| PBGD | Acute intermittent | GCGguuagu | IVS1 + 3G > U | 1 | +3 | |
| | porphyria | | | | | |
| PBGD | Acute intermittent | CAUguaggg | IVS10 − 1G > U | 10 | −1 | |
| | porphyria | | | | | |
| PCCA | | GGUguaagu | Non-mutated 5′ bulge | 14 | | |
| PCCA | | AAGguaugg | Non-mutated 5′ bulge | 18 | | |
| PDH1 | | AAGguacag | Non-mutated 5′ bulge | 11 | | |
| PGK1 | Phosphoglycerate | AAGuuagga | IVS4 + 1G > U | 4 | +1 | |
| | kinase deficiency | | | | | |
| PHEX | | AGAgugagu | Non-mutated 5′ bulge | 4 | | |
| PHEX | | AGAgugagu | Non-mutated 5′ bulge | 14 | | |
| PKD2 | | AGUguaagu | Non-mutated 5′ bulge | 13 | | |
| PKLR | | CAGgucugga | Non-mutated 5′ bulge | 7 | | |
| PKLR | | GCGguggga | IVS9 + 3A > G | 9 | +3 | |
| PLEKHM1 | | AGAgugagu | Non-mutated 5′ bulge | 4 | | |
| PLKR | | AGUgugagu | Non-mutated 5′ bulge | 25 | | |
| POMT2 | | GGAguaagg | Non-mutated 5′ bulge | 3 | | |
| POMT2 | | CAGguaaugu | Non-mutated 5′ bulge | 10 | | |
| POMT2 | | AGAguaagu | Non-mutated 5′ bulge | 11 | | |
| POMT2 | | AGUgugagu | Non-mutated 5′ bulge | 14 | | |
| PRDM1 | | CAGgugcgc | Non-mutated 5′ bulge | 6 | | |
| PRKAR1A | | GAGgugaag | IVS8 + 3A > G | 8 | +3 | |
| PROC | | ACAgugagg | IVS3 + 3A > G | 3 | +3 | |
| PSEN1 | | CAGguacag | Non-mutated 5′ bulge | 3 | | |
| PTCH1 | | GAGgugugu | Non-mutated 5′ bulge | 1 | | |
| PTEN | Cowden syndrome | GAGgcaggu | IVS4 + 2U > C | 4 | +2 | |
| PTEN | Cowden syndrome | AAGauuugu | IVS7 + 1G > A | 7 | +1 | |
| PYGM | Myophosphorylase | ACCaugagu | IVS14 + 1G > A | 14 | +1 | |
| | deficiency | | | | | |
| | (McArdle disease) | | | | | |
| RP6KA3 | | GAGguguau | IVS6 + 3A > G | 6 | +3 | |
| RPGR | Retinitis | CAGgugua | +3A > G | | | −0.1 |
| | pigmentosa | | Mutated 5′ bulge | | | |
| RPGR | | AAGguuugg | Non-mutated 5′ bulge | 3 | | |
| RPGR | | CAGguauag | Non-mutated 5′ bulge | 4 | | |
| RPGR | | CAGguguag | IVS4 + 3A > G | 4 | +3 | |
| RPGR | X-linked retinitis | CUGuugaga | IVS5 + 1G > U | 5 | +1 | |
| | pigmentosa (RP3) | | | | | |
| RPGR | | AGGgugcaa | IVS10 + 3A > G | 10 | +3 | |
| RSK2 | | GAGguauau | IVS6 + 3A > G | 6 | +3 | |
| SBCAD | | GGGguacau | IVS3 + 3A > G | 3 | +3 | |
| SCN5A | | GGCguaagu | Non-mutated 5′ bulge | 4 | | |
| SCN5A | | CAGgugugu | Non-mutated 5′ bulge | 8 | | |
| SERPINA1 | Risk for | AAGuuaagg | IVS2 + 1G > U | 2 | +1 | |
| | emphysema | | | | | |
| SH2D1A | Lymphoproliferative | GAUguaua | −1G > U | | | −4.9 |
| | syndrome: X- | | Mutated 5′ bulge | | | |
| | linked | | | | | |
| SLC12A3 | | GGCguaagu | Non-mutated 5′ bulge | 22 | | |
| SLC6A8 | | GGAgugagu | Non-mutated 5′ bulge | 3 | | |
| SLC6A8 | | ACGguagcu | IVS10 + 5G > C | 10 | +5 | |
| SMN2 | Spinal muscular | GGAguaagu | IVS7 + 6C > U | 7 | +6 | |
| | atrophy | | Mutation inducing loss | | | |
| | | | of U1 snRNA affinity | | | |
| SPINK5 | | CAGguaau | IVS2 + 5G > A | 2 | +5 | |
| SPINK5 | | AAGguagua | Non-mutated 5′ bulge | 20 | | |
| SPTA1 | | AAGguauau | Non-mutated 5′ bulge | 3 | | |
| SPTA1 | | CAGguagag | Non-mutated 5′ bulge | 27 | | |
| SPTA1 | | UAGguauga | Non-mutated 5′ bulge | 41 | | |
| TP53 | | GAGgucuggu | Non-mutated 5′ bulge | 5 | | |
| TP53 | Colorectal tumors | AUGgugacc | IVS5 + 5G > C | 5 | +5 | |
| TP53 | Squamous cell | GAAgucugg | IVS6 − 1G > A | 6 | −1 | |
| | carcinoma | | | | | |
| TP53 | Squamous cell | GAGaucugg | IVS6 + 1G > A | 6 | +1 | |
| | carcinoma | | | | | |
| TRAPPC2 | Spondyloepiphy seal | AAGguacgg | +4U > C | | | 0 |
| | dysplasia tarda | | Mutated 5′ bulge | | | |
| TRAPPC2 | | AAGguaugg | Non-mutated 5′ bulge | 4 | | |
| TSC1 | | AUGguaaaa | Non-mutated 5′ bulge | 9 | | |
| TSC1 | | AAGguaaugua | Non-mutated 5′ bulge | 14 | | |
| TSC2 | Tuberous sclerosis | AGAgugaau | + 5G > A | | | −4.6 |
| | | | Mutated 5′ bulge | | | |
| TSC2 | Familial tuberous | AAGgaugag | IVS37 + 2 ins [A] | 37 | +2 ins | |
| | sclerosis | | | | | |
| TSHB | | CGGguauau | IVS2 + 5G > A | 2 | +5 | |
| UGT1A1 | Crigler-Najjar | CAGcugugu | IVS1 + 1G > C | 1 | +1 | |
| | syndrome type 1 | | | | | |
| USH2A | | CAGguauug | Non-mutated 5′ bulge | 19 | | |
| USH2A | | CAGguaaugu | Non-mutated 5′ bulge | 28 | | |
| USH2A | | AAGguaaag | Non-mutated 5′ bulge | 31 | | |
| USH2A | | GGAguaagu | Non-mutated 5′ bulge | 34 | | |
| USH2A | | AGAgugagc | Non-mutated 5′ bulge | 39 | | |
| USH2A | | AUGguaugu | Non-mutated 5′ bulge | 70 | | |

TABLE 2
Exemplary mutated authentic splice site targets and corresponding activated cryptic
splice site targets
Mutated Authentic Splice Site Targets and Corresponding Activated Cryptic Splice Site Targets

| | | Mutated | | | Authentic | Cryptic Splice Site |
|---|---|---|---|---|---|---|
| | | Authentic | Authentic Splice | | Splice Site | sequence |
| | | Splice Site | Site | | Mutation | (Cryptic Splice Site |
| Gene | Disease | Sequence | Mutation | Exon | Location | Location) |
| HBB | Beta- | CACguuggu | IVS1 − 1G > C | 1 | −1 | GUGgugagg (IVS1 − 16) |
| | thalassemia | CAGguuggc | IVS1 + 6U > C | 1 | +6 | AUGguuaag (IVS2 + 48) |
| | | CAGauuggu | IVS1 + 1G > A | 1 | +1 | AAGgugaac (IVS1 − 38) |
| | | CAGuuuggu | IVS1 + 1G > U | 1 | +1 | AAGgugaag (Exon2 − 135) |
| | | CAGgcuggu | IVS1 + 2U > C | 1 | +2 | |
| | | CAGguugau | IVS1 + 5G > A | 1 | +5 | |
| | | CAGguugcu | IVS1 + 5G > C | 1 | +5 | |
| | | CAGguuguu | IVS1 + 5G > U | 1 | +5 | |
| | | AGGgugucu | IVS2 del[+4: +5] | 2 | del[+4: +5] | |
| PBGD | Acute | GCGaugagu | IVS1 + 1G > A | 1 | +1 | CGGgugggg (Exon 10 − 9) |
| | intermittent | CAUguaggg | IVS10 − 1G > U | 10 | −1 | |
| | porphyria | GCGgagagu | IVS1 + 2U > A | 1 | +2 | |
| | | GCGgugacu | IVS1 + 5G > C | 1 | +5 | |
| | | GCGguuagu | IVS1 + 3G > U | 1 | +3 | |
| HBA2 | Alpha- | GAGgcuccc | IVS1 del[+2: +6] | 1 | del[+2: +6] | GGGguaagg (Exon1 − 49) |
| | thalassemia | | | | | |
| AR | Androgen | CUGuuaag | IVS4 + 1G > U | 4 | +1 | |
| | Sensitivity | | | | | |
| ATM | Ataxia- | CAGauaacu | IVS45 + 1G > A | 45 | +1 | AGAgugacu (IVS45 + 72) |
| | telangiectasia | | | | | |
| BRCA1 | Breast Cancer | UUUgugagc | IVS16 + 6U > C | 16 | +6 | UAUguaaga (Exon5 − 22) |
| | | AGGguauau | IVS5 − 2A > G | 5 | −2 | UAGguauug (IVS16 + 70) |
| CYP27A1 | Cerebrotendinous | GAGguagga | IVS6 − 2C > A | 6 | −2 | GUGgugggu (Exon6 − 89) |
| | xanthomatosis | GCAguagga | IVS6 − 1G > A | 6 | −1 | |
| FAH | Chronic | CCGgugaau | IVS12 + 5G > A | 12 | +5 | GAGgugggu (IVS112 + 106) |
| | Tyrosinemia | | | | | |
| | Type 1 | | | | | |
| TP53 | Colorectal | AUGgugacc | IVS5 + 5G > C | 5 | +5 | |
| | tumors | | | | | |
| FGA | Common | GAGuuaagu | IVS4 + 1G > U | 4 | +1 | GGAguuaag (Exon4 − 6) |
| | congenital | | | | | UAAguauua (Exon4 − 36) |
| | afibrinogenemia | | | | | |
| PTEN | Cowden | AAGauuugu | IVS7 + 1G > A | 7 | +1 | CAUguaagg (IVS7 + 76) |
| | syndrome | GAGgcaggu | IVS4 + 2U > C | 4 | +2 | |
| UGT1A1 | Crigler-Najjar | CAGcugugu | IVS1 + 1G > C | 1 | +1 | GAGgugacu (Exon1 − 141) |
| | syndrome type | | | | | |
| | 1 | | | | | |
| CFTR | Cystic Fibrosis | CACgugagc | IVS20 − 1G > C | 20 | −1 | AUUgugagg (Exon4 − 93) |
| | | AAGuuaaua | IVS4 + 1G > U | 4 | +1 | |
| COL7A1 | Dominant | AGGgugagg | Exon73 del[−98: | 73 | del[−98: −71] | CUGguauuc (Exon73 − 62) |
| | Dystrophic | | −71] | | | |
| | epidermolysis | | | | | |
| | bullosa | | | | | |
| KRT5 | Dowling- | AAGaugagc | IVS1 + 1G > A | 1 | +1 | AGGgugagg (Exon1 − 66) |
| | Meara | | | | | |
| | epidermolysis | | | | | |
| | bullosa | | | | | |
| | simplex | | | | | |
| DMD | Duchenne and | GCUguaacu | IVS64 + 5G > C | 64 | +5 | AAGggaaaa |
| | Becker | | | | | (IVS26 + 2U > G) |
| | muscular | | | | | |
| | dystrophy | | | | | |
| COL3A1 | Ehlers-Danlos | GAUaugagu | IVS42 + 1G > A | 42 | +1 | GGAguaagc (IVS16 + 24) |
| | syndrome IV | CCUauaagu | IVS16 + 1G > A | 16 | +1 | |
| | | CGCauaagu | IVS20 + 1G > A | 20 | +1 | |
| LPL | Familial | ACGauaagg | IVS2 + 1G > A | 2 | +1 | CAGguggga (IVS2 + 143) |
| | hypercholes- | | | | | GAGguuggu (IVS2 |
| | terolemia | | | | | +247) |
| | | | | | | AGAgugagg (IVS2 + 383) |
| LDLR | Familial | GAGgcgugg | IVS12 + 2U > C | 12 | +2 | UACguacga (IVS12 + 12) |
| | hypercholes- | | | | | |
| | terolemia | | | | | |
| TSC2 | Familial | AAGgaugag | IVS37 + 2 ins[A] | 37 | +2 ins | CCGgugagg (Exon37 − 29) |
| | tuberous | | | | | |
| | sclerosis | | | | | |
| F7 | FVII | UGGgugggug | IVS7 + 7A > G | 7 | +7 | UGGgugggu (IVS7 + 38) |
| | deficiency | UGGguggau | IVS7 + 5G > A | 7 | +5 | |
| | | UGGguacca | IVS7del[+3: +6] | 7 | del[+3: +6] | |
| ITGB3 | Glanzmann | GAUaugagu | IVS4 + 1G > A | 4 | +1 | CAGgugugg (IVS4 + 28) |
| | thrombasthenia | | | | | |
| C3 | Hereditary C3 | UGGauaagg | IVS18 + 1G > A | 18 | +1 | GAAgugagu (Exon 18 − 61) |
| | deficiency | | | | | |
| HMGCL | Hereditary HL | ACGcuaagc | IVS7 + 1G > C | 7 | +1 | GGGguauuu (IVS7 + 79) |
| | deficiency | | | | | |
| APOB | Homozygous | AAGgcaaaa | IVS24 + 2U > C | 24 | +2 | |
| | hypobetalipo- | | | | | |
| | proteinemia | | | | | |
| LMNA | Hutchinson- | CAAgugagu | IVS11 − 1G > A | 11 | −1 | CAGgugggc (Exon 11) |
| | (HGPS) | CAGgugacu | IVS11 + 5G > C | 11 | +5 | CAGgugggc (Exon 11) |
| | Gilford | CAGaugagu | IVS11 + 1G > A | 11 | +1 | CAGgugggc (Exon 11) |
| | progeria | CAGgcgagu | IVS11 + 2U > C | 11 | +2 | CAGgugggc (Exon 11) |
| | syndrome | | | | | |
| HPRT1 | Lesch-Nyhan | GAAggaagu | IVS5 + 2U > G | 5 | +2 | AAGguaagc (IVS5 + 68) |
| | syndrome | GAAgugugu | IVS5 + 3: 4AA > GU | 5 | +3 | |
| | | GAAguaaau | IVS5 + 5G > A | 5 | +5 | |
| | | GAAuaaguu | IVS5del[G1] | 5 | del[1] | |
| ITGB2 | Leukocyte | UUCauaagu | IVS7 + 1G > A | 7 | +1 | AGGgugggg (IVS7 + 65) |
| | adhesion | | | | | |
| | deficiency | | | | | |
| FBN1 | Marfan | UAGaugcgu | IVS46 + 1G > A | 46 | +1 | GAAgucagu (IVS46 + 34) |
| | syndrome | | | | | |
| GCK | Maturity onset | | | | | CCUgugagg (Exon4 − 24) |
| | diabetes of the | | | | | |
| | young | | | | | |
| | (MODY) | | | | | |
| COL6A1 | Mild Bethlem | GGGaugagu | IVS3 + 1G > A | 3 | +1 | CAAguacuu (Exon3 − 66) |
| | myopathy | | | | | |
| IDS | Mucopolysac- | AUUuuaagc | IVS7 − 1: | 7 | −1 | CUGgugagu (IVS7 + 23) |
| | charidosis type | | +1GG > UU | | | |
| | II (Hunter | | | | | |
| | syndrome) | | | | | |
| GHV | Mutation in | UUUauaagc | IVS2 + 1G > A | 2 | +1 | UGGguaaug (IVS2 + 13) |
| | placenta | | | | | |
| YGM | Myophosphorylase | ACCaugagu | IVS14 + 1G > A | 14 | +1 | CAGgugaag (Exon 14 − 67) |
| | deficiency | | | | | |
| | (McArdle | | | | | |
| | disease) | | | | | |
| NF1 | Neurofibromatosis | AAAauaagu | IVS28 + 1G > A | 28 | +1 | AACguuaag (Exon27b − 69) |
| | type I | GAGguaaga | IVS27b | 27b | del[+1: +10] | AAGguauuc (Exon28 − 4) |
| | | | del[+1: +10] | | | |
| NF2 | Neurofibromatosis | GAGgugagg | IVS12del[−14: | 12 | del[−14: +2] | GAUguacgg (Exon7 − 23) |
| | type II | | +2] | | | AAGgugcug (Exon 12 − 38) |
| | | GAGaugagg | IVS12 + 1G > A | 12 | +1 | GAGgugcug (Exon 12 − 53) |
| | | CGGguguau | IVS7 + 5G > A | 7 | +5 | ACGguguga (Exon7 − 28) |
| PGK1 | Phosphoglycerate | AAGuuagga | IVS4 + 1G > U | 4 | +1 | GGGgugagg (IVS4 + 31) |
| | kinase | | | | | |
| | deficiency | | | | | |
| CYP19 | Placental | UGUgcaagu | IVS6 + 2U > C | 6 | +2 | |
| | aromatase | | | | | |
| | deficiency | | | | | |
| PKD1 | Polycystic | | | | | CAGguggcg (Exon43 − 66) |
| | kidney disease | | | | | |
| | 1 | | | | | |
| COL7A1 | Recessive | GUAgugagu | IVS95 − 1G > A | 95 | −1 | GGGgucagu (Exon95 − 7) |
| | dystrophic | AGGgugauc | IVS3 − 2A > G | 3 | −2 | UCCgugagc (Exon 3 − 104) |
| | epidermolysis | | | | | |
| | bullosa | | | | | |
| COL7A1 | Risk for | AAGuuaagg | IVS2 + 1G > U | 2 | +1 | AGGguacuc (Exon2 − 84) |
| | emphysema | | | | | |
| COL7A1 | Sandhoff | UUGguaaca | IVS8 + 5G > C | 8 | +5 | AAUguuggu (Exon8 − 4) |
| | disease | | | | | |
| MTHFR | Severe | CAGaugagg | IVS4 + 1G > A | 4 | +1 | |
| | deficiency of | | | | | |
| | MTHFR | | | | | |
| F5 | Severe factor | CAUguauuu | IVS10 − 1G > U | 10 | −1 | UCUguaaga (Exon10 − 35) |
| | V deficiency | | | | | |
| COL1A1 | Severe type III | CCUaugagu | IVS8 + 1G > A | 8 | +1 | UUGguaaga (IVS8 G + |
| | osteogenesis | CCUgugaau | IVS8 + 5G > A | 8 | +5 | 97exon 8 ± 26) |
| | imperfecta | | | | | CUGgugagc (IVS8 + 97) |
| | | | | | | CUGgugaca (Exon34 − 8) |
| HPRT1 | Somatic | GUGgugagc | IVS1del[−2: +34] | 1 | del[−2: +34] | CAGguggcg (IVS1 + 50) |
| | mutations in | GUGgugauc | IVS1 + 5G > U | 1 | +5 | |
| | kidney tubular | | | | | |
| | epithelial cells | | | | | |
| TP53 | Squamous cell | GAAgucugg | IVS6 − 1G > A | 6 | −1 | |
| | carcinoma | GAGaucugg | IVS6 + 1G > A | 6 | +1 | |
| HXA | Tay-Sachs | GACaugagg | IVS9 + 1 G > A | 9 | +1 | AGGgugggu (IVS9 + 18) |
| | Syndrome | | | | | |
| ABCD1 | X-linked | GAAguggg | IVS1 − 1G > A | 1 | −1 | CAGguuggg (IVS1 + 10) |
| | adrenoleuko- | | | | | |
| | dystrophy (X- | | | | | |
| | ALD) | | | | | |
| RPGR | X-linked | CUGuugaga | IVS5 + 1G > U | 5 | +1 | CAUguaauu (Exon5 − 76) |
| | retinitis | | | | | |
| | pigmentosa | | | | | |
| | (RP3) | | | | | |

### NMR

Nuclear Magnetic Resonance (NMR) spectroscopy can be a powerful analytical technique used to determine qualitative and quantitative information about organic molecules. NMR can be used to solve and provide valuable information about the structure of a variety of chemical and biological molecules, ranging from small organic compounds to complex polymers such as proteins and nucleic acids. In NMR, a sample is placed in a magnetic field and is subjected to radiofrequency (RF) excitation at a characteristic frequency called Larmor frequency (f):

$$f = \frac{γ}{2 π} {B}_{0}$$

where γ is the gyromagnetic ratio of nuclei and B₀ is the magnetic field strength. The nuclei in the magnetic field absorb the energy provided and become energized. The frequency of the radiation necessary for absorption depends on the type of nuclei to be excited, (e.g., ¹H or ¹³C, or ¹⁵N), the frequency will typically also depend on the chemical environment of the nucleus (e.g., the presence of various chemical electronegative groups, salts, pH of solution, and the presence of binding agents), and lastly, the frequency may also depend on the spatial location in the magnetic field if the magnetic field is not uniform, i.e., the field is not homogeneous.

In various embodiments, the methods for determining a 2-D structure and/or a 3-D atomic structure utilize NMR devices having a commercially available spectrometer frequencies, for example, at a ¹H Larmor frequency of greater than about 1 GHz, about 1 GHz, from about 1 GHz to about 20 MHz, or about 900 MHz, about 800 MHz, about 700 MHz, about 600 MHz, about 500 MHz, about 400 MHz, about 300 MHz, about 200 MHz, about 100 MHz, about 75 MHz, about 50 MHz, or about 20 MHz, can be used to determine the structure of a biomolecule, for example, a polynucleotide. Solely for the purpose of convenience, the disclosure of the present methods will be exemplified with the use of polynucleotides, but the methods described herein are applicable to determine the interactions or structure of a protein or a polypeptide as the target or desired biomolecule of interest. Methods for selectively labeling proteins and polypeptides are known in the art. In some embodiments, the methods of the present technology can be performed using an NMR module operable to provide a ¹H Larmor frequency of 300 MHz or less.

In some embodiments, a lower magnetic fields (for example, 300 MHz or less) can be used, which can significantly shorten the repetition delay and the total experimental time can be reduced to ¼-⅕ of that of high fields because the repetition delay depends on Ti relaxation time which is significantly shorter at low magnetic field (i.e., Ti relaxation time at 100 MHz is more than 6 times shorter than that of 600 MHz for molecules of correlation time of 4-8 ns (oligonucleotides of 25-50 bases)). This Ti relaxation time difference at between high and low magnetic fields becomes larger as molecular weight or size of a molecule increases. Within given time, 4-5 times more measurements can be repeated and added at low magnetic fields to yield signal-to-noise gain of factor of 2.

In some embodiments, there are unexpected advantages using a low field NMR device, for example, an NMR device having a spectrometer frequency of 300 MHz or less. In some embodiments, the methods are derived from the surprising finding that low field NMR can be employed to obtain structurally detailed information concerning a complex structure, such as a polynucleotide. Combining the use of low field NMR (i.e., a ¹H Larmor frequency of 300 MHz or less) with selective labeling of the sample provides a sufficient resolution that permits NMR studies of complex 3-D structures using chemical shift information.

In some embodiments, the methods of the present disclosure utilize a low field NMR. These methods illustratively include interrogation of the target or selected polynucleotide selectively labeled with one or more nucleotides using a static magnetic field and reference frequency of 300 MHz or less, or about 299 MHz or less, or about 250 MHz or less, or about 225 MHz or less, or about 200 MHz or less, or less than about 175 MHz, or less than about 150 MHz, or less than about 125 MHz, or less than about 100 MHz, preferably, ranging from about 20 MHz to about 300 MHz, or from about 20 MHz to about 299 MHz, or from about 50 MHz to about 275 MHz, or from about 75 MHz to about 250 MHz, or from about 75 MHz to about 225 MHz, or from about 75 MHz to about 200 MHz, or from about 75 MHz to about 175 MHz, or from about 100 MHz to about 300 MHz, or from about 125 MHz to about 275 MHz, or from about 20 MHz to about 250 MHz, or from about 20 MHz to about 225 MHz, or from about 20 MHz to about 200 MHz, or from about 20 MHz to about 150 MHz, or from about 20 MHz to about 100 MHz.

In some embodiments a number of small molecule bound bimolecular structures can be determined for uses comprising computer aided drug discovery efforts, which commonly rely on biomolecular structures determined when bound to a small molecule.

In order to identify which small molecules interact with the biomolecule, in some embodiments, one synthesizes a uniformly isotopically labeled biomolecular sample, individually or in a combinatorial manner mix each small molecule at a ratio that one would expect to see changes in NMR signals for relatively tight binding small molecules (for a low μM K_{d}, a ratio of 2:1 or 4:1 could be used), collect the NMR data such as chemical shifts, resonance intensities, and/or NOEs, compare the NMR data of the biomolecule in the presence of the small molecule to the NMR data of the biomolecule in the absence of the small molecule, and select small molecules that cause significant changes in the NMR data. In some embodiments, changes in NMR data comprise a portion of a chemical shift linewidth, for example a one linewidth. In some embodiments, changes in NMR data comprise a significant reduction in an NOE and/or a resonance intensity when comparing the biomolecule NMR data in the absence and presence of the small molecule is significant). In various embodiments, NMR data of the small molecule could be monitored and similar perturbations observed on addition of the biomolecule of interest, where, in some embodiments, the biomolecule is non-isotopically labeled. In various embodiments, the same solution conditions (e.g., buffer or solubilization solution) for each sample are used to minimize random noise due to differences in solution environments.

### Methods

In some aspects, the methods described herein fits within the drug discovery paradigm used in pharmaceutical and biotech industries. In a first example, the subject matter described herein exploits nucleic acid (e.g., RNA) plasticity to solve atomic-resolution nucleic acid (e.g., RNA) structures and uncover binding pockets optimized to identify key small molecule-nucleic acid (e.g., RNA) interactions. In various embodiments, these binding pockets afford efficient hit identification with atomic-level guidance during target screening. In a second example, in pursuing small molecules for hit-to-lead studies and lead optimization, the atomic-level interactions enable medicinal chemists to rationally design new compounds. In some embodiments, this affords accurate and efficient target validation.

In some aspects, the present disclosure provides a method for determining the 2-dimensional (2-D) or 3-dimensional (3-D) atomic resolution structure of a polynucleotide. The method includes providing a polynucleotide sample comprising a polynucleotide, the polynucleotide comprising none or at least one nucleotide isotopically labeled with one or more atomic labels selected from the group consisting of ²H, ¹³C, ¹⁵N, ¹⁹F and ³¹P. In some embodiments, the method further comprises obtaining a NMR spectrum of the polynucleotide sample using a NMR device. In some embodiments, the method further comprises determining a chemical shift of the one or more atoms or a subset of atoms with close molecular interactions. In some embodiments, the method further comprises determining a 2-D or a 3-D atomic resolution structure of the polynucleotide from the chemical shifts.

In some embodiments, a first NMR spectrum can be obtained for a first complex in the sample, and a second NMR spectrum can be obtained for a second complex in the sample. The second complex can contain one or more molecules (e.g. polynucleotide, polypeptide, or small molecule) more than the first complex. In some embodiments, the method further comprises comparing the first and the second NMR spectrum. In some embodiments, a NMR spectrum is obtained for a polynucleotide sample without a small molecule. In some embodiments, a NMR spectrum is obtained for a polynucleotide sample containing a small molecule. In some embodiments, the method comprises selecting or identifying a binding agent based on comparing different NMR spectrums. In some embodiments, the method comprises selecting or identifying a small molecule based on comparing different NMR spectrums.

In some embodiments, the method to determine the 2-D or 3-D structure of a polynucleotide may need interrogation of multiple polynucleotides having the same nucleotide sequence, but differing from each other in that each polynucleotide is isotopically labeled on a different nucleotide. In other words, the method determines the chemical shifts of multiple polynucleotides, each polynucleotide having the identical nucleotide sequence as the first polynucleotide analyzed, and each polynucleotide is synthesized with a different nucleotide labeled with the one or more atomic labels. For example, if the polynucleotide has 5 nucleotides, the method would require 5 polynucleotide samples, each polynucleotide labeled with the one or more atomic labels on a different nucleotide. In this same 5-mer polynucleotide example, the method may utilize a smaller number of distinct polynucleotides that the number of nucleotides presents in the nucleotide sequence, by strategically labeling one or more nucleotides in the polynucleotide with one or more atomic labels as described herein. In some embodiments, the polynucleotide sample has only one polynucleotide with one nucleotide labeling pattern. In other embodiments, the polynucleotide sample may contain two or more polynucleotides, each having a different nucleotide labeled with one or more atomic labels.

In some aspects, the method obtains a NMR spectrum of the polynucleotide sample by interrogating the polynucleotide sample with a NMR spectrometer frequency ranging from about 1 GHz to about 20 MHz. In one of these aspects, the NMR spectrometer frequency is 300 MHz or less, for example, from about 20 MHz to about 100 MHz.

In some embodiments, the NMR interrogation includes one or more of the following 6 steps. First, in some embodiments, comprises a temperature regulation step. In this aspect, the liquid sample containing the polynucleotide of interest in the appropriate chemical environment is transferred to a sample conduit and fills the analysis volume with sample for NMR interrogation. Second, in some embodiments, the sample in the sample conduit is equilibrated at a selected temperature ranging from 0 to 60° C. Third, in some embodiments, a tuning and matching step can be performed. This process adjusts the resonant circuit frequency and impedance until they coincide with the frequency of the pulses transmitted to the circuit and impedance of the transmission line (typically 50 ohm). For best signal-to-noise and minimal RF coil heating, the tuning and matching can be done for each sample. But with pre-adjustment during manufacturing process, minor or no adjustment is necessary for low field magnets. Fourth, in some embodiments, a locking step is performed. In this process, the ²H signal is found from deuterated solvent for internal feedback mechanism by which magnetic field drift can be compensated. The ²H signal (for example, 30.7 MHz at 200 MHz spectrometer) being distant from ¹H signal is acquired and processed independently. Lock signal also serves as chemical shift reference.

Fifth, in some embodiments, prior to acquiring NMR data on the sample being interrogated is a shimming step. In some embodiments, the interrogation step may require creating a homogeneous magnetic field at the analysis volume by controlling electric currents in a set of coils which generate small static magnetic fields of different geometries and strength and correct inhomogeneity of the B₀. For NMR interrogation of biomolecules of the present disclosure, it is preferred to have at least 50 ppb (part per billion) of field homogeneity when analyzing samples using NMR.

Sixth, in some embodiments, a sequence of precise pulses and delays are applied to ¹H and ¹³C transmission lines connected to each resonant circuit around the analysis volume to manipulate spin quantum states of nuclei in the sample. As a result, only the desired signals such as ¹H nuclei spins attached to ¹³C are selected and measured excluding all other ¹H nuclei spins attached to other nuclei, or using shaped pulses (selective pulses) nuclei having certain chemical shift range are detected. Many different types of pulse sequences can be applicable for different purposes including a variety of HSQC, HMQC, COSY, TOCSY, NOESY, ROESY for structural determinations of biomolecules in 1-D, 2-D, and 3-D experimental settings. In some embodiments, after the pulse sequence, the same resonant circuits (including the 2 or more RF coils) are sensing fluctuation of magnetic field around analysis volume (called FID; free induction decay) as electric voltage which is digitized and recorded for predefined duration. To improve the signal-to-noise (S/N), a set of pulsing and recording steps are repeated multiple times and added with some delay in between, called relaxation delay which allow spin systems to return to initial state before starting pulsing.

In some aspects, the present disclosure provides methods for determining the structure of a target biomolecule when mixed with a small molecule, biomolecule, ligand or other chemical entity (collectively referred to as a binding agent) that could interact with the biomolecule of interest. Chemical shift changes on the addition of the binding agent indicate that the biomolecule may be interacting with the binding agent. The chemical shifts in the presence of the binding agent can be collected and used to determine the biomolecular structure of the biomolecule and the bound binding agent. In some embodiments of this aspect, the method includes the steps of providing a polynucleotide sample comprising a plurality of polynucleotides, the plurality of polynucleotides having an identical nucleotide sequence, wherein each polynucleotide comprises at least one nucleotide isotopically labeled with one or more atomic labels selected from the group consisting of ²H, ¹³C, ¹⁵N, ¹⁹F and ³¹P; admixing the polynucleotide sample with the binding agent forming a plurality of bound complexes; obtaining a NMR spectrum of the bound complexes using a NMR device; determining a chemical shift of the one or more atomic labels; and determining the 3-D atomic resolution structure of the polynucleotides from the chemical shifts.

In some embodiments of the present methods, the target polynucleotide is analyzed by creating a plurality of polynucleotides all having the same nucleotide sequence but differing in the location(s) of isotopically labeled nucleotide(s). In some embodiments, the secondary structure of the polynucleotide is used to determine the placement of the labeled nucleotide or nucleotides to reduce the number of polynucleotide samples. Taking the primary sequence of the polynucleotide, the secondary structure is predicted. Then a plurality of secondary structure predictions can be computed using a secondary structure prediction algorithm (e.g., nearest neighbor algorithm) or computer program. The method then uses an alignment step with the top 10 or so secondary structure predictions and then determines the sites that exhibit the greatest variance in secondary structure. Then the site or sites in the polynucleotide sequence that exhibit largest variance are labeled isotopically for NMR detection or a derivative, wherein one or more nucleotides are labeled per polynucleotide. The labeling scheme can be informed from the chemical shift database whereby multiple isotopic labels can be incorporated into a polynucleotide while maximizing chemical shift dispersion.

In some embodiments, the present disclosure provides a method for determining one or more specific isotopic labeling positions of one or more nucleotides within a polynucleotide sequence for the determination of 3-D atomic resolution structure or collecting other NMR interaction data of a polynucleotide. The method includes providing one or more polynucleotides each of the one or more polynucleotides having an identical polynucleotide sequence, wherein each of the one or more polynucleotides comprises one or more nucleotides labeled with an isotopic label comprising, ²H, ¹³C, ¹⁵N, ¹⁹F or ³¹P; predicting a plurality of structures of the polynucleotide sequence using a computational algorithm (e.g., MC-Sym|MC-fold); identifying one or more region(s) on each of the plurality of polynucleotide structures that exhibit a large structural variation using metrics comprising an S2<0.8 and/or RMSF>0.5 Å; calculating a plurality of chemical shifts from regions of the predicted structures having a large structural variation using a chemical shift predictor; such as Nymirum's RANDOM FOREST™ Predictors (RAMSEY), SHIFTS, NUCHEMICS, and QM methods from the predicted structures; and determining one or more specific isotopic labeling positions on each of the polynucleotide sample(s) such that the chemical shift dispersion is maximized and the number of samples is minimized. The MC-Fold|MC-Sym pipeline is a web-hosted service for RNA secondary and tertiary structure prediction. The pipeline means that the input sequence to MC-Fold outputs secondary structures that are directly inputted to MC-Sym, which outputs tertiary structures.

In some aspects, the present invention provides a NMR device that is small enough to sit on top of a standard laboratory bench. In some embodiments of the second aspect, the NMR device includes a housing; a sample handling device operable to receive a sample comprising a polynucleotide; and an NMR module. The NMR module may include a sample conduit comprising an analysis volume operable to receive at least a portion of the sample from the sample handling device; a plurality of radiofrequency coils disposed proximately to the analysis volume, each coil operable to generate a distinct excitation frequency pulse across the analysis volume to generate nuclear magnetic resonance of the nuclei of the polynucleotide in the analysis volume; and at least one magnet operable to provide a static magnetic field across the analysis volume and the radiofrequency coils. The NMR module may have a ¹H Larmor frequency of 300 MHz or less and the RF coils are operable to transmit the excitation frequency pulse to the analysis volume and detect signals from NMR produced by the nuclei of the polynucleotide contained in the analysis volume. Optionally, the device further comprises a heating and cooling device in thermal coupling with the analysis volume. In this regard, the NMR device can employ the use of a sample conduit or analysis volume heating and cooling device for heating the sample containing the biomolecule, for example a protein or a nucleic acid, for example, an RNA polynucleotide to anneal the polynucleotide and bring the polynucleotide into a relaxed or stable conformation prior to acquisition of NMR spectra.

In certain embodiments, the method the step of providing the polynucleotide sample includes determining one or more 2-D or 3-D models of the polynucleotide sequence using a 2-D or 3-D structure predicting algorithm, respectively; identifying one or more structural heterogeneous regions on each of the one or more 2-D or 3-D models of the polynucleotide sequence; calculating one or more chemical shifts from the one or more structural heterogeneous regions; and synthesizing a polynucleotide comprising one or more nucleotides having one or more atomic labels positioned at one or more nuclei which results in a polynucleotide having a minimized chemical shift overlap.

In some embodiments, determining the 3-D atomic resolution structure includes generating a plurality of theoretical structural polynucleotide 2-D models using the nucleotide sequence and one or more 2-D structure predicting algorithms; generating a plurality of theoretical structural polynucleotide 3-D models using a 3-D structure predicting algorithm using the plurality of theoretical structural polynucleotide 2-D models and optionally one or more known or assumed polynucleotide 2-D model; generating a predicted chemical shift set for each of the plurality of theoretical structural polynucleotide 3-D models; comparing the predicted chemical shift set to the chemical shift(s) of the one or more atoms; and selecting one or more theoretical structural polynucleotide 3-D model having an agreement (e.g., the best agreement) between the respective predicted chemical shift set and the chemical shift(s) of the one or more atomic labels as the one or more 3-D atomic resolution structures. In some embodiments, the predicted chemical shift set is generated by comparing each theoretical structural polynucleotide 3-D model with a NMR-data polynucleotide structure database. In some embodiments, generating the predicted chemical shift set includes calculating a polynucleotide structural metric comprising atomic coordinates, stacking interactions, magnetic susceptibility, electromagnetic fields, or dihedral angles from one or more experimentally determined polynucleotide 3-D structures; generating a set of mathematical functions or objects that describe relationships between experimental chemical shifts and the polynucleotide structural metric of the experimentally determined 3-D polynucleotide structures using a regression algorithm; calculating a polynucleotide structural metric for each of the theoretical structural polynucleotide 3-D models; and inputting the polynucleotide structural metric for each of the theoretical structural polynucleotide 3-D models into the set of mathematical functions or objects to generate the predicted chemical shift set.

In some embodiments, the regression algorithm is machine learning algorithm comprising a Random Forest algorithm. In some embodiments, determining the experimental chemical shift set comprises modeling the chemical shift set using a NMR spectrometer frequency from about 1 GHz to about 20 MHz.

In some embodiments, determining the 3-D atomic resolution structure includes generating a plurality of theoretical structural polynucleotide 2-D models using the nucleotide sequence and one or more 2-D structure predicting algorithms; generating a plurality of theoretical structural polynucleotide 3-D models using a 3-D structure predicting algorithm using the plurality of theoretical structural polynucleotide 2-D models and optionally one or more known or assumed polynucleotide 2-D model; generating a predicted chemical shift set for each of the plurality of theoretical structural polynucleotide 3-D models; comparing the predicted chemical shift set to the chemical shift(s) of the one or more atoms; and selecting one or more theoretical structural polynucleotide 3-D model having an agreement (e.g., the best agreement) between the respective predicted chemical shift set and the chemical shift(s) of the one or more atomic labels as the one or more 3-D atomic resolution structures.

In some embodiments, the method also includes the step of identifying a binding pocket in the one or more 3-D atomic resolution structures. In some embodiments, the method also includes the step of associating another molecule with the identified binding pocket of each of the one or more 3-D atomic resolution structures. In some embodiments, the method also includes the step of refining the associated another molecule and binding pocket of each of the one or more 3-D atomic resolution structures using a modeling software that performs one or more functions comprising energy minimization and/or a molecular dynamics simulation. In some embodiments, the method also includes the step of identifying a binding pocket in the one or more refined 3-D atomic resolution structures. In some embodiments, the method also includes the step of using one or more coordinates of the associated another molecule in the refined 3-D structures and binding pocket of each of the one or more 3-D atomic resolution structures. In some embodiments, the predicted chemical shift set is generated by comparing each theoretical structural polynucleotide 3-D model with a NMR-data polynucleotide structure database.

In some embodiments, generating the predicted chemical shift set includes calculating a polynucleotide structural metric comprising atomic coordinates, stacking interactions, magnetic susceptibility, electromagnetic fields, or dihedral angles from one or more experimentally determined polynucleotide 3-D structures; generating a set of mathematical functions or objects that describe relationships between experimental chemical shifts and the polynucleotide structural metric of the experimentally determined 3-D polynucleotide structures using a regression algorithm; calculating a polynucleotide structural metric for each of the theoretical structural polynucleotide 3-D models; and inputting the polynucleotide structural metric for each of the theoretical structural polynucleotide 3-D models into the set of mathematical functions or objects to generate the predicted chemical shift set.

In some embodiments, structural dynamics can be determined by obtaining structural information by NMR in a temporal manner. For example, in binding a small molecule to a target polynucleotide, structural information of the small molecule binding to the target polynucleotide can be determined at different times by NMR after contacting the small molecule to the target polynucleotide. The structural information can be obtained by taking NMR spectrum at different time points. The NMR spectrum taken at different time points can be used to calculate the chemical shifts, and the chemical shifts can be compared in order to determine a binding kinetics.

In some embodiments, binding kinetics between a small molecule and a target polynucleotide can be determined by various methods in the art. For example, kinetics assays for measuring binding kinetics include, but are not limited to, surface plasmon resonance (SPR), Bio-Layer Interferometry (BLI) technology (Octet Systems), isothermal titration calorimetry (ITC), or fluorescence anisotropy. In some embodiments, one or more of the binding kinetics assay are used to confirm the identified small molecule and the target polynucleotide.

Binding kinetics of RNA splicing can broadly encompass the mechanism by which alternative splicing machinery function in conjunction with the structural RNA and execute the function of pre-mRNA splicing, excising of introns and fusion of exons to produce the final mature mRNA isoform. The kinetics of splicing can be a highly dynamic process involved both positive and negative regulators of exon inclusion, such that the overall net effect can be exon inclusion or exon inclusion. Binding agents, such as small molecules, can interact with this process and influence the exonic splicing towards one direction by impacting the affinity of particularly relevant trans-acting binding factors that form the spliceosomal complex. Binding kinetics can be reflected by various parameters, including kₒₙ, k_{off}, and K_{d}. Lower K_{d} usually indicates stronger binding, therefore higher binding affinity.

Binding kinetics of a small molecule binding to a target can be used to determine whether the small molecule is a strong binder or not. Binding kinetics of a polynucleotides binding to another polynucleotide (e.g. a target polynucleotide) with or without a small molecule can be used to determine whether two polynucleotides bind stronger or weaker in the presence of the small molecule. Binding kinetics of a protein binding to a target polynucleotide with or without a small molecule can be used to infer whether the protein binds stronger or weaker in the presence of the small molecule. K_{d} can be determined by various the concentrations of the binding agent in the presence of constant concentration of a target. For example, in determining the K_{d} of a small molecule binding to a target mRNA or RNA-RNA duplex, the concertation of a small molecule can be changed. K_{d} can also be determined by measuring kₒₙ and k_{off} during a binding process, which can be used to calculate K_{d}.

In some embodiments, the binding kinetics between a binding agent and a target polynucleotide can be determined. In some embodiments, the binding kinetics between a binding agent and a RNA-RNA complex can be determined. In some embodiments, the binding kinetics between a binding agent and a RNA-protein complex can be determined. For example, the binding kinetics between a small molecule and a target polynucleotide (e.g. mRNA) can be determined to infer how strong the binding is.

In some embodiments, the binding kinetics of a polynucleotide binding to a target polynucleotide to form a RNA-RNA duplex with or without a small molecule binding agent can be determined. In some embodiments, the binding kinetics of a polynucleotide binding to a target polynucleotide with and without a small molecule binding agent are determined, and the binding kinetics with and without the small molecule can be compared to infer whether the polynucleotide binds to the target polynucleotide stronger or weaker with the small molecule.

In some embodiments, the binding kinetics of a protein or protein component/polypeptide binding to a target RNA to form a protein-RNA complex with or without a small molecule binding agent can be determined. In some embodiments, the binding kinetics of a protein or polypeptide binding to a target polynucleotide with and without a small molecule binding agent are determined, and the binding kinetics with and without the small molecule can be compared to infer whether the protein binds to the target polynucleotide stronger or weaker with the small molecule.

In some embodiments, the binding kinetics of a protein-RNA complex binding to a target RNA to form a complex with or without a small molecule binding agent can be determined. In some embodiments, the binding kinetics of a protein-RNA complex binding to a target polynucleotide with and without a small molecule binding agent are determined, and the binding kinetics with and without the small molecule can be compared to infer whether the protein-RNA complex binds to the target polynucleotide stronger or weaker with the small molecule.

In some embodiments, small molecule binding agents are selected by NMR assay and then tested in the kinetics assay. For example, the kinetics assay can be used to measure the binding kinetics of two or more different molecules against the same target (e.g. RNA, RNA-RNA complex, or RNA-protein complex) and compare the K_{d} to infer which small molecules are strong binders. The kinetics assay can serve as secondary screening assay following the NMR initial screening. In some embodiments, the kinetics assay can also serve as initial screening assay and followed by NMR for structural determination.

In some embodiments, the binding kinetics is measured by SPR and/or BLI. In such cases, a polynucleotide is immobilized on a surface. In some situations, the target polynucleotide (e.g. target mRNA) is immobilized on a surface. In some situations, a polynucleotide such as a snRNA is immobilized on a surface. The method to immobilize a polynucleotide on a surface can include labeling the polynucleotide with biotin, and conjugate the surface with streptavidin, thereby immobilizing the polynucleotide through biotin-streptavidin interaction.

In some embodiments, the binding kinetics is measured by fluorescence anisotropy, wherein a polynucleotide can be labeled with a fluorophore. In some other embodiments, the binding kinetics is measured by ITC.

In any of the above mentioned embodiments, the kinetics assay can be tested in the presence of one or more polynucleotide molecules, or one or more polypeptides or a portion thereof. For example, U1 snRNP binding to a target mRNA containing 5′ss can be tested in the presence of one or more auxiliary splicing factors or proteins involved in the splicing. The proteins used herein can comprise a portion, for example a domain, of the proteins.

Also provided herein are methods to determine the specificity of a small molecule. For example, a small molecule selected by an initial NMR screening can be tested in any of the above mentioned kinetic assays to determine the binding affinity of the small molecule against different targets. The target can be a target mRNA bound with a snRNA in the presence or absence of a protein or a portion thereof. In some embodiments, the specificity of the small molecule is tested against different RNA-RNA duplexes comprising a target mRNA (e.g. 5′ss) and a snRNA (e.g. U1 snRNA). In some embodiments, the specificity of the small molecule is tested against different protein-RNA complexes comprising a target mRNA (e.g. 5′ss), a snRNA (e.g. U1 snRNA) and a protein or a protein domain (e.g. U1-C zinc finger domain).

Virtual screening or structure-based drug design can be performed following the NMR study. In the above mentioned NMR studies, 3-dimensional structural model can be generated for each target polynucleotide in the presence of any binding partners (e.g. a polynucleotide, or a polypeptide). For example, 3-dimensional structural model can be generated to a target mRNA bound with a snRNA or a portion thereof and a binding pocket can be identified for the RNA-RNA duplex. For another example, 3-dimensional structural model can be generated to a target mRNA bound with a snRNA in the presence of a protein binding partner or a domain of the protein, and a binding pocket can be identified for the RNA-protein complex. The identified binding pocket can be further used for structure-based drug design or virtual screening process. Structure-based drug design (or direct drug design) can rely on knowledge of the 3-dimensional structure of the biological target molecule (e.g. mRNA) obtained through methods such as x-ray crystallography or NMR spectroscopy. If an experimental structure of a target is not available, it may be possible to create a homology model of the target based on the experimental structure of a related molecule. Using the structure of the biological target, candidate drugs that are predicted to bind with high affinity and selectivity to the target may be designed using interactive graphics and the intuition of a medicinal chemist. Alternatively various automated computational procedures may be used to suggest new drug candidates.

Current methods for structure-based drug design can be divided roughly into three main categories. The first method is identification of new ligands for a given receptor by searching large databases of 3D structures of small molecules to find those fitting the binding pocket of a target using fast approximate docking programs. A second category is de novo design of new ligands. In this method, ligand molecules are built up within the constraints of the binding pocket by assembling small pieces in a stepwise manner. These pieces can be either individual atoms or molecular fragments. The key advantage of such a method is that novel structures, not contained in any database, can be suggested. A third method is the optimization of known ligands by evaluating proposed analogs within the binding pocket. The structure-based drug can be aided by computer programs (e.g. GOLD), therefore, it can be referred to a virtual screening process. As used herein, virtual screen or screening can broadly cover all the above method structure-based drug design categories. In one aspect of the present disclosure, a virtual screening process is provided to select small molecule or fragments thereof for de novo drug design and/or lead optimization. In some embodiments, the present disclosure provides a method comprising: identifying one or more binding pockets formed by a target polynucleotide and a first polynucleotide, wherein the target polynucleotide contains a splice site, a branch point (BP), an exonic splicing enhancer (ESE), an exonic splicing silencer (ESS), an intronic splicing enhancer (ISE), an intronic splicing silencer (ISS), or a polypyrimidine tract, or any combinations thereof; and virtually screening one or more small molecules or fragments thereof against the one or more binding pockets, wherein the virtual screening process identifies putative small molecule or fragment hits. In some embodiments, a first and a second small molecule hit can be identify through virtual screening process, and the binding kinetics of the first and the second small molecule hit can be determined. In some embodiments, the binding kinetics of the first and the second small molecule can be compared to infer the binding affinity of the small molecule hit and select a stronger small molecule (i.e. higher binding affinity). The binding kinetics can be determined by various assays, including surface plasmon resonance (SPR), Bio-Layer Interferometry (BLI) technology (Octet Systems), isothermal titration calorimetry (ITC), or fluorescence anisotropy.

### Small Molecules and Splicing

Diseases associated with changes to RNA transcript amount are often treated with a focus on the aberrant protein expression. However, if the processes responsible for the aberrant changes in RNA levels, such as components of the splicing process or associated transcription factors or associated stability factors, could be targeted by treatment with a small molecule, it would be possible to restore protein expression levels such that the unwanted effects of the expression of aberrant levels of RNA transcripts or associated proteins. The present disclosure provides methods of modulating the amount of RNA transcripts encoded by certain genes as a way to prevent or treat diseases associated with aberrant expression of the RNA transcripts or associated proteins.

In various embodiments, the present disclosure provides methods to identify small molecule binding agents that bind to a target polynucleotide, for example, an mRNA. In some embodiments, the present disclosure provides methods to identify small molecule binding agents that bind to a polynucleotide-protein complex, for example a complex formed by a pre-mRNA and a protein involved in splicing. In various embodiments, the present disclosure provides a screening method to select small molecule binding agents that can bind to a polynucleotide-protein complex. In various embodiments, the present disclosure provides screening methods to select small molecule binding agents that can correct aberrant RNA splicing. In various embodiments, the present disclosure provides methods to select small molecule binding agents by NMR.

Aberrant splicing can happen in pre-mRNA transcribed from various genes, including, but not limited to, ABCA4, ABCB4, ABCD1, ACADSB, ADA, ADAMTS13, AGL, ALB, ALDH3A2, ALG6, APC, APOB, AR, ATM, ATP7A, ATR, B2M, BMP2K, BRCA1, BRCA2, BTK, C3, CAT, CD46, CDH1, CDH23, CFTR, CHM, COL11A1, COL11A2, COL1A1, COL1A2, COL2A1, COL3A1, COL4A5, COL6A1, COL6A3, COL7A1, COL9A2, COLQ, CUL4B, CYBB, CYP17, CYP19, CYP27, CYP27A1, DES, DMD, DYSF, EGFR, EMD, ETV4, F13A1, F5, F7, F8, FAH, FANCA, FANCC, FANCG, FBN1, FECH, FGA, FGFR2, FGG, FIX, FLNA, FOXM1, FRAS1, GALC, GH1, GHV, HADHA, HBA2, HBB, HEXA, HEXB, HLCS, HMBS, HMGCL, HNF1A, HPRT1, HPRT2, HSF4, HSPG2, HTT, IDS, IKBKAP, INSR, ITGB2, ITGB3, JAG1, KRAS, KRT5, L1CAM, LAMA3, LDLR, LMNA, LPL, MADD, MAPT, MLH1, MSH2, MST1R, MTHFR, MUT, MVK, NF1, NF2, OAT, OPA1, OTC, PAH, PBGD, PCCA, PDH1, PGK1, PHEX, PKD2, PKLR, PLEKHM1, PLKR, POMT2, PRDM1, PRKAR1A, PROC, PSEN1, PTCH1, PTEN, PYGM, RP6KA3, RPGR, RSK2, SBCAD, SCN5A, SERPINA1, SLC12A3, SLC6A8, SMN2, SPINK5, SPTA1, TP53, TRAPPC2, TSC1, TSC2, TSHB, UGT1A1, and USH2A.

Exemplary diseases caused by those aberrant splicing can include cystic Fibrosis, myotonia congenita, protoporphyria (erythropoietic), lymphoproliferative syndrome (X-linked), neurofibromatosis, retinitis pigmentosa, spondyloepiphyseal dysplasia tarda, epilepsy (progressive myoclonus), Rubinstein-Taybi syndrome, muscular dystrophy (merosin deficient), occipital horn syndrome, medium-chain acyl-CoA DH deficiency, tuberous sclerosis, Frontotemporal dementia with Parkinsonism, osteogenesis imperfecta, myotonia congenita, occipital horn syndrome, familial dysautonomia, spinal muscular atrophy, cancer, hypoxanthine phosphoribosyltransferase deficiency, Ehlers-Danlos syndrome, Fanconi anemia, Marfan syndrome, thrombotic thrombocytopenic purpura, glycogen storage disease Type III, and atypical hemolytic uremic syndrome (aHUS).

In some embodiments, the non-cancer diseases and/or associated conditions therewith that can be prevented/treated in accordance with the present disclosure include non-cancer condition or disease is selected from the group consisting of Hutchinson-Gilford progeria syndrome (HGPS), Limb girdle muscular dystrophy type 1B, Familial partial lipodystrophy type 2, Frontotemporal dementia with parkinsonism chromosome 17, Neonatal Hypoxia-Ischemia, Familial Dysautonomia, Hypoxanthine phosphoribosyltransferase deficiency, Ehlers-Danlos syndrome, Occipital Horn Syndrome, Fanconi Anemia, Marfan Syndrome, thrombotic thrombocytopenic purpura, glycogen Storage Disease Type III, Tyrosinemia (type I), Menkes Disease, Analbuminemia, Congenital acetylcholinesterase deficiency, Haemophilia B deficiency (coagulation factor IX deficiency), Recessive dystrophic epidermolysis bullosa, Dominant dystrophic epidermolysis bullosa, Somatic mutations in kidney tubular epithelial cells, X-linked adrenoleukodystrophy (X-ALD), FVII deficiency, Homozygous hypobetalipoproteinemia, Ataxia-telangiectasia, Androgen Sensitivity, Common congenital afibrinogenemia, Risk for emphysema, Mucopolysaccharidosis type II (Hunter syndrome), Severe type III osteogenesis imperfecta, Ehlers-Danlos syndrome IV, Glanzmann thrombasthenia, Mild Bethlem myopathy, Dowling-Meara epidermolysis bullosa simplex, Severe deficiency of MTHFR, Acute intermittent porphyria, Tay-Sachs Syndrome, Myophosphorylase deficiency (McArdle disease), Chronic Tyrosinemia Type 1, Mutation in placenta, Leukocyte adhesion deficiency, Hereditary C3 deficiency, Placental aromatase deficiency, Cerebrotendinous xanthomatosis, Duchenne and Becker muscular dystrophy, Severe factor V deficiency, Alpha-thalassemia, Beta-thalassemia, Hereditary HL deficiency, Lesch-Nyhan syndrome, Familial hypercholesterolemia, Phosphoglycerate kinase deficiency, Cowden syndrome, X-linked retinitis pigmentosa (RP3), Crigler-Najjar syndrome type 1, Chronic tyrosinemia type I, Sandhoff disease, Maturity onset diabetes of the young (MODY), Familial tuberous sclerosis, Polycystic kidney disease 1, Primary Hyperthyroidism, cystic fibrosis, Spinal muscular atrophy, neurofibromatosis, Neurofibromatosis type I and Neurofibromatosis type II.

In specific embodiments, the cancer treated by the compounds of the present disclosure is leukemia, acute myeloid leukemia, colon cancer, gastric cancer, macular degeneration, acute monocytic leukemia, breast cancer, hepatocellular carcinoma, cone-rod dystrophy, alveolar soft part sarcoma, myeloma, skin melanoma, prostatitis, pancreatitis, pancreatic cancer, retinitis, adenocarcinoma, adenoiditis, adenoid cystic carcinoma, cataract, retinal degeneration, gastrointestinal stromal tumor, Wegener's granulomatosis, sarcoma, myopathy, prostate adenocarcinoma, Hodgkin's lymphoma, ovarian cancer, non-Hodgkin's lymphoma, multiple myeloma, chronic myeloid leukemia, acute lymphoblastic leukemia, renal cell carcinoma, transitional cell carcinoma, colorectal cancer, chronic lymphocytic leukemia, anaplastic large cell lymphoma, kidney cancer, breast cancer, cervical cancer.

In specific embodiments, the cancer prevented and/or treated in accordance with the present disclosure is basal cell carcinoma, goblet cell metaplasia, or a malignant glioma, cancer of the liver, breast, lung, prostate, cervix, uterus, colon, pancreas, kidney, stomach, bladder, ovary, or brain.

In specific embodiments, the cancer prevented and/or treated in accordance with the present disclosure include, but are not limited to, cancer of the head, neck, eye, mouth, throat, esophagus, esophagus, chest, bone, lung, kidney, colon, rectum or other gastrointestinal tract organs, stomach, spleen, skeletal muscle, subcutaneous tissue, prostate, breast, ovaries, testicles or other reproductive organs, skin, thyroid, blood, lymph nodes, kidney, liver, pancreas, and brain or central nervous system.

Specific examples of cancers that can be prevented and/or treated in accordance with present disclosure include, but are not limited to, the following: renal cancer, kidney cancer, glioblastoma multiforme, metastatic breast cancer; breast carcinoma; breast sarcoma; neurofibroma; neurofibromatosis; pediatric tumors; neuroblastoma; malignant melanoma; carcinomas of the epidermis; leukemias such as but not limited to, acute leukemia, acute lymphocytic leukemia, acute myelocytic leukemias such as myeloblastic, promyelocytic, myelomonocytic, monocytic, erythroleukemia leukemias and myclodysplastic syndrome, chronic leukemias such as but not limited to, chronic myelocytic (granulocytic) leukemia, chronic lymphocytic leukemia, hairy cell leukemia; polycythemia vera; lymphomas such as but not limited to Hodgkin's disease, non-Hodgkin's disease; multiple myelomas such as but not limited to smoldering multiple myeloma, nonsecretory myeloma, osteosclerotic myeloma, plasma cell leukemia, solitary plasmacytoma and extramedullary plasmacytoma; Waldenstrom's macroglobulinemia; monoclonal gammopathy of undetermined significance; benign monoclonal gammopathy; heavy chain disease; bone cancer and connective tissue sarcomas such as but not limited to bone sarcoma, myeloma bone disease, multiple myeloma, cholesteatoma-induced bone osteosarcoma, Paget's disease of bone, osteosarcoma, chondrosarcoma, Ewing's sarcoma, malignant giant cell tumor, fibrosarcoma ofbone, chordoma, periosteal sarcoma, soft-tissue sarcomas, angiosarcoma (hemangiosarcoma), fibrosarcoma, Kaposi's sarcoma, leiomyosarcoma, liposarcoma, lymphangio sarcoma, neurilemmoma, rhabdomyosarcoma, and synovial sarcoma; brain tumors such as but not limited to, glioma, astrocytoma, brain stem glioma, ependymoma, oligodendroglioma, nonglial tumor, acoustic neurinoma, craniopharyngioma, medulloblastoma, meningioma, pineocytoma, pineoblastoma, and primary brain lymphoma; breast cancer including but not limited to adenocarcinoma, lobular (small cell) carcinoma, intraductal carcinoma, medullary breast cancer, mucinous breast cancer, tubular breast cancer, papillary breast cancer, Paget's disease (including juvenile Paget's disease) and inflammatory breast cancer; adrenal cancer such as but not limited to pheochromocytom and adrenocortical carcinoma; thyroid cancer such as but not limited to papillary or follicular thyroid cancer, medullary thyroid cancer and anaplastic thyroid cancer; pancreatic cancer such as but not limited to, insulinoma, gastrinoma, glucagonoma, vipoma, somatostatin-secreting tumor, and carcinoid or islet cell tumor; pituitary cancers such as but limited to Cushing's disease, prolactin-secreting tumor, acromegaly, and diabetes insipius; eye cancers such as but not limited to ocular melanoma such as iris melanoma, choroidal melanoma, and cilliary body melanoma, and retinoblastoma; vaginal cancers such as squamous cell carcinoma, adenocarcinoma, and melanoma; vulvar cancer such as squamous cell carcinoma, melanoma, adenocarcinoma, basal cell carcinoma, sarcoma, and Paget's disease; cervical cancers such as but not limited to, squamous cell carcinoma, and adenocarcinoma; uterine cancers such as but not limited to endometrial carcinoma and uterine sarcoma; ovarian cancers such as but not limited to, ovarian epithelial carcinoma, borderline tumor, germ cell tumor, and stromal tumor; cervical carcinoma; esophageal cancers such as but not limited to, squamous cancer, adenocarcinoma, adenoid cyctic carcinoma, mucoepidermoid carcinoma, adenosquamous carcinoma, sarcoma, melanoma, plasmacytoma, verrucous carcinoma, and oat cell (small cell) carcinoma; stomach cancers such as but not limited to, adenocarcinoma, fungating (polypoid), ulcerating, superficial spreading, diffusely spreading, malignant lymphoma, liposarcoma, fibrosarcoma, and carcinosarcoma; colon cancers; KRAS mutated colorectal cancer; colon carcinoma; rectal cancers; liver cancers such as but not limited to hepatocellular carcinoma and hepatoblastoma, gallbladder cancers such as adenocarcinoma; cholangiocarcinomas such as but not limited to pappillary, nodular, and diffuse; lung cancers such as KRAS-mutated non-small cell lung cancer, non-small cell lung cancer, squamous cell carcinoma (epidermoid carcinoma), adenocarcinoma, large-cell carcinoma and small-cell lung cancer; lung carcinoma; testicular cancers such as but not limited to germinal tumor, seminoma, anaplastic, classic (typical), spermatocytic, nonseminoma, embryonal carcinoma, teratoma carcinoma, choriocarcinoma (yolk-sac tumor), prostate cancers such as but not limited to, androgen-independent prostate cancer, androgen-dependent prostate cancer, adenocarcinoma, leiomyosarcoma, and rhabdomyosarcoma; penal cancers; oral cancers such as but not limited to squamous cell carcinoma; basal cancers; salivary gland cancers such as but not limited to adenocarcinoma, mucoepidermoid carcinoma, and adenoidcystic carcinoma; pharynx cancers such as but not limited to squamous cell cancer, and verrucous; skin cancers such as but not limited to, basal cell carcinoma, squamous cell carcinoma and melanoma, superficial spreading melanoma, nodular melanoma, lentigo malignant melanoma, acrallentiginous melanoma; kidney cancers such as but not limited to renal cell cancer, adenocarcinoma, hypernephroma, fibrosarcoma, transitional cell cancer (renal pelvis and/or uterer); renal carcinoma; Wilms' tumor; bladder cancers such as but not limited to transitional cell carcinoma, squamous cell cancer, adenocarcinoma, carcinosarcoma. In addition, cancers include myxosarcoma, osteogenic sarcoma, endotheliosarcoma, lymphangioendotheliosarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma and papillary adenocarcinomas.

In certain embodiments, cancers that can be prevented and/or treated in accordance with the present disclosure include, the following: pediatric solid tumor, Ewing's sarcoma, Wilms tumor, neuroblastoma, neurofibroma, carcinoma of the epidermis, malignant melanoma, cervical carcinoma, colon carcinoma, lung carcinoma, renal carcinoma, breast carcinoma, breast sarcoma, metastatic breast cancer, HIV-related Kaposi's sarcoma, prostate cancer, androgen-independent prostate cancer, androgen-dependent prostate cancer, neurofibromatosis, lung cancer, non-small cell lung cancer, KRAS-mutated non-small cell lung cancer, malignant melanoma, melanoma, colon cancer, KRAS-mutated colorectal cancer, glioblastoma multiforme, renal cancer, kidney cancer, bladder cancer, ovarian cancer, hepatocellular carcinoma, thyroid carcinoma, rhabdomyosarcoma, acute myeloid leukemia, and multiple myeloma.

In some embodiments, cancers and conditions associated therewith that are prevented and/or treated in accordance with the present disclosure are triple negative breast cancer, metastatic colorectal cancer, endometrial cancer, metastatic melanoma, hereditary nonpolyposis colorectal cancer, adenocarcinoma, sarcoma, melanoma, liver cancer, hepatocellular carcinoma, hepatoblastoma, liver carcinoma, prostate cancer, prostate adenocarcinoma, androgen-independent prostate cancer, androgen-dependent prostate cancer, leiomyosarcoma, rhabdomyosarcoma, prostate carcinoma, brain cancer, glioma, astrocytoma, brain stem glioma, ependymoma, oligodendroglioma, nonglial tumor, acoustic neurinoma, craniopharyngioma, medulloblastoma, meningioma, pineocytoma, pineoblastoma, primary brain lymphoma, anaplastic astrocytoma, juvenile pilocytic astrocytoma, a mixture of oligodendroglioma and astrocytoma elements, breast cancer, metastatic breast cancer, breast carcinoma, breast sarcoma, adenocarcinoma, lobular (small cell) carcinoma, intraductal carcinoma, medullary breast cancer, mucinous breast cancer, tubular breast cancer, papillary breast cancer, Paget's disease, juvenile Paget's disease, inflammatory breast cancer, lung cancer, KRAS-mutated non-small cell lung cancer, non-small cell lung cancer, squamous cell carcinoma (epidermoid carcinoma), adenocarcinoma, large-cell carcinoma, small cell lung cancer, lung carcinoma, colon cancer, KRAS mutated colorectal cancer, colon carcinoma, pancreatic cancer, insulinoma, gastrinoma, glucagonoma, vipoma, somatostatin-secreting tumor, carcinoid tumor, islet cell tumor, pancreas carcinoma, skin cancer, skin melanoma, basal cell carcinoma, squamous cell carcinoma, melanoma, superficial spreading melanoma, nodular melanoma, lentigo malignant melanoma, acrallentiginous melanoma, skin carcinoma, cervical cancer, cervical cancer, squamous cell carcinoma, adenocarcinoma, cervical carcinoma, ovarian cancer, ovarian epithelial carcinoma, borderline tumor, germ cell tumor, stromal tumor, ovarian carcinoma, cancer of the mouth, blood cancer, leukemia, acute myeloid leukemia, acute monocytic leukemia, chronic myeloid leukemia, acute lymphoblastic leukemia, chronic lymphocytic leukemia, acute leukemia, acute lymphocytic leukemia, acute myelocytic leukemia, myeloblastic leukemia, promyelocytic leukemia, myelomonocytic leukemia, monocytic leukemia, erythroleukemia, myclodysplastic syndrome, chronic leukemia, chronic myelocytic (granulocytic) leukemia, chronic lymphocytic leukemia, hairy cell leukemia, plasma cell leukemia, cancer of the nervous system, cancer of the central nervous system, a primary central nervous system (CNS) lymphoma, a CNS germ cell tumor, goblet cell metaplasia, kidney cancer, renal cell cancer, adenocarcinoma, hypernephroma, fibrosarcoma, transitional cell cancer (renal pelvis and/or uterer), bladder cancer, transitional cell carcinoma, squamous cell cancer, adenocarcinoma, carcinosarcoma, stomach cancer, stomach cancer, adenocarcinoma, fungating (polypoid), ulcerating, superficial spreading, diffusely spreading, malignant lymphoma, liposarcoma, fibrosarcoma, carcinosarcoma, uterine cancer, endometrial carcinoma, uterine sarcoma, cancer of the esophagus, squamous cancer, adenocarcinoma, adenoid cyctic carcinoma, mucoepidermoid carcinoma, adenosquamous carcinoma, sarcoma, melanoma, plasmacytoma, verrucous carcinoma, and oat cell(small cell) carcinoma, esophageal carcinomas, cancer of the rectum, colorectal cancer, rectal cancers, colorectal carcinoma, gallbladder cancer, adenocarcinoma, cholangiocarcinoma, pappillary cholangiocarcinoma, nodular cholangiocarcinoma, diffuse cholangiocarcinoma, testicular cancer, germinal tumor, seminoma, anaplastic testicular cancer, classic (typical) testicular cancer, spermatocytic testicular cancer, nonseminoma testicular cancer, embryonal carcinoma, teratoma carcinoma, choriocarcinoma (yolk-sac tumor), gastric cancer, gastrointestinal stromal tumor, cancer of other gastrointestinal tract organs, gastric carcinomas, bone cancer, connective tissue sarcoma, bone sarcoma, myeloma bone disease, multiple myeloma, cholesteatoma-induced bone osteosarcoma, Paget's disease of bone, osteosarcoma, chondrosarcoma, Ewing's sarcoma, malignant giant cell tumor, fibrosarcoma of bone, chordoma, periosteal sarcoma, soft-tissue sarcoma, angiosarcoma (hemangiosarcoma), fibrosarcoma, Kaposi's sarcoma, leiomyosarcoma, liposarcoma, lymphangiosarcoma, neurilemmoma, rhabdomyosarcoma, synovial sarcoma, Hodgkin's lymphoma, non-Hodgkin's lymphoma, anaplastic large cell lymphoma, cancer of the lymph node, lymphangioendotheliosarcoma, myeloma, multiple myeloma, smoldering multiple myeloma, nonsecretory myeloma, osteosclerotic myeloma, solitary plasmacytoma, extramedullary plasmacytoma, alveolar soft part sarcoma, adenoid cystic carcinoma, renal cell carcinoma, transitional cell carcinoma, germ cell cancer, a malignant glioma, renal carcinoma, vaginal cancer, squamous cell carcinoma, adenocarcinoma, melanoma, vulvar cancer, squamous cell carcinoma, melanoma, adenocarcinoma, sarcoma, Paget's disease, cancer of other reproductive organs, thyroid cancer, papillary thyroid cancer, follicular thyroid cancer, medullary thyroid cancer, anaplastic thyroid cancer, thyroid carcinoma, salivary gland cancer, adenocarcinoma, mucoepidermoid carcinoma, eye cancer, ocular melanoma, iris melanoma, choroidal melanoma, cilliary body melanoma, retinoblastoma, penal cancers, oral cancer, squamous cell carcinoma, basal cancer, pharynx cancer, squamous cell cancer, verrucous pharynx cancer, Wilms' tumor, cancer of the head, cancer of the neck, cancer of the eye, cancer of the throat, cancer of the chest, cancer of the spleen, cancer of skeletal muscle, cancer of subcutaneous tissue, adrenal cancer, pheochromocytoma, adrenocortical carcinoma, pituitary cancer, Cushing's disease, prolactin-secreting tumor, acromegaly, diabetes insipidus, myxosarcoma, osteogenic sarcoma, endotheliosarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma, papillary adenocarcinomas, ependyoma, optic nerve glioma, primitive neuroectodermal tumor, rhabdoid tumor, renal cancer, glioblastoma multiforme, neurofibroma, neurofibromatosis, pediatric cancer, neuroblastoma, malignant melanoma, carcinoma of the epidermis, polycythemia vera, Waldenstrom's macroglobulinemia, monoclonal gammopathy of undetermined significance, benign monoclonal gammopathy, heavy chain disease, pediatric solid tumor, Ewing's sarcoma, Wilms tumor, carcinoma of the epidermis, HIV-related Kaposi's sarcoma, rhabdomyosarcoma, thecomas, arrhenoblastomas, endometrial carcinoma, endometrial hyperplasia, endometriosis, fibrosarcomas, choriocarcinoma, nasopharyngeal carcinoma, laryngeal carcinoma, hepatoblastoma, Kaposi's sarcoma, hemangioma, cavernous hemangioma, hemangioblastoma, retinoblastoma, glioblastoma, Schwannoma, neuroblastoma, rhabdomyosarcoma, osteogenic sarcoma, leiomyosarcoma, urinary tract carcinoma, abnormal vascular proliferation associated with phakomatoses, edema (such as that associated with brain tumors), Meigs' syndrome, pituitary adenoma, primitive neuroectodermal tumor, medullblastoma, and acoustic neuroma.

In certain embodiments, cancers and conditions associated therewith that are prevented and/or treated in accordance with the present disclosure are breast carcinomas, lung carcinomas, gastric carcinomas, esophageal carcinomas, colorectal carcinomas, liver carcinomas, ovarian carcinomas, thecomas, arrhenoblastomas, cervical carcinomas, endometrial carcinoma, endometrial hyperplasia, endometriosis, fibrosarcomas, choriocarcinoma, head and neck cancer, nasopharyngeal carcinoma, laryngeal carcinomas, hepatoblastoma, Kaposi's sarcoma, melanoma, skin carcinomas, hemangioma, cavernous hemangioma, hemangioblastoma, pancreas carcinomas, retinoblastoma, astrocytoma, glioblastoma, Schwannoma, oligodendroglioma, medulloblastoma, neuroblastomas, rhabdomyosarcoma, osteogenic sarcoma, leiomyosarcomas, urinary tract carcinomas, thyroid carcinomas, Wilm's tumor, renal cell carcinoma, prostate carcinoma, abnormal vascular proliferation associated with phakomatoses, edema (such as that associated with brain tumors), or Meigs' syndrome. In specific embodiment, the cancer an astrocytoma, an oligodendroglioma, a mixture of oligodendroglioma and an astrocytoma elements, an ependymoma, a meningioma, a pituitary adenoma, a primitive neuroectodermal tumor, a medullblastoma, a primary central nervous system (CNS) lymphoma, or a CNS germ cell tumor.

In specific embodiments, the cancer treated in accordance with the present disclosure is an acoustic neuroma, an anaplastic astrocytoma, a glioblastoma multiforme, or a meningioma.

In other specific embodiments, the cancer treated in accordance with the present disclosure is a brain stem glioma, a craniopharyngioma, an ependyoma, a juvenile pilocytic astrocytoma, a medulloblastoma, an optic nerve glioma, primitive neuroectodermal tumor, or a rhabdoid tumor.

In some aspects of the present disclosure, small molecules identified by the screening methods can be formulated for administration to a mammal by intravenous administration, subcutaneous administration, oral administration, inhalation, nasal administration, dermal administration, or ophthalmic administration. In one aspect, small molecules identified by the screening methods can be used to treat a disease or condition that can be treated by modulating RNA splicing of a protein associated with the disease or condition.

In some embodiments, a small molecule identified by the present disclosure has a molecular weight of at most about 2000 Daltons, 1500 Daltons, 1000 Daltons or 900 Daltons. In some embodiments, a small molecule identified by the present disclosure has a molecular weight of at least 100 Daltons, 200 Daltons, 300 Daltons, 400 Daltons or 500 Daltons. In some embodiments, a small molecule identified by the present disclosure does not comprise a phosphodiester linkage.

The small molecules identified in the present disclosure can be used to modulate aberrant splicing caused by mutation in 5′ss, cryptic 5′ss, 3′ss, cryptic 3′ss, ESE, ESS, ISE, and/or ISS. The modulation can include both enhance/activate and prevent/inhibit. In some embodiments, the modulation can be enhancement/activation, wherein the small molecule stabilizes or enhances binding of one polynucleotide or polypeptide binding to a target polynucleotide. For example, small molecules can bind to target mRNAs and therefore promote the binding of additional polynucleotide or polypeptide binding to the target polynucleotide. In some cases, the small molecules can promote the binding of an RNA binding to a target mRNA. In some cases, the small molecule can promote the binding of a protein or portion thereof binding to a target mRNA. In some cases, the small molecules can promote the binding of a protein or a portion thereof binding to a target RNA-RNA duplex. In some cases, the small molecules can promote the binding of a protein-RNA complex (e.g. snRNP) binding to a target mRNA. In some cases, the small molecules can promote the binding of a protein or a portion thereof binding to a target RNA-RNA duplex by changing secondary or tertiary structure or molecular moiety of the target mRNA. For example, small molecules can promote binding of a polynucleotide and/or a polypeptide binding to a target mRNA containing a 5′ss or 3′ss or a portion thereof; thereby facilitating inclusion of the adjacent exon.

In some embodiments, the modulation can be prevention/inhibition, wherein the small molecule destabilizes or prevents one polynucleotide or polypeptide from binding to a target polynucleotide. For example, small molecules can bind to target mRNAs and therefore prevent additional polynucleotide or polypeptide from binding to the target polynucleotide. In some cases, the small molecules can prevent a RNA from binding to a target mRNA. In some cases, the small molecules can prevent a protein or a portion thereof from binding to a target mRNA. In some cases, the small molecules can prevent a protein or a portion thereof from binding to a target RNA-RNA duplex. In some cases, the small molecules can prevent a protein-RNA complex (e.g. snRNP) from binding to a target mRNA. In some cases, the small molecules can promote the binding of a protein or a portion thereof binding to a target RNA-RNA duplex by changing secondary or tertiary structure or molecular moiety of the target mRNA. For example, small molecules can prevent a polynucleotide and/or a polypeptide binding to a target mRNA containing a cryptic 5′ss or cryptic 3′ss or a portion thereof; thereby facilitating inclusion of the adjacent exon. For example, small molecules can prevent a polynucleotide and/or a polypeptide binding to a target mRNA containing an authentic 5′ss or authentic 3′ss or a portion thereof; thereby facilitating the loss of an exon.

The small molecules identified in the present disclosure can be used to treat a disease or condition associated with aberrant splicing in one or more proteins. The small molecules identified in the present disclosure may be used to modulate splicing, for example modulating the amount of RNA transcripts generated. In some embodiments, the small molecules identified in the present disclosure may be used to modulate splicing not related to any mutation in the cis-acting elements.

In some embodiments, a small molecule identified in the present disclosure modulates splicing of a splice site sequence comprising a sequence GGA/gugagu, AGA/gugagu, AGA/gugagu, AGA/gugagu, AGA/gugagu, AGA/gugagu, AGA/gugagc, AGA/gugagu, AGA/gugagu, GGA/gugagu, CGA/guccgu, GGAguaagu, GGA/guaagu, AGA/guaagu, AGA/guaagu, AGA/guaagu, AGA/guaagu, AGA/guaagu, AGA/guaagu, AGA/guaaga, AGA/guaagu, AGA/guaagu, AGA/guaagu, GGA/guaagu, AGA/guaagg, AGA/guaagu, AGA/guaagu, AGA/guaagu, GGA/guaagu, AGA/guaaga, AGA/guaagu, AGA/guaagu, AGA/guaagu, GGA/guaagg, AGA/guaagu, AGA/guaagu, GGA/guaagu, AGA/guaagu, AGA/guaaga, AGA/guaagu, AGA/guagau, UGA/gugaau, GGA/guuagu, AGA/guaggu, AGA/guaggu, GGA/guaggu, or AGA/gugcgu. In some embodiments, a small molecule identified in the present disclosure modulates splicing of a splice site sequence comprising a sequence ACA/gugagg, AAA/auaagu, GAA/ggaagu, GAA/guaaau, GCA/guagga, CAA/gugagu, GUA/gugagu, GAA/guggg, CCA/guaaac, UUA/guaaau, CAA/guaaac, ACA/guaaau, GAA/guaaac, UCA/guaaac, UCA/guaaau, GCA/guaaau, ACA/guaaau, CAA/guaagc, CAA/guaagg, UCA/guaagu, AUA/gugaau, CAA/gugaaa, CCA/gugaga, UCA/gugauu, GAA/gugugu, GAA/uaaguu, CAA/guaugu, AAA/guaugu, CAA/guauuu, ACA/guuagu, GCA/guuagu, or ACA/guuuga. In some embodiments, a small molecule identified in the present disclosure modulates splicing of a splice site sequence comprising a sequence CAA/guaacu, AUA/gucagu, GAA/gucugg, AAA/guacau. In some embodiments, a small molecule identified in the present disclosure modulates splicing of a splice site sequence comprising a sequence NNBgunnnn, NNBhunnnn, or NNBgvnnnn In some embodiments, a small molecule identified in the present disclosure modulates splicing of a splice site sequence comprising a sequence NNBgurrrn, NNBguwwdn, NNBguvmvn, NNBguvbbn, NNBgukddn, NNBgubnbd, NNBhunngn, NNBhurmhd, or NNBgvdnvn. In those embodiments, N (or n) is A, U, G or C; B (or b) is C, G, or U; H (or h) is A, C, or U; d is a, g, or u; m is a or c; r is a or g; v is a, c or g; k is g or u; w is a or u. In some embodiments, a small molecule identified in the present disclosure modulates splicing of a splice site sequence comprising a sequence CAC/gugagc, UCC/gugagc, AGC/gugagu, AGC/gugagu, AGG/gugagg, GUG/gugagc, GAG/gugagg, CCG/gugagg, UUG/gugagc, GUG/gugagu, UUU/gugagc, UUU/gugagc, GAU/gugagg, AGU/gugagu, AGU/gugagu, AGU/gugagu, AGU/gugagu, AGC/guaagu, GGC/guaagu, AAC/guaagu, GGC/guaagu, AGC/guaagg, GGC/guaagu, AGC/guaagu, GGC/guaagu, GGC/guaagu, AGC/guaagu, GAG/guaaga, CAG/guaagu, AGU/guaagc, AAU/guaagc, AAU/guaagg, CCU/guaagc, AGU/guaagu, GGU/guaagu, AGU/guaagu, AGU/guaagu, AGU/guaagu, GAU/guaagu, UCC/gugaau, CCG/gugaau, ACG/gugaac, CUG/gugaau, AGG/gugaau, UUG/gugaau, CCG/gugaau, GAG/gugaag, CCU/gugaau, CGU/gugaau, CCU/gugaau, GAG/guagga, CAU/guaggg, UGG/guggau, CAG/guggau, UGG/guggau, CGG/gugggu, GCG/guggga, UGG/guggggg, UGG/gugggug, CGU/gugggu, AUC/gguaaaa, GGG/guaaau, GCG/guaaaa, CAG/guaaag, UGG/guaaag, AAG/guaaag, AAG/guaaau, CAG/guaaag, UAG/guaaag, UUG/guaaag, GAG/guaaag, CAG/guaaag, AUG/guaaaa, AAG/guaaag, CAG/guaaag, CAG/guaaaa, GAG/guaaag, AAG/guaaag, UGU/guaaau, GUU/guaaau, GUU/guaaau, UCU/guaaau, GCU/guaaau, GAU/guaaau, GCU/guaaau, UCU/guaaau, ACU/guaaau, CCU/guaaau, CCU/guaaau, ACU/guaaau, AAU/guaaau, AGG/guagac, UUG/guagau, CAG/guagag, AAG/guagag, AAU/gugagu, CAG/gugagc, AAG/gugggu, AAG/guaggg, CAG/guaggc, or AGC/guaggu. In some embodiments, a small molecule identified in the present disclosure modulates splicing of a splice site sequence comprising a sequence CAG/guaau, CAG/guaaugu, CAG/guaaugu, CAG/guaaugu, CAG/guaaugu, GAG/guaauac, GAG/guaauau, GAG/guaaugu, AAG/guaauaa, AAG/guaaugu, AAG/guaaugu, AAG/guaaugua, AAG/guaaugu, AAG/guaaugu, GCU/guaauu, CCU/guaauu, GAU/guaauu, CAU/guaauu, AAU/guaauu, AGG/guauau, CAG/guauau, UAG/guauau, CAG/guauau, CGG/guauau, GAG/guauau, CGG/guauau, CAG/guauag, AAG/guauau, CAG/guauag, AAG/guauac, UAG/guauau, CAG/guauag, CAG/guauau, AAG/guuaag, AUC/guuaga, GCG/guuagu, AAG/guuagc, UGG/guuagu, GCG/guuagu, CUG/guuugu, CUG/guauga, CAG/guauga, UAG/guauga, AAG/guaugg, AAG/guauga, GAG/guaugg, CAG/guauga, CAG/guaugg, AAG/guaugg, UGG/guaugc, CAG/guaugu, AUG/guaugu, AAG/guaugu, AAG/guaugg, CAG/guaugg, GAG/guauga, CGG/guaugg, AAU/guaugu, AAG/guauuu, AUG/guauuu, UAG/guauug, AAG/guauuu, CAG/guauug, CAG/guauug, CAU/guauuu, ACU/guauu, AAG/guuuau, AAG/guuuaa, CAG/guuugg, CAG/guuugg, CAG/guuugc, AAG/guuugg, AAG/guuugg, or UGG/guaugc. In some embodiments, a small molecule identified in the present disclosure modulates splicing of a splice site sequence comprising a sequence CCG/guaacu, UUG/guaaca, AUG/guaacc, GGG/guaacu, AAG/guaaca, AAG/guaacu, UUG/guaaca, GCU/guaacu, ACU/guaacu, GCU/guaacu, UAG/guaccc, AAG/guaccu, CAG/guaccg, UGG/guacca, CAG/gucaau, AAG/gucaau, AAG/gucaag, AUG/guacau, GGG/guacau, UUG/guacau, CAG/guacag, CAG/guacag, CAG/guacag, CAG/guacag, AAG/guacag, CAG/guacag, GAG/guacaa, AAG/guacag, CAG/guacaa, UGU/guacau, CAG/gugcac, GGG/gugcau, CUG/gugcau, UAG/gugcau, CAG/gugcag, CAG/gugcag, AGG/gugcaa, AAC/gugacu, UCC/gugacu, CCG/gugacu, GCG/gugacu, GGG/gugacg, GGG/gugacg, GCG/gugacu, AUG/gugacc, GAU/gugacu, GGC/gucagu, or UAG/gucaga. In some embodiments, a small molecule identified in the present disclosure modulates splicing of a splice site sequence comprising a sequence AAG/guacgg, AAG/guacgg, AAG/guacug, AAG/guagcg, AAG/guagua, AAG/guagua, AAG/guagua, AAG/guagug, AAG/guauca, AAG/guaucg, AAG/guaucu, AAG/gucucu, AAG/gugccu, AAG/guggua, AAG/guguua, ACG/guagcu, AGC/guacgu, CAG/guacug, CAG/guagua, CAG/guagug, CAG/guagug, CAG/guaucc, CAG/gugcgc, or GAG/gugccu. In some embodiments, a small molecule identified in the present disclosure modulates splicing of a splice site sequence comprising a sequence CGG/guguau, AAG/guguau, GAG/guguac, CAG/guguau, UAG/guguau, CAG/guguag, GAG/guguau, AAG/gugugc, CAG/guguga, AAG/gugugu, CAG/guguga, CAG/gugugu, UGG/gugugg, CUG/guguga, CGG/gugugu, GAG/gugugc, CAG/guguga, AAU/gugugu, CAG/gugugu, CAG/gugugu, GAG/gugugu, CAG/guuguu, CAG/guuguc, GUG/guugua, CAG/guuguu, AAC/gugauu, CAG/gugaua, AGG/gugauc, GUG/gugauc, CCU/gugauu, GAU/gugauu, CAC/guuggu, CAG/guuggc, AAG/guuagc, or CAG/guugau. In some embodiments, a small molecule identified in the present disclosure modulates splicing of a splice site sequence comprising a sequence AUG/gucauu, CGG/gucauaauc, AAG/gucugu, AAG/gucuggg, CAG/gucugga, CAG/gucuggu, CAG/gucuga, GAG/gucuggu, AAG/gugucu, AAG/gugucu, AGG/gugucu, CUG/gugcuu, CAG/gucuuu, CAG/guugcu, GAG/gugcug, or CAG/gugcug. In some embodiments, a small molecule identified in the present disclosure modulates splicing of a splice site sequence comprising a sequence CGC/auaagu, UUC/auaagu, UGG/auaagg, ACG/auaagg, GUU/auaagu, CCU/auaagu, UUU/auaagc, GAG/aucugg, AAC/augagga, GAC/augagg, ACC/augagu, GGG/augagu, AAG/augagc, CAG/augagg, GAG/augagg, GCG/augagu, AAG/gaugag, CCU/augagu, GAU/augagu, GAU/augagu, UAG/augcgu, CAG/auuggu, AAG/auuugu, ACG/cuaagc, CAG/cugugu, CUG/uuaag, GAG/uuaagu, AAG/uuaagg, AUU/uuaagc, CUG/uugaga, CAG/uuuggu, or GGG/auaagu. In some embodiments, a small molecule identified in the present disclosure modulates splicing of a splice site sequence comprising a sequence CAG/auaacu, GAG/cugcag, or AAG/uuaaua. In some embodiments, a small molecule identified in the present disclosure modulates splicing of a splice site sequence comprising a sequence GCG/gagagu, AAG/ggaaaa, AUC/gguaaaa, AAG/gcaaaa, UGU/gcaagu, GAG/gcaggu, GAG/gcgugg, GAG/gcuccc, CAG/gcuggu, or AAG/gaugag.

Exemplary small molecules that could be identified by the present disclosure are summarized in Table 3.

TABLE 3
Exemplary small molecule structures

| SMSM# | Compound Name | Compound Structure |
|---|---|---|
| 1 | (4-(1H-pyrazol-4-yl)phen- yl)(2-(piperazin-1-yl)- pyridin-4-yl)methanone | 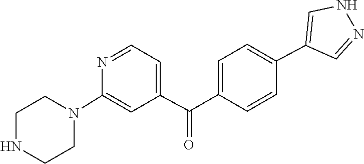 [SMILES: O=C(c1ccc(-c2cn[nH]c2)cc1)c1ccnc(N2CCNCC2)c1] |
| 2 | (4-(1H-pyrazol-4-yl)phen- yl)(2-(4-aminopiperidin- 1-yl)pyridin-4-yl)meth- anone | 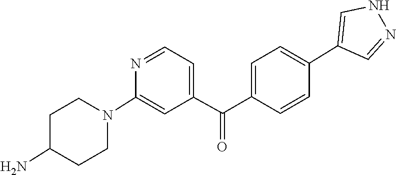 [SMILES: NC1CCN(c2cc(C(=O)c3ccc(-c4cn[nH]c4)cc3)ccn2)CC1] |
| 3 | (4-(1H-pyrazol-4-yl)phen- yl)(2-(3-aminoazetidin- 1-yl)pyridin-4-yl)meth- anone | 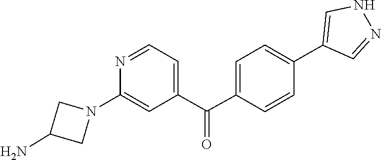 [SMILES: NC1CN(c2cc(C(=O)c3ccc(-c4cn[nH]c4)cc3)ccn2)C1] |
| 4 | (4-(1H-pyrazol-4-yl)phen- yl)(2-(3-aminopyrrolidin- 1-yl)pyridin-4-yl)meth- anone | 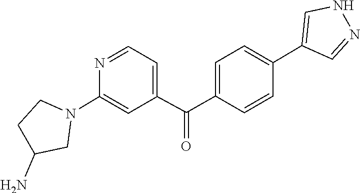 [SMILES: NC1CCN(c2cc(C(=O)c3ccc(-c4cn[nH]c4)cc3)ccn2)C1] |
| 5 | (2-methylbenzo[d]oxazol- 6-yl)(2-(piperazin-1-yl)- pyridin-4-yl)methanone | 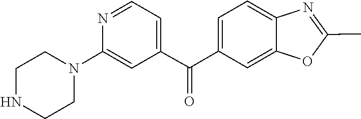 [SMILES: Cc1nc2ccc(C(=O)c3ccnc(N4CCNCC4)c3)cc2o1] |
| 6 | (2-(4-aminopiperidin- 1-yl)pyridin-4-yl)(2- methylbenzo[d]oxazol- 6-yl)methanone | 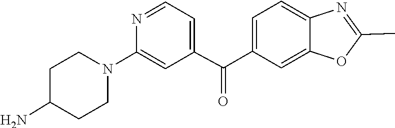 [SMILES: Cc1nc2ccc(C(=O)c3ccnc(N4CCC(N)CC4)c3)cc2o1] |
| 7 | (3-(2H-tetrazol-5-yl)- bicyclo[1.1.1]pentan- 1-yl)(2-(piperazin-1- yl)pyridin-4-yl)meth- anone | 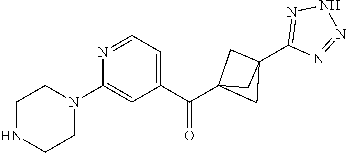 [SMILES: O=C(c1ccnc(N2CCNCC2)c1)C12CC(c3nn[nH]n3)(C1)C2] |
| 8 | 2-(4-(1H-pyrazol-4-yl)- phenoxy)-4-(piperazin- 1-yl)-1,3,5-triazine | 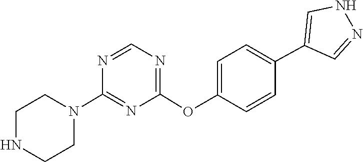 [SMILES: c1nc(Oc2ccc(-c3cn[nH]c3)cc2)nc(N2CCNCC2)n1] |
| 9 | 1-(4-(4-(1H-pyrazol-4- yl)phenoxy)-1,3,5- triazin-2-yl)-piperidin- 4-amine | 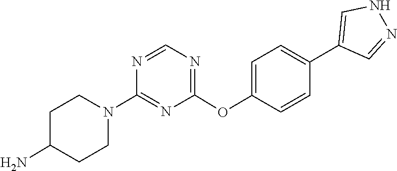 [SMILES: NC1CCN(c2ncnc(Oc3ccc(-c4cn[nH]c4)cc3)n2)CC1] |
| 10 | 2-methyl-6-((4-(piperazin- 1-yl)-1,3,5-triazin-2-yl)- oxy)benzo[d]oxazole | 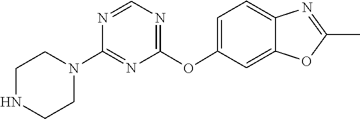 [SMILES: Cc1nc2ccc(Oc3ncnc(N4CCNCC4)n3)cc2o1] |
| 11 | 1-(4-((2-methylbenzo[d]- oxazol-6-yl)oxy)-1,3,5- triazin-2-yl)piperidin-4- amine | 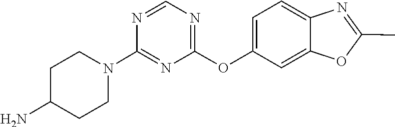 [SMILES: Cc1nc2ccc(Oc3ncnc(N4CCC(N)CC4)n3)cc2o1] |
| 12 | 2-methyl-N-(4-(piperazin- 1-yl)-1,3,5-triazin-2-yl)- benzo[d]oxazol-6-amine | 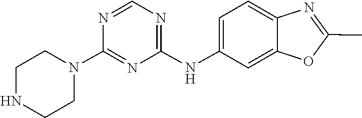 [SMILES: Cc1nc2ccc(Nc3ncnc(N4CCNCC4)n3)cc2o1] |
| 13 | N-(4-(4-aminopiperidin- 1-yl)-1,3,5-triazin-2-yl)- 2-methylbenzo[d]oxazol- 6-amine | 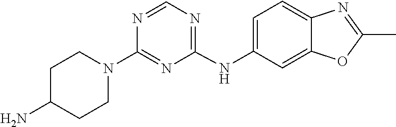 [SMILES: Cc1nc2ccc(Nc3ncnc(N4CCC(N)CC4)n3)cc2o1] |
| 14 | N-(4-(1H-pyrazol-4-yl)- phenyl)-4-(piperazin-1- yl)-1,3,5-triazin-2-amine | 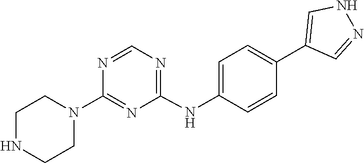 [SMILES: c1nc(Nc2ccc(-c3cn[nH]c3)cc2)nc(N2CCNCC2)n1] |
| 15 | N-(4-(1H-pyrazol-4-yl)- phenyl)-4-(4-amino- piperidin-1-yl)-1,3,5- triazin-2-amine | 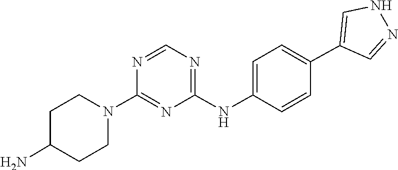 [SMILES: NC1CCN(c2ncnc(Nc3ccc(-c4cn[nH]c4)cc3)n2)CC1] |
| 16 | 2-methyl-5-((6-(piperazin- 1-yl)pyridazin-3-yl)oxy)- benzo[d]oxazole | 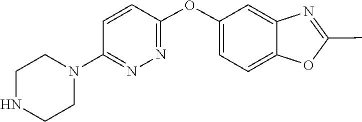 [SMILES: Cc1nc2cc(Oc3ccc(N4CCNCC4)nn3)ccc2o1] |
| 17 | 1-(6-((2-methylbenzo- [d]oxazol-5-yl)oxy)- pyridazin-3-yl)piperidin- 4-amine | 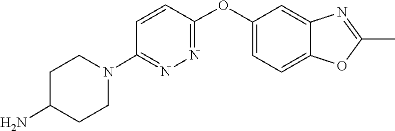 [SMILES: Cc1nc2cc(Oc3ccc(N4CCC(N)CC4)nn3)ccc2o1] |
| 18 | 3-(3-(1H-pyrazol-4-yl)- phenoxy)-6-(piperazin- 1-yl)pyridazine | 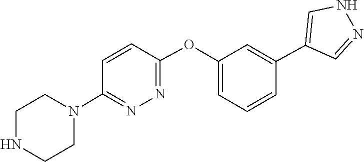 [SMILES: c1cc(Oc2ccc(N3CCNCC3)nn2)cc(-c2cn[nH]c2)c1] |
| 19 | 1-(6-(3-(1H-pyrazol-4- yl)phenoxy)pyridazin- 3-yl)piperidin-4-amine | 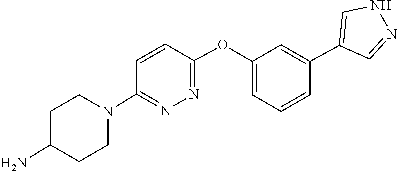 [SMILES: NC1CCN(c2ccc(Oc3cccc(-c4cn[nH]c4)c3)nn2)CC1] |
| 20 | 1-(6-(3-(1H-pyrazol-4- yl)phenoxy)pyridazin- 3-yl)piperidin-3-amine | 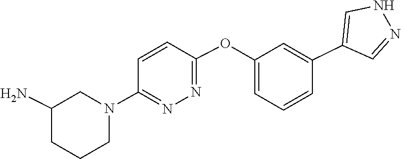 [SMILES: NC1CCCN(c2ccc(Oc3cccc(-c4cn[nH]c4)c3)nn2)C1] |
| 21 | 2-methyl-N-(6-(pipera- zin-1-yl)pyridazin-3- yl)benzo[d]oxazol-5- amine | 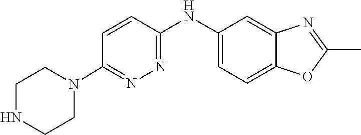 [SMILES: Cc1nc2cc(Nc3ccc(N4CCNCC4)nn3)ccc2o1] |
| 22 | N-(6-(4-aminopiperidin- 1-yl)pyridazin-3-yl)-2- methylbenzo[d]oxazol- 5-amine | 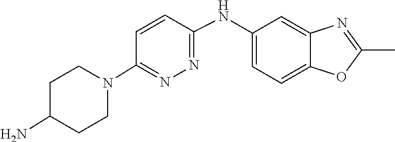 [SMILES: Cc1nc2cc(Nc3ccc(N4CCC(N)CC4)nn3)ccc2o1] |
| 23 | N-(3-(1H-pyrazol-4- yl)phenyl)-6-(piperazin- 1-yl)pyridazin-3-amine | 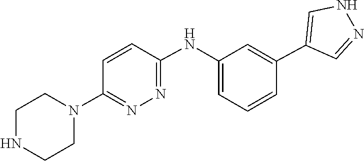 [SMILES: c1cc(Nc2ccc(N3CCNCC3)nn2)cc(-c2cn[nH]c2)c1] |
| 24 | N-(3-(1H-pyrazol-4- yl)phenyl)-6-(4- aminopiperidin-1-yl)- pyridazin-3-amine | 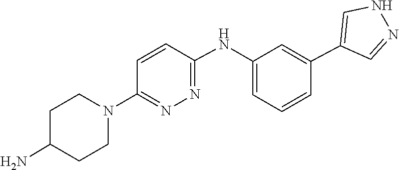 [SMILES: NC1CCN(c2ccc(Nc3cccc(-c4cn[nH]c4)c3)nn2)CC1] |
| 25 | N-(3-(1H-pyrazol-4- yl)phenyl)-6-(3- aminopiperidin-1-yl)- pyridazin-3-amine | 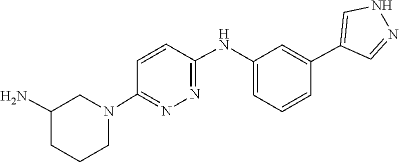 [SMILES: NC1CCCN(c2ccc(Nc3cccc(-c4cn[nH]c4)c3)nn2)C1] |
| 26 | 3-(piperazin-1-yl)-8- (1H-pyrazol-4-yl)- 5H-chromeno[2,3-c]- pyridin-5-one | 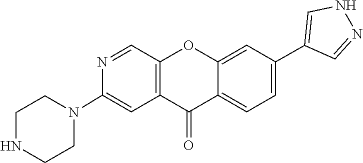 [SMILES: O=c1c2ccc(-c3cn[nH]c3)cc2oc2cnc(N3CCNCC3)cc12] |
| 27 | 3-(methyl(piperidin-4- yl)amino)-8-(1H- pyrazol-4-yl)-5H- chromeno[2,3-c]- pyridin-5-one | 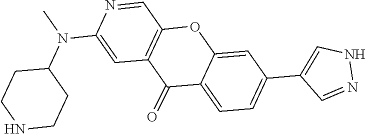 [SMILES: CN(c1cc2c(=O)c3ccc(-c4cn[nH]c4)cc3oc2cn1)C1CCNCC1] |
| 28 | 3-(3-aminopiperidin-1- yl)-8-(1H-pyrazol-4- yl)-5H-chromeno[2,3- c]pyridin-5-one | 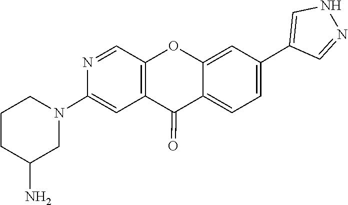 [SMILES: NC1CCCN(c2cc3c(=O)c4ccc(-c5cn[nH]c5)cc4oc3cn2)C1] |
| 29 | 3-(4-aminopiperidin-1- yl)-8-(1H-pyrazol-4- yl)-5H-chromeno- [2,3-c] pyridin-5-one | 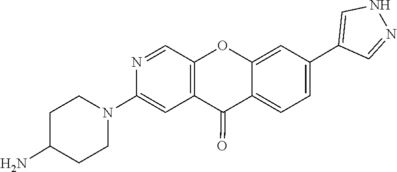 [SMILES: NC1CCN(c2cc3c(=O)c4ccc(-c5cn[nH]c5)cc4oc3cn2)CC1] |
| 30 | 3-(piperazin-1-yl)-8- (1H-tetrazol-5-yl)- 5H-chromeno[2,3-c]- pyridin-5-one | 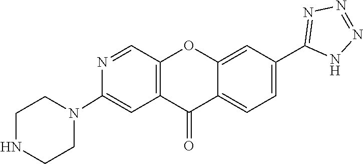 [SMILES: O=c1c2ccc(-c3nnn[nH]3)cc2oc2cnc(N3CCNCC3)cc12] |
| 31 | 3-(methyl(piperidin-4- yl)amino)-8-(1H- tetrazol-5-yl)-5H- chromeno[2,3-c]- pyridin-5-one | 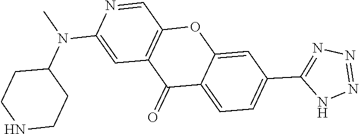 [SMILES: CN(c1cc2c(=O)c3ccc(-c4nnn[nH]4)cc3oc2cn1)C1CCNCC1] |
| 32 | 3-(4-aminopiperidin- 1-yl)-8-(1H-tetrazol-5- yl)-5H-chromeno[2,3- c]pyridin-5-one | 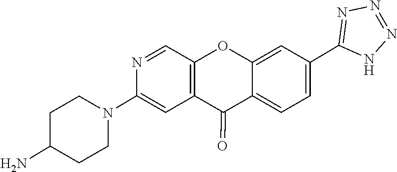 [SMILES: NC1CCN(c2cc3c(=O)c4ccc(-c5nnn[nH]5)cc4oc3cn2)CC1] |
| 33 | N1-(2-aminopyrimidin- 5-yl)-N4-methyl-N4- (piperidin-4-yl)- terephthalamide | 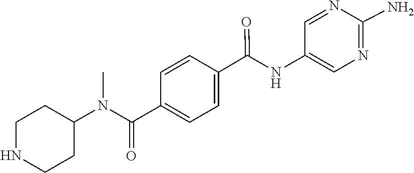 [SMILES: CN(C(=O)c1ccc(C(=O)Nc2cnc(N)nc2)cc1)C1CCNCC1] |
| 34 | N1-(2-aminopyrimidin- 5-yl)-N1,N4-dimethyl- N4-(piperidin-4-yl)- terephthalamide | 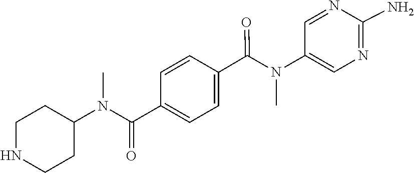 [SMILES: CN(C(=O)c1ccc(C(=O)N(C)C2CCNCC2)cc1)c1cnc(N)nc1] |
| 35 | N1,N4-dimethyl-N1- (piperidin-4-yl)-N4- (1H-pyrazol-4-yl)- terephthalamide | 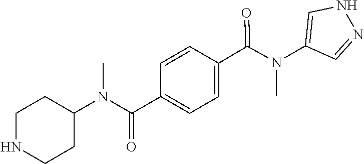 [SMILES: CN(C(=O)c1ccc(C(=O)N(C)C2CCNCC2)cc1)c1cn[nH]c1] |
| 37 | N1-methyl-N1- (piperidin-4-yl)-N4- (1H-pyrazol-4-yl)- terephthalamide | 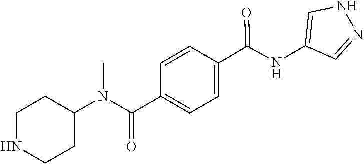 [SMILES: CN(C(=O)c1ccc(C(=O)Nc2cn[nH]c2)cc1)C1CCNCC1] |
| 38 | N1,N4-dimethyl-N1- (piperidin-3-yl)-N4- (1H-pyrazol-4-yl)- terephthalamide | 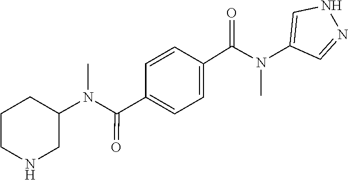 [SMILES: CN(C(=O)c1ccc(C(=O)N(C)C2CCCNC2)cc1)c1cn[nH]c1] |
| 39 | N1-methyl-N1- (piperidin-3-yl)-N4- (1H-pyrazol-4-yl)- terephthalamide | 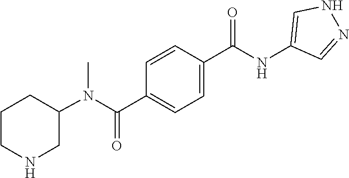 [SMILES: CN(C(=O)c1ccc(C(=O)Nc2cn[nH]c2)cc1)C1CCCNC1] |
| 40 | N1-methyl-N1- (piperidin-4-yl)-N4- (1H-tetrazol-5-yl)- terephthalamide | 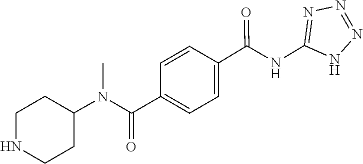 [SMILES: CN(C(=O)c1ccc(C(=O)Nc2nnn[nH]2)cc1)C1CCNCC1] |
| 41 | N1-methyl-N4-(5- methyl-1,2,4- oxadiazol-3-yl)-N1- (piperidin-4-yl)- terephthalamide | 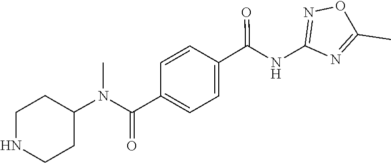 [SMILES: Cc1nc(NC(=O)c2ccc(C(=O)N(C)C3CCNCC3)cc2)no1] |
| 42 | N1,N4-dimethyl-N1- (1H-pyrazol-4-yl)- N4-(pyrrolidin-3-yl)- terephthalamide | 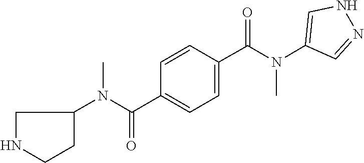 [SMILES: CN(C(=O)c1ccc(C(=O)N(C)C2CCNC2)cc1)c1cn[nH]c1] |
| 43 | N1-(azetidin-3-yl)- N1,N4-dimethyl-N4- (1H-pyrazol-4-yl)- terephthalamide | 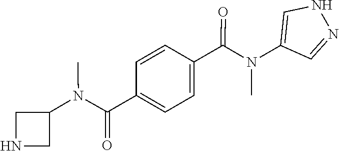 [SMILES: CN(C(=O)c1ccc(C(=O)N(C)C2CNC2)cc1)c1cn[nH]c1] |
| 44 | N1-(2-aminopyrimidin- 5-yl)-N4-(azetidin-3- yl)-N1,N4-dimethyl- terephthalamide | 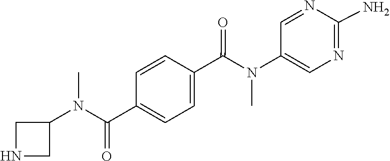 [SMILES: CN(C(=O)c1ccc(C(=O)N(C)C2CNC2)cc1)c1cnc(N)nc1] |
| 45 | N2-(piperidin-4-yl)- N5-(1H-pyrazol-4-yl)- pyrazine-2,5-dicarbox- amide | 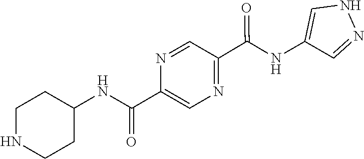 [SMILES: O=C(Nc1cn[nH]c1)c1cnc(C(=O)NC2CCNCC2)cn1] |
| 46 | N1,N3-dimethyl- N1-(piperidin-4-yl)- N3-(1H-pyrazol-4-yl)- bicyclo[1.1.1]pentane- 1,3-dicarboxamide | 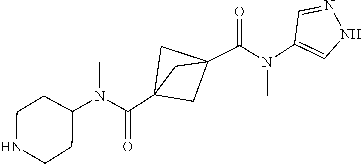 [SMILES: CN(C(=O)C12CC(C(=O)N(C)C3CCNCC3)(C1)C2)c1cn[nH]c1] |
| 47 | N1-methyl-N1- (piperidin-4-yl)-N3- (1H-pyrazol-4-yl)- bicyclo[1.1.1]pentane- 1,3-dicarboxamide | 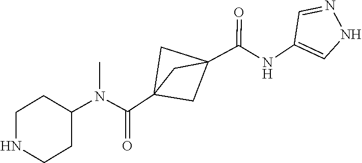 [SMILES: CN(C(=O)C12CC(C(=O)Nc3cn[nH]c3)(C1)C2)C1CCNCC1] |
| 48 | N1-methyl-N3-(1H- pyrazol-4-yl)-N1- (pyrrolidin-3-yl)- bicyclo[1.1.1]pentane- 1,3-dicarboxamide | 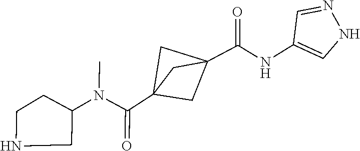 [SMILES: CN(C(=O)C12CC(C(=O)Nc3cn[nH]c3)(C1)C2)C1CCNC1] |
| 49 | N1-(3-aminocyclohex- yl)-N1-methyl-N3-(1H- pyrazol-4-yl)bicyclo- [1.1.1]pentane-1,3- dicarboxamide | 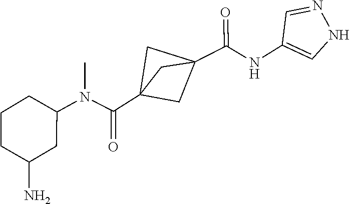 [SMILES: CN(C(=O)C12CC(C(=O)Nc3cn[nH]c3)(C1)C2)C1CCCC(N)C1] |
| 50 | N1-methyl-N1-(piperi- din-4-yl)-N3-(1H- tetrazol-5-yl)bicyclo- [1.1.1]pentane-1,3- dicarboxamide | 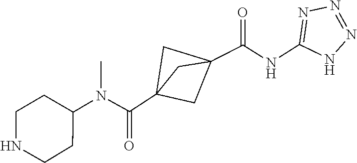 [SMILES: CN(C(=O)C12CC(C(=O)Nc3nnn[nH]3)(C1)C2)C1CCNCC1] |
| 51 | N1,N3-dimethyl-N1- (piperidin-4-yl)-N3- (1H-tetrazol-5-yl)- bicyclo[1.1.1]pentane- 1,3-dicarboxamide | 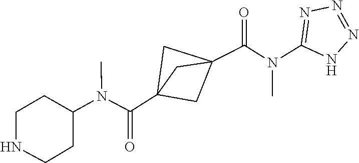 [SMILES: CN(C(=O)C12CC(C(=O)N(C)C3CCNCC3)(C1)C2)c1nnn[nH]1] |
| 52 | N1-(2-aminopyrimidin- 5-yl)-N3-methyl-N3- (piperidin-4-yl)bicyclo- [1.1.1]pentane-1,3- dicarboxamide | 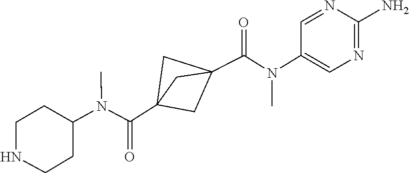 [SMILES: CN(C(=O)C12CC(C(=O)N(C)C3CCNCC3)(C1)C2)c1cnc(N)nc1] |
| 53 | N1-methyl-N3-(5- methyl-1,2,4- oxadiazol-3-yl)-N1- (piperidin-4-yl)- bicyclo[1.1.1]- pentane-1,3- dicarboxamide | 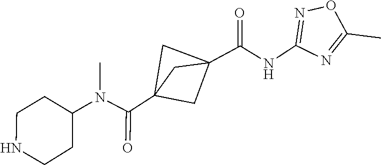 [SMILES: Cc1nc(NC(=O)C23CC(C(=O)N(C)C4CCNCC4)(C2)C3)no1] |
| 54 | 6-(6-methoxy-3,4- dihydroisoquinolin- 2(1H)-yl)-N-methyl- N-(piperidin-4-yl)- pyridazin-3-amine | 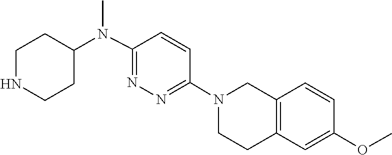 [SMILES: COc1ccc2c(c1)CCN(c1ccc(N(C)C3CCNCC3)nn1)C2] |
| 55 | 6-(6-(methyl(piperidin- 4-yl)amino)pyridazin- 3-yl)-5,6,7,8-tetrahydro- 1,6-naphthyridin-2(1H)- one | 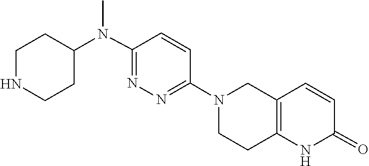 [SMILES: CN(c1ccc(N2CCc3[nH]c(=O)ccc3C2)nn1)C1CCNCC1] |
| 56 | 2-(6-(methyl(piperidin- 4-yl)amino)pyridazin- 3-yl)-1,2,3,4-tetrahydro- isoquinoline-6-carbox- amide | 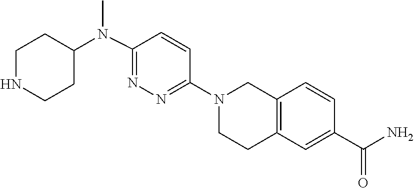 [SMILES: CN(c1ccc(N2CCc3cc(C(N)=O)ccc3C2)nn1)C1CCNCC1] |
| 57 | 6-(4-(4H-1,2,4-triazol- 4-yl)piperidin-1-yl)-N- methyl-N-(piperidin-4- yl)pyridazin-3-amine | 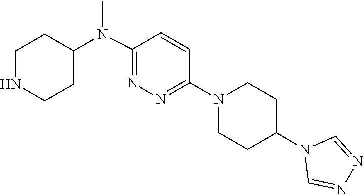 [SMILES: CN(c1ccc(N2CCC(n3cnnc3)CC2)nn1)C1CCNCC1] |
| 58 | 6-methoxy-2-(6-(methyl- (piperidin-4-yl)amino)- pyridazin-3-yl)isoquin- oline-1,3(2H,4H)-dione | 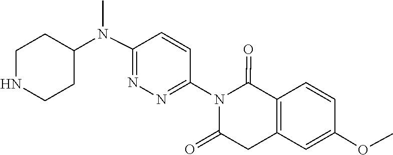 [SMILES: COc1ccc2c(c1)CC(=O)N(c1ccc(N(C)C3CCNCC3)nn1)C2=O] |
| 59 | 6-methoxy-2-(6-(methyl- (piperidin-4-yl)amino)- pyridazin-3-yl)-1,4- dihydroisoquinolin- 3(2H)-one | 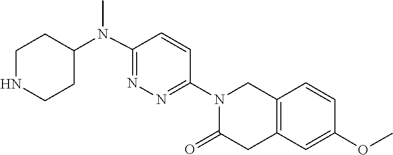 [SMILES: COc1ccc2c(c1)CC(=O)N(c1ccc(N(C)C3CCNCC3)nn1)C2] |
| 60 | 6-methoxy-2-(6-(methyl- (piperidin-4-yl)amino)- pyridazin-3-yl)iso- indolin-1-one | 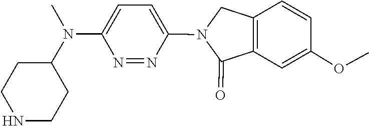 [SMILES: COc1ccc2c(c1)C(=O)N(c1ccc(N(C)C3CCNCC3)nn1)C2] |
| 61 | 5-methoxy-2-(6-(methyl- (piperidin-4-yl)amino)- pyridazin-3-yl)isoindolin- 1-one | 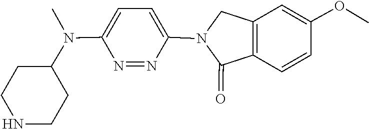 [SMILES: COc1ccc2c(c1)CN(c1ccc(N(C)C3CCNCC3)nn1)C2=O] |
| 62 | 3-hydroxy-6-methoxy- 2-(6-(methyl(piperidin- 4-yl)amino)pyridazin- 3-yl)quinazolin-4(3H)- one | 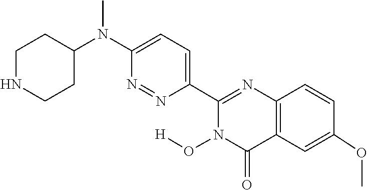 [SMILES: COc1ccc2nc(-c3ccc(N(C)C4CCNCC4)nn3)n(O)c(=O)c2c1] |
| 63 | 3-hydroxy-6-methoxy- 2-(6-(methyl(piperidin- 4-yl)amino)pyridazin- 3-yl)pyrido[3,4-d]- pyrimidin-4(3H)-one | 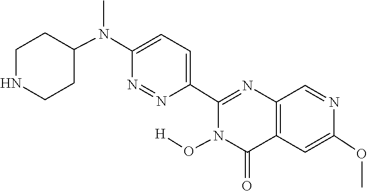 [SMILES: COc1cc2c(=O)n(O)c(-c3ccc(N(C)C4CCNCC4)nn3)nc2cn1] |
| 64 | 3-hydroxy-2-(6-(methyl- (piperidin-4-yl)amino)- pyridazin-3-yl)-3,7- dihydropyrido[3,4-d]- pyrimidine-4,6-dione | 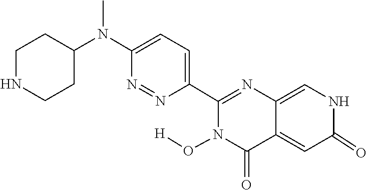 [SMILES: CN(c1ccc(-c2nc3c[nH]c(=O)cc3c(=O)n2O)nn1)C1CCNCC1] |
| 65 | 3-hydroxy-6-methoxy- 2-(6-(methyl(piperidin- 4-yl)amino)pyridazin- 3-yl)pyrido[3,2-d]- pyrimidin-4(3H)-one | 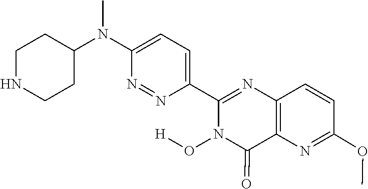 [SMILES: COc1ccc2nc(-c3ccc(N(C)C4CCNCC4)nn3)n(O)c(=O)c2n1] |
| 66 | 3-hydroxy-2-(6-(methyl- (piperidin-4-yl)amino)- pyridazin-3-yl)-3,5- dihydropyrido[3,2-d]- pyrimidine-4,6-dione | 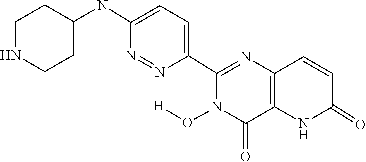 |
| 67 | 5-(6-(((1r,4r)-4- aminocyclohexyl)- (methyl)amino)- pyridazin-3-yl)-6- hydroxy-2,6-dihydro- 7H-pyrazolo[4,3-d]- pyrimidin-7-one | 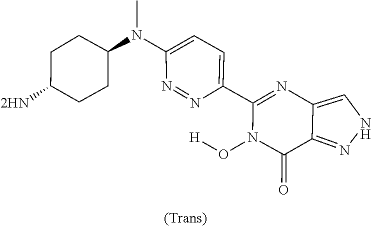 |
| 68 | 5-(6-(((1s,4s)-4- aminocyclohexyl)- (methyl)amino)- pyridazin-3-yl)-6- hydroxy-2,6-dihydro- 7H-pyrazolo[4,3-d]- pyrimidin-7-one | 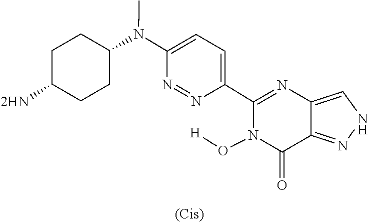 |
| 69 | 6-(6-(((1r,4r)-4- aminocyclohexyl)- (methyl)amino)- pyridazin-3-yl)-5- hydroxy-2,5-dihydro- 4H-pyrazolo[3,4-d]- pyrimidin-4-one | 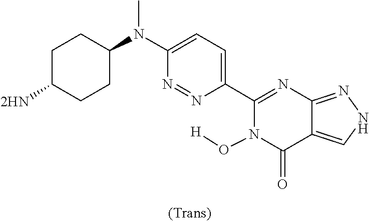 |
| 70 | 6-(6-(((1s,4s)-4- aminocyclohexyl)- (methyl)amino)- pyridazin-3-yl)-5- hydroxy-2,5-dihydro- 4H-pyrazolo[3,4-d]- pyrimidin-4-one | 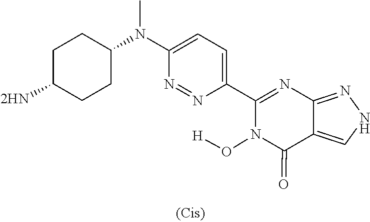 |
| 71 | 2-(5-(methyl(piperidin- 4-yl)amino)pyrazin- 2-yl)-5-(1H-pyrazol- 4-yl)phenol | 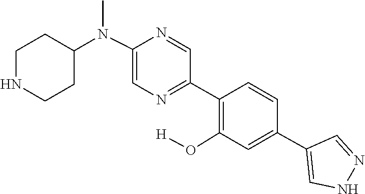 [SMILES: CN(c1cnc(-c2ccc(-c3cn[nH]c3)cc2O)cn1)C1CCNCC1] |
| 72 | 5-(3-hydroxy-4-(5- (methyl(piperidin-4- yl)amino)pyrazin-2- yl)phenyl)pyrimidin- 2(1H)-one | 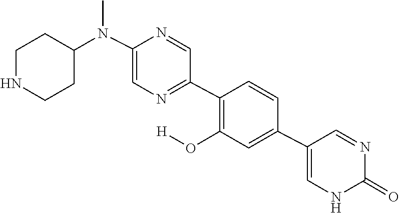 [SMILES: CN(c1cnc(-c2ccc(-c3cnc(=O)[nH]c3)cc2O)cn1)C1CCNCC1] |
| 73 | 7-methoxy-3-(5- (methyl(piperidin-4- yl)amino)pyrazin-2- yl)naphthalen-2-ol | 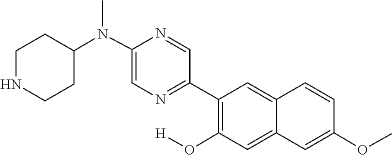 [SMILES: COc1ccc2cc(-c3cnc(N(C)C4CCNCC4)cn3)c(O)cc2c1] |
| 74 | 2-(5-(1H-pyrazol-4- yl)pyrimidin-2-yl)-5- (methyl(piperidin-4- yl)amino)phenol | 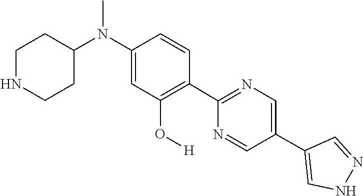 [SMILES: CN(c1ccc(-c2ncc(-c3cn[nH]c3)cn2)c(O)c1)C1CCNCC1] |
| 75 | 2′-(2-hydroxy-4- (methyl(piperidin-4-yl)- amino)phenyl)[5,5′- bipyrimidin]-2(1H)-one | 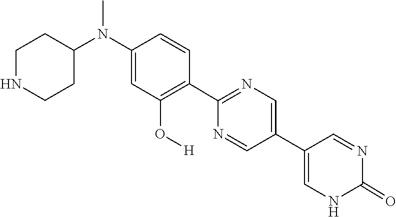 [SMILES: CN(c1ccc(-c2ncc(-c3cnc(=O)[nH]c3)cn2)c(O)c1)C1CCNCC1] |
| 76 | 2-(6-methoxyquin- azolin-2-yl)-5-(methyl- (piperidin-4-yl)amino)- phenol | 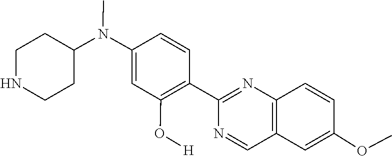 [SMILES: COc1ccc2nc(-c3ccc(N(C)C4CCNCC4)cc3O)ncc2c1] |
| 77 | 2-(2-hydroxy-4-(methyl- (piperidin-4-yl)amino)- phenyl)-2,6-dihydro- pyrrolo[3,4-c]pyrazole- 5(4H)-carboxamide | 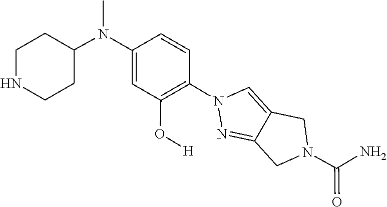 [SMILES: CN(c1ccc(-n2cc3c(n2)CN(C(N)=O)C3)c(O)c1)C1CCNCC1] |
| 78 | (E)-N′-hydroxy-N- methyl-6-(methyl- (piperidin-4-yl)amino)- N-(2-oxo-1,2-dihydro- pyrimidin-5-yl)pyrid- azine-3-carboximid- amide | 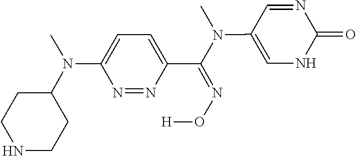 [SMILES: CN(/C(=N/O)c1ccc(N(C)C2CCNCC2)nn1)c1cnc(=O)[nH]c1] |
| 79 | (E)-N-(1H-benzo[d]- [1,2,3]triazol-6-yl)-N′- hydroxy-N-methyl-6- (methyl(piperidin-4- yl)amino)pyridazine- 3-carboximidamide | 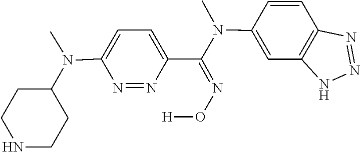 [SMILES: CN(/C(=N/O)c1ccc(N(C)C2CCNCC2)nn1)c1ccc2nn[nH]c2c1] |
| 80 | (E)-N′-hydroxy-N- methyl-6-(methyl(piper- idin-4-yl)amino)-N- (tetrazolo[1,5-a]pyridin- 6-yl)pyridazine-3- carboximidamide | 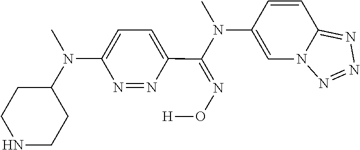 [SMILES: CN(/C(=N/O)c1ccc(N(C)C2CCNCC2)nn1)c1ccc2nnnn2c1] |
| 81 | (E)-N′-hydroxy-N- methyl-6-(methyl- (piperidin-4-yl)amino)- N-(2-methylbenzo[d]- oxazol-6-yl)pyridazine- 3-carboximidamide | 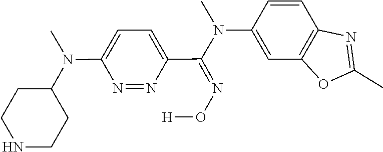 [SMILES: Cc1nc2ccc(N(C)/C(=N/O)c3ccc(N(C)C4CCNCC4)nn3)cc2o1] |
| 82 | (E)-N′-hydroxy-N- methyl-6-(methyl- (piperidin-4-yl)- amino)-N-(2- methylbenzo[d]- oxazol-5-yl)pyrid- azine-3-carbox- imidamide | 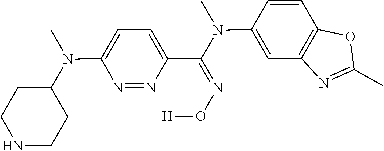 [SMILES: Cc1nc2cc(N(C)/C(=N/O)c3ccc(N(C)C4CCNCC4)nn3)ccc2o1] |
| 83 | (E)-N′-hydroxy-N-(4- hydroxyphenyl)-N- methyl-6-(methyl- (piperidin-4-yl)amino)- pyridazine-3-carbox- imidamide | 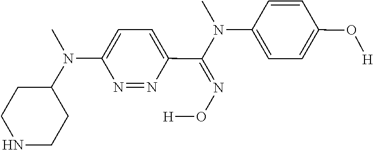 [SMILES: CN(/C(=N/O)c1ccc(N(C)C2CCNCC2)nn1)c1ccc(O)cc1] |
| 84 | 5-((4-methoxyphenyl)- ethynyl)-N-methyl-N- (piperidin-4-yl)pyrazin- 2-amine | 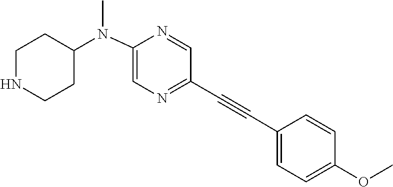 [SMILES: COc1ccc(C#Cc2cnc(N(C)C3CCNCC3)cn2)cc1] |
| 85 | 5-((6-(methyl(piperidin- 4-yl)amino)pyridazin-3- yl)ethynyl)pyrimidin- 2(1H)-one | 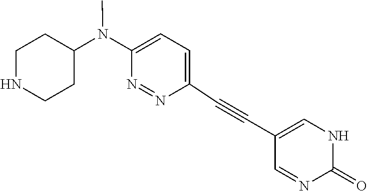 [SMILES: CN(c1ccc(C#Cc2cnc(=O)[nH]c2)nn1)C1CCNCC1] |
| 86 | 6-((1H-pyrazol-4-yl)- ethynyl)-N-methyl-N- (piperidin-4-yl)pyrid- azin-3-amine | 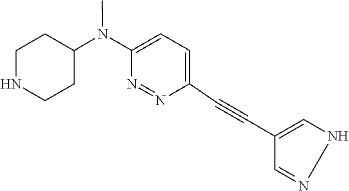 [SMILES: CN(c1ccc(C#Cc2cn[nH]c2)nn1)C1CCNCC1] |
| 87 | (E)-5-(2-(6-(methyl- (piperidin-4-yl)amino)- pyridazin-3-yl)vinyl)- pyrimidin-2(1H)-one | 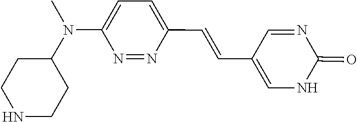 [SMILES: CN(c1ccc(/C=C/c2cnc(=O)[nH]c2)nn1)C1CCNCC1] |
| 88 | (E)-5-(2-(6-(methyl- (piperidin-4-yl)amino)- pyridazin-3-yl)vinyl)- pyridin-2(1H)-one | 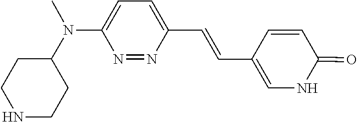 [SMILES: CN(c1ccc(/C=C/c2ccc(=O)[nH]c2)nn1)C1CCNCC1] |
| 89 | (E)-N-methyl-N- (piperidin-4-yl)-6-(2- (tetrazolo[1,5-a]- pyridin-6-yl)vinyl)- pyridazin-3-amine | 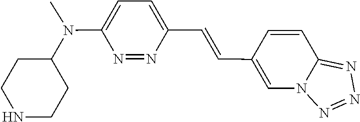 [SMILES: CN(c1ccc(/C=C/c2ccc3nnnn3c2)nn1)C1CCNCC1] |
| 90 | N-(2-(methyl(piperidin- 4-yl)amino)pyrimidin- 5-yl)-4,6-dihydro- pyrrolo[3,4-c]pyrazole- 5(1H)-carboxamide | 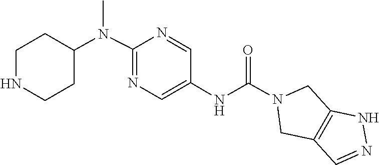 [SMILES: CN(c1ncc(NC(=O)N2Cc3cn[nH]c3C2)cn1)C1CCNCC1] |
| 91 | 2-methyl-N-(2-(methyl- (piperidin-4-yl)amino)- pyrimidin-5-yl)-4,6- dihydro-5H-pyrrolo- [3,4-d]oxazole-5- carboxamide | 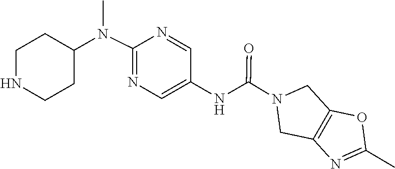 |
| 92 | N-(2-(methyl(piperidin- 4-yl)amino)pyrimidin- 5-yl)-4,6-dihydro-5H- pyrrolo[3,4-d]thiazole- 5-carboxamide | 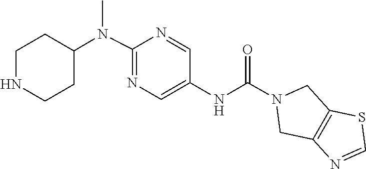 [SMILES: CN(c1ncc(NC(=O)N2Cc3ncsc3C2)cn1)C1CCNCC1] |
| 93 | N-methyl-N-(6-(methyl- (piperidin-4-yl)amino)- pyridazin-3-yl)-4-(1H- pyrazol-4-yl)benzamide | 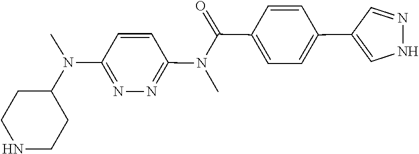 [SMILES: CN(C(=O)c1ccc(-c2cn[nH]c2)cc1)c1ccc(N(C)C2CCNCC2)nn1] |
| 94 | N-methyl-N-(6-(methyl- (piperidin-4-yl)amino)- pyridazin-3-yl)-6-oxo- 1,6-dihydropyridine-3- carboxamide | 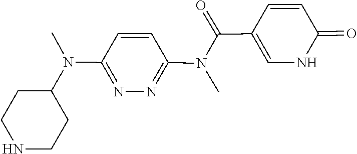 [SMILES: CN(C(=O)c1ccc(=O)[nH]c1)c1ccc(N(C)C2CCNCC2)nn1] |
| 95 | 4-hydroxy-N-methyl- N-(6-(methyl(piperidin- 4-yl)amino)pyridazin-3- yl)benzamide | 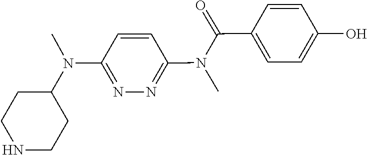 [SMILES: CN(C(=O)c1ccc(O)cc1)c1ccc(N(C)C2CCNCC2)nn1] |
| 96 | 4-methoxy-N-methyl- N-(6-(methyl(piperidin- 4-yl)amino)pyridazin-3- yl)benzamide | 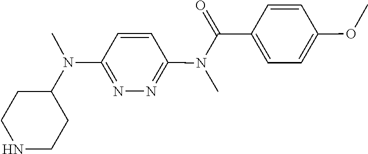 [SMILES: COc1ccc(C(=O)N(C)c2ccc(N(C)C3CCNCC3)nn2)cc1] |
| 97 | 2-(methyl(piperidin- 4-yl)amino)-N-(1H- pyrazol-4-yl)quin- azoline-6-carboxamide | 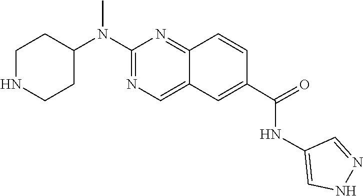 |
| 98 | N-methyl-2-(methyl- (piperidin-4-yl)amino)- N-(1H-pyrazol-4-yl)- quinazoline-6-carbox- amide | 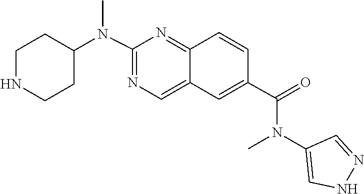 [SMILES: CN(C(=O)c1ccc2nc(N(C)C3CCNCC3)ncc2c1)c1cn[nH]c1] |
| 99 | N-methyl-2-(methyl- (piperidin-4-yl)amino)- N-(1H-pyrazol-4-yl)- quinoline-6-carbox- amide | 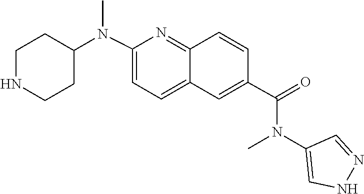 [SMILES: CN(C(=O)c1ccc2nc(N(C)C3CCNCC3)ccc2c1)c1cn[nH]c1] |
| 100 | N-methyl-6-(methyl- (piperidin-4-yl)amino)- N-(1H-pyrazol-4-yl)- 2-naphthamide | 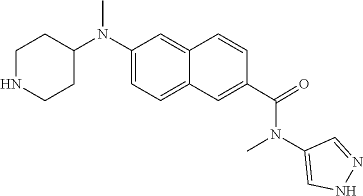 [SMILES: CN(C(=O)c1ccc2cc(N(C)C3CCNCC3)ccc2c1)c1cn[nH]c1] |
| 101 | N-methyl-6-(methyl- (piperidin-4-yl)amino)- N-(1H-pyrazol-4-yl) quinoline-2-carbox- amide | 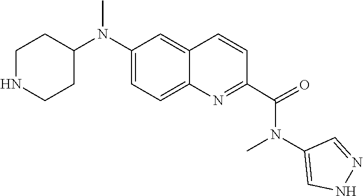 [SMILES: CN(C(=O)c1ccc2cc(N(C)C3CCNCC3)ccc2n1)c1cn[nH]c1] |
| 102 | N-methyl-2-(methyl- (piperidin-4-yl)amino)- N-(1H-pyrazol-4-yl)- quinoxaline-6-carbox- amide | 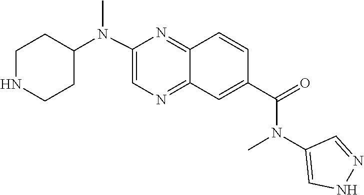 [SMILES: CN(C(=O)c1ccc2nc(N(C)C3CCNCC3)cnc2c1)c1cn[nH]c1] |
| 103 | N-methyl-2-(methyl- (piperidin-4-yl)amino)- N-(2-oxo-1,2-dihydro- pyrimidin-5-yl)quin- oline-6-carboxamide | 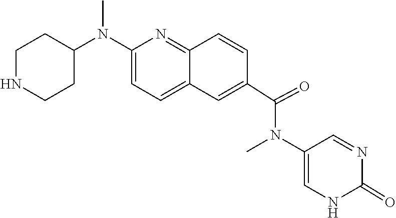 [SMILES: CN(C(=O)c1ccc2nc(N(C)C3CCNCC3)ccc2c1)c1cnc(=O)[nH]c1] |
| 104 | (E)-6-(2-(1H-pyrazol- 4-yl)vinyl)-N-methyl- N-(piperidin-4-yl)- quinazolin-2-amine | 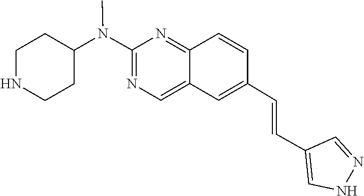 [SMILES: CN(c1ncc2cc(/C=C/c3cn[nH]c3)ccc2n1)C1CCNCC1] |
| 105 | (E)-7-(2-(1H-pyrazol- 4-yl)vinyl)-N-methyl- N-(piperidin-4-yl)- pyrido[2,3-b]pyrazin- 3-amine | 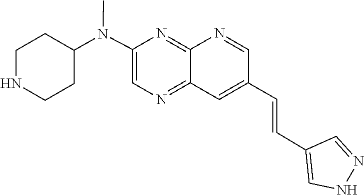 [SMILES: CN(c1cnc2cc(/C=C/c3cn[nH]c3)cnc2n1)C1CCNCC1] |
| 106 | (E)-7-(2-(1H-pyrazol- 4-yl)vinyl)-3-(piperidin- 4-yloxy)pyrido[2,3-b]- pyrazine | 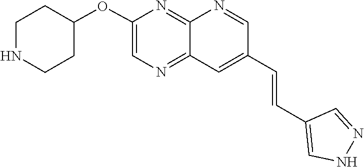 [SMILES: C(=C\c1cnc2nc(OC3CCNCC3)cnc2c1)/c1cn[nH]c1] |
| 107 | (E)-6-(2-(1H-pyrazol-4- yl)vinyl)-N-methyl-N- (piperidin-4-yl)-1,8- naphthyridin-2-amine | 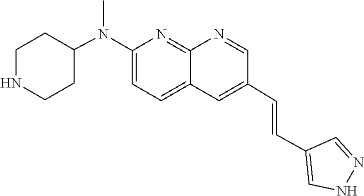 [SMILES: CN(c1ccc2cc(/C=C/c3cn[nH]c3)cnc2n1)C1CCNCC1] |
| 108 | (E)-7-(2-(1H-pyrazol- 4-yl)vinyl)-N-methyl- N-(piperidin-4-yl)-1,8- naphthyridin-3-amine | 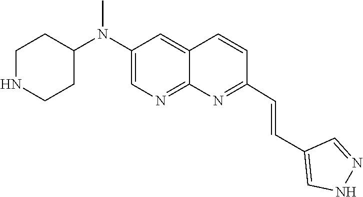 [SMILES: CN(c1cnc2nc(/C=C/c3cn[nH]c3)ccc2c1)C1CCNCC1] |
| 109 | (E)-5-(2-(2-(methyl- (piperidin-4-yl)amino)- quinazolin-6-yl)vinyl)- pyrimidin-2(1H)-one | 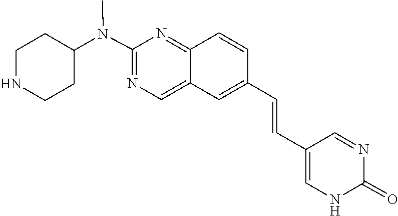 [SMILES: CN(c1ncc2cc(/C=C/c3cnc(=O)[nH]c3)ccc2n1)C1CCNCC1] |
| 110 | N-methyl-6-((methyl- (1H-pyrazol-4-yl)- amino)methyl)-N- (piperidin-4-yl)- quinazolin-2-amine | 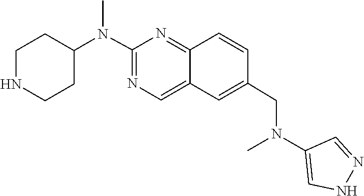 [SMILES: CN(Cc1ccc2nc(N(C)C3CCNCC3)ncc2c1)c1cn[nH]c1] |
| 111 | N-methyl-N-(piperidin- 4-yl)-6-(1,4,6,7-tetra- hydro-5H-pyrazolo[4,3- c]pyridin-5-yl)-1,5- naphthyridin-2-amine | 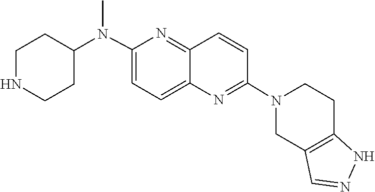 [SMILES: CN(c1ccc2nc(N3CCc4[nH]ncc4C3)ccc2n1)C1CCNCC1] |
| 112 | 6-(1H-benzo[d][1,2,3]- triazol-6-yl)-N-methyl- N-(piperidin-4-yl)quin- azolin-2-amine | 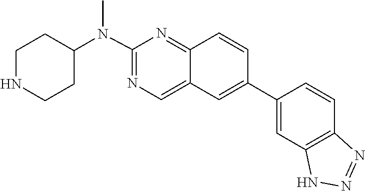 [SMILES: CN(c1ncc2cc(-c3ccc4nn[nH]c4c3)ccc2n1)C1CCNCC1] |
| 113 | N-methyl-N-(piperidin- 4-yl)-6-(tetrazolo[1,5- a]pyridin-6-yl)quin- azolin-2-amine | 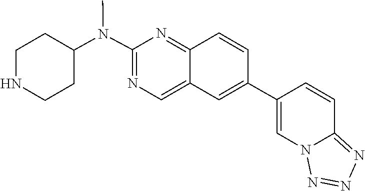 |
| 114 | 5-(2-(methyl(piperidin- 4-yl)amino)quinazolin- 6-yl)pyridin-2(1H)- one | 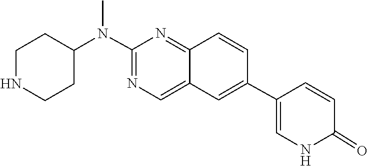 [SMILES: CN(c1ncc2cc(-c3ccc(=O)[nH]c3)ccc2n1)C1CCNCC1] |
| 115 | 5-(2-(methyl(piperidin- 4-yl)amino)quinazolin- 6-yl)pyrimidin-2(1H)- one | 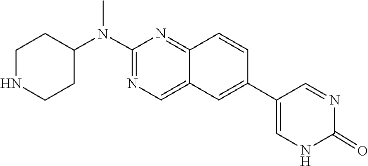 [SMILES: CN(c1ncc2cc(-c3cnc(=O)[nH]c3)ccc2n1)C1CCNCC1] |
| 116 | 6-(2-(methyl(piperidin- 4-yl)amino)quinazolin- 6-yl)benzo[d]oxazol- 2(3H)-one | 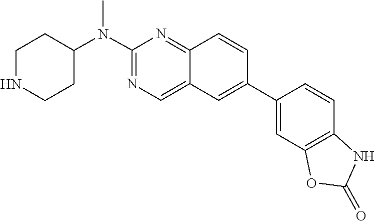 [SMILES: CN(c1ncc2cc(-c3ccc4[nH]c(=O)oc4c3)ccc2n1)C1CCNCC1] |
| 117 | 2-(1H-benzo[d][1,2,3]- triazol-6-yl)-N-methyl- N-(piperidin-4-yl)pyrido- [3,4-d]pyrimidin-6- amine | 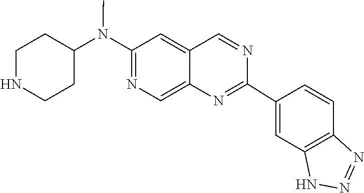 [SMILES: CN(c1cc2cnc(-c3ccc4nn[nH]c4c3)nc2cn1)C1CCNCC1] |
| 118 | 5-(6-(methyl(piperidin- 4-yl)amino)pyrido[3,4- d]pyrimidin-2-yl)- pyridin-2(1H)-one | 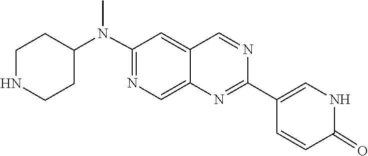 [SMILES: CN(c1cc2cnc(-c3ccc(=O)[nH]c3)nc2cn1)C1CCNCC1] |
| 119 | 2-(1H-benzo[d][1,2,3]- triazol-6-yl)-6-(methyl- (piperidin-4-yl)amino)- pyrido[3,4-d]pyrimidin- 4(3H)-one | 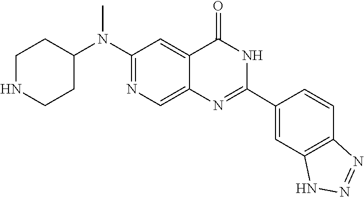 [SMILES: CN(c1cc2c(=O)[nH]c(-c3ccc4nn[nH]c4c3)nc2cn1)C1CCNCC1] |
| 120 | 5-(6-(methyl(piperidin- 4-yl)amino)quinolin-2- yl)pyridin-2(1H)-one | 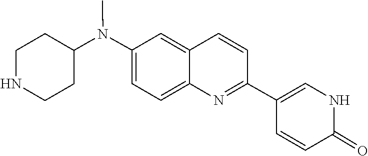 [SMILES: CN(c1ccc2nc(-c3ccc(=O)[nH]c3)ccc2c1)C1CCNCC1] |
| 121 | N-methyl-N-(piperidin- 4-yl)-2-(tetrazolo[1,5- a]pyridin-7-yl)quinolin- 6-amine | 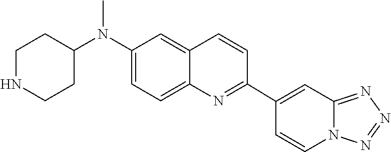 [SMILES: CN(c1ccc2nc(-c3ccn4nnnc4c3)ccc2c1)C1CCNCC1] |
| 122 | 3-(6-(methyl(piperidin- 4-yl)amino)quinolin-2- yl)bicyclo[1.1.1]pentane- 1-carboxamide | 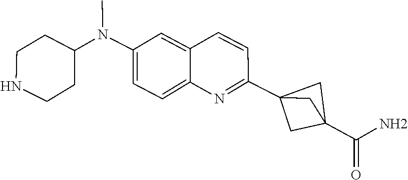 [SMILES: CN(c1ccc2nc(C34CC(C(N)=O)(C3)C4)ccc2c1)C1CCNCC1] |
| 123 | 3-(6-(methyl(piperidin- 4-yl)amino)-4-oxo-3,4- dihydropyrido[3,4-d]- pyrimidin-2-yl)bicyclo- [1.1.1]pentane-1-carbox- amide | 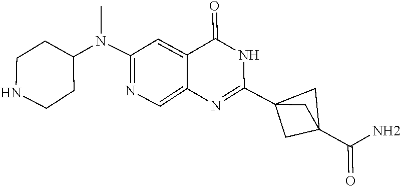 [SMILES: CN(c1cc2c(=O)[nH]c(C34CC(C(N)=O)(C3)C4)nc2cn1)C1CCNCC1] |
| 124 | 3-(6-(methyl(piperidin- 4-yl)amino)pyrido[3,4- d]pyrimidin-2-yl)bicyclo- [1.1.1]pentane-1-carbox- amide | 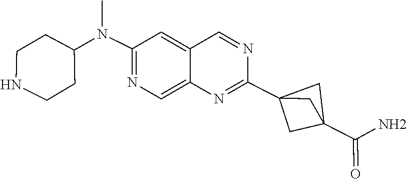 [SMILES: CN(c1cc2cnc(C34CC(C(N)=O)(C3)C4)nc2cn1)C1CCNCC1] |
| 125 | N-hydroxy-3-(6-(methyl- (piperidin-4-yl)amino)- pyrido[3,4-d]pyrimidin- 2-yl)bicyclo[1.1.1]- pentane-1-carboxamide | 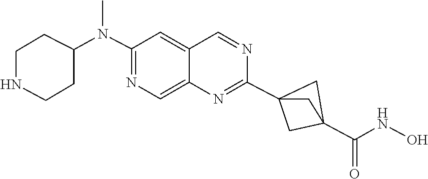 [SMILES: CN(c1cc2cnc(C34CC(C(=O)NO)(C3)C4)nc2cn1)C1CCNCC1] |
| 126 | N-methoxy-3-(6-(methyl- (piperidin-4-yl)amino)- pyrido[3,4-d]pyrimidin- 2-yl)bicyclo[1.1.1]- pentane-1-carboxamide | 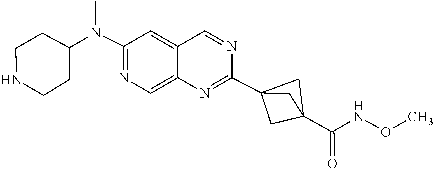 [SMILES: CONC(=O)C12CC(c3ncc4cc(N(C)C5CCNCC5)ncc4n3)(C1)C2] |
| 127 | 2-(2,6-dihydropyrrolo- [3,4-c]pyrazol-5(4H)- yl)-N-methyl-N- (piperidin-4-yl)pyrido- [3,4-d]pyrimidin-6- amine | 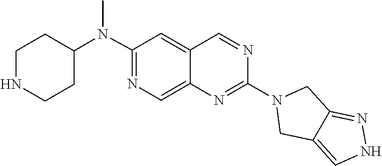 [SMILES: CN(c1cc2cnc(N3Cc4c[nH]nc4C3)nc2cn1)C1CCNCC1] |
| 128 | 1-(6-(methyl(piperidin- 4-yl)amino)quinazolin- 2-yl)pyridin-4(1H)- one | 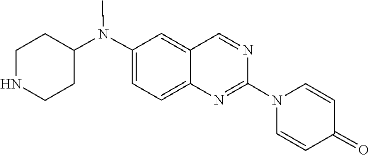 [SMILES: CN(c1ccc2nc(-n3ccc(=O)cc3)ncc2c1)C1CCNCC1] |
| 129 | l-(6-(methyl(piperidin- 4-yl)amino)quinazolin- 2-yl)piperidin-4-one | 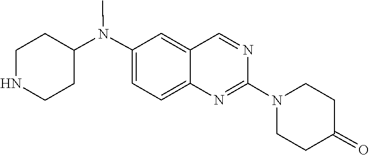 [SMILES: CN(c1ccc2nc(N3CCC(=O)CC3)ncc2c1)C1CCNCC1] |
| 130 | (6-(2-hydroxy-4-(1H- pyrazol-4-yl)phenyl)- pyridazin-3-yl)- (piperazin-1-yl)meth- anone | 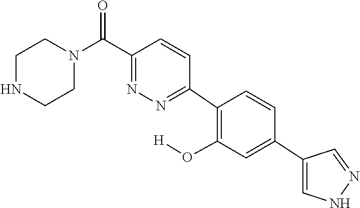 [SMILES: O=C(c1ccc(-c2ccc(-c3cn[nH]c3)cc2O)nn1)N1CCNCC1] |
| 131 | (6-(2-hydroxy-4-(1H- pyrazol-4-yl)phenyl)- pyridazin-3-yl)(2,2,6,6- tetramethylpiperidin- 4-yl)methanone | 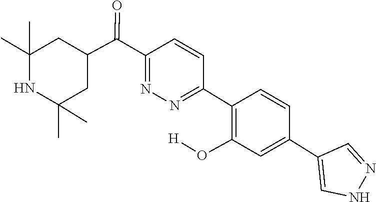 [SMILES: CC1(C)CC(C(=O)c2ccc(-c3ccc(-c4cn[nH]c4)cc3O)nn2)CC(C)(C)N1] |
| 132 | 5-(1H-pyrazol-4-yl)- 2-(6-((2,2,6,6-tetra- methylpiperidin-4- yl)thio)pyridazin-3- yl)phenol | 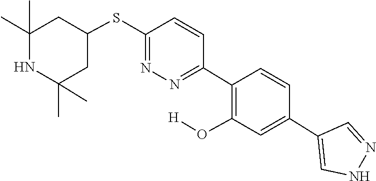 [SMILES: CC1(C)CC(Sc2ccc(-c3ccc(-c4cn[nH]c4)cc3O)nn2)CC(C)(C)N1] |
| 133 | 2-(6-(cyclopropyl- (2,2,6,6-tetramethyl- piperidin-4-yl)amino)- pyridazin-3-yl)-5-(1H- pyrazol-4-yl)phenol | 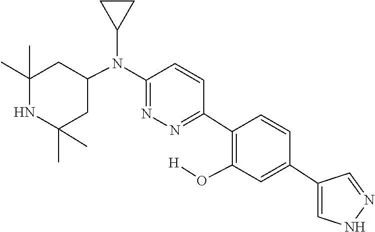 [SMILES: CC1(C)CC(N(c2ccc(-c3ccc(-c4cn[nH]c4)cc3O)nn2)C2CC2)CC(C)(C)N1] |
| 134 | 2-(6-(cyclobutyl- (2,2,6,6-tetramethyl- piperidin-4-yl)amino)- pyridazin-3-yl)-5-(1H- pyrazol-4-yl)phenol | 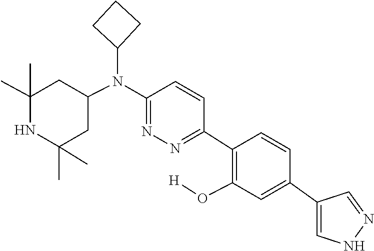 [SMILES: CC1(C)CC(N(c2ccc(-c3ccc(-c4cn[nH]c4)cc3O)nn2)C2CCC2)CC(C)(C)N1] |
| 135 | 2-(tetramethylpiperi- din-4-yl)amino)pyrid- azin-6-(methoxy- (2,2,6,6-3-yl)-5-(1H- pyrazol-4-yl)phenol | 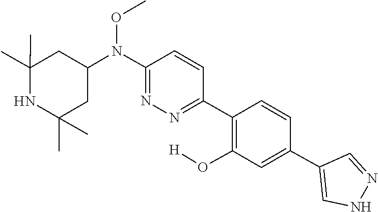 [SMILES: CON(c1ccc(-c2ccc(-c3cn[nH]c3)cc2O)nn1)C1CC(C)(C)NC(C)(C)C1] |
| 136 | 2-(6-(octahydro-1H- pyrrolo[3,2-c]pyridin- 1-yl)pyridazin-3-yl)- 5-(1H-pyrazol-4-yl)- phenol | 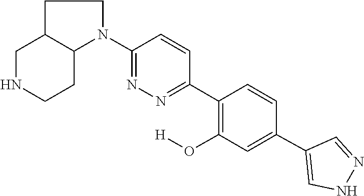 [SMILES: Oc1cc(-c2cn[nH]c2)ccc1-c1ccc(N2CCC3CNCCC32)nn1] |
| 137 | 2-(6-(octahydro-1,6- naphthyridin-1(2H)- yl)pyridazin-3-yl)- 5-(1H-pyrazol-4-yl)- phenol | 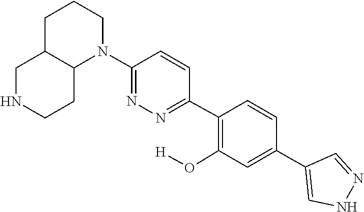 [SMILES: Oc1cc(-c2cn[nH]c2)ccc1-c1ccc(N2CCCC3CNCCC32)nn1] |
| 138 | 2-(6-(1,7-diazaspiro- [3.5]nonan-1-yl)pyrid- azin-3-yl)-5-(1H- pyrazol-4-yl)phenol | 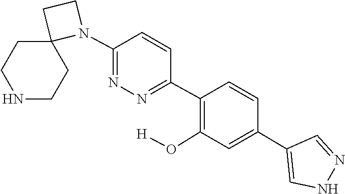 [SMILES: Oc1cc(-c2cn[nH]c2)ccc1-c1ccc(N2CCC23CCNCC3)nn1] |
| 139 | 2-(6-(piperidin-4- ylthio)pyridazin-3- yl)-5-(1H-pyrazol-4- yl)phenol | 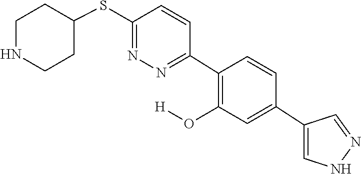 [SMILES: Oc1cc(-c2cn[nH]c2)ccc1-c1ccc(SC2CCNCC2)nn1] |
| 140 | 2-(6-((2-methoxy- ethoxy)(2,2,6,6- tetramethylpiperidin- 4-yl)amino)pyridazin- 3-yl)-5-(1H-pyrazol- 4-yl)phenol | 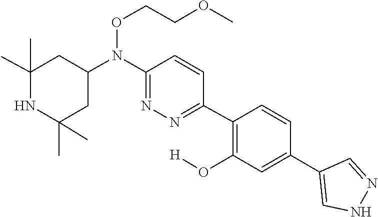 [SMILES: COCCON(c1ccc(-c2ccc(-c3cn[nH]c3)cc2O)nn1)C1CC(C)(C)NC(C)(C)C1] |
| 141 | 5-(1H-pyrazol-4-yl)- 2-(6-((2,2,6,6-tetra- methylpiperidin-4- ylidene)methyl)- pyridazin-3-yl)phenol | 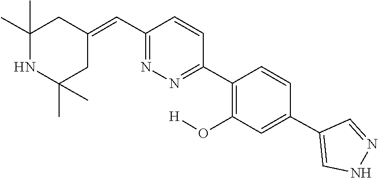 [SMILES: CC1(C)CC(=Cc2ccc(-c3ccc(-c4cn[nH]c4)cc3O)nn2)CC(C)(C)N1] |
| 142 | (6-(2-hydroxy-4- (1H-pyrazol-4-yl)- phenyl)pyridazin-3- yl)(piperidin-4-yl)- methanone | 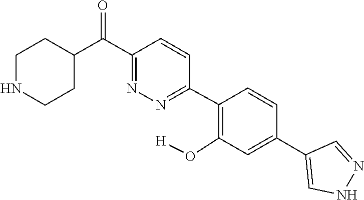 [SMILES: O=C(c1ccc(-c2ccc(-c3cn[nH]c3)cc2O)nn1)C1CCNCC1] |
| 143 | 2-(6-(hydroxy(2,2,6,6- tetramethylpiperidin- 4-yl)methyl)pyridazin- 3-yl)-5-(1H-pyrazol- 4-yl)phenol | 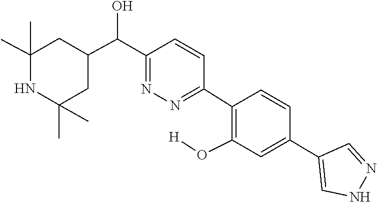 [SMILES: CC1(C)CC(C(O)c2ccc(-c3ccc(-c4cn[nH]c4)cc3O)nn2)CC(C)(C)N1] |
| 144 | 2-(6-(methoxy(2,2,6,6- tetramethylpiperidin- 4-yl)methyl)pyridazin- 3-yl)-5-(1H-pyrazol-4- yl)phenol | 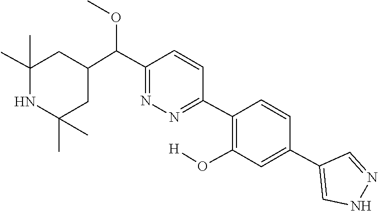 [SMILES: COC(c1ccc(-c2ccc(-c3cn[nH]c3)cc2O)nn1)C1CC(C)(C)NC(C)(C)C1] |
| 145 | (6-(2-hydroxy-4-(1H- pyrazol-4-yl)phenyl)- pyridazin-3-yl)(3,3,5,5- tetramethylpiperazin- 1-yl)methanone | 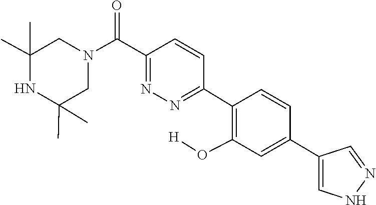 [SMILES: CC1(C)CN(C(=O)c2ccc(-c3ccc(-c4cn[nH]c4)cc3O)nn2)CC(C)(C)N1] |
| 146 | 5-(1H-pyrazol-4-yl)- 2-(6-((2,2,6,6-tetra- methylpiperidin-4-yl)- (trifluoromethyl)amino)- pyridazin-3-yl)phenol | 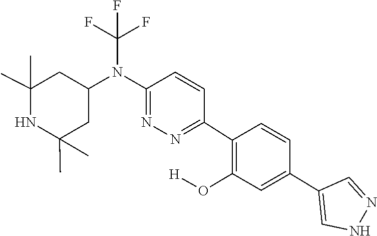 [SMILES: CC1(C)CC(N(c2ccc(-c3ccc(-c4cn[nH]c4)cc3O)nn2)C(F)(F)F)CC(C)(C)N1] |
| 147 | 2-(6-((2-fluoroethyl)- (2,2,6,6-tetramethyl- piperidin-4-yl)amino)- pyridazin-3-yl)-5-(1H- pyrazol-4-yl)phenol | 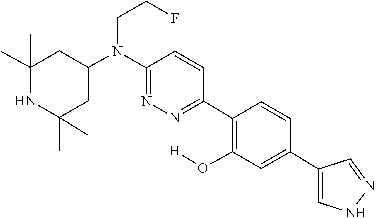 [SMILES: CC1(C)CC(N(CCF)c2ccc(-c3ccc(-c4cn[nH]c4)cc3O)nn2)CC(C)(C)N1] |
| 148 | 5-(1H-pyrazol-4-yl)- 2-(6-((2,2,6,6-tetra- methylpiperidin-4-yl)- (2,2,2-trifluoroethyl)- amino)pyridazin-3- yl)phenol | 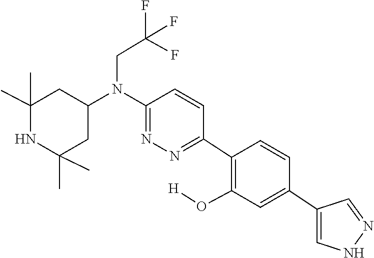 [SMILES: CC1(C)CC(N(CC(F)(F)F)c2ccc(-c3ccc(-c4cn[nH]c4)cc3O)nn2)CC(C)(C)N1] |
| 149 | 2-(6-((3-fluoropropyl)- (2,2,6,6-tetramethyl- piperidin-4-yl)amino)- pyridazin-3-yl)-5-(1H- pyrazol-4-yl)phenol | 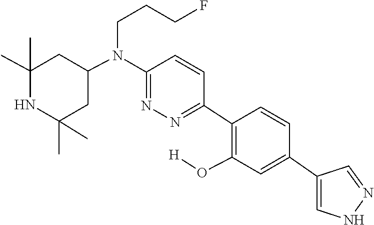 [SMILES: CC1(C)CC(N(CCCF)c2ccc(-c3ccc(-c4cn[nH]c4)cc3O)nn2)CC(C)(C)N1] |
| 150 | 5-(1H-pyrazol-4-yl)- 2-(6-((2,2,6,6-tetra- methylpiperidin-4-yl)- (3,3,3-trifluoropropyl)- amino)pyridazin-3-yl)- phenol | 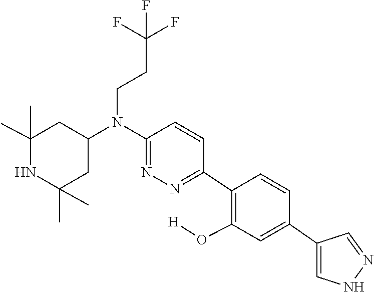 [SMILES: CC1(C)CC(N(CCC(F)(F)F)c2ccc(-c3ccc(-c4cn[nH]c4)cc3O)nn2)CC(C)(C)N1] |
| 151 | 2-(6-((2-methoxyethyl)- (2,2,6,6-tetramethyl- piperidin-4-yl)amino)- pyridazin-3-yl)-5-(1H- pyrazol-4-yl)phenol | 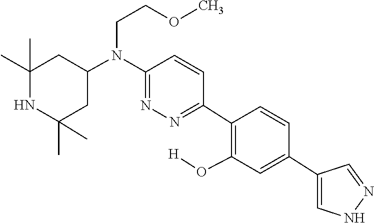 [SMILES: COCCN(c1ccc(-c2ccc(-c3cn[nH]c3)cc2O)nn1)C1CC(C)(C)NC(C)(C)C1] |
| 152 | 3-(6-((2-fluoroethyl)- (2,2,6,6-tetramethyl- piperidin-4-yl)amino)- pyridazin-3-yl)-7-meth- oxynaphthalen-2-ol | 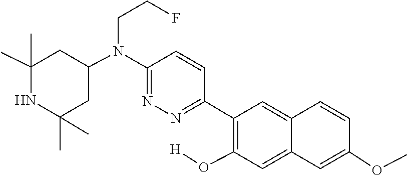 |
| 153 | 2-(6-((6-azabicyclo- [3.1.1]heptan-3-yl)(2- fluoroethyl)amino)- pyridazin-3-yl)-5-(1H- pyrazol-4-yl)phenol | 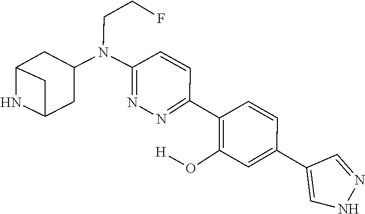 [SMILES: Oc1cc(-c2cn[nH]c2)ccc1-c1ccc(N(CCF)C2CC3CC(C2)N3)nn1] |
| 154 | 2-(6-((8-azabicyclo- [3.2.1]octan-3-yl)(2- fluoroethyl)amino)- pyridazin-3-yl)-5-(1H- pyrazol-4-yl)phenol | 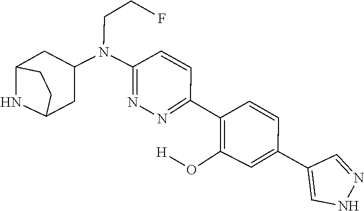 [SMILES: Oc1cc(-c2cn[nH]c2)ccc1-c1ccc(N(CCF)C2CC3CCC(C2)N3)nn1] |
| 155 | 2-(6-((2-fluoroethyl)- (2,2,6,6-tetramethyl- piperidin-4-yl)amino)- pyridazin-3-yl)-5-(1- methyl-1H-pyrazol- 4-yl)phenol | 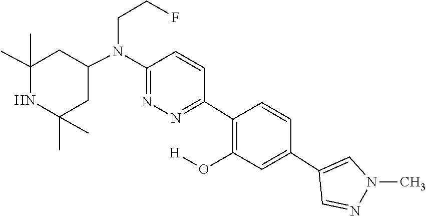 |
| 156 | 2-(6-((2-fluoroethyl)- (2,2,6,6-tetramethyl- piperidin-4-yl)amino)- pyridazin-3-yl)-5-(5- methyl-1H-pyrazol-4- yl)phenol | 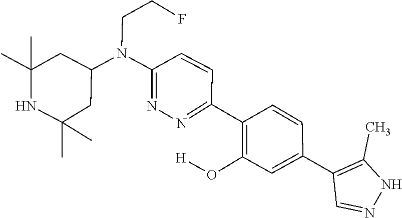 [SMILES: Cc1[nH]ncc1-c1ccc(-c2ccc(N(CCF)C3CC(C)(C)NC(C)(C)C3)nn2)c(O)c1] |
| 157 | 2-(6-((2-fluoroethyl)- (2,2,6,6-tetramethyl- piperidin-4-yl)amino)- pyridazin-3-yl)-5-(5- methyloxazol-2-yl)- phenol | 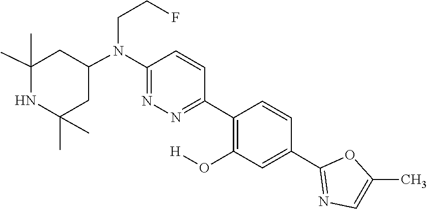 |
| 158 | 2-(6-((2-fluoroethyl)- (2,2,6,6-tetramethyl- piperidin-4-yl)amino)- pyridazin-3-yl)-5-(1H- pyrazol-1-yl)phenol | 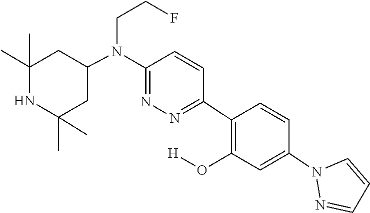 [SMILES: CC1(C)CC(N(CCF)c2ccc(-c3ccc(-n4cccn4)cc3O)nn2)CC(C)(C)N1] |
| 159 | 5-(4-(6-((2-fluoroethyl)- (2,2,6,6-tetramethyl- piperidin-4-yl)amino)- pyridazin-3-yl)-3- hydroxyphenyl)- pyridin-2(1H)-one | 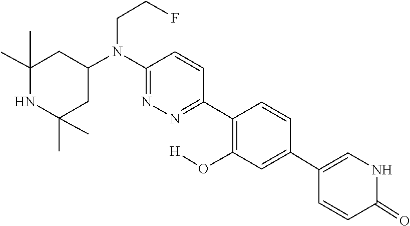 [SMILES: CC1(C)CC(N(CCF)c2ccc(-c3ccc(-c4ccc(=O)[nH]c4)cc3O)nn2)CC(C)(C)N1] |
| 160 | 5-(4-(6-((2-fluoroethyl)- (2,2,6,6-tetramethyl- piperidin-4-yl)amino)- pyridazin-3-yl)-3- hydroxyphenyl)pyrim- idin-2(1H)-one | 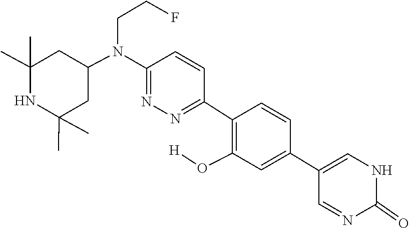 [SMILES: CC1(C)CC(N(CCF)c2ccc(-c3ccc(-c4cnc(=O)[nH]c4)cc3O)nn2)CC(C)(C)N1] |
| 161 | 2-(6-((2-methoxyeth- oxy)(2,2,6,6-tetramethyl- piperidin-4-yl)methyl)- pyridazin-3-yl)-5-(1H- pyrazol-4-yl)phenol | 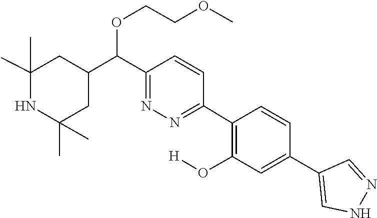 [SMILES: COCCOC(c1ccc(-c2ccc(-c3cn[nH]c3)cc2O)nn1)C1CC(C)(C)NC(C)(C)C1] |
| 162 | (3,8-diazabicyclo[3.2.1]- octan-3-yl)(6-(2- hydroxy-4-(1H-pyrazol- 4-yl)phenyl)pyridazin- 3-yl)methanone | 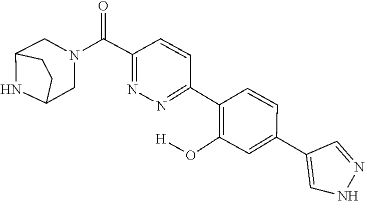 [SMILES: O=C(c1ccc(-c2ccc(-c3cn[nH]c3)cc2O)nn1)N1CC2CCC(C1)N2] |
| 163 | (3,6-diazabicyclo[3.1.1]- heptan-3-yl)(6-(2- hydroxy-4-(1H-pyrazol- 4-yl)phenyl)pyridazin- 3-yl)methanone | 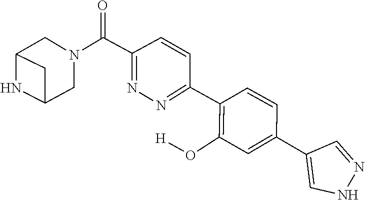 [SMILES: O=C(c1ccc(-c2ccc(-c3cn[nH]c3)cc2O)nn1)N1CC2CC(C1)N2] |

### EXAMPLES

### Example 1

The example provides an exemplary experimental plan using the methods provided herein to identify a binding agent binding to a target RNA. The experiment comprises the following steps:

Step 1 can include RNA duplex formation and NMR screening. NMR spectra with and without small molecule can be compared to determine whether the small molecule binds to the RNA duplex. In order to identify splicing modifiers of the target genes described herein, a library of compounds can be tested for their ability to bind the RNA duplex. In this case, a 2D ¹H—¹H TOCSY fingerprint of the free RNA duplex will be recorded and compared with the same fingerprint after addition of the candidate molecules. By comparing these two fingerprint spectra, one could quickly notice whether they show difference or not. If the addition of the candidate molecule induced changes of the chemical shifts of the RNA, this will support a direct interaction between the molecule and the RNA duplex. From comparing the chemical shifts and fingerprints from the two different spectra, we can determine and identify small molecules that bind to the RNA duplex or do not bind to the RNA duplex.

Step 2 can include binding specificity and effect of U1-C zinc finger domain. The screening will be based on the comparison between the free RNA and after addition of the small molecule. RNA duplex binders will be selected for further investigations. First, the strength of the interaction can be determined. By performing a titration of the RNA by the small molecule of interest, one can determine the strength of the interaction. Second, the specificity of the interaction can be determined, because the small molecule of interest can be tested against several different RNA duplexes, one can test the specificity of the identified interaction by testing the hit molecule on other RNA duplexes. Thirdly, the specificity and unique binding position of the small molecules binders on the RNA duplexes can be elucidated by comparing various RNA binders with each other. Finally, the zinc finger of U1-C can be added in the assay and offer the possibility to test how it influences or competes with the interaction of the RNA duplex—small molecule.

Step 3 can include NMR structure determination of RNA duplex—small molecule complex. The most promising small molecule—RNA duplex will be selected for structure determination using solution state NMR. In order to solve the structure of such a complex, access to high magnetic field NMR spectrometer is crucial to perform the resonance assignment but also to identify NOE-derived distances to drive structure calculations. NMR 900 MHz spectrometer or higher may be required to be used to collect data in order to solve the structure of such complex.

### Example 2

This example provides a method to use an mRNA fragment containing an exon-intron boundary with up to 200 nucleotides in length. In some experiments, the mRNA will not be labeled. ¹H spectrum will be obtained for unlabeled targets. In some other cases, the exonic/intronic nucleotides involved in the 8-12 nucleotides of the 5′ss sequence can be isotopically labeled for measurement with the NMR. This can enable us to preserve secondary structure of the mRNA while not losing any of the resolution of the experiment and the ability to determine compound binding with the rest of the sequence. The duplex RNA between the 5′-end of U1 (5′-AUAC_{ψψ}ACCUG-3′) and the 5′ss of the various targets (see Tables 1-2) can be formed by adding the U1 snRNA and the 5′ss in about equimolar amounts in NMR buffering. The experiment comprises the following steps: 1) Optionally, radiolabeling a section of the mRNA sequence in this case the 5′ss while the larger region of mRNA sequence remains unlabeled (but provides for 2-D/3-D structural sophistication); 2) obtaining a NMR spectrum of the polynucleotide sample, e.g. duplex RNA, using a NMR device; 3) introducing the U1 protein and then the small molecule of interests to determine a chemical shift of one or more atoms of the 5′ss duplex with snRNA; 4) measuring chemical shift changes upon the addition of the U1 protein indicating that the mRNA may be interacting with the U1 protein or not; 5) measuring chemical shift changes upon the addition of the small molecule and the U1 protein indicating that the mRNA may be interacting with the small molecule and protein differently from the addition of the U1 protein alone; and 6) collecting the chemical shifts in the presence of the U1 protein and/or the small molecule. The chemical shifts can be used to determine the bimolecular structure of the mRNA and the bound small molecule. From the NMR spectra, a 2-D or 3-D atomic resolution of the structure of the 5′ss and the small molecule can be computationally modeled. A plurality of secondary structure predictions can be computed using a secondary structure prediction algorithm (e.g., nearest neighbor algorithm) or computer program. The MC-Fold|MC-Sym pipeline is a web-hosted service for RNA secondary and tertiary structure prediction. The pipeline means that the input sequence to MC-Fold outputs secondary structures that are directly inputted to MC-Sym, which outputs tertiary structures.

### Example 3

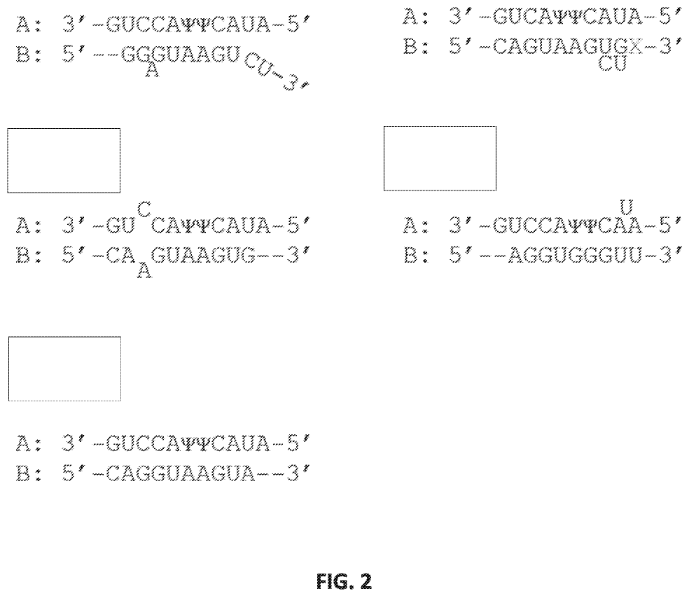

This example provides exemplary experimental procedure for NMR preparation of RNA and RNA-compound complex samples. RNA for survival of motor neuron (SMN) protein is used as an example here. SMN 5′ss RNA (5′-GGAGUAAGUCU), U1 snRNA (5′-GAUACUUACCUG) and SMN ssRNA/U1 snRNP-linked RNA (5′-GGAGUAAGUCU-GAUACUUACCUG) can be synthesized by TriLink BioTechnologies or Integrated DNA Technologies. The dsRNA can be prepared by mixing equimolar concentrations of SMN ssRNA and U1 snRNA in NMR buffer (20 mM potassium phosphate, pH 6.2, 100 mM KCl and 0.1 mM EDTA). Different RNA-RNA duplex can be used for this experiment and there are examples in FIG. 2. The mixture can be heated to 60° C. for 5 min and then cooled to room temperature. The samples for one-dimensional NMR binding studies can be made with 100 μM compound and 5 μM dsRNA in D2O buffer. SMN ssRNA/U1 snRNP-linked RNA can be used for the computational modeling structure determination after confirmation that the stem-loop base pairing patterns are the same as those of the SMN ssRNA/snRNP RNA dsRNA by TOCSY. The samples for TOCSY with SMN ssRNA and U1 snRNA in D₂O or H₂O buffer can be heated to 85° C. for 5 min and then cooled to room temperature. The SMN ssRNA-U1 snRNA-NVS-SM2 complex can be prepared by adding 10 mM DMSO-d6 stock solution of NVS-SM2 to 350-500 μM of dsRNA until the compound concentration reached saturation.

### Example 4

NMR experiments can be performed on AVANCE III 600 MHz or 800 MHz spectrometers (Bruker). The sample temperature can be 20° C. for binding experiments with the dsRNA and 5-37° C. for structure determination experiments including ¹D ¹H, and 2-D COSY and TOCSY with RNA-11 and RNA-12. The model was assembled from a data set that included analysis of TOCSY spectra.

NMR spectra can be acquired at 303 K and 313 K for RNA-protein complexes or 313 K for all other protein complexes on Bruker Avance III 500, 600, 700 or 900 MHz spectrometers equipped with cryoprobes and on a Bruker Avance III 750 MHz spectrometer with a room temperature probe. Spectra can be processed with Topspin 2.1 or Topspin 3.0 and analyzed in Sparky 3.0. ¹H, ¹³C and ¹⁵N assignments of RNA and protein can be achieved by standard methods in the art. For modeling of the RNA-protein complex, intramolecular distance restraints derived from HHC- and HHN-3D-NOESY experiments as well as residual dipolar couplings measured for backbone amides and RNA-C1′-H1′, C5-H5, C6-H6, C8-H8 and C2-H2 bonds can be used. Intermolecular distance restraints can be extracted from 3-D ¹³C—F₁-edited, F3-filtered-NOESY-HSQCs and 2-D ¹H—¹H F₁—¹³C-filtered, F₂—¹³C-edited NOESY spectra recorded on complexes reconstituted either from ¹³C¹⁵N-labeled protein and unlabeled RNA or from ¹⁵N-labeled protein and ¹³C¹⁵N-labeled RNA.

### Example 5

This example provides exemplary modeling strategy. Modeling of RNA-protein complex can be implemented with a combination of different software classically required for structure prediction and determination of protein-RNA complexes. The Atnos/Candid-program suite and artificial RRM NOESY matrices can be used to generate peak lists corresponding to intramolecular NOESY patterns typical for the RRM fold. CYANA 3.0 and more particularly the CYANA noeassign command can be used to integrate distance and angle restraints and to calculate models. For modeling, CUR-MS/MS-data can be inserted as ambiguous distance restraints because crosslinking sites define various distances between base rings of nucleic acids and side chains of amino acids, respectively. Intramolecular restraints can be derived from published protein structures in RCSB Protein Data Bank (PDB) and RNA structures predicted by MC-FOLD and MC-SYM. Additional specific protein-RNA contacts extracted from available complex structures can be integrated as unambiguous distance restraints. For all models, about 200 structures per cycle can be calculated and about 20 of lowest energy can be selected as a starting ensemble for the next cycle. For modeling RNA-protein complexes, the CYANA noeassign calculation can be initiated with the average protein-RNA complex structure from PDB in cycle 1 excluding the RNA moiety. The final 20 lowest energy models obtained with CYANA noeassign can be refined with the amber 12 force field to avoid steric clashes and to improve electrostatic and hydrophobic protein-RNA contacts.

### Example 6

This example shows binding kinetics by SPR analysis of U1 snRNP binding to RNA. Biotinylated RNAs (5′-biotinTEG/UCUAAGGCGUAAGUCUGCCAG-3′, and 5′-biotinTEG/UCUAAGCAGUAAGUCUGCCAG-3′) can be synthesized by Integrated DNA Technologies. Initial SPR studies with compound only in the association phase can be performed on a Biacore T100 at 25° C. RNA will be diluted into SPR buffer (38 mM HEPES, pH 7.6, 60 mM KCl, 0.12 mM EDTA, 3.2 MgCl2, 0.05% P20), heated to 90° C., slowly cooled to room temperature and centrifuged for 10 min at 14,000 g, and a target level of 110 relative units (RU) will be captured onto a streptavidin-coated SA chip (GE Healthcare). U1 snRNP will be diluted 1:50 with SPR buffer containing either DMSO or compound. Final DMSO concentration will be 0.5%, and the running buffer will be adjusted to the same percentage. The surface will be regenerated with 1 M NaCl, 10 mM NaOH. Co-injection experiments will be performed under the same buffer conditions on a ProteOn XPR36 at 25° C. using a NLC chip (Bio-Rad) with a minimum of 25 RUs of target RNA loaded on the surface. The ProteOn's co-inject function allowed testing of NVS-SM2 or DMSO in both the association and dissociation phases. Dissociation rate constants are independent of analyte concentration and can be measured using the ProteOn software from two duplicate injections. All data will be double referenced to a protein-only surface as well as a buffer injection, and a DMSO correction for excluded volume will be performed.

### Example 7

The example shows binding kinetics by SPR analysis of U1 snRNA binding to RNA. SPR studies will be performed on a ProteOn XPR36 at 20° C. using a NLC chip (BioRad) with a minimum of 300 RUs of target RNA loaded on the surface. U1 snRNA (5′-AUACUUACCUG-3′) will be diluted to 1 μM with SPR buffer containing either DMSO or compound. The co-inject feature will be used so that the association and dissociation phases contained either DMSO or compound. Surface regeneration and referencing will be performed as above Example 5.

### Example 8

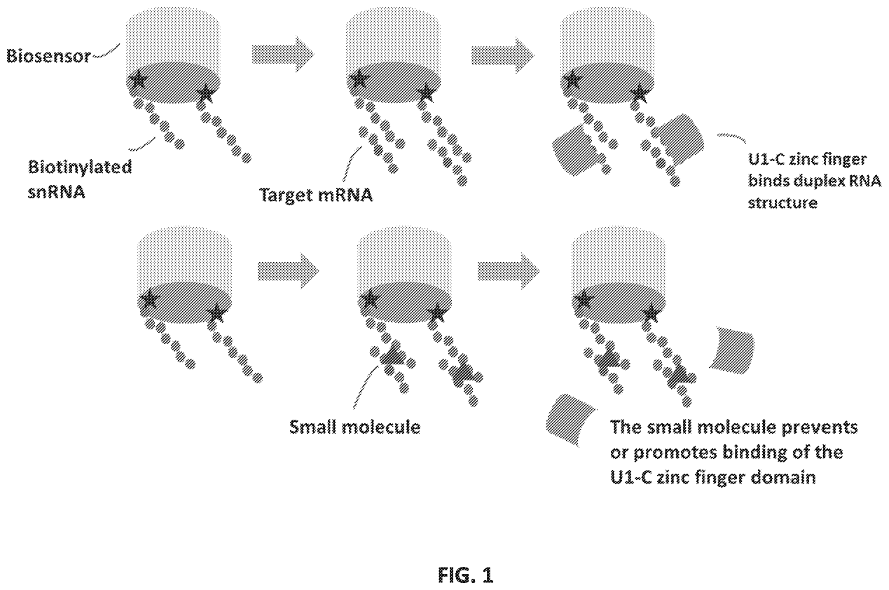

FIG. 1 shows a schematic of a binding kinetics assay by Bio-Layer Interferometry (BLI). In this exemplary experimental design, snRNA is immobilized on a surface through, for example, biotin-streptavidin interaction. In the solution, target mRNA and U1-C zinc finger domain are added and they bind to the immobilized snRNA to form a complex. In the presence of the small molecule binder, it can bind to the RNA-RNA duplex and destabilized the protein-RNA complex by preventing protein from binding to the RNA-RNA duplex. Various concentrations of the small molecule can be titrated into the same target complex (e.g. mRNA-snRNA-U1-C) in order to determine a binding kinetics. K_{d} can be determined with the small molecule titration.

### Example 9

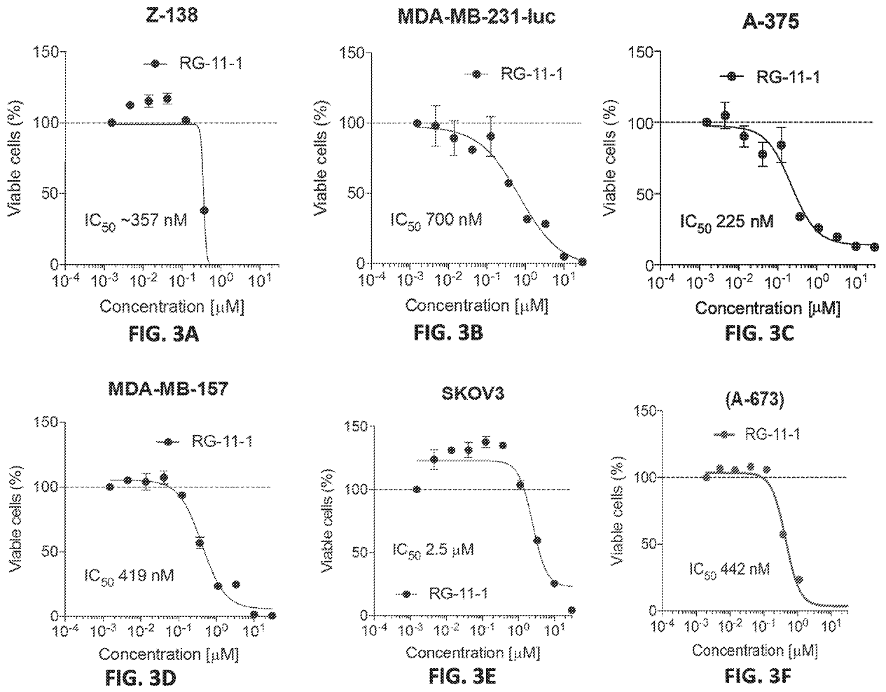

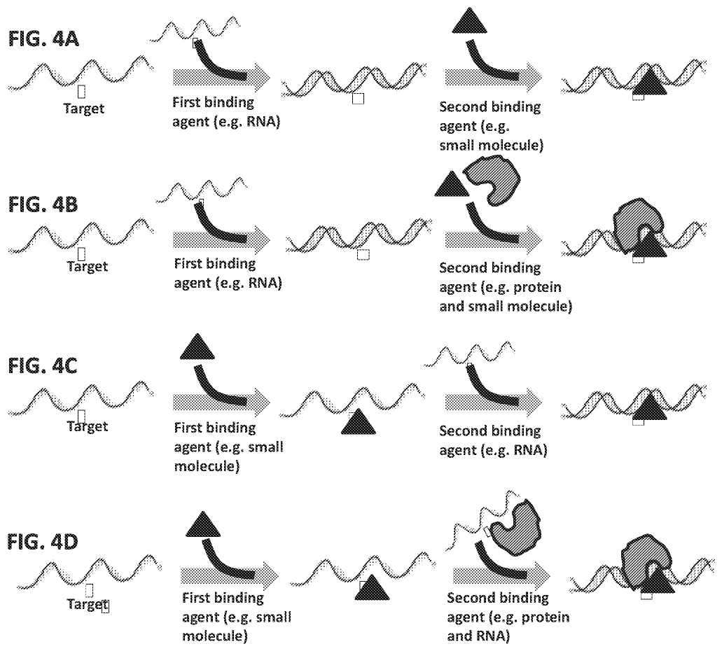

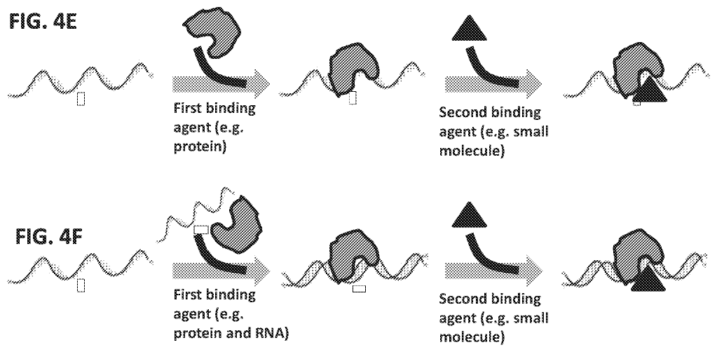

The small molecule of interest disclosed herein can be tested in cell-based assay for efficiency measurement, for example, IC₅₀. To measure cell viability, cells were plated in 96-well plastic tissue culture plates at a density of 5×10³ cells/well. Twenty-four hours after plating, cells were treated with RG-11-1 compound. After 72 hours, the cell culture media was removed and plates were stained with 100 mL/well of a solution containing 0.5% crystal violet and 25% methanol, rinsed with deionized water, dried overnight, and resuspended in 100 ml citrate buffer (0.1 M sodium citrate in 50% ethanol) to assess plating efficiency. Intensity of crystal violet staining, assessed at :570 nm and quantified using a Vmax Kinetic Microplate Reader and Softmax software (Molecular Devices Corp., Menlo Park, Calif.), was directly proportional to cell number. Data were normalized to vehicle-treated cells and are presented in FIG. 3A-F as the mean±SE from representative experiments.

### Example 10

For example, the disclosed methods can be used to select small molecule binding agents for modulating splicing of mRNA expressed from FOXM1 gene. The exemplary small molecules can target 5′ss of FOXM1 mRNA (5′ss of exon 9). They may also target some other elements of mRNA or target other mRNA for other genes. Exemplary structures are summarized herein:

In one aspect, a compound that could be identified by the present disclosed methods has the structure of Formula (I), or a pharmaceutically acceptable salt or solvate thereof:

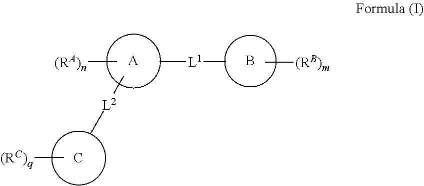

wherein,
ring A is aryl or heteroaryl;
each R^{A} is independently selected from H, D, halogen, —CN, —OH, —OR¹, —SR¹, —S(═O)R¹, —S(═O)₂R¹, —NHS(═O)₂R¹, —S(═O)₂N(R¹)₂, —C(═O)R¹, —OC(═O)R¹, substituted or unsubstituted C₁-C₆alkyl, substituted or unsubstituted C₃-C₆cycloalkyl, substituted or unsubstituted C₂-C₆alkenyl, substituted or unsubstituted C₂-C₆alkynyl, substituted or unsubstituted C₁-C₆fluoroalkyl, and substituted or unsubstituted C₁-C₆heteroalkyl;
L¹ is —X¹-L³- or -L³-X¹—;
   X¹ is —O—, —S—, —S(═O)—, —S(═O)₂—, —S(═O)₂NR¹—, —CH₂—, —C(═O)—, —C(═O)O—, —OC(═O)—, —C(═O)NR¹—, —NR¹C(═O)—, —OC(═O)NR¹—, —NR¹C(═O)O—, —NR¹C(═O)NR¹—, —NR¹S(═O)₂—, or —NR¹—;
   L³ is absent or substituted or unsubstituted C₁-C₄alkylene;
ring B is monocyclic carbocycle, bicyclic carbocycle, monocyclic heterocycle, or bicyclic heterocycle;
each R^{B} is independently selected from H, D, halogen, —CN, —OH, —OR¹, —SR¹, —S(═O)R¹, —S(═O)₂R¹, —N(R¹)₂, —NR¹S(═O)₂R¹, —S(═O)₂N(R¹)₂, —C(═O)R¹, —OC(═O)R¹, —CO₂R¹, —OCO₂R¹, —C(═O)N(R¹)₂, —OC(═O)N(R¹)₂, —NR¹C(═O)N(R¹)₂, NR¹⁰C(═N—CN)N(R¹)₂, —NR¹C(═O)R¹, —NR¹C(═O)OR¹, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, substituted or unsubstituted aryl, and substituted or unsubstituted monocyclic heteroaryl;
each R¹ is independently H, substituted or unsubstituted C₁-C₆alkyl, substituted or unsubstituted C₁-C₆fluoroalkyl, substituted or unsubstituted C₁-C₆heteroalkyl, substituted or unsubstituted phenyl, or substituted or unsubstituted heteroaryl;
L² is —X²-L⁴-, or -L⁴-X²—;
   X² is absent, —O—, —S—, —S(═O)—, —S(═O)₂—, —CH₂—, —CH═CH—, —C≡C—, —C(═O)—, —C(═O)O—, —OC(═O)—, —OC(═O)O—, —C(═O)NR¹—, —NR¹C(═O)—, —OC(═O)NR¹—, —NR¹C(═O)O—, —NR¹C(═O)NR¹—, —NR¹S(═O)₂—, —S(═O)₂NR¹—, or —NR¹—;
L⁴ is absent or substituted or unsubstituted C₁-C₃alkylene;
ring C is monocyclic carbocycle, bicyclic carbocycle, monocyclic heterocycle, or bicyclic heterocycle;
each R^{C} is independently selected from H, D, halogen, —CN, —OH, —OR¹, —SR¹, —S(═O)R¹, —S(═O)₂R¹, —N(R¹)₂, —CH₂—N(R¹)₂, —NHS(═O)₂R¹, —S(═O)₂N(R¹)₂, —C(═O)R¹, —OC(═O)R¹, —CO₂R¹, —OCO₂R¹, —C(═O)N(R¹)₂, —OC(═O)N(R¹)₂, —NR¹C(═O)N(R¹)₂, —NR¹C(═O)R¹¹, —NR¹C(═O)OR¹, substituted or unsubstituted C₁-C₆heteroalkyl, substituted or unsubstituted C₃-C₈cycloalkyl, and substituted or unsubstituted C₂-C₈heterocycloalkyl;
n is 0, 1, or 2;
m is 0, 1, or 2; and
q is 0, 1, 2, 3, 4, 5, or 6.

In another aspect, a compound that could be identified by the present disclosed methods has the structure of Formula (II), or a pharmaceutically acceptable salt or solvate thereof:

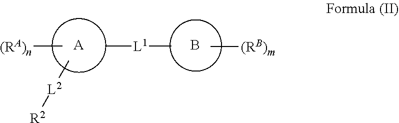

wherein,
ring A is aryl or heteroaryl;
each R^{A} is independently selected from H, D, halogen, —CN, —OH, —OR¹, —SR¹, —S(═O)R¹, —S(═O)₂R¹, —NHS(═O)₂R¹, —S(═O)₂N(R¹)₂, —C(═O)R¹, —OC(═O)R¹, substituted or unsubstituted C₁-C₆alkyl, substituted or unsubstituted C₃-C₆cycloalkyl, substituted or unsubstituted C₂-C₆alkenyl, substituted or unsubstituted C₂-C₆alkynyl, substituted or unsubstituted C₁-C₆fluoroalkyl, and substituted or unsubstituted C₁-C₆heteroalkyl;
L¹ is —X¹-L³-, or -L³-X¹—;
   X¹ is absent, —O—, —S—, —S(═O)—, —S(═O)₂—, —S(═O)₂NR¹—, —CH₂—, —C(═O)—, —C(═O)O—, —OC(═O)—, —C(═O)NR¹—, —NR¹C(═O)—, —OC(═O)NR¹—, —NR¹C(═O)O—, —NR¹C(═O)NR¹—, —NR¹S(═O)₂—, or —NR—;
   L³ is absent or substituted or unsubstituted C₁-C₄alkylene;
ring B is monocyclic carbocycle, bicyclic carbocycle, monocyclic heterocycle, or bicyclic heterocycle;
each R^{B} is independently selected from H, D, halogen, —CN, —OH, —OR¹, —SR¹, —S(═O)R¹, —N(R¹)₂, —S(═O)₂R¹, —NR¹S(═O)₂R¹, —S(═O)₂N(R¹)₂, —C(═O)R¹, —OC(═O)R¹, —CO₂R¹, —OCO₂R¹, —C(═O)N(R¹)₂, —OC(═O)N(R¹)₂, —NR¹C(═O)N(R¹)₂, NR¹⁰C═N—CN)N(R¹)₂, —NR¹C(═O)R¹, —NR¹C(═O)OR¹, substituted or unsubstituted C₁-C₆alkyl, substituted or unsubstituted C₁-C₆fluoroalkyl, substituted or unsubstituted C₁-C₆heteroalkyl, substituted or unsubstituted aryl, and substituted or unsubstituted monocyclic heteroaryl;
each R¹ is independently H, substituted or unsubstituted C₁-C₆alkyl, substituted or unsubstituted C₁-C₆fluoroalkyl, substituted or unsubstituted C₁-C₆heteroalkyl, substituted or unsubstituted phenyl, or substituted or unsubstituted heteroaryl;
L² is —X²-L⁴-, or -L⁴-X²—;
   X² is absent, —O—, —S—, —S(═O)—, —S(═O)₂—, —S(═O)₂NR¹—, —CH₂—, —CH═CH—, —C≡C—, —C(═O)—, —C(═O)O—, —OC(═O)—, —OC(═O)O—, —C(═O)NR¹—, —NR¹C(═O)—, —OC(═O)NR¹—, —NR¹C(═O)O—, —NR¹C(═O)NR¹—, —NR¹S(═O)₂—, or —NR¹—;
L⁴ is absent or substituted or unsubstituted C₁-C₃alkylene;
R² is independently selected from H, D, —F, —CN, —OH, —OR¹, —SR¹, —S(═O)R¹, —S(═O)₂R¹, —N(R¹)₂, —NHS(═O)₂R¹, —S(═O)₂N(R¹)₂, —C(═O)R¹, —OC(═O)R¹, —CO₂R¹, —OCO₂R¹, —C(═O)N(R¹)₂, —OC(═O)N(R¹)₂, —NR¹C(═O)N(R¹)₂, —NR¹C(═O)R¹¹, —NR¹C(═O)OR¹, substituted or unsubstituted C₁-C₆alkyl, substituted or unsubstituted C₁-C₆heteroalkyl, substituted or unsubstituted C₃-C₈cycloalkyl, substituted or unsubstituted C₂-C₆alkynyl, and substituted or unsubstituted C₁-C₆fluoroalkyl;
n is 0, 1, or 2; and
m is 0, 1, or 2.

In some embodiments, a compound that could be identified herein has the structure of Formula (III), or a pharmaceutically acceptable salt or solvate thereof:

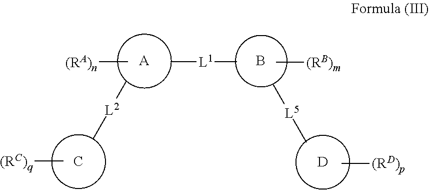

wherein,
ring A is aryl or heteroaryl;
each R^{A} is independently selected from H, D, halogen, —CN, —OH, —OR¹, —SR¹, —S(═O)R¹, —S(═O)₂R¹, —NHS(═O)₂R¹, —S(═O)₂N(R¹)₂, —C(═O)R¹, —OC(═O)R¹, substituted or unsubstituted C₁-C₆alkyl, substituted or unsubstituted C₃-C₆cycloalkyl, substituted or unsubstituted C₂-C₆alkenyl, substituted or unsubstituted C₂-C₆alkynyl, substituted or unsubstituted C₁-C₆fluoroalkyl, and substituted or unsubstituted C₁-C₆heteroalkyl;
L¹ is —X¹-L³-, or -L³-X¹—;
   X¹ is absent, —O—, —S—, —S(═O)—, —S(═O)₂—, —S(═O)₂NR¹—, —CH₂—, —C(═O)—, —C(═O)O—, —OC(═O)—, —C(═O)NR¹—, —NR¹C(═O)—, —OC(═O)NR¹—, —NR¹C(═O)O—, —NR¹C(═O)NR¹—, —NR¹S(═O)₂—, or —NR¹—;
   L³ is absent or substituted or unsubstituted C₁-C₄alkylene;
ring B is aryl or heteroaryl;
each R^{B} is independently selected from H, D, halogen, —CN, —OH, —OR¹, —SR¹, —S(═O)R¹, —N(R¹)₂, —S(═O)₂R¹, —NR¹S(═O)₂R¹, —S(═O)₂N(R¹)₂, —C(═O)R¹, —OC(═O)R¹, —CO₂R¹, —OCO₂R¹, —C(═O)N(R¹)₂, —OC(═O)N(R¹)₂, —NR¹C(═O)N(R¹)₂, NR¹⁰C(═N—CN)N(R¹)₂, —NR¹C(═O)R¹, —NR¹C(═O)OR¹, substituted or unsubstituted C₁-C₆alkyl, substituted or unsubstituted C₁-C₆fluoroalkyl, substituted or unsubstituted C₁-C₆heteroalkyl, substituted or unsubstituted aryl, and substituted or unsubstituted monocyclic heteroaryl;
each R¹ is independently H, substituted or unsubstituted C₁-C₆alkyl, substituted or unsubstituted C₁-C₆fluoroalkyl, substituted or unsubstituted C₁-C₆heteroalkyl, substituted or unsubstituted phenyl, or substituted or unsubstituted heteroaryl;
L² is —X²-L⁴-, or -L⁴-X²—;
   X² is absent, —O—, —S—, —S(═O)—, —S(═O)₂—, —S(═O)₂NR¹—, —CH₂—, —CH═CH—, —C≡C—, —C(═O)—, —C(═O)O—, —OC(═O)—, —OC(═O)O—, —C(═O)NR¹—, —NR¹C(═O)—, —OC(═O)NR¹—, —NR¹C(═O)O—, —NR¹C(═O)NR¹—, —NR¹S(═O)₂—, or —NR¹—;
L⁴ is absent or substituted or unsubstituted C₁-C₃alkylene;
ring C is monocyclic carbocycle, bicyclic carbocycle, monocyclic heterocycle, or bicyclic heterocycle;
each R^{C} is independently selected from H, D, halogen, —CN, —OH, —OR¹, —SR¹, —S(═O)R¹, —S(═O)₂R¹, —N(R¹)₂, —CH₂—N(R¹)₂, —NHS(═O)₂R¹, —S(═O)₂N(R¹)₂, —C(═O)R¹, —OC(═O)R¹, —CO₂R¹, —OCO₂R¹, —C(═O)N(R¹)₂, —C(═O)N(R¹)₂, —NR¹C(═O)N(R¹)₂, —NR¹C(═O)R¹¹, —NR¹C(═O)OR¹, substituted or unsubstituted C₁-C₆alkyl, substituted or unsubstituted C₁-C₆heteroalkyl, substituted or unsubstituted C₃-C₈cycloalkyl, substituted or unsubstituted C₂-C₆alkynyl, and substituted or unsubstituted C₁-C₆fluoroalkyl;
ring D is monocyclic carbocycle or monocyclic heterocycle;
each R^{D} is independently selected from H, D, halogen, —CN, —OH, —OR¹, —SR¹, —S(═O)R¹, —S(═O)₂R¹, —NHS(═O)₂R¹, —S(═O)₂N(R¹)₂, —C(═O)R¹, —OC(═O)R¹, substituted or unsubstituted C₁-C₆alkyl, substituted or unsubstituted C₃-C₆cycloalkyl, substituted or unsubstituted C₂-C₆alkenyl, substituted or unsubstituted C₂-C₆alkynyl, substituted or unsubstituted C₁-C₆fluoroalkyl, and substituted or unsubstituted C₁-C₆heteroalkyl;
L⁵ is —X³-L⁶-, or -L⁶-X³—;
   X³ is absent, —O—, —S—, —S(═O)—, —S(═O)₂—, —S(═O)₂NR¹—, —CH₂—, —C(═O)—, —C(═O)O—, —OC(═O)—, —C(═O)NR¹—, —NR¹C(═O)—, —OC(═O)NR¹—, —NR¹C(═O)O—, —NR¹C(═O)NR¹—, —NR¹S(═O)₂—, or —NR¹—;
L⁶ is absent or substituted or unsubstituted C₁-C₄alkylene;
n is 0, 1, or 2;
m is 0, 1, or 2;
q is 0, 1, 2, 3, 4, 5, or 6; and
p is 0, 1, 2, 3, or 4.

In another aspect, a compound that could be identified herein has the structure of Formula (IV), or a pharmaceutically acceptable salt or solvate thereof:

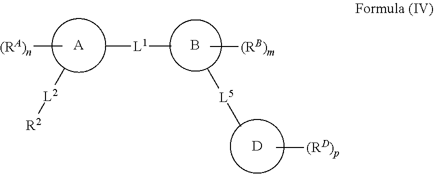

wherein,
ring A is aryl or heteroaryl;
each R^{A} is independently selected from H, D, halogen, —CN, —OH, —OR¹, —SR¹, —S(═O)R¹, —S(═O)₂R¹, —NHS(═O)₂R¹, —S(═O)₂N(R¹)₂, —C(═O)R¹, —OC(═O)R¹, substituted or unsubstituted C₁-C₆alkyl, substituted or unsubstituted C₃-C₆cycloalkyl, substituted or unsubstituted C₂-C₆alkenyl, substituted or unsubstituted C₂-C₆alkynyl, substituted or unsubstituted C₁-C₆fluoroalkyl, and substituted or unsubstituted C₁-C₆heteroalkyl;
L¹ is —X¹-L³-, or -L³-X¹—;
   X¹ is absent, —O—, —S—, —S(═O)—, —S(═O)₂—, —S(═O)₂NR¹—, —CH₂—, —C(═O)—, —C(═O)O—, —OC(═O)—, —C(═O)NR¹—, —NR¹C(═O)—, —OC(═O)NR¹—, —NR¹C(═O)O—, —NR¹C(═O)NR¹—, —NR¹S(═O)₂—, or —NR¹—;
   L³ is absent or substituted or unsubstituted C₁-C₄alkylene;
ring B is monocyclic carbocycle, bicyclic carbocycle, monocyclic heterocycle, or bicyclic heterocycle;
each R^{B} is independently selected from H, D, halogen, —CN, —OH, —OR¹, —SR¹, —S(═O)R¹, —N(R¹)₂, —S(═O)₂R¹, —NR¹S(═O)₂R¹, —S(═O)₂N(R¹)₂, —C(═O)R¹, —OC(═O)R¹, —CO₂R¹, —OCO₂R¹, —C(═O)N(R¹)₂, —OC(═O)N(R¹)₂, —NR¹C(═O)N(R¹)₂, NR¹⁰C(═N—CN)N(R¹)₂, —NR¹C(═O)R¹, —NR¹C(═O)OR¹, substituted or unsubstituted C₁-C₆alkyl, substituted or unsubstituted C₁-C₆fluoroalkyl, substituted or unsubstituted C₁-C₆heteroalkyl, substituted or unsubstituted aryl, and substituted or unsubstituted monocyclic heteroaryl;
each R¹ is independently H, substituted or unsubstituted C₁-C₆alkyl, substituted or unsubstituted C₁-C₆fluoroalkyl, substituted or unsubstituted C₁-C₆heteroalkyl, substituted or unsubstituted phenyl, or substituted or unsubstituted heteroaryl;
L² is —X²-L⁴-, or -L⁴-X²—;
   X² is absent, —O—, —S—, —S(═O)—, —S(═O)₂—, —S(═O)₂NR¹—, —CH₂—, —CH═CH—, —C≡C—, —C(═O)—, —C(═O)O—, —OC(═O)—, —OC(═O)O—, —C(═O)NR¹—, —NR¹C(═O)—, —OC(═O)NR¹—, —NR¹C(═O)O—, —NR¹C(═O)NR¹—, —NR¹S(═O)₂—, or —NR¹—;
L⁴ is absent or substituted or unsubstituted C₁-C₃alkylene;
R² is independently selected from H, D, —F, —CN, —OH, —OR¹, —SR¹, —S(═O)R¹, —S(═O)₂R¹, —N(R¹)₂, —NHS(═O)₂R¹, —S(═O)₂N(R¹)₂, —C(═O)R¹, —OC(═O)R¹, —CO₂R¹, —OCO₂R¹, —C(═O)N(R¹)₂, —OC(═O)N(R¹)₂, —NR¹C(═O)N(R¹)₂, —NR¹C(═O)R¹¹, —NR¹C(═O)OR¹, substituted or unsubstituted C₁-C₆alkyl, substituted or unsubstituted C₁-C₆heteroalkyl, substituted or unsubstituted C₃-C₈cycloalkyl, substituted or unsubstituted C₂-C₆alkynyl, and substituted or unsubstituted C₁-C₆fluoroalkyl;
ring D is monocyclic carbocycle or monocyclic heterocycle;
each R^{D} is independently selected from H, D, halogen, —CN, —OH, —OR¹, —SR¹, —S(═O)R¹, —S(═O)₂R¹, —NHS(═O)₂R¹, —S(═O)₂N(R¹)₂, —C(═O)R¹, —OC(═O)R¹, substituted or unsubstituted C₁-C₆alkyl, substituted or unsubstituted C₃-C₆cycloalkyl, substituted or unsubstituted C₂-C₆alkenyl, substituted or unsubstituted C₂-C₆alkynyl, substituted or unsubstituted C₁-C₆fluoroalkyl, and substituted or unsubstituted C₁-C₆heteroalkyl;
L⁵ is —X³-L⁶-, or -L⁶-X³—;
   X³ is absent, —O—, —S—, —S(═O)—, —S(═O)₂—, —S(═O)₂NR¹—, —CH₂—, —C(═O)—, —C(═O)O—, —OC(═O)—, —C(═O)NR¹—, —NR¹C(═O)—, —OC(═O)NR¹—, —NR¹C(═O)O—, —NR¹C(═O)NR¹—, —NR¹S(═O)₂—, or —NR¹—;
L⁶ is absent or substituted or unsubstituted C₁-C₄alkylene;
n is 0, 1, or 2;
m is 0, 1, or 2; and
p is 0, 1, 2, 3, or 4.

In one aspect, a compound that could be identified herein has the structure of Formula (V), or a pharmaceutically acceptable salt or solvate thereof:

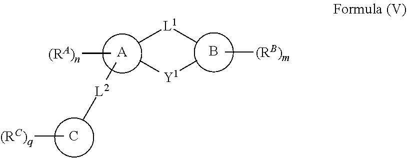

wherein,
ring A is aryl or heteroaryl;
each R^{A} is independently selected from H, D, halogen, —CN, —OH, —OR¹, —SR¹, —S(═O)R¹, —S(═O)₂R¹, —NHS(═O)₂R¹, —S(═O)₂N(R¹)₂, —C(═O)R¹, —OC(═O)R¹, substituted or unsubstituted C₁-C₆alkyl, substituted or unsubstituted C₃-C₆cycloalkyl, substituted or unsubstituted C₂-C₆alkenyl, substituted or unsubstituted C₂-C₆alkynyl, substituted or unsubstituted C₁-C₆fluoroalkyl, and substituted or unsubstituted C₁-C₆heteroalkyl;
L¹ is —X¹-L³- or -L³-X¹—;
   X¹ is —O—, —S—, —S(═O)—, —S(═O)₂—, —S(═O)₂NR¹—, —CH₂—, —C(═O)—, —C(═O)O—, —OC(═O)—, —C(═O)NR¹—, —NR¹C(═O)—, —NR¹S(═O)₂—, or —NR¹—;
   L³ is absent or substituted or unsubstituted C₁-C₂alkylene;
Y¹ is —W¹—Y²— or —Y²—W¹—;
   W¹ is —O—, —S—, —S(═O)—, —S(═O)₂—, —S(═O)₂NR¹—, —CH₂—, —C(═O)—, —C(═O)O—, —OC(═O)—, —C(═O)NR¹—, —NR¹C(═O)—, —NR¹S(═O)₂—, or —NR¹—;
   Y² is absent or substituted or unsubstituted C₁-C₂alkylene;
ring B is aryl or heteroaryl;
each R^{B} is independently selected from H, D, halogen, —CN, —OH, —OR¹, —SR¹, —S(═O)R¹, —S(═O)₂R¹, —N(R¹)₂, —NR¹S(═O)₂R¹, —S(═O)₂N(R¹)₂, —C(═O)R¹, —OC(═O)R¹, —CO₂R¹, —OCO₂R¹, —C(═O)N(R¹)₂, —OC(═O)N(R¹)₂, —NR¹C(═O)N(R¹)₂, NR¹⁰C(═N—CN)N(R¹)₂, —NR¹C(═O)R¹, —NR¹C(═O)OR¹, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, substituted or unsubstituted aryl, and substituted or unsubstituted monocyclic heteroaryl;
each R¹ is independently H, substituted or unsubstituted C₁-C₆alkyl, substituted or unsubstituted C₁-C₆fluoroalkyl, substituted or unsubstituted C₁-C₆heteroalkyl, substituted or unsubstituted phenyl, or substituted or unsubstituted heteroaryl;
L² is —X²-L⁴-, or -L⁴-X²—;
   X² is absent, —O—, —S—, —S(═O)—, —S(═O)₂—, —CH₂—, —CH═CH—, —C≡—, —C(═O)—, —C(═O)O—, —OC(═O)—, —OC(═O)O—, —C(═O)NR¹—, —NR¹C(═O)—, —OC(═O)NR¹—, —NR¹C(═O)O—, —NR¹C(═O)NR¹—, —NR¹S(═O)₂—, —S(═O)₂NR¹—, or —NR¹—;
L⁴ is absent or substituted or unsubstituted C₁-C₃alkylene;
ring C is monocyclic carbocycle, bicyclic carbocycle, monocyclic heterocycle, or bicyclic heterocycle;
each R^{C} is independently selected from H, D, halogen, —CN, —OH, —OR¹, —SR¹, —S(═O)R¹, —S(═O)₂R¹, —N(R¹)₂, —CH₂—N(R¹)₂, —NHS(═O)₂R¹, —S(═O)₂N(R¹)₂, —C(═O)R¹, —OC(═O)R¹, —CO₂R¹, —OCO₂R¹, —C(═O)N(R¹)₂, —OC(═O)N(R¹)₂, —NR¹C(═O)N(R¹)₂, —NR¹C(═O)R¹¹, —NR¹C(═O)OR¹, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, and substituted or unsubstituted C₂-C₈heterocycloalkyl;
n is 0, 1, or 2;
m is 0, 1, or 2; and
q is 0,1,2,3,4,5, or6.

In another aspect, a compound that could be identified herein has the structure of Formula (VI), or a pharmaceutically acceptable salt or solvate thereof:

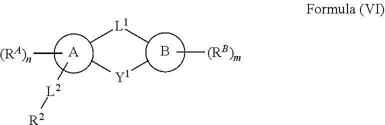

wherein,
ring A is aryl or heteroaryl;
each R^{A} is independently selected from H, D, halogen, —CN, —OH, —OR¹, —SR¹, —S(═O)R¹, —S(═O)₂R¹, —NHS(═O)₂R¹, —S(═O)₂N(R¹)₂, —C(═O)R¹, —OC(═O)R¹, substituted or unsubstituted C₁-C₆alkyl, substituted or unsubstituted C₃-C₆cycloalkyl, substituted or unsubstituted C₂-C₆alkenyl, substituted or unsubstituted C₂-C₆alkynyl, substituted or unsubstituted C₁-C₆fluoroalkyl, and substituted or unsubstituted C₁-C₆heteroalkyl;
L¹ is —X¹-L³- or -L³-X¹—;
   X¹ is —O—, —S—, —S(═O)—, —S(═O)₂—, —S(═O)₂NR¹—, —CH₂—, —C(═O)—, —C(═O)O—, —OC(═O)—, —C(═O)NR¹—, —NR¹C(═O)—, —NR¹S(═O)₂—, or —NR¹—;
   L³ is absent or substituted or unsubstituted C₁-C₂alkylene;
Y¹ is —W¹—Y²— or Y²—W¹—;
   W¹ is —O—, —S—, —S(═O)—, —S(═O)₂—, —S(═O)₂NR¹—, —CH₂—, —C(═O)—, —C(═O)O—, —OC(═O)—, —C(═O)NR¹—, —NR¹C(═O)—, —NR¹S(═O)₂—, or —NR¹—;
   Y² is absent or substituted or unsubstituted C₁-C₂alkylene;
ring B is monocyclic carbocycle, bicyclic carbocycle, monocyclic heterocycle, or bicyclic heterocycle;
each R^{B} is independently selected from H, D, halogen, —CN, —OH, —OR¹, —SR¹, —S(═O)R¹, —N(R¹)₂, —S(═O)₂R¹, —NR¹S(═O)₂R¹, —S(═O)₂N(R¹)₂, —C(═O)R¹, —OC(═O)R¹, —CO₂R¹, —OCO₂R¹, —C(═O)N(R¹)₂, —OC(═O)N(R¹)₂, —NR¹C(═O)N(R¹)₂, NR¹⁰C(═N—CN)N(R¹)₂, —NR¹C(═O)R¹, —NR¹C(═O)OR¹, substituted or unsubstituted C₁-C₆alkyl, substituted or unsubstituted C₁-C₆fluoroalkyl, substituted or unsubstituted C₁-C₆heteroalkyl, substituted or unsubstituted aryl, and substituted or unsubstituted monocyclic heteroaryl;
each R¹ is independently H, substituted or unsubstituted C₁-C₆alkyl, substituted or unsubstituted C₁-C₆fluoroalkyl, substituted or unsubstituted C₁-C₆heteroalkyl, substituted or unsubstituted phenyl, or substituted or unsubstituted heteroaryl;
L² is —X²-L⁴-, or -L⁴-X²—;
   X² is absent, —O—, —S—, —S(═O)—, —S(═O)₂—, —S(═O)₂NR¹—, —CH₂—, —CH═CH—, —C≡C—, —C(═O)—, —C(═O)O—, —OC(═O)—, —OC(═O)O—, —C(═O)NR¹—, —NR¹C(═O)—, —OC(═O)NR¹—, —NR¹C(═O)O—, —NR¹C(═O)NR¹—, —NR¹S(═O)₂—, or —NR¹—;
L⁴ is absent or substituted or unsubstituted C₁-C₃alkylene;
R² is independently selected from H, D, —F, —CN, —OH, —OR¹, —SR¹, —S(═O)R¹, —S(═O)₂R¹, —N(R¹)₂, —NHS(═O)₂R¹, —S(═O)₂N(R¹)₂, —C(═O)R¹, —OC(═O)R¹, —CO₂R¹, —OCO₂R¹, —C(═O)N(R¹)₂, —OC(═O)N(R¹)₂, —NR¹C(═O)N(R¹)₂, —NR¹C(═O)R¹¹, —NR¹C(═O)OR¹, substituted or unsubstituted C₁-C₆alkyl, substituted or unsubstituted C₁-C₆heteroalkyl, substituted or unsubstituted C₃-C₈cycloalkyl, substituted or unsubstituted C₂-C₆alkynyl, and substituted or unsubstituted C₁-C₆fluoroalkyl;
n is 0, 1, or 2; and
m is 0, 1, or 2.

In another aspect, a compound that could be identified herein has the structure of Formula (VII), or a pharmaceutically acceptable salt or solvate thereof:

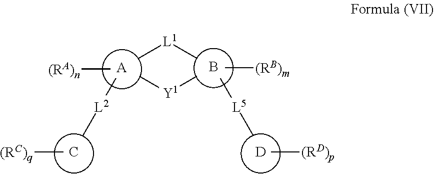

wherein,
ring A is aryl or heteroaryl;
each R^{A} is independently selected from H, D, halogen, —CN, —OH, —OR¹, —SR¹, —S(═O)R¹, —S(═O)₂R¹, —NHS(═O)₂R¹, —S(═O)₂N(R¹)₂, —C(═O)R¹, —OC(═O)R¹, substituted or unsubstituted C₁-C₆alkyl, substituted or unsubstituted C₃-C₆cycloalkyl, substituted or unsubstituted C₂-C₆alkenyl, substituted or unsubstituted C₂-C₆alkynyl, substituted or unsubstituted C₁-C₆fluoroalkyl, and substituted or unsubstituted C₁-C₆heteroalkyl;
L¹ is —X¹-L³- or -L³-X¹—;
   X¹ is —O—, —S—, —S(═O)—, —S(═O)₂—, —S(═O)₂NR¹—, —CH₂—, —C(═O)—, —C(═O)O—, —OC(═O)—, —C(═O)NR¹—, —NR¹C(═O)—, —NR¹S(═O)₂—, or —NR¹—;
   L³ is absent or substituted or unsubstituted C₁-C₂alkylene;
Y¹ is —W¹—Y²— or —Y²—W¹—;
   W¹ is —O—, —S—, —S(═O)—, —S(═O)₂—, —S(═O)₂NR¹—, —CH₂—, —C(═O)—, —C(═O)O—, —OC(═O)—, —C(═O)NR¹—, —NR¹C(═O)—, —NR¹S(═O)₂—, or —NR¹—;
   Y² is absent or substituted or unsubstituted C₁-C₂alkylene;
ring B is aryl or heteroaryl;
each R^{B} is independently selected from H, D, halogen, —CN, —OH, —OR¹, —SR¹, —S(═O)R¹, —N(R¹)₂, —S(═O)₂R¹, —NR¹S(═O)₂R¹, —S(═O)₂N(R¹)₂, —C(═O)R¹, —OC(═O)R¹, —CO₂R¹, —OCO₂R¹, —C(═O)N(R¹)₂, —OC(═O)N(R¹)₂, —NR¹C(═O)N(R¹)₂, NR¹⁰C(═N—CN)N(R¹)₂, —NR¹C(═O)R¹, —NR¹C(═O)OR¹, substituted or unsubstituted C₁-C₆alkyl, substituted or unsubstituted C₁-C₆fluoroalkyl, substituted or unsubstituted C₁-C₆heteroalkyl, substituted or unsubstituted aryl and substituted or unsubstituted monocyclic heteroaryl;
each R¹ is independently H, substituted or unsubstituted C₁-C₆alkyl, substituted or unsubstituted C₁-C₆fluoroalkyl, substituted or unsubstituted C₁-C₆heteroalkyl, substituted or unsubstituted phenyl, or substituted or unsubstituted heteroaryl;
L² is —X²-L⁴-, or -L⁴-X²—;
   X² is absent, —O—, —S—, —S(═O)—, —S(═O)₂—, —S(═O)₂NR¹—, —CH₂—, —CH═CH—, C≡C—, —C(═O)—, —C(═O)O—, —OC(═O)—, —OC(═O)O—, —C(═O)NR¹—, —NR¹C(═O)—, —OC(═O)NR¹—, —NR¹C(═O)O—, —NR¹C(═O)NR¹—, —NR¹S(═O)₂—, or —NR¹—;
L⁴ is absent or substituted or unsubstituted C₁-C₃alkylene;
ring C is monocyclic carbocycle, bicyclic carbocycle, monocyclic heterocycle, or bicyclic heterocycle;
each R^{C} is independently selected from H, D, halogen, —CN, —OH, —OR¹, —SR¹, —S(═O)R¹, —S(═O)₂R¹, —N(R¹)₂, —CH₂—N(R¹)₂, —NHS(═O)₂R¹, —S(═O)₂N(R¹)₂, —C(═O)R¹, —OC(═O)R¹, —CO₂R¹, —OCO₂R¹, —C(═O)N(R¹)₂, —OC(═O)N(R¹)₂, —NR¹C(═O)N(R¹)₂, —NR¹C(═O)R¹¹, —NR¹C(═O)OR¹, substituted or unsubstituted C₁-C₆alkyl, substituted or unsubstituted C₁-C₆heteroalkyl, substituted or unsubstituted C₃-C₈cycloalkyl, substituted or unsubstituted C₂-C₆alkynyl, and substituted or unsubstituted C₁-C₆fluoroalkyl;
ring D is monocyclic carbocycle or monocyclic heterocycle;
each R^{D} is independently selected from H, D, halogen, —CN, —OH, —OR¹, —SR¹, —S(═O)R¹, —S(═O)₂R¹, —NHS(═O)₂R¹, —S(═O)₂N(R¹)₂, —C(═O)R¹, —OC(═O)R¹, substituted or unsubstituted C₁-C₆alkyl, substituted or unsubstituted C₃-C₆cycloalkyl, substituted or unsubstituted C₂-C₆alkenyl, substituted or unsubstituted C₂-C₆alkynyl, substituted or unsubstituted C₁-C₆fluoroalkyl, and substituted or unsubstituted C₁-C₆heteroalkyl;
L⁵ is —X³-L⁶-, or -L⁶-X³—;
   X³ is absent, —O—, —S—, —S(═O)—, —S(═O)₂—, —S(═O)₂NR¹—, —CH₂—, —C(═O)—, —C(═O)O—, —OC(═O)—, —C(═O)NR¹—, —NR¹C(═O)—, —OC(═O)NR¹—, —NR¹C(═O)O—, —NR¹C(═O)NR¹—, —NR¹S(═O)₂—, or —NR¹—;
L⁶ is absent or substituted or unsubstituted C₁-C₄alkylene;
n is 0, 1, or 2;
m is 0, 1, or 2;
q is 0, 1, 2, 3, 4, 5, or 6; and
p is 0, 1, 2, 3, or 4.

In another aspect, a compound that could be identified herein that has the structure of Formula (VIII), or a pharmaceutically acceptable salt or solvate thereof:

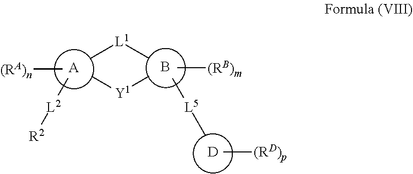

wherein,
ring A is aryl or heteroaryl;
each R^{A} is independently selected from H, D, halogen, —CN, —OH, —OR¹, —SR¹, —S(═O)R¹, —S(═O)₂R¹, —NHS(═O)₂R¹, —S(═O)₂N(R¹)₂, —C(═O)R¹, —OC(═O)R¹, substituted or unsubstituted C₁-C₆alkyl, substituted or unsubstituted C₃-C₆cycloalkyl, substituted or unsubstituted C₂-C₆alkenyl, substituted or unsubstituted C₂-C₆alkynyl, substituted or unsubstituted C₁-C₆fluoroalkyl, and substituted or unsubstituted C₁-C₆heteroalkyl;
L¹ is —X¹-L³- or -L³-X¹—;
   X¹ is —O—, —S—, —S(═O)—, —S(═O)₂—, —S(═O)₂NR¹—, —CH₂—, —C(═O)—, —C(═O)O—, —OC(—O)—, —C(═O)NR¹—, —NR¹C(═O)—, —NR¹S(═O)₂—, or —NR¹—;
   L³ is absent or substituted or unsubstituted C₁-C₂alkylene;
Y¹ is —W¹—Y²— or —Y²—W¹—;
   W¹ is —O—, —S—, —S(═O)—, —S(═O)₂—, —S(═O)₂NR¹—, —CH₂—, —C(═O)—, —C(═O)O—, —OC(═O)—, —C(═O)NR¹—, —NR¹C(═O)—, —NR¹S(═O)₂—, or —NR¹—;
   Y² is absent or substituted or unsubstituted C₁-C₂alkylene;
ring B is monocyclic carbocycle, bicyclic carbocycle, monocyclic heterocycle, or bicyclic heterocycle;
each R^{B} is independently selected from H, D, halogen, —CN, —OH, —OR¹, —SR¹, —S(═O)R¹, —N(R¹)₂, —S(═O)₂R¹, —NR¹S(═O)₂R¹, —S(═O)₂N(R¹)₂, —C(═O)R¹, —OC(═O)R¹, —CO₂R¹, —OCO₂R¹, —C(═O)N(R¹)₂, —OC(═O)N(R¹)₂, —NR¹C(═O)N(R¹)₂, NR¹⁰C(═N—CN)N(R¹)₂, —NR¹C(═O)R¹, —NR¹C(═O)OR¹, substituted or unsubstituted C₁-C₆alkyl, substituted or unsubstituted C₁-C₆fluoroalkyl, substituted or unsubstituted C₁-C₆heteroalkyl, substituted or unsubstituted aryl, and substituted or unsubstituted monocyclic heteroaryl;
each R¹ is independently H, substituted or unsubstituted C₁-C₆alkyl, substituted or unsubstituted C₁-C₆fluoroalkyl, substituted or unsubstituted C₁-C₆heteroalkyl, substituted or unsubstituted phenyl, or substituted or unsubstituted heteroaryl;
L² is —X²-L⁴-, or -L⁴-X²—;
   X² is absent, —O—, —S—, —S(═O)—, —S(═O)₂—, —S(═O)₂NR¹—, —CH₂—, —CH═CH—, —C≡C—, —C(═O)—, —C(═O)O—, —OC(═O)—, —OC(═O)O—, —C(═O)NR¹—, —NR¹C(═O)—, —OC(═O)NR¹—, —NR¹C(═O)O—, —NR¹C(═O)NR¹—, —NR¹S(═O)₂—, or —NR¹—;
L⁴ is absent or substituted or unsubstituted C₁-C₃alkylene;
R² is independently selected from H, D, —F, —CN, —OH, —OR¹, —SR¹, —S(═O)R¹, —S(═O)₂R¹, —N(R¹)₂, —NHS(═O)₂R¹, —S(═O)₂N(R¹)₂, —C(═O)R¹, —OC(═O)R¹, —CO₂R¹, —OCO₂R¹, —C(═O)N(R¹)₂, —OC(═O)N(R¹)₂, —NR¹C(═O)N(R¹)₂, —NR¹C(═O)R¹¹, —NR¹C(═O)OR¹, substituted or unsubstituted C₁-C₆alkyl, substituted or unsubstituted C₁-C₆heteroalkyl, substituted or unsubstituted C₃-C₈cycloalkyl, substituted or unsubstituted C₂-C₆alkynyl, and substituted or unsubstituted C₁-C₆fluoroalkyl;
ring D is monocyclic carbocycle, bicyclic carbocycle, monocyclic heterocycle, or bicyclic heterocycle;
each R^{D} is independently selected from H, D, halogen, —CN, —OH, —OR¹, —SR¹, —S(═O)R¹, —S(═O)₂R¹, —NHS(═O)₂R¹, —S(═O)₂N(R¹)₂, —C(═O)R¹, —OC(═O)R¹, substituted or unsubstituted C₁-C₆alkyl, substituted or unsubstituted C₃-C₆cycloalkyl, substituted or unsubstituted C₂-C₆alkenyl, substituted or unsubstituted C₂-C₆alkynyl, substituted or unsubstituted C₁-C₆fluoroalkyl, substituted or unsubstituted C₁-C₆heteroalkyl;
L⁵ is —X³-L⁶-, or -L⁶-X³—;
   X³ is absent, —O—, —S—, —S(═O)—, —S(═O)₂—, —S(═O)₂NR¹—, —CH₂—, —C(═O)—, —C(═O)O—, —OC(═O)—, —C(═O)NR¹—, —NR¹C(═O)—, —OC(═O)NR¹—, —NR¹C(═O)O—, —NR¹C(═O)NR¹—, —NR¹S(═O)₂—, or —NR¹—;
L⁶ is absent or substituted or unsubstituted C₁-C₄alkylene;
n is 0, 1, or 2;
m is 0, 1, or 2; and
p is 0, 1, 2, 3, or 4.

In one aspect, a compound that could be identified herein has the structure of Formula (IX), or a pharmaceutically acceptable salt or solvate thereof:

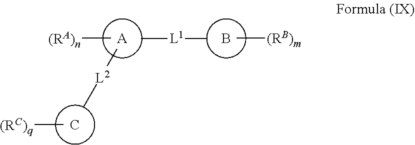

wherein,
ring A is aryl or heteroaryl;
each R^{A} is independently selected from H, D, halogen, —CN, —OH, —OR¹, —SR¹, —S(═O)R¹, —S(═O)₂R¹, —NHS(═O)₂R¹, —S(═O)₂N(R¹)₂, —C(═O)R¹, —OC(═O)R¹, substituted or unsubstituted C₁-C₆alkyl, substituted or unsubstituted C₃-C₆cycloalkyl, substituted or unsubstituted C₂-C₆alkenyl, substituted or unsubstituted C₂-C₆alkynyl, substituted or unsubstituted C₁-C₆fluoroalkyl, and substituted or unsubstituted C₁-C₆heteroalkyl;
L¹ is —X¹-L³- or -L³-X¹—;
   X¹ is —S(═O)₂NR¹—, —C(═O)O—, —OC(═O)—, —C(═O)NR¹—, —NR¹C(═O)—, —OC(═O)NR¹—, —NR¹C(═O)O—, —NR¹C(═O)NR¹—, or —NR¹S(═O)₂—;
   L³ is absent or substituted or unsubstituted C₁-C₂alkylene;
ring B is aryl or heteroaryl;
each R^{B} is independently selected from H, D, halogen, —CN, —OH, —OR¹, —SR¹, —S(═O)R¹, —S(═O)₂R¹, —N(R¹)₂, —NR¹S(═O)₂R¹, —S(═O)₂N(R¹)₂, —C(═O)R¹, —OC(═O)R¹, —CO₂R¹, —OCO₂R¹, —C(═O)N(R¹)₂, —OC(═O)N(R¹)₂, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, substituted or unsubstituted aryl, and substituted or unsubstituted monocyclic heteroaryl;
each R¹ is independently H, substituted or unsubstituted C₁-C₆alkyl, substituted or unsubstituted C₁-C₆fluoroalkyl, substituted or unsubstituted C₁-C₆heteroalkyl, substituted or unsubstituted phenyl, or substituted or unsubstituted heteroaryl;
L² is —X²-L⁴-, or -L⁴-X²—;
   X² is absent, —O—, —S—, —S(═O)—, —S(═O)₂—, —CH₂—, —CH═CH—, —C≡—, —C(═O)—, —C(═O)O—, —OC(═O)—, —OC(═O)O—, —C(═O)NR¹—, —NR¹C(═O)—, —OC(═O)NR¹—, —NR¹C(═O)O—, —NR¹C(═O)NR¹—, —NR¹S(═O)₂—, —S(═O)₂NR¹—, or —NR¹—;
L⁴ is absent or substituted or unsubstituted C₁-C₃alkylene;
ring C is monocyclic carbocycle, bicyclic carbocycle, monocyclic heterocycle, or bicyclic heterocycle;
each R^{C} is independently selected from H, D, halogen, —CN, —OH, —OR¹, —SR¹, —S(═O)R¹, —S(═O)₂R¹, —N(R¹)₂, —CH₂—N(R¹)₂, —NHS(═O)₂R¹, —S(═O)₂N(R¹)₂, —C(═O)R¹, —OC(═O)R¹, —CO₂R¹, —OCO₂R¹, —C(═O)N(R¹)₂, —OC(═O)N(R¹)₂, —NR¹C(═O)N(R¹)₂, —NR¹C(═O)R¹¹, —NR¹C(═O)OR¹, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, and substituted or unsubstituted C₂-C₈heterocycloalkyl;
n is 0, 1, or 2;
m is 0, 1, or 2; and
q is 0, 1, 2, 3, 4, 5, or 6.

In one aspect, described herein is a compound that has the structure of Formula (X), or a pharmaceutically acceptable salt or solvate thereof:

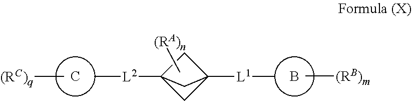

wherein,
each R^{A} is independently selected from H, D, halogen, —CN, —OH, —OR¹, —SR¹, —S(═O)R¹, —S(═O)₂R¹, —NHS(═O)₂R¹, —S(═O)₂N(R¹)₂, —C(═O)R¹, —OC(═O)R¹, substituted or unsubstituted C₁-C₆alkyl, substituted or unsubstituted C₃-C₆cycloalkyl, substituted or unsubstituted C₂-C₆alkenyl, substituted or unsubstituted C₂-C₆alkynyl, substituted or unsubstituted C₁-C₆fluoroalkyl, and substituted or unsubstituted C₁-C₆heteroalkyl;
L¹ is —X¹-L³- or -L³-X¹—;
   X¹ is —S(═O)₂NR¹—, —C(═O)O—, —OC(═O)—, —C(═O)NR¹—, —NR¹C(═O)—, —OC(═O)NR¹—, —NR¹C(═O)O—, —NR¹C(═O)NR¹—, or —NR¹S(=O)₂—;
   L³ is absent or substituted or unsubstituted C₁-C₂alkylene;
ring B is aryl or heteroaryl;
each R^{B} is independently selected from H, D, halogen, —CN, —OH, —OR¹, —SR¹, —S(═O)R¹, —S(═O)₂R¹, —N(R¹)₂, —NR¹S(═O)₂R¹, —S(═O)₂N(R¹)₂, —C(═O)R¹, —OC(═O)R¹, —CO₂R¹, —OCO₂R¹, —C(═O)N(R¹)₂, —OC(═O)N(R¹)₂, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, substituted or unsubstituted aryl, and substituted or unsubstituted monocyclic heteroaryl;

each R¹ is independently H, substituted or unsubstituted C₁-C₆alkyl, substituted or unsubstituted C₁-C₆fluoroalkyl, substituted or unsubstituted C₁-C₆heteroalkyl, substituted or unsubstituted phenyl, or substituted or unsubstituted heteroaryl;
L² is —X²-L⁴-, or -L⁴-X²—;
   X² is absent, —O—, —S—, —S(═O)—, —S(═O)₂—, —CH₂—, —CH═CH—, —C≡C—, —C(═O)—, —C(═O)O—, —OC(═O)—, —OC(═O)O—, —C(═O)NR¹—, —NR¹C(═O)—, —OC(═O)NR¹—, —NR¹C(═O)O—, —NR¹C(═O)NR¹—, —NR¹S(═O)₂—, —S(═O)₂NR¹—, or —NR¹—;
L⁴ is absent or substituted or unsubstituted C₁-C₃alkylene;
ring C is monocyclic carbocycle, bicyclic carbocycle, monocyclic heterocycle, or bicyclic heterocycle;
each R^{C} is independently selected from H, D, halogen, —CN, —OH, —OR¹, —SR¹, —S(═O)R¹, —S(═O)₂R¹, —N(R¹)₂, —CH₂—N(R¹)₂, —NHS(═O)₂R¹, —S(═O)₂N(R¹)₂, —C(═O)R¹, —OC(═O)R¹, —CO₂R¹, —OCO₂R¹, —C(═O)N(R¹)₂, —OC(═O)N(R¹)₂, —NR¹C(═O)N(R¹)₂, —NR¹C(═O)R¹¹, —NR¹C(═O)OR¹, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, and substituted or unsubstituted C₂-C₈heterocycloalkyl;
n is 0, 1, or 2;
m is 0, 1, or 2; and
q is 0, 1, 2, 3, 4, 5, or 6.

In one aspect, a compound that could be identified herein has the structure of Formula (XI), or a pharmaceutically acceptable salt or solvate thereof:

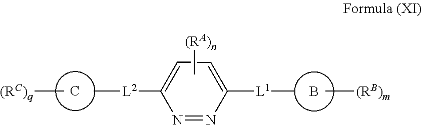

wherein,
each R^{A} is independently selected from H, D, halogen, —CN, —OH, —OR¹, —SR¹, —S(═O)R¹, —S(═O)₂R¹, —NHS(═O)₂R¹, —S(═O)₂N(R¹)₂, —C(═O)R¹, —OC(═O)R¹, substituted or unsubstituted C₁-C₆alkyl, substituted or unsubstituted C₃-C₆cycloalkyl, substituted or unsubstituted C₂-C₆alkenyl, substituted or unsubstituted C₂-C₆alkynyl, substituted or unsubstituted C₁-C₆fluoroalkyl, and substituted or unsubstituted C₁-C₆heteroalkyl;
L¹ is —X¹-L³- or -L³-X¹—;
   X¹ is —S(═O)₂NR¹—, —C(═O)O—, —OC(═O)—, —C(═O)NR¹—, —NR¹C(═O)—, —OC(═O)NR¹—, —NR¹C(═O)O—, —NR¹C(═O)NR¹—, or —NR¹S(═O)₂—;
   L³ is absent or substituted or unsubstituted C₁-C₂alkylene;
ring B is monocyclic heterocycle or bicyclic heterocycle;
each R^{B} is independently selected from H, D, halogen, —CN, —OH, —OR¹, —SR¹, —S(═O)R¹, —S(═O)₂R¹, —N(R¹)₂, —NR¹S(═O)₂R¹, —S(═O)₂N(R¹)₂, —C(═O)R¹, —OC(═O)R¹, —CO₂R¹, —OCO₂R¹, —C(═O)N(R¹)₂, —OC(═O)N(R¹)₂, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, substituted or unsubstituted aryl and substituted or unsubstituted monocyclic heteroaryl;
each R¹ is independently H, substituted or unsubstituted C₁-C₆alkyl, substituted or unsubstituted C₁-C₆fluoroalkyl, substituted or unsubstituted C₁-C₆heteroalkyl, substituted or unsubstituted phenyl, or substituted or unsubstituted heteroaryl;
L² is 13 X²-L⁴-, or -L⁴-X²—;
   X² is absent, —O—, —S—, —S(═O)-, —S(═O)₂—, —CH₂—, —CH═CH—, —C≡C—, —C(═O)—, —C(═O)O—, —OC(═O)—, —OC(═O)O—, —C(═O)NR¹—, —NR¹C(═O)—, —OC(═O)NR¹—, —NR¹C(═O)O—, —NR¹C(═O)NR¹—, —NR¹S(═O)₂—, —S(═O)₂NR¹—, or —NR¹—;
   L⁴ is absent or substituted or unsubstituted C₁-C₃alkylene;
ring C is monocyclic carbocycle, bicyclic carbocycle, monocyclic heterocycle, or bicyclic heterocycle;
each R^{C} is independently selected from H, D, F, —CN, —OH, —OR¹, —SR¹, —S(═O)R¹, —S(═O)₂R¹, —N(R¹)₂, —CH₂—N(R¹)₂, —NHS(═O)₂R¹, —S(═O)₂N(R¹)₂, —C(═O)R¹, —OC(═O)R¹, —CO₂R¹, —OCO₂R¹, —C(═O)N(R¹)₂, —OC(═O)N(R¹)₂, —NR¹C(═O)N(R¹)₂, —NR¹C(═O)R¹¹, —NR¹C(═O)OR¹, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, and substituted or unsubstituted C₂-C₈heterocycloalkyl;
n is 0, 1, or 2;
m is 0, 1, or 2; and
q is 0, 1, 2, 3, 4, 5, or 6.

In one aspect, a compound that could be identified herein has the structure of Formula (XII), or a pharmaceutically acceptable salt or solvate thereof:

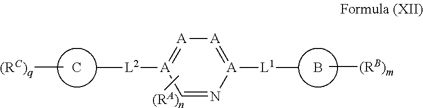

wherein,
each A is independently N or CR^{A};
each R^{A} is independently selected from H, D, halogen, —CN, —OH, —OR¹, ═O, —SR¹, —S(═O)R¹, —S(═O)₂R¹, —N(R¹)₂, —NR¹S(═O)(═NR¹)R², —NR¹S(═O)₂R², —S(═O)₂N(R¹)₂, —C(═O)R¹, —OC(═O)R¹, —CO₂R¹, —OCO₂R¹, —C(═O)N(R¹)₂, —OC(═O)N(R¹)₂, —NR¹C(═O)R¹, —P(═O)(R²)₂, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, substituted or unsubstituted C₂-C₇heterocycloalkyl, substituted or unsubstituted aryl, and substituted or unsubstituted monocyclic heteroaryl;
L¹ is —X¹-L³- or -L³-X¹—;
   X¹ is absent, —O—, —S—, —S(═O)—, —S(═O)₂—, —S(═O)(═NR¹)—, —CH₂—, —C(═O)—, —C(═O)O—, —OC(═O)—, —C(═O)NR¹—, —NR¹C(═O)—, —OC(═O)NR¹—, —NR¹C(═O)O—, —NR¹C(═O)NR¹—, —S(═O)₂NR¹—, —NR¹S(═O)₂—, —NR¹—, —P(═O)R²—, —P(═O)(N(R¹)₂)—, or —P(═O)(CR¹₃)—;
   L³ is absent or substituted or unsubstituted C₁-C₂alkylene;
ring B is monocyclic carbocycle, bicyclic carbocycle, monocyclic heterocycle, or bicyclic heterocycle;
each R^{B} is independently selected from H, D, halogen, —CN, —OH, —OR¹, ═O, —SR¹, —S(═O)R¹, —S(═O)₂R¹, —N(R¹)₂, —NR¹S(═O)(═NR¹)R², —NR¹S(═O)₂R², —S(═O)₂N(R¹)₂, —C(═O)R¹, —OC(═O)R¹, —CO₂R¹, —OCO₂R¹, —C(═O)N(R¹)₂, —OC(═O)N(R¹)₂, —NR¹C(═O)R¹, —P(═O)(R²)₂, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, substituted or unsubstituted C₂-C₇heterocycloalkyl, substituted or unsubstituted aryl, and substituted or unsubstituted monocyclic heteroaryl;
each R¹ is independently H, D, substituted or unsubstituted C₁-C₆alkyl, substituted or unsubstituted C₁-C₆haloalkyl, substituted or unsubstituted C₁-C₆heteroalkyl, substituted or unsubstituted aryl, or substituted or unsubstituted heteroaryl;
each R² is independently H, D, substituted or unsubstituted C₁-C₆alkyl, substituted or unsubstituted C₁-C₆fluoroalkyl, substituted or unsubstituted aryl, substituted or unsubstituted monocyclic heteroaryl, —OH, —OR¹, —N(R¹)₂, —CH₂OR¹, —C(═O)OR¹, —OC(═O)R¹, —C(═O)N(R¹)₂, or —NR¹C(═O)R¹;
L² is —X²-L⁴- or -L⁴-X²—;
   X² is —O—, —S—, —S(═O)—, —S(═O)₂—, —S(═O)(═NR¹)—, —CH₂—, —CH═CH—, —C≡C—, —C(═O)—, —C(═O)O—, —OC(═O)—, —OC(═O)O—, —C(═O)C(═O)—, —C(═O)NR¹—, —NR¹C(═O)—, —OC(═O)NR¹—, —NR¹C(═O)O—, —NR¹C(═O)NR¹—, —NR¹S(═O)₂—, —S(═O)₂NR¹—, —NR¹—, —P(═O)R²—, —P(═O)(N(R¹)₂)—, or —P(═O)(CR¹₃)—;
   L⁴ is absent or substituted or unsubstituted C₁-C₂alkylene;
ring C is monocyclic carbocycle, bicyclic carbocycle, monocyclic heterocycle, or bicyclic heterocycle;
each R^{C} is independently selected from H, D, F, —CN, —OH, —OR¹, —SR¹, —S(═O)R¹, —S(═O)₂R¹, —N(R¹)₂, —CH₂—N(R¹)₂, —NHS(═O)₂R¹, —S(═O)₂N(R¹)₂, —C(═O)R¹, —OC(═O)R¹, —CO₂R¹, —OCO₂R¹, —C(═O)N(R¹)₂, —OC(═O)N(R¹)₂, —NR¹C(═O)N(R¹)₂, —NR¹C(═O)R¹, —NR¹C(═O)OR¹, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, and substituted or unsubstituted C₂-C₈heterocycloalkyl;
n is 0, 1, 2, or 3;
m is 0, 1, 2, or 3; and
q is 0, 1, 2, 3, 4, 5, or 6.

In another aspect, a compound that could be identified herein has the structure of Formula (XIII), or a pharmaceutically acceptable salt or solvate thereof:

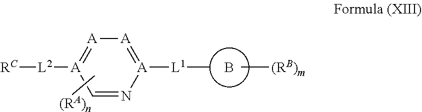

wherein,
each A is independently N or CR^{A};
each R^{A} is independently selected from H, D, halogen, —CN, —OH, —OR¹, ═O, —SR¹, —S(═O)R¹, —S(═O)₂R¹, —N(R¹)₂, —NR¹S(═O)(═NR¹)R², —NR¹S(—O)₂R², —S(═O)₂N(R¹)₂, —C(═O)R¹, —OC(═O)R¹, —CO₂R¹, —OCO₂R¹, —C(═O)N(R¹)₂, —OC(═O)N(R¹)₂, —NR¹C(═O)R¹, —P(═O)(R²)₂, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, substituted or unsubstituted C₂-C₇heterocycloalkyl, substituted or unsubstituted aryl, and substituted or unsubstituted monocyclic heteroaryl;
L¹ is —X¹-L³- or -L³-X¹—;
   X¹ is absent, —O—, —S—, —S(═O)—, —S(═O)₂—, —S(═O)(—NR¹)—, —CH₂—, —C(═O)—, —C(═O)O—, —OC(═O)—, —C(═O)NR¹—, —NR¹C(═O)—, —OC(═O)NR¹—, —NR¹C(═O)O—, —NR¹C(═O)NR¹—, —S(═O)₂NR¹—, —NR¹S(═O)₂—, —NR¹—, —P(═O)R²—, —P(═O)(N(R¹)₂)—, or —P(═O)(CR¹₃)—;
   L³ is absent or substituted or unsubstituted C₁-C₂alkylene;
ring B is monocyclic carbocycle, bicyclic carbocycle, monocyclic heterocycle, or bicyclic heterocycle;
each R^{B} is independently selected from H, D, halogen, —CN, —OH, —OR¹, ═O, —SR¹, —S(═O)R¹, —S(═O)₂R¹, —N(R¹)₂, —NR¹S(═O)(═NR¹)R², —NR¹S(═O)₂R², —S(═O)₂N(R¹)₂, —C(═O)R¹, —OC(═O)R¹, —CO₂R¹, —OCO₂R¹, —C(═O)N(R¹)₂, —OC(═O)N(R¹)₂, —NR¹C(═O)R¹, —P(═O)(R²)₂, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, substituted or unsubstituted C₂-C₇heterocycloalkyl, substituted or unsubstituted aryl, and substituted or unsubstituted monocyclic heteroaryl;
each R¹ is independently H, D, substituted or unsubstituted C₁-C₆alkyl, substituted or unsubstituted C₁-C₆haloalkyl, substituted or unsubstituted C₁-C₆heteroalkyl, substituted or unsubstituted aryl, or substituted or unsubstituted heteroaryl;
each R² is independently H, D, substituted or unsubstituted C₁-C₆alkyl, substituted or unsubstituted C₁-C₆fluoroalkyl, substituted or unsubstituted aryl, substituted or unsubstituted monocyclic heteroaryl, —OH, —OR¹, —N(R¹)₂, —CH₂OR¹, —C(═O)OR¹, —OC(═O)R¹, —C(═O)N(R¹)₂, or —NR¹C(═O)R¹;
L² is —X²-L⁴- or -L⁴-X²—;
   X² is —O—, —S—, —S(═O)—, —S(═O)₂—, —S(═O)(═NR¹)—, —CH₂—, —CH═CH—, —C≡—, —C(═O)—, —C(═O)O—, —OC(═O)—, —OC(═O)O—, —C(═O)C(═O)—, —C(═O)NR¹—, —NR¹C(═O)—, —OC(═O)NR¹—, —NR¹C(═O)O—, —NR¹C(═O)NR¹—, —NR¹S(═O)₂—, —S(═O)₂NR¹—, —NR¹—, —P(═O)R²—, —P(═O)(N(R¹)₂)—, or —P(═O)(CR¹₃)—;
   L⁴ is absent or substituted or unsubstituted C₁-C₂alkylene;
R^{C} is —CN, —OR¹, —SR¹, —S(═O)R¹, —S(═O)₂R¹, —N(R¹)₂, —CH₂—N(R¹)₂, —NHS(═O)₂R¹, —S(═O)₂N(R¹)₂, —C(═O)R¹, —OC(═O)R¹, —CO₂R¹, —OCO₂R¹, —C(═O)N(R¹)₂, —OC(═O)N(R¹)₂, —NR¹C(═O)N(R¹)₂, —NR¹C(═O)R¹, —NR¹C(═O)OR¹, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, and substituted or unsubstituted C₂-C₈heterocycloalkyl;
n is 0, 1, 2, or 3; and
m is 0, 1, 2, or 3.

In one aspect, a compound that could be identified herein has the structure of Formula (XIV), or a pharmaceutically acceptable salt or solvate thereof:

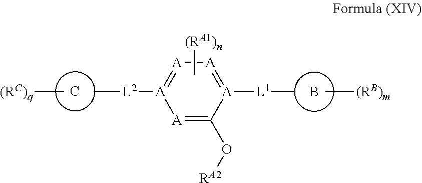

wherein,
each A is independently N or CR^{A1};
each R^{A1} is independently selected from H, D, halogen, —CN, —OH, —OR¹, ═O, —SR¹, —S(═O)R¹, —S(═O)₂R¹, —N(R¹)₂, —NR¹S(═O)(═NR¹)R², —NR¹S(═O)₂R², —S(═O)₂N(R¹)₂, —C(═O)R¹, —OC(═O)R¹, —CO₂R¹, —OCO₂R¹, —C(═O)N(R¹)₂, —OC(═O)N(R¹)₂, —NR¹C(═O)R¹, —P(═O)(R²)₂, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, substituted or unsubstituted C₂-C₇heterocycloalkyl, substituted or unsubstituted aryl, and substituted or unsubstituted monocyclic heteroaryl;
R^{A2} is H, D, substituted or unsubstituted C₁-C₆alkyl, substituted or unsubstituted C₃-C₆cycloalkyl, substituted or unsubstituted C₁-C₆fluoroalkyl, or substituted or unsubstituted C₁-C₆heteroalkyl;
L¹ is —X¹-L³- or -L³-X¹—;
   X¹ is absent, —O—, —S—, —S(═O)—, —S(═O)₂—, —S(═O)(=NR¹)—, —CH₂—, —C(═O)—, —C(═O)O—, —OC(═O)—, —C(═O)NR¹—, —NR¹C(═O)—, —OC(═O)NR¹—, —NR¹C(═O)O—, —NR¹C(═O)NR¹—, —S(═O)₂NR¹—, —NR¹S(═O)₂—, —NR¹—, —P(═O)R²—, —P(═O)(N(R¹)₂)—, or —P(═O)(CR¹₃)—;
   L³ is absent or substituted or unsubstituted C₁-C₂alkylene;
ring B is a monocyclic carbocycle, bicyclic carbocycle, monocyclic heterocycle, or bicyclic heterocycle;
each R^{B} is independently selected from H, D, halogen, —CN, —OH, —OR¹, ═O, —SR¹, —S(═O)R¹, —S(═O)₂R¹, —N(R¹)₂, —NR¹S(═O)(═NR¹)R², —NR¹S(═O)₂R², —S(═O)₂N(R¹)₂, —C(═O)R¹, —OC(═O)R¹, —CO₂R¹, —OCO₂R¹, —C(═O)N(R¹)₂, —OC(═O)N(R¹)₂, —NR¹C(═O)R¹, —P(═O)(R²)₂, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, substituted or unsubstituted C₂-C₇heterocycloalkyl, substituted or unsubstituted aryl, and substituted or unsubstituted monocyclic heteroaryl;
each R¹ is independently H, D, substituted or unsubstituted C₁-C₆alkyl, substituted or unsubstituted C₁-C₆haloalkyl, substituted or unsubstituted C₁-C₆heteroalkyl, substituted or unsubstituted aryl, or substituted or unsubstituted heteroaryl;
each R² is independently H, D, substituted or unsubstituted C₁-C₆alkyl, substituted or unsubstituted C₁-C₆fluoroalkyl, substituted or unsubstituted aryl, substituted or unsubstituted monocyclic heteroaryl, —OH, —OR¹, —N(R¹)₂, —CH₂OR¹, —C(═O)OR¹, —OC(═O)R¹, —C(═O)N(R¹)₂, or —NR¹C(═O)R¹;
L² is —X²-L⁴- or -L⁴-X²—;
   X² is —O—, —S—, —S(═O)—, —S(═O)₂—, —S(═O)(═NR¹)—, —CH₂—, —CH═CH—, —C≡C—C, —C(═O)—, —C(═O)O—, —OC(═O)—, —OC(═O)O—, —C(═O)C(═O)—, —C(═O)NR¹—, —NR¹C(═O)—, —OC(═O)NR¹—, —NR¹C(═O)O—, —NR¹C(═O)NR¹—, —NR¹S(═O)₂—, —S(═O)₂NR¹—, —NR¹—, —P(═O)R²—, —P(═O)(N(R¹)₂)—, or —P(═O)(CR¹₃)—;
   L⁴ is absent or substituted or unsubstituted C₁-C₂alkylene;
ring C is monocyclic carbocycle, bicyclic carbocycle, monocyclic heterocycle, or bicyclic heterocycle;
each R^{C} is independently selected from H, D, F, —CN, —OH, —OR¹, —SR¹, —S(═O)R¹, —S(═O)₂R¹, —N(R¹)₂, —CH₂—N(R¹)₂, —NHS(═O)₂R¹, —S(═O)₂N(R¹)₂, —C(═O)R¹, —OC(═O)R¹, —CO₂R¹, —OCO₂R¹, —C(═O)N(R¹)₂, —OC(═O)N(R¹)₂, —NR¹C(═O)N(R¹)₂, —NR¹C(═O)R¹, —NR¹C(═O)OR¹, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, and substituted or unsubstituted C₂-C₈heterocycloalkyl;
n is 0, 1, 2, or 3;
m is 0, 1, 2, or 3; and
q is 0, 1, 2, 3, 4, 5, or 6.

In another aspect, a compound that could be identified herein has the structure of Formula (XV), or a pharmaceutically acceptable salt or solvate thereof:

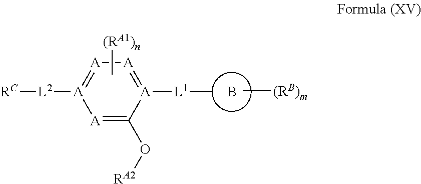

wherein,
each A is independently N or CR^{A1};
each R^{A1} is independently selected from H, D, halogen, —CN, —OH, —OR¹, ═O, —SR¹, —S(═O)R¹, —S(═O)₂R¹, —N(R¹)₂, —NR¹S(═O)(═NR¹)R², —NR¹S(═O)₂R², —S(═O)₂N(R¹)₂, —C(═O)R¹, —OC(═O)R¹, —CO₂R¹, —OCO₂R¹, —C(═O)N(R¹)₂, —OC(═O)N(R¹)₂, —NR¹C(═O)R¹, —P(═O)(R²)₂, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, substituted or unsubstituted C₂-C₇heterocycloalkyl, substituted or unsubstituted aryl, and substituted or unsubstituted monocyclic heteroaryl;
R^{A2} is H, D, substituted or unsubstituted C₁-C₆alkyl, substituted or unsubstituted C₃-C₆cycloalkyl, substituted or unsubstituted C₁-C₆fluoroalkyl, or substituted or unsubstituted C₁-C₆heteroalkyl;
L¹ is —X¹-L³- or -L³-X¹—;
   X¹ is absent, —O—, —S—, —S(═O)—, —S(═O)₂—, —S(═O)(═NR¹)—, —CH₂—, —C(═O)—, —C(═O)O—, —OC(═O)—, —C(═O)NR¹—, —NR¹C(═O)—, —OC(═O)NR¹—, —NR¹C(═O)O—, —NR¹C(═O)NR¹—, —S(═O)₂NR¹—, —NR¹S(═O)₂—, —NR¹—, —P(═O)R²—, —P(═O)(N(R¹)₂)—, or —P(═O)(CR¹₃)—;
   L³ is absent or substituted or unsubstituted C₁-C₂alkylene;
ring B is a monocyclic carbocycle, bicyclic carbocycle, monocyclic heterocycle, or bicyclic heterocycle;
each R^{B} is independently selected from H, D, halogen, —CN, —OH, —OR¹, ═O, —SR¹, —S(═O)R¹, —S(═O)₂R¹, —N(R¹)₂, —NR¹S(═O)(═NR¹)R², —NR¹S(═O)₂R², —S(═O)₂N(R¹)₂, —C(═O)R¹, —OC(═O)R¹, —CO₂R¹, —OCO₂R¹, —C(═O)N(R¹)₂, —OC(═O)N(R¹)₂, —NR¹C(═O)R¹, —P(═O)(R²)₂, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, substituted or unsubstituted C₂-C₇heterocycloalkyl, substituted or unsubstituted aryl, and substituted or unsubstituted monocyclic heteroaryl;
each R¹ is independently H, D, substituted or unsubstituted C₁-C₆alkyl, substituted or unsubstituted C₁-C₆haloalkyl, substituted or unsubstituted C₁-C₆heteroalkyl, substituted or unsubstituted aryl, or substituted or unsubstituted heteroaryl;
each R² is independently H, D, substituted or unsubstituted C₁-C₆alkyl, substituted or unsubstituted C₁-C₆fluoroalkyl, substituted or unsubstituted aryl, substituted or unsubstituted monocyclic heteroaryl, —OH, —OR¹, —N(R¹)₂, —CH₂OR¹, —C(═O)OR¹, —OC(═O)R¹, —C(═O)N(R¹)₂, or —NR¹C(═O)R¹;
L² is —X²-L⁴- or -L⁴-X²—;
   X² is —O—, —S—, —S(═O)—, —S(═O)₂—, —S(═O)(═NR¹)—, —CH₂—, —CH═CH—, —C≡C—, —C(═O)—, —C(═O)O—, —OC(═O)—, —OC(═O)O—, —C(═O)C(═O)—, —C(═O)NR¹—, —NR¹C(═O)—, —OC(═O)NR¹—, —NR¹C(═O)O—, —NR¹C(═O)NR¹—, —NR¹S(═O)₂—, —S(═O)₂NR¹—, —NR¹—, —P(═O)R²—, —P(═O)(N(R¹)₂)—, or —P(═O)(CR¹₃)—;
   L⁴ is absent or substituted or unsubstituted C₁-C₂alkylene;
R^{C} is —CN, —OR¹, —SR¹, —S(═O)R¹, —S(═O)₂R¹, —N(R¹)₂, —CH₂—N(R¹)₂, —NHS(═O)₂R¹, —S(═O)₂N(R¹)₂, —C(═O)R¹, —OC(═O)R¹, —CO₂R¹, —OCO₂R¹, —C(═O)N(R¹)₂, —OC(═O)N(R¹)₂, —NR¹C(═O)N(R¹)₂, —NR¹C(═O)R¹, —NR¹C(═O)OR¹, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, or substituted or unsubstituted C₂-C₈heterocycloalkyl;
n is 0, 1, 2, or 3; and
m is 0, 1, 2, or 3.

In one aspect, a compound that could be identified herein has the structure of Formula (XVI), or a pharmaceutically acceptable salt or solvate thereof:

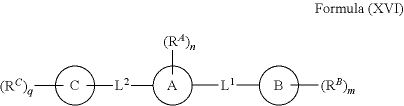

wherein,
ring A is a 6-membered aryl or 6-membered heteroaryl;
each R^{A} is independently selected from H, D, halogen, —CN, —OH, —OR¹, ═O, —SR¹, —S(═O)R¹, —S(═O)₂R¹, —N(R¹)₂, —NR¹S(═O)(═NR¹)R², —NR¹S(═O)₂R², —S(═O)₂N(R¹)₂, —C(═O)R¹, —OC(═O)R¹, —CO₂R¹, —OCO₂R¹, —C(═O)N(R¹)₂, —OC(═O)N(R¹)₂, —NR¹C(═O)R¹, —P(═O)(R²)₂, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, substituted or unsubstituted C₂-C₇heterocycloalkyl, substituted or unsubstituted aryl, and substituted or unsubstituted monocyclic heteroaryl;
L¹ is —X^{1A}-L³-X^{1B}—, -L³-X^{1A}—X^{1B}—, or —X^{1A}—X^{1B}-L³-;
   X^{1A} is absent, —O—, —S—, —S(═O)—, —S(═O)₂—, —S(═O)(—NR¹)—, —CH₂—, —C(═O)—, —C(═N—OR²)—, —C(═O)O—, —OC(═O)—, —C(═O)NR¹—, —NR¹C(═O)—, —OC(═O)NR¹—, —NR¹C(═O)O—, —NR¹C(═O)NR¹—, —S(═O)₂NR¹—, —NR¹S(═O)₂—, —NR¹—, —NOR¹—, —P(═O)R²—, —P(═O)(N(R¹)₂)—, —P(═O)(CR¹₃)—, —CR²═CR²—, —N═CR²—, —CR²═N—, or —NR²—NR²—;
   L³ is absent, substituted or unsubstituted C₁-C₂alkylene, or

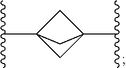 [SMILES: *C12CC(*)(C1)C2]

X^{1B} is absent, —O—, —S—, —S(═O)—, —S(═O)₂—, —S(═O)(═NR¹)—, —CH₂—, —C(═O)—, —C(═N—OR²)—, —C(═O)O—, —OC(═O)—, —C(═O)NR¹—, —NR¹C(═O)—, —OC(═O)NR¹—, —NR¹C(═O)O—, —NR¹C(═O)NR¹—, —S(═O)₂NR¹—, —NR¹S(═O)₂—, —NR¹—, —NOR¹—, —P(═O)R²—, —P(═O)(N(R¹)₂)—, —P(═O)(CR¹₃)—, —CR²═CR²—, —N═CR²—, —CR²═N—, or —NR²—NR²—;
ring B is a monocyclic carbocycle, bicyclic carbocycle, monocyclic heterocycle, or bicyclic heterocycle;
each R^{B} is independently selected from H, D, halogen, —CN, —OH, —OR¹, ═O, —SR¹, —S(═O)R¹, —S(═O)₂R¹, —N(R¹)₂, —NR¹S(═O)(═NR¹)R², —NR¹S(═O)₂R², —S(═O)₂N(R¹)₂, —C(═O)R¹, —OC(═O)R¹, —CO₂R¹, —OCO₂R¹, —C(═O)N(R¹)₂, —OC(═O)N(R¹)₂, —NR¹C(═O)R¹, —P(═O)(R²)₂, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, substituted or unsubstituted C₂-C₇heterocycloalkyl, substituted or unsubstituted aryl, and substituted or unsubstituted monocyclic heteroaryl;
each R¹ is independently H, D, substituted or unsubstituted C₁-C₆alkyl, substituted or unsubstituted C₁-C₆fluoroalkyl, substituted or unsubstituted C₁-C₆heteroalkyl, substituted or unsubstituted aryl, or substituted or unsubstituted heteroaryl;
each R² is independently H, D, substituted or unsubstituted C₁-C₆alkyl, substituted or unsubstituted C₁-C₆fluoroalkyl, substituted or unsubstituted aryl, substituted or unsubstituted monocyclic heteroaryl, —OH, —OR¹, —N(R¹)₂, —CH₂OR¹, —C(═O)OR¹, —OC(═O)R¹, —C(═O)N(R¹)₂, or —NR¹C(═O)R¹;
L² is —X²-L⁴- or -L⁴-X²—;
   X² is —O—, —S—, —S(═O)—, —S(═O)₂—, —S(═O)(═NR¹)—, —CH₂—, —CH═CH—, —C≡C—, —C(═O)—, —C(═O)O—, —OC(═O)—, —OC(═O)O—, —C(═O)C(═O)—, —C(═O)NR¹—, —NR¹C(═O)—, —OC(═O)NR¹—, —NR¹C(═O)O—, —NR¹C(═O)NR¹—, —NR¹S(═O)₂—, —S(═O)₂NR¹—, —NR¹—, —P(═O)R²—, —P(═O)(N(R¹)₂)—, or —P(═O)(CR¹₃)—;
   L⁴ is absent or substituted or unsubstituted C₁-C₂alkylene;
ring C is monocyclic carbocycle, bicyclic carbocycle, monocyclic heterocycle, or bicyclic heterocycle;
each R^{C} is independently selected from H, D, F, —CN, —OH, —OR¹, —SR¹, —S(═O)R¹, —S(═O)₂R¹, —N(R¹)₂, —CH₂—N(R¹)₂, —NHS(═O)₂R¹, —S(═O)₂N(R¹)₂, —C(═O)R¹, —OC(═O)R¹, —CO₂R¹, —OCO₂R¹, —C(═O)N(R¹)₂, —OC(═O)N(R¹)₂, —NR¹C(═O)N(R¹)₂, —NR¹C(═O)R¹, —NR¹C(═O)OR¹, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, and substituted or unsubstituted C₂-C₈heterocycloalkyl;
n is 0, 1, 2, or 3;
m is 0, 1, 2, or 3; and
q is 0, 1, 2, 3, 4, 5, or 6.

In one aspect, a compound that could be identified herein has the structure of Formula (XVII), or a pharmaceutically acceptable salt or solvate thereof:

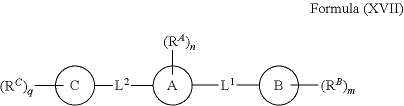

wherein,
ring A is a bicyclic carbocycle or bicyclic heterocycle;
each R^{A} is independently selected from H, D, halogen, —CN, —OH, —OR¹, ═O, —SR¹, —S(═O)R¹, —S(═O)₂R¹, —N(R¹)₂, —NR¹S(═O)(═NR¹)R², —NR¹S(═O)₂R², —S(═O)₂N(R¹)₂, —C(═O)R¹, —OC(═O)R¹, —CO₂R¹, —OCO₂R¹, —C(═O)N(R¹)₂, —OC(═O)N(R¹)₂, —NR¹C(═O)R¹, —P(═O)(R²)₂, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, substituted or unsubstituted C₂-C₇heterocycloalkyl, substituted or unsubstituted aryl, and substituted or unsubstituted monocyclic heteroaryl;
L¹ is —X¹-L³- or -L³-X¹—;
   X¹ is absent, —O—, —S—, —S(═O)—, —S(═O)₂—, —S(═O)(═NR¹)—, —CH₂—, —C(═O)—, —C(═N—OR²)—, —C(═O)O—, —OC(═O)—, —C(═O)C(═O)—, —C(═O)NR¹—, —NR¹C(═O)—, —OC(═O)NR¹—, —NR¹C(═O)O—, —NR¹C(═O)NR¹—, —S(═O)₂NR¹—, —NR¹S(═O)₂—, —NR¹—, —NOR¹—, —P(═O)R²—, —P(═O)(N(R¹)₂)—, —P(═O)(CR¹₃)—, —CR²═CR²—, —N═CR²—, —CR²═N—, or —NR²—NR²—;
   L³ is absent, substituted or unsubstituted C₁-C₂alkylene, or

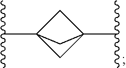 [SMILES: *C12CC(*)(C1)C2]

ring B is a monocyclic carbocycle, bicyclic carbocycle, monocyclic heterocycle, or bicyclic heterocycle;
each R^{B} is independently selected from H, D, halogen, —CN, —OH, —OR¹, ═O, —SR¹, —S(═O)R¹, —S(═O)₂R¹, —N(R¹)₂, —NR¹S(═O)(═NR¹)R², —NR¹S(═O)₂R², —S(═O)₂N(R¹)₂, —C(═O)R¹, —OC(═O)R¹, —CO₂R¹, —OCO₂R¹, —C(═O)N(R¹)₂, —OC(═O)N(R¹)₂, —NR¹C(═O)R¹, —P(═O)(R²)₂, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, substituted or unsubstituted C₂-C₇heterocycloalkyl, substituted or unsubstituted aryl, and substituted or unsubstituted monocyclic heteroaryl;
each R¹ is independently H, D, substituted or unsubstituted C₁-C₆alkyl, substituted or unsubstituted C₁-C₆fluoroalkyl, substituted or unsubstituted C₁-C₆heteroalkyl, substituted or unsubstituted aryl, or substituted or unsubstituted heteroaryl;
each R² is independently H, D, substituted or unsubstituted C₁-C₆alkyl, substituted or unsubstituted C₁-C₆fluoroalkyl, substituted or unsubstituted aryl, substituted or unsubstituted monocyclic heteroaryl, —OH, —OR¹, —N(R¹)₂, —CH₂OR¹, —C(═O)OR¹, —OC(═O)R¹, —C(═O)N(R¹)₂, or —NR¹C(═O)R¹;
L² is —X²-L⁴- or -L⁴-X²—;
   X² is —O—, —S—, —S(═O)—, —S(═O)₂—, —S(═O)(═NR¹)—, —CH₂—, —CH═CH—, —C≡C—, —C(═O)—, —C(═O)O—, —OC(═O)—, —OC(═O)O—, —C(═O)C(═O)—, —C(═O)NR¹—, —NR¹C(═O)—, —OC(═O)NR¹—, —NR¹C(═O)O—, —NR¹C(═O)NR¹—, —NR¹S(═O)₂—, —S(═O)₂NR¹—, —NR¹—, —P(═O)OR¹—, —P(═O)(N(R¹)₂)—, or —P(═O)(CR¹₃)—;
   L⁴ is absent or substituted or unsubstituted C₁-C₂alkylene;
ring C is monocyclic carbocycle, bicyclic carbocycle, monocyclic heterocycle, or bicyclic heterocycle;
each R^{C} is independently selected from H, D, F, —CN, —OH, —OR¹, —SR¹, —S(═O)R¹, —S(═O)₂R¹, —N(R¹)₂, —CH₂—N(R¹)₂, —NHS(═O)₂R¹, —S(═O)₂N(R¹)₂, —C(═O)R¹, —OC(═O)R¹, —CO₂R¹, —OCO₂R¹, —C(═O)N(R¹)₂, —OC(═O)N(R¹)₂, —NR¹C(═O)N(R¹)₂, —NR¹C(═O)R¹, —NR¹C(═O)OR¹, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, and substituted or unsubstituted C₂-C₈heterocycloalkyl;
n is 0, 1, 2, or 3;
m is 0, 1, 2, or 3; and
q is 0, 1, 2, 3, 4, 5, or 6.

In another aspect, a compound that could be identified herein has the structure of Formula (XVIII), or a pharmaceutically acceptable salt or solvate thereof:

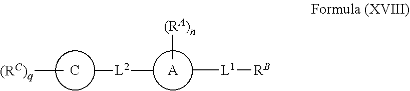

wherein,
ring A is a bicyclic carbocycle or bicyclic heterocycle;
each R^{A} is independently selected from H, D, halogen, —CN, —OH, —OR¹, ═O, —SR¹, —S(═O)R¹, —S(═O)₂R¹, —N(R¹)₂, —NR¹S(═O)(═NR¹)R², —NR¹S(═O)₂R², —S(═O)₂N(R¹)₂, —C(═O)R¹, —OC(═O)R¹, —CO₂R¹, —OCO₂R¹, —C(═O)N(R¹)₂, —OC(═O)N(R¹)₂, —NR¹C(═O)R¹, —P(═O)(R²)₂, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, substituted or unsubstituted C₂-C₇heterocycloalkyl, substituted or unsubstituted aryl, and substituted or unsubstituted monocyclic heteroaryl;
L¹ is —X¹-L³- or -L³-X¹—;
   X¹ is absent, —O—, —S—, —S(═O)—, —S(═O)₂—, —S(═O)(═NR¹)—, —CH₂—, —C(═O)—, —C(═N—OR²)—, —C(═O)O—, —OC(═O)—, —C(═O)C(═O)—, —C(═O)NR¹—, —NR¹C(═O)—, —OC(═O)NR¹—, —NR¹C(═O)O—, —NR¹C(═O)NR¹—, —S(═O)₂NR¹—, —NR¹S(═O)₂—, —NR¹—, —NOR¹—, —P(═O)R²—, —P(═O)(N(R¹)₂)—, —P(═O)(CR¹₃)—, —CR²═CR²—, —N═CR²—, —CR²═N—, —C≡C—, or —NR²—NR²—;
   L³ is absent, substituted or unsubstituted C₁-C₂alkylene, or

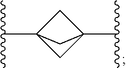 [SMILES: *C12CC(*)(C1)C2]

each R^{B} is independently selected from H, D, halogen, —CN, —OH, —OR¹, ═O, —SR¹, —S(═O)R¹, —S(═O)₂R¹, —N(R¹)₂, —NR¹S(═O)(═NR¹)R², —NR¹S(═O)₂R², —S(═O)₂N(R¹)₂, —C(═O)R¹, —OC(═O)R¹, —CO₂R¹, —OCO₂R¹, —C(═O)N(R¹)₂, —OC(═O)N(R¹)₂, —NR¹C(═O)R¹, —P(═O)(R²)₂, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, substituted or unsubstituted C₂-C₇heterocycloalkyl, substituted or unsubstituted aryl, and substituted or unsubstituted monocyclic heteroaryl;
each R¹ is independently H, D, substituted or unsubstituted C₁-C₆alkyl, substituted or unsubstituted C₁-C₆fluoroalkyl, substituted or unsubstituted C₁-C₆heteroalkyl, substituted or unsubstituted aryl, or substituted or unsubstituted heteroaryl;
each R² is independently H, D, substituted or unsubstituted C₁-C₆alkyl, substituted or unsubstituted C₁-C₆fluoroalkyl, substituted or unsubstituted aryl, substituted or unsubstituted monocyclic heteroaryl, —OH, —OR¹, —N(R¹)₂, —CH₂OR¹, —C(═O)OR¹, —OC(═O)R¹, —C(═O)N(R¹)₂, or —NR¹C(═O)R¹;
L² is —X²-L⁴- or -L⁴-X²—;
   X² is —O—, —S—, —S(═O)—, —S(═O)₂—, —S(═O)(═NR¹)—, —CH₂—, —CH═CH—, —C≡C—, —C(═O)—, —C(═O)O—, —OC(═O)—, —OC(═O)O—, —C(═O)C(═O)—, —C(═O)NR¹—, —NR¹C(═O)—, —OC(═O)NR¹—, —NR¹C(═O)O—, —NR¹C(═O)NR¹—, —NR¹S(═O)₂—, —S(═O)₂NR¹—, —NR¹—, —P(═O)OR¹—, —P(═O)(N(R¹)₂)—, or —P(═O)(CR¹₃)—;
   L⁴ is absent or substituted or unsubstituted C₁-C₂alkylene;
ring C is monocyclic carbocycle, bicyclic carbocycle, monocyclic heterocycle, or bicyclic heterocycle;
each R^{C} is independently selected from H, D, F, —CN, —OH, —OR¹, —SR¹, —S(═O)R¹, —S(═O)₂R¹, —N(R¹)₂, —CH₂—N(R¹)₂, —NHS(═O)₂R¹, —S(═O)₂N(R¹)₂, —C(═O)R¹, —OC(═O)R¹, —CO₂R¹, —OCO₂R¹, —C(═O)N(R¹)₂, —OC(═O)N(R¹)₂, —NR¹C(═O)N(R¹)₂, —NR¹C(═O)R¹, —NR¹C(═O)OR¹, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, and substituted or unsubstituted C₂-C₈heterocycloalkyl;
n is 0, 1, 2, or 3; and
q is 0, 1, 2, 3, 4, 5, or 6.

### Example 11

To develop or screen for new SMN2 splicing modifiers, the molecular basis for SMN2 specific splicing correction mediated by Compound A were investigated. The ability of the splicing modifier Compound A to bind to the RNA duplex formed by the 5′-end of U1 snRNA and the 5′-splice site of SMN2 exon 7 was first verified. Then, the solution structure of the complex Compound A-RNA duplex was solved by means of solution state NMR spectroscopy. By comparing to the solution structures of the free RNA duplex and in complex with the splicing modifier, the mechanism of action of Compound A was determined. Compound A interacts with the RNA duplex at the level of the exon-intron in the major groove and pulls the unpaired adenine into the RNA helix base stack. The splicing modifier transforms the weak 5′-splice site of SMN2 exon 7 into a stronger one. The structure of the complex revealed that Compound A repairs the bulge at position -1 to correct the splicing of SMN2 exon 7.

Spinal Muscular Atrophy (SMA) is an autosomal recessive neuromuscular disease that represents the leading genetic cause of infant mortality. The disorder can be characterized by progressive degeneration of motor neurons from the spinal cord and brain stem, resulting in muscle weakness and atrophy. SMA is caused by the genetic homozygous inactivation of the survival of motor neuron-1 gene (SMN1), the main source of SMN protein that is a ubiquitously expressed and involved in multiple cellular processes. Although a paralog gene SMN2 is found in the human genome, it differs by several silent mutations (including the C6T mutation in exon 7) that mainly triggers the production of a different mRNA isoform lacking exon 7 and encoding for an unstable protein. Reduced amount of functional SMN protein can impair motor neuron functions, however, the exact mechanism remains unclear. As SMN2 still produces small amounts of functional SMN protein (˜20%) but not enough to compensate the loss of SMN1, all SMA patients have at least one copy of the SMN2 gene and the severity of the disease inversely correlates with the SMN2 gene copy number. Recently, splicing modifiers that promote SMN2 E7 inclusion have been discovered. They can increase the production of functional SMN protein and the survival of SMA-model mice. The splicing modifiers can act at the pre-mRNA splicing level with a high specificity for the SMN2 E7 and may favor the early steps of spliceosome assembly by stabilizing a specific enhancer complex at the 5′-SS E7. To deeply understand how the splicing correction is driven at the atomic level and to develop new therapeutic molecules, the molecular mechanisms of the SMN2 splicing correction mediated by Compound A were investigated.

Compound A Binds the RNA Duplex Formed by the U1 snRNA 5′-End and the 5′-Splice Site of SMN2 Exon 7.

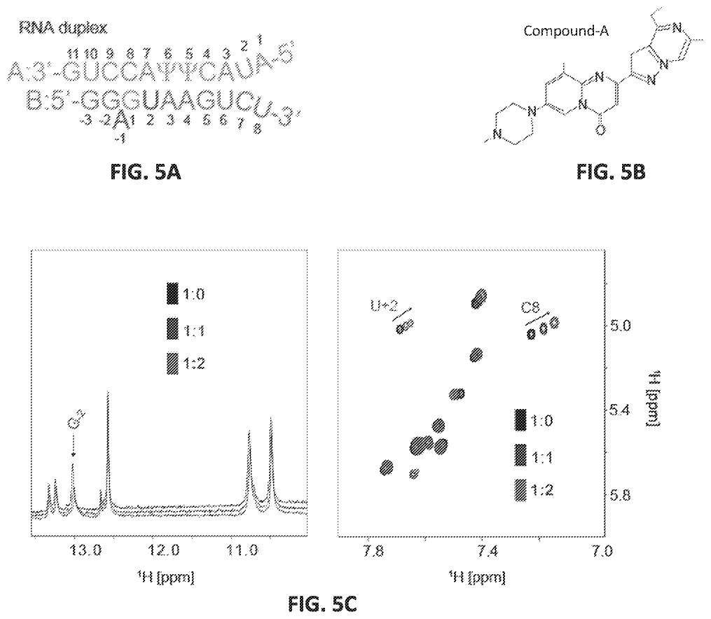

Compound A acts at the pre-mRNA level and should favor a splicing enhancer complex at the 5′-splice site of SMN2 exon 7. To evaluate the binding of Compound A on the RNA duplex upon spliceosome assembly, in vitro binding assays were performed by means of solution state NMR. The RNA duplex was prepared at 250 μM in MES d-8 5 mM pH 5.5, NaCl 50 mM and references spectra (1D ¹H and 2D ¹H—¹H TOCSY) were recorded on the 600 MHz AVIII HD spectrometer equipped with a cryo-probed. Compound A was then dissolved in the same buffer was added to the RNA sample. Upon addition of the splicing modifier, the resonances of the RNA experienced chemical shift changed, in line with a direct interaction between both partners (FIG. 5C). Notably, chemical shift changes were observed for the aromatic protons H5-H6 of U₊₂ and C8 and for the imino proton of G₋₂. Altogether, these protons define the molecule binding pocket on the RNA which locates on the major groove at the exon-intron junction.

### Identification of Intermolecular NOE-Derived Distances Between Compound A and the RNA Duplex

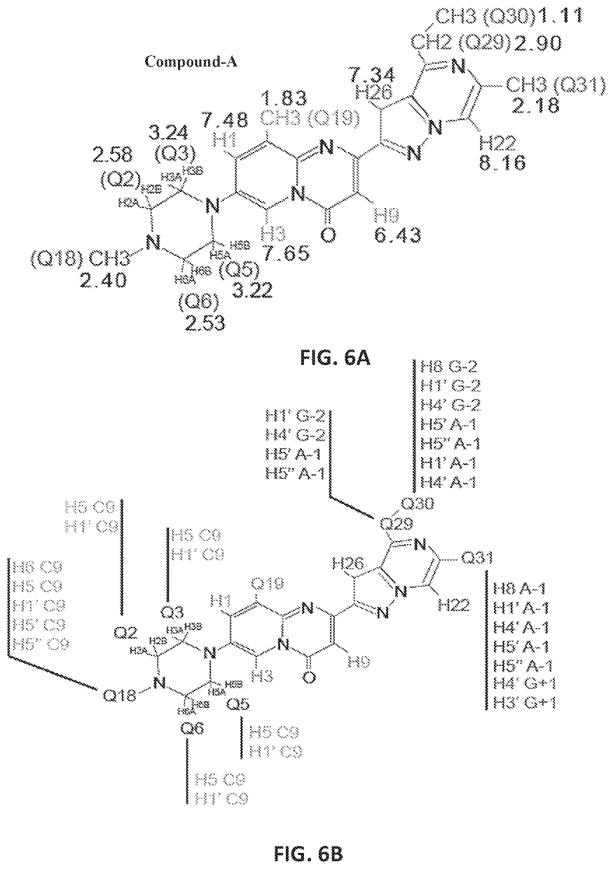

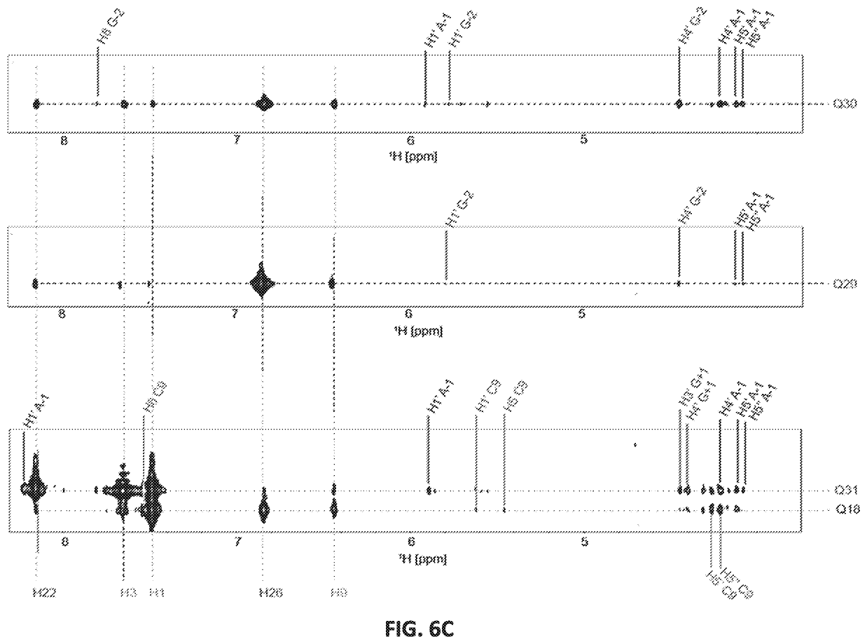

To obtain structural insights into the specific splicing correction induced by Compound A, the solution structure of the RNA duplex bound to Compound A was investigated. As a first step, the proton resonances of the Compound A were assigned (FIG. 6A). Using a chemical shift prediction tool (nmrdb.com), the chemical shifts of Compound A were identified on the homonuclear NMR spectra of the complex. Once the resonances of Compound A assigned, the 2D ¹H—¹H TOCSY and NOESY spectra were analyzed to identify the RNA duplex resonances and the intermolecular NOEs which correspond to correlations between one proton of the splicing modifier and one proton of the RNA duplex. As Compound A contains 4 methyl groups, a large number of intermolecular contacts were identified (30 intermolecular distances) (FIG. 6B). The first cycle is the main provider of intermolecular NOEs and it shows that this part of the molecule interacts with the region G₋₁-G₊₁ of the 5′-splice site. The central aromatic cycle does not provide any intermolecular restraints while the piperazine moiety is in closed proximity of the C9 from the U1 snRNA 5′-end. Experimental data showing the presence of the intermolecular NOEs on the NOESY spectra are illustrated in FIG. 6C. These intermolecular NOEs were then transformed into NOE-derived distances and used to drive the structure calculation of the complex Compound A-RNA duplex.

### Solution Structure of the Compound A-RNA Duplex Complex

The solution structure of the Compound A-RNA duplex complex was solved using 316 intramolecular distances for the RNA duplex, 18 constraints to maintain the base pairing, 146 angular restraints to ensure the ribose puckers and 30 intermolecular NOEs. The structure of the RNA was computed using a semi-automated approach for the RNA part using CYANA NOEASSIGN that analyzed the NMR data based on the chemical shift provided and coupled this interpretation to torsion angle simulated annealing. The program performs seven cycles of NOE assignment, calibration, structure calculation and evaluation of the agreement between the structure and the experimental data. The output from the automatic structure calculation was then combined with manually integrated intermolecular NOE-derived distances to calculate the structure of the complex still in the torsion-angle space. Once low target function was achieved, the structure was refined in by simulated annealing in the Cartesian space using the SANDER module of AMBER12. This structure was then utilized to develop and screen for new SMN2 splicing modifiers.

By solving the solution structure of the Compound A splicing modifier bound to the RNA duplex formed upon recognition of the 5′-splice site of SMN2 exon 7 and U1 snRNP, it as determined found that Compound A stabilizes the unpaired adenine at the exon-intron junction into the RNA helix base stack. The conformational switch of the adenine mimics a strong 5′-splice site and induces the specific splicing correction. The atomic details of the Compound A binding pocket exemplefy the ability to rationally design new splicing modifiers to SMN2 and other targets.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method comprising:
(a) providing a polynucleotide sample comprising a target polynucleotide;
(b) contacting to the target polynucleotide a first binding agent, a second binding agent, or both;
wherein the target polynucleotide and the first binding agent form a first complex,
wherein the second binding agent and the first complex form a second complex; and
(c) obtaining a nuclear magnetic resonance (NMR) spectrum of the first complex, the second complex, or both using a NMR device.

2. (canceled)

3. The method of claim 1, wherein the target polynucleotide is a precursor messenger RNA (pre-mRNA) or a portion thereof.

4. (canceled)

5. The method of claim 1, wherein the target polynucleotide contains a splice site or a portion thereof, wherein the splice site or the portion thereof is a 5′ splice site, a cryptic 5′ splice site, a 3′ splice site, or a cryptic 3′ spice site, or any combinations thereof.

6-14. (canceled)

15. The method of claim 1, wherein the first binding agent comprises a first polynucleotide, a first polypeptide, or a combination thereof.

16. (canceled)

17. The method of claim 15, wherein the first polynucleotide is a small nuclear RNA (snRNA) or a portion thereof.

18-19. (canceled)

20. The method of claim 15, wherein the first polypeptide is a small nuclear ribonucleoprotein (snRNP) or a portion thereof.

21-23. (canceled)

24. The method of claim 1, wherein the first binding agent comprises a small molecule.

25-33. (canceled)

34. The method of claim 1, wherein the first complex comprises a binding pocket, wherein the binding pocket comprises a bulge, or a mutation, or a stem-loop, or any combinations thereof.

35-126. (canceled)

127. A method comprising:
(a) identifying one or more binding pockets formed by a target polynucleotide and a first polynucleotide, wherein the target polynucleotide contains a sequence of a splice site, a branch point (BP), an exonic splicing enhancer (ESE), an exonic splicing silencer (ESS), an intronic splicing enhancer (ISE), an intronic splicing silencer (ISS), or a polypyrimidine tract, or any combinations thereof; and
(b) virtually screening one or more small molecules or fragments thereof against the one or more binding pockets, wherein the virtual screening process identifies a putative small molecule or fragment hits.

128-129. (canceled)

130. The method of claim 127, wherein the method further comprises testing one or more small molecule or fragment hits from the virtual screen using an experimental assay.

131-132. (canceled)

133. The method of claim 127, wherein the target polynucleotide is a pre-mRNA.

134. The method of claim 127, wherein the splice site is a 5′ splice site, a cryptic 5′ splice site, a 3′ splice site, or a cryptic 3′ splice site.

135-142. (canceled)

143. The method of claim 127, wherein the method further comprises identifying a first putative small molecule or and a second putative small molecule.

144. The method of claim 143, wherein the method further comprises determining a first binding kinetics of the first putative small molecule or fragment hit binding to the target polynucleotide, and a second binding kinetics of the second putative small molecule or fragment hit binding to the target polynucleotide.

145-146. (canceled)

147. A method of selecting a binding agent to a target polynucleotide, comprising:
a. contacting to a sample containing the target polynucleotide a binding agent,
wherein the target polynucleotide contains a splice site, a branch point (BP), an exonic splicing enhancer (ESE), an exonic splicing silencer (ESS), an intronic splicing enhancer (ISE), an intronic splicing silencer (ISS), or a polypyrimidine tract, or any combinations thereof,
b. obtaining a structure of the binding agent and the target polynucleotide in a first assay;
c. obtaining a binding kinetics of the binding agent in a second assay; and
d. selecting the binding agent based on the structure and the binding kinetics.

148-150. (canceled)

151. The method of claim 147, wherein the binding agent is a small molecule.

152. The method of claim 147, wherein the sample further comprises a first polynucleotide.

153. (canceled)

154. The method of claim 147, wherein the first polynucleotide is a small nuclear RNA (snRNA) or a portion thereof.

155. (canceled)

156. The method of claim 152, wherein the target and the first polynucleotide form a duplex, wherein the duplex contains a binding pocket comprising a bulge, a mutation, a stem-loop, or any combination thereof.

157-159. (canceled)

160. The method of claim 147, wherein the sample further comprises a ribonucleoprotein.

161-178. (canceled)
